(19) United States
(12) Patent Application Publication
(10) Pub. No.: US 2012/0088662 A1
(43) Pub. Date: Apr. 12, 2012

(54) FUNGICIDAL MIXTURES

(75) Inventors: Jochen Dietz, Karlsruhe (DE); Egon Haden, Ludwigshafen (DE); Jens Renner, Dirmstein (DE); Sarah Ulmschneider, Dirmstein (DE); Alice Glättli, Frankfurt (DE); Marianna Vrettou-Schultes, Mannheim (DE); Silke Stolz, Neidenfels (DE)

(73) Assignee: BASF SE, Ludwigshafen (DE)

(21) Appl. No.: 13/377,965

(22) PCT Filed: Jun. 11, 2010
(86) PCT No.: PCT/EP2010/058257

§ 371 Date: Dec. 13, 2011

(30) Foreign Application Priority Data
Jun. 18, 2009 (EP) 09163122.6
Dec. 15, 2009 (EP) 09179324.0

(51) Int. Cl. C07D 405/06; A01N 43/40; A01N 43/54; A01N 25/26; A01P 21/00; A01P 13/00; A01P 7/04; A01N 43/653; A01P 3/00

(52) U.S. Cl. 504/100; 548/264.4; 514/384; 548/264.2; 514/355; 514/275

(57) ABSTRACT

The present invention relates to fungicidal mixtures comprising, as active components,

1) azolylmethyloxiranes of the general formula I

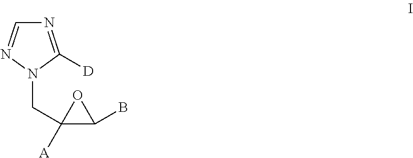

in which the variables have the meanings described in the application, and
2) a fungicidal compound II, where the compounds II of component 2 are selected from among the compounds described in the application,
and to the use of the fungicidal mixtures for controlling phytopathogenic fungi and to the compositions comprising them.

The present invention relates to fungicidal compositions, in particular fungicidal compositions comprising, as active components,

1) azolylmethyloxiranes of the general formula I

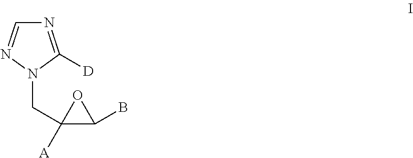

in which the variables have the following meanings:
A is phenyl, 2-fluorophenyl, 2-chlorophenyl, 4-fluorophenyl, 4-chlorophenyl, 4-bromophenyl, 3-chlorophenyl, 2,4-dichlorophenyl, 3,4-dichlorophenyl, 3,5-dichlorophenyl, 4-methylphenyl, 4-tert-butylphenyl;
B is 2-fluorophenyl, 2-chlorophenyl, 2-bromophenyl;
D is —S—R, where
   R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
   R³ is C₁-C₈-alkyl, C₁-C₈-haloalkyl, C₁-C₈-alkoxy, C₁-C₈-haloalkoxy or NA³A⁴; and
   R⁴ is C₁-C₈-alkyl, phenyl-C₁-C₈-alkyl or phenyl, where the phenyl groups are in each case unsubstituted or substituted by one, two or three groups independently of one another selected from the group consisting of halogen and C₁-C₄-alkyl;
   a group DI

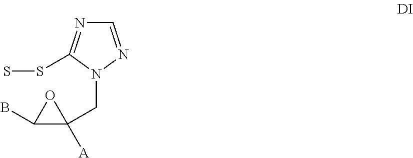

where A and B are as defined above;
a group DII

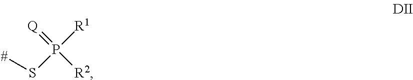

where # denotes the point of attachment to the triazolyl ring and Q, R¹ and
R² are as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl; or
a group SM, where M is as defined below:
M is an alkali metal cation, an equivalent of an alkaline earth metal cation, an equivalent of a copper, zinc, iron or nickel cation or an ammonium cation of the formula (E)

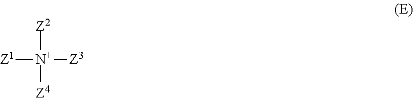

in which
Z¹ and Z² independently are hydrogen or C₁-C₈-alkyl;
Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;
   and agriculturally acceptable salts thereof, and
   2) a compound II,
   where the compound II of component 2 is selected from the following compounds:
   A) strobilurins:
azoxystrobin, dimoxystrobin, coumoxystrobin, coumethoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, methyl 2-(ortho-((2,5-dimethylphenyloxymethylene)phenyl)-3-methoxyacrylate, methyl 3-methoxy-2-(2-(N-(4-methoxyphenyl)-cyclopropanecarboximidoylsulfanylmethyl)phenyl)acrylate, 2-(2-(3-(2,6-dichloro-phenyl)-1-methylallylideneaminooxymethyl)phenyl)-2-methoxyimino-N-methyl-acetamide;
   B) carboxamides:
carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methylthiazole-5-carboxanilide, 2-chloro-N-(1,1,3-trimethylindan-4-yl)nicotinamide, N-(3′,4′,5′-trifluorobiphenyl-2-yl)-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3-dimethylbutyl)phenyl)-1,3-dimethyl-5-fluoro-1H-pyrazole-4-carboxamide (penflufen), N-(2-(1,3,3-trimethylbutyl)phenyl)-1,3-dimethyl-5-fluoro-1H-pyrazole-4-carboxamide;
carboxylic acid morpholides: dimethomorph, flumorph, pyrimorph;
benzamides: flumetover, fluopicolide, fluopyram, zoxamide, N-(3-ethyl-3,5,5-trimethylcyclohexyl)-3-formylamino-2-hydroxybenzamide;
other carboxamides: carpropamid, diclocymet, mandipropamid, oxytetracyclin, silthiofam, N-(6-methoxypyridin-3-yl)cyclopropanecarboxamide;
   C) azoles:
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chlorophenyl)-2-([1,2,4]triazol-1-yl)cycloheptanol;
imidazoles: cyazofamid, imazalil, imazalil sulfate, pefurazoate, prochloraz,
triflumizole;
benzimidazoles: benomyl, carbendazim, fuberidazole, thiabendazole;
others: ethaboxam, etridiazole, hymexazole, 2-(4-chlorophenyl)-N-[4-(3,4-dimethoxyphenyl)isoxazol-5-yl]-2-prop-2-ynyloxyacetamide;
   D) nitrogenous heterocyclyl compounds
pyridines: fluazinam, pyrifenox, 3-[5-(4-chlorophenyl)-2,3-dimethylisoxazolidin-3-yl]pyridine, 3-[5-(4-methylphenyl)-2,3-dimethylisoxazolidin-3-yl]pyridine, 2,3,5,6-tetrachloro-4-methanesulfonylpyridine, 3,4,5-trichloropyridine-2,6-dicarbonitrile, N-(1-(5-bromo-3-chloropyridin-2-yl)ethyl)-2,4-dichloronicotinamide, N-((5-bromo-3-chloropyridin-2-yl)methyl)-2,4-dichloronicotinamide;
pyrimidines: bupirimate, cyprodinil, diflumetorim, fenarimol, ferimzone, mepanipyrim, nitrapyrin, nuarimol, pyrimethanil;
   piperazines: triforine;
pyrroles: fludioxonil, fenpiclonil;
   morpholines: aldimorph, dodemorph, dodemorph acetate, fenpropimorph,
   tridemorph;
   piperidines: fenpropidin;
   dicarboximides: fluoroimide, iprodione, procymidone, vinclozolin;
nonaromatic 5-membered heterocycles: famoxadone, fenamidone, flutianil, octhilinone, probenazole, S-allyl 5-amino-2-isopropyl-3-oxo-4-ortho-tolyl-2,3-dihydropyrazole-1-thiocarboxylate;
   others: acibenzolar-5-methyl, amisulbrom, anilazine, blasticidin-S, captafol, captan, chinomethionat, dazomet, debacarb, diclomezine, difenzoquat, difenzoquatmethylsulfate, fenoxanil, folpet, oxolinic acid, piperalin, proquinazid, pyroquilon, quinoxyfen, triazoxide, tricyclazol, 2-butoxy-6-iodo-3-propylchromen-4-one, 5-chloro-1-(4,6-dimethoxypyrimidin-2-yl)-2-methyl-1H-benzoimidazole, 5-chloro-7-(4-methylpiperidin-1-yl)-6-(2,4,6-trifluorophenyl)-[1,2,4]triazolo[1,5-a]pyrimidine, 5-ethyl-6-octyl-[1,2,4]triazolo[1,5-a]pyrimidin-7-ylamine;
   E) carbamates and dithiocarbamates
thio- and dithiocarbamates: ferbam, mancozeb, maneb, metam, methasulfocarb, metiram, propineb, thiram, zineb, ziram;
carbamates: diethofencarb, benthiavalicarb, iprovalicarb, propamocarb, propamocarb hydrochloride, valiphenal, 4-fluorophenyl N-(1-(1-(4-cyanophenyl)ethanesulfonyl)but-2-yl)carbamate;
   F) other fungicides
guanidines: dodine, dodine free base, guazatine, guazatine acetate, iminoctadine, iminoctadine triacetate, iminoctadine tris(albesilate);
antibiotics: kasugamycin, kasugamycin hydrochloride hydrate, polyoxins, streptomycin, validamycin A;
nitrophenyl derivatives: binapacryl, dicloran, dinobuton, dinocap, nitrothal isopropyl, tecnazene;
organometallic compounds: fentin salts, such as, for example, fentin acetate, fentin chloride, fentin hydroxide;
sulfur-containing heterocyclyl compounds: dithianon, isoprothiolane;
organophosphorus compounds: edifenphos, fosetyl, fosetyl aluminum, iprobenfos, phosphorous acid and its salts, pyrazophos, tolclofos-methyl;
organochlorine compounds: chlorothalonil, dichlofluanid, dichlorophen, flusulfamide, hexachlorobenzene, pencycuron, pentachlorophenol and its salts, phthalide, quintozene, thiophanate methyl, tolylfluanid, N-(4-chloro-2-nitrophenyl)-N-ethyl-4-methylbenzenesulfonamide;
inorganic active compounds: phosphorous acid and its salts, Bordeaux mixture, copper salts, such as, for example, copper acetate, copper hydroxide, copper oxychloride, basic copper sulfate, sulfur;
others: biphenyl, bronopol, cyflufenamid, cymoxanil, diphenylamine, metrafenone, mildiomycin, oxine-copper, prohexadione-calcium, spiroxamine, tolylfluanid, N-(cyclopropylmethoxyimino-(6-difluoromethoxy-2,3-difluorophenyl)methyl)-2-phenylacetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformannidine, N′-(5-difluoromethyl-2-methyl-4-(3-trimethylsilanylpropoxy)phenyl)-N-ethyl-N-methylformamidine, methyl N-(1,2,3,4-tetrahydronaphthalen-1-yl)-2-{1-[2-(5-methyl-3-trifluoromethylpyrazol-1-yl)acetyl]piperidin-4-yl}thiazole-4-carboxamide, methyl(R)-N-(1,2,3,4-tetrahydronaphthalen-1-yl)-2-{1-[2-(5-methyl-3-trifluoromethylpyrazol-1-yl)acetyl]-piperidin-4-yl}thiazole-4-carboxamide, 6-tert-butyl-8-fluoro-2,3-dimethylquinolin-4-yl acetate, 6-tert-butyl-8-fluoro-2,3-dimethylquinolin-4-yl methoxyacetate, N-methyl-2-{1-[2-(5-methyl-3-trifluoromethyl-1H-pyrazol-1-yl)acetyl]piperidin-4-yl}-N-[(1R)-1,2,3,4-tetrahydronaphthalen-1-yl]-4-thiazolecarboxamide;
   G) growth regulators
   abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfid, indole-3-acetic acid, maleic hydrazide, mefluidide, mepiquat (mepiquat chloride), metconazole, naphthalene acetic acid, N-6-benzyladenine, paclobutrazole, prohexadione (prohexadione-calcium), prohydrojasmon, thidiazuron, triapenthenol, tributyl phosphorotrithioate, 2,3,5-triiodobenzoic acid, trinexapac-ethyl and uniconazole;
   H) herbicides
acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid analogs: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-p-tefuryl;
bipyridyls: diquat, paraquat;
carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine,
trifluralin;
diphenyl ethers: acifluorfen, aclonifen, bifenox, diclofop, ethoxyfen, fomesafen, lactofen, oxyfluorfen;
hydroxybenzonitriles: bromoxynil, dichlobenil, ioxynil;
imidazolinones: imazamethabenz, imazamox, imazapic, imazapyr, imazaquin, imazethapyr;
phenoxyacetic acids: clomeprop, 2,4-dichlorophenoxyacetic acid (2,4-D), 2,4-DB, dichlorprop, MCPA, MCPA-thioethyl, MCPB, mecoprop;
pyrazines: chloridazon, flufenpyr-ethyl, fluthiacet, norflurazone, pyridate;
pyridines: aminopyralid, clopyralid, diflufenican, dithiopyr, fluridone, fluoroxypyr, picloram, picolinafen, thiazopyr;
sulfonylureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propylimidazo[1,2-b]pyridazin-3-yl)sulfonyl)-3-(4,6-dimethoxypyrimidin-2-yl)urea;
triazines: ametryn, atrazine, cyanazine, dimethametryn, ethiozine, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other inhibitors of acetolactate synthase: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methylarsenic acid, naptalam, oxadiargyl, oxadiazon, oxaziclomefone, pentoxazone, pinoxaden, pyraclonil, pyraflufen-ethyl, pyrasulfotol, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxyethoxymethyl)-6-trifluoromethylpyridine-3-carbonyl]bicyclo[3.2.1]oct-3-en-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclopropylpyrimidine-4-carboxylate, 6-chloro-3-(2-cyclopropyl-6-methylphenoxy)-pyridazin-4-ol, 4-amino-3-chloro-6-(4-chlorophenyl)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate;
   I) insecticides
organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, betacyfluthrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin,
inhibitors of insect growth: a) chitin synthesis inhibitors: benzoylureas: chlorfluazuron, cyromazine, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]triazinane;
GABA antagonists: endosulfan, ethiprol, fipronil, vaniliprol, pyrafluprol, pyriprol, 5-amino-1-(2,6-dichloro-4-methylphenyl)-4-sulfinamoyl-1H-pyrazole-3-thiocarboxamide;
macrocyclic lactones: abamectin, emamectin, milbemectin, lepimectin, spinosad, spinetoram;
mitochondrial electron transport chain inhibitor (METI) I acaricides: fenazaquin, pyridaben, tebufenpyrad, tolfenpyrad, flufenerim;
METI II and III substances: acequinocyl, fluacyprim, hydramethylnon;
decouplers: chlorfenapyr;
inhibitors of oxidative phosphorylation: cyhexatin, diafenthiuron, fenbutatin oxide, propargite;
insect molting inhibitors: cryomazine;
mixed function oxidase inhibitors: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamid, chlorantraniliprol, cyazypyr (HGW86); cyenopyrafen, flupyrazofos, cyflumetofen, amidoflumet, imicyafos, bistrifluoron, and pyrifluquinazon, [(3S,4R,4aR,6S,6aS,12R,12aS,12bS)-3-cyclopropanecarbonyloxy)-6,12-dihydroxy-4,6a,12b-trimethyl-11-oxo-9-(pyridin-3-yl)-1,2,3,4,4a,5,6,6a,12a,12b-decahydro-11H,12H-benzo[f]pyrano[4,3-b]chromen-4-yl]methyl cyclopropanecarboxylate,
   in a synergistically effective amount.

The invention furthermore also relates to compositions in which component 2) is defined as follows:

Biological products for controlling fungi, plant strengthening products: Ampelomyces quisqualis (for example the product AQ 10® from Intrachem Bio GmbH & Co. KG, Germany), Aspergillus flavus (for example the product AFLAGUARD® from Syngenta, Switzerland), Aureobasidium pullulans (for example the product BOTECTOR® from bio-ferm GmbH, Germany), Bacillus pumilus (for example strain NRRL No. B-30087 in SONATA° and BALLAD° Plus from AgraQuest Inc., USA), Bacillus subtilis (for example strain NRRL No. B-21661 in RHAPSODY®, SERENADE® MAX and SERENADE° ASO from AgraQuest Inc., USA), Bacillus subtilis var. amyloliquefaciens FZB24 (for example the product TAEGRO® from Novozyme Biologicals, Inc., USA), Candida oleophila I-82 (for example the product ASPIRE° from Ecogen Inc., USA), Candida saitoana (for example the product BIOCURE® (as a mixture with Lysozym) and BIOCOAT® from Micro Flo Company, USA (BASF SE) and Arysta), chitosan (for example ARMOUR-ZEN from BotriZen Ltd., New Zealand), Clonostachys rosea f. catenulata, also referred to as Gliocladium catenulatum (for example strain J1446: PRESTOP® from Verdera, Finland), Coniothyrium minitans (for example the product CONTANS® from Prophyta, Germany), Cryphonectria parasitica (for example the product Endothia parasitica from CNICM, France), Cryptococcus albidus (for example the product YIELD PLUS® from Anchor Bio-Technologies, South Africa), Fusarium oxysporum (for example the products BIOFOX® from S.I.A.P.A., Italy, and FUSACLEAN® from Natural Plant Protection, France), Metschnikowia fructicola (for example the product SHEMER® from Agrogreen, Israel), Microdochium dimerum (for example the product ANTIBOT® from Agrauxine, France), Phlebiopsis gigantea (for example the product ROTSOP® from Verdera, Finland), Pseudozyma flocculosa (for example the product SPORODEX® from Plant Products Co. Ltd., Canada), Pythium oligandrum DV74 (for example the product POLYVERSUM° from Remeslo SSRO, Biopreparaty, Czech Republic), Reynoutria sachlinensis (for example the product REGALIA® from Marrone BioInnovations, USA), Talaromyces flavus V117b (for example the product PROTUS® from Prophyta, Germany), Trichoderma asperellum SKT-1 (for example the product ECO-HOPE® from Kumiai Chemical Industry Co., Ltd., Japan), T. atroviride LC52 (for example the product SENTINEL® from Agrimm Technologies Ltd, New Zealand), T. harzianum T-22 (for example the product PLANTSHIELD® from BioWorks Inc., USA), T. harzianum TH 35 (for example the product ROOT PRO® from Mycontrol Ltd., Israel), T. harzianum T-39 (for example the products TRICHODEX® and TRICHODERMA 2000® from Mycontrol Ltd., Israel and Makhteshim Ltd., Israel), T. harzianum and T. viride (for example the product TRICHOPEL from Agrimm Technologies Ltd, New Zealand), T. harzianum ICC012 and T. viride ICC080 (for example the product REMEDIER® WP from Isagro Ricerca, Italy), T. polysporum and T. harzianum (for example the product BINAB® from BINAB Bio-Innovation AB, Sweden), T. stromaticum (for example the product TRICOVAB® from C.E.P.L.A.C., Brazil), T. virens GL-21 (for example the product SOILGARD® from Certis LLC, USA), T. viride (for example the products TRIECO® from Ecosense Labs. (India) Pvt. Ltd., India and BIO-CURE® F. from T. Stanes & Co. Ltd., India), T. viride TV1 (for example the product T. viride TV1 from Agribiotec srl, Italy), Ulocladium oudemansii HRU3 (for example the product BOTRY-ZEN® from Botry-Zen Ltd, New Zealand); where components 1) and 2) are present in a synergistically effective amount.

The invention furthermore relates to the use of the fungicidal mixtures for controlling phytopathogenic fungi and preparations or compositions comprising them. The invention furthermore also relates to seed comprising the fungicidal mixtures. The invention furthermore also relates to methods for controlling phytopathogenic fungi, wherein the fungi or the materials, plants, the soil or seeds to be protected from fungal attack are treated with an effective amount of a fungicidal mixture according to the invention. The invention furthermore also relates to processes for preparing the mixtures according to the invention.

The mixtures comprising at least one compound of the formula I (component 1) and at least one further active compound II (component 2 and optional component 3) are mixtures according to the invention. Mixtures according to the invention are furthermore mixtures comprising at least one compound of the formula I (component I) and three further independently selected different active compounds II (components 2, 3 and 4). Particularly preferably, the mixtures according to the invention are binary mixtures. Likewise preferably, the mixtures according to the invention are ternary mixtures.

In addition to the 3 components mentioned above, the mixtures according to the invention may also comprise at least one further compound II as additional components (for example component 4 or components 4 and 5 etc.), where compound II of the additional components (for example component 4 or components 4 and 5) is selected from the compounds mentioned above of groups A) to I), with the proviso that the components are not identical.

In addition, the present invention relates to synergistic mixtures which, in addition to the 3 components defined above, comprise a further compound II as 4th component, where this component 4 is selected from the compounds defined above of groups A) to I), with the proviso that components 2, 3 and 4 are not identical.

In a preferred embodiment, the invention relates to fungicidal mixtures comprising

1) azolylmethyloxiranes of the general formula I as described above, and
2) a compound II, and
3) a further compound II,
where the compounds II of components 2 and 3 independently of one another are selected from the compounds of groups A to I as described above, with the proviso that component 2 and component 3 are not identical,
in a synergistically effective amount.

The invention relates in particular also to fungicidal mixtures comprising

1) azolylmethyloxiranes of the general formula I as described above, and
2) a compound II, and
3) a further compound II, and
4) a further compound II,
where the compounds II of components 2, 3 and 4 independently of one another are selected from the compounds of groups A to I as described above, with the proviso that components 2, 3 and 4 are not identical,
in a synergistically effective amount.

Azolylmethyloxiranes of component 1, their preparation and their use in crop protection are known from DE19520097 and from the applications WO97/41107, WO97/42178, WO97/43269, WO97/44331, WO97/44332 and WO99/05149. Azolylmethyloxiranes of the general formula I are the compounds I according to the invention. However, some of the compounds of the formula I are novel. Accordingly, the present invention also provides compounds of the formula 1 in which the variables have the following meanings:
A is phenyl, 2-fluorophenyl, 2-chlorophenyl, 4-fluorophenyl, 4-chlorophenyl, 4-bromophenyl, 3-chlorophenyl, 2,4-dichlorophenyl, 3,4-dichlorophenyl, 3,5-dichlorophenyl, 4-methylphenyl, 4-tert-butylphenyl;
B is 2-fluorophenyl, 2-chlorophenyl, 2-bromophenyl;
D is —S—C₂H₅; and agriculturally acceptable salts thereof. These compounds are also referred to as compounds I-A.

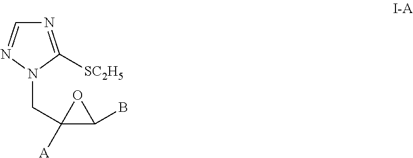

The present invention furthermore provides the use of the compounds I-A for controlling phytopathogenic fungi, and preparations or compositions comprising them. Furthermore, the invention also relates to seed comprising the compounds of the formula I-A according to the invention. Furthermore, the invention also relates to methods for controlling phytopathogenic fungi, wherein the fungi or the materials, plants, the soil or seeds to be protected from fungal attack are treated with an effective amount of a compound of the formula I-A according to the invention. The invention furthermore also relates to processes for preparing the compounds of the formula I-A according to the invention.

Furthermore, the invention relates to further certain compounds of the formula I (=compounds of the formula I according to the invention), to their use for controlling phytopathogenic fungi and to preparations or compositions comprising them. Furthermore, the invention also relates to seed comprising the compounds of the formula I according to the invention. Furthermore, the invention also relates to methods for controlling phytopathogenic fungi, wherein the fungi or the materials, plants, the soil or seeds to be protected from fungal attack are treated with an effective amount of a compound of the formula I according to the invention. The invention furthermore also relates to processes for preparing the compounds of the formula I according to the invention.

The preparation of the compositions comprising certain compounds of the formula I is carried out in a known manner as stated for the preparation of the compositions of the mixtures according to the invention, in the form of compositions comprising, in addition to the active compound or the active compounds, a solvent or a solid carrier. With respect to the customary ingredients of such compositions, reference is made to what was said about the compositions comprising the mixtures according to the invention. The compositions for mixtures of active compounds are suitable as fungicides for controlling harmful fungi. With respect to the use as fungicides (plant diseases to be treated, plants to be treated, type of application, effects), reference is made to what was said about the compositions comprising the mixtures according to the invention.

The active compounds specified above as component 2 (and optional further component 3 and component 4 and as “further active compound” (compounds II)), their preparation, and their action against harmful fungi are known (cf.: http://www.alanwood.net/pesticides/); they are available commercially. The compounds with IUPAC nomenclature, their preparation, and their fungicidal activity are likewise known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

With a view to reducing the application rates and broadening the activity spectrum of the known compounds, it was an object of the present invention to provide mixtures which, at a reduced total amount of active compounds applied, show improved activity against harmful fungi, in particular for certain indications.

Furthermore, in particular at low application rates, the fungicidal activity of the compounds known from the prior art is sometimes unsatisfactory. Accordingly, it was another object of the present invention to provide novel compounds which, preferably, have improved properties such as better fungicidal action and/or better toxicological properties. Owing to the general problem that, in practice, fungicidal compounds frequently eventually lead to resistances, it is furthermore an object of the present invention to provide novel alternative compounds which can be used effectively as fungicides in agriculture. Surprisingly, this object was achieved with the novel compounds described herein.

Accordingly we have found the mixtures defined at the outset. The present invention therefore relates in particular also to fungicidal compositions which comprise at least one compound of the general formula I and at least one further active compound (component 2 and optional component 3), for example one or more, for example 1 or 2, active compounds of groups A to I mentioned above and, if appropriate, one or more agriculturally suitable carriers. Furthermore, the present invention also relates to fungicidal compositions comprising at least one compound of the general formula I and at least three further active compounds (components 2, 3 and 4) of groups A to I mentioned above and, if appropriate, one or more agriculturally suitable carriers. Moreover, it has been found that simultaneous, that is joint or separate, application of compound I and one or more compounds II, or compound I and compounds) II applied in succession, allows better control of harmful fungi than with the individual compounds (synergistic mixtures). As mentioned above, these mixtures are of interest with a view to reducing the application rates, since many show, at a reduced total amount of active compounds applied, an improved activity against harmful fungi, in particular for certain indications. Simultaneous, that is joint or separate, application of compound I and one or more compounds II can increase the fungicidal activity in a superadditive manner.

In the sense of the present application, joint application means that at least one compound I and the at least one further active compound II are present simultaneously at the site of action (i.e. the plant-damaging fungi to be controlled and their habitat, such as infected plants, plant propagation materials, in particular seed, soils, materials or spaces and also the plants, plant propagation materials, in particular seed, soils, materials or spaces to be protected from fungal attack) in an amount sufficient for effective control of fungal growth. This can be achieved by applying the compounds I and at least one further active compound II jointly in a joint active compound preparation or in at least two separate active compound preparations simultaneously, or by applying the active compounds successively to the site of action, the time interval between the individual active compound applications being chosen such that the active compound applied first is, at the time of application of the further active compound(s), present at the site of action in a sufficient amount. The order in which the active compounds are applied is of minor importance.

In a preferred embodiment, the mixtures are binary mixtures, i.e. compositions according to the invention comprising one compound I and one further active compound II (component 2), for example one active compound from groups A) to I). Here, the weight ratio of compound I to further active compound II depends on the properties of the active compounds in question; usually, it is in the range of from 1:100 to 100:1, frequently in the range of from 1:50 to 50:1, preferably in the range of from 1:20 to 20:1, particularly preferably in the range of from 1:10 to 10:1, in particular in the range of from 1:3 to 3:1. It may be preferable for the weight ratio to be in the range of from 1:2 to 2:1.

In a further preferred embodiment, the mixtures are ternary mixtures, i.e. compositions according to the invention comprising one active compound I and one 1st further active compound (component 2) and one 2nd further active compound (component 3), for example two different active compounds from groups A) to I). Here, the weight ratio of compound I to the 1st further active compound (component 2) depends on the properties of the active compounds in question; preferably, it is in the range of from 1:100 to 100:1, preferably in the range of from 1:50 to 50:1 and in particular in the range of from 1:20 to 20:1, specifically from 1:10 to 10:1. It may be preferable for the weight ratio to be in the range of from 1:3 to 3:1, in particular from 1:2 to 2:1. The weight ratio of compound I to the 2nd further active compound (component 3) is preferably in the range of from 1:100 to 100:1, preferably in the range of from 1:50 to 50:1 and in particular in the range of from 1:20 to 20:1, specifically from 1:10 to 10:1. It may be preferable for the weight ratio to be in the range of from 1:3 to 3:1, in particular from 1:2 to 2:1. The weight ratio of 1st further active compound (component 2) to the 2nd further active compound (component 3) is preferably in the range of from 1:100 to 100:1, preferably in the range of from 1:50 to 50:1 and in particular in the range of from 1:20 to 20:1, specifically from 1:10 to 10:1. It may be preferable for the weight ratio to be in the range of from 1:3 to 3:1, in particular from 1:2 to 2:1.

In a further preferred embodiment, the mixtures are quaternary mixtures, i.e. compositions according to the invention comprising one active compound I and one 1st further active compound II (component 2), a 2nd further active compound II (component 3) and a 3rd further active compound II (component 4), where these three active compounds II are different active compounds selected independently from groups A) to I). Here, the weight ratio of compound Ito the 1st further active compound (component 2) depends on the properties of the active compounds in question; preferably, it is in the range of from 1:100 to 100:1, preferably in the range of from 1:50 to 50:1 and in particular in the range of from 1:20 to 20:1, specifically from 1:10 to 10:1. It may be preferable for the weight ratio to be in the range of from 1:3 to 3:1, in particular from 1:2 to 2:1. The weight ratio of compound Ito the 2nd further active compound (component 3) is preferably in the range of from 1:100 to 100:1, preferably in the range of from 1:50 to 50:1, in particular in the range of from 1:20 to 20:1, specifically from 1:10 to 10:1. It may be preferable for the weight ratio to be in the range of from 1:3 to 3:1, in particular from 1:2 to 2:1. The weight ratio of compound Ito the 3rd further active compound (component 4) is preferably in the range of from 1:100 to 100:1, preferably in the range of from 1:50 to 50:1, in particular in the range of from 1:20 to 20:1, specifically from 1:10 to 10:1. It may be preferable for the weight ratio to be in the range of from 1:3 to 3:1, in particular from 1:2 to 2:1. The weight ratio of 1st further active compound (component 2) to 2nd further active compound (component 3) is preferably in the range of from 1:100 to 100:1, preferably in the range of from 1:50 to 50:1, in particular in the range of from 1:20 to 20:1, specifically from 1:10 to 10:1. It may be preferable for the weight ratio to be in the range of from 1:3 to 3:1, in particular from 1:2 to 2:1. The weight ratio of 1st further active compound (component 2) to 3rd further active compound (component 4) is preferably in the range of from 1:100 to 100:1, preferably in the range of from 1:50 to 50:1, in particular in the range of from 1:20 to 20:1, specifically from 1:10 to 10:1. It may be preferable for the weight ratio to be in the range of from 1:3 to 3:1, in particular from 1:2 to 2:1.The weight ratio of 2nd further active compound (component 2) to 3rd further active compound (component 4) is preferably in the range of from 1:100 to 100:1, preferably in the range of from 1:50 to 50:1, in particular in the range of from 1:20 to 20:1, specifically from 1:10 to 10:1. It may be preferable for the weight ratio to be in the range of from 1:3 to 3:1, in particular from 1:2 to 2:1.

The components of the composition according to the invention can be packaged and used individually or as a ready-mix or as a kit of parts.

In one embodiment of the invention, the kits may comprise one or more, and even all, components which may be used for preparing an agrochemical composition according to the invention. For example, these kits may comprise one or more fungicide components and/or an adjuvant component and/or an insecticide component and/or a growth regulator component and/or a herbicide. One or more components may be present combined or preformulated with one another. In the embodiments where more than two components are provided in a kit, the components can be present combined with one another and packaged in a single container, such as a vessel, a bottle, a tin, a bag, a sack or a canister. In other embodiments, two or more components of a kit may be packaged separately, i.e. not preformulated or mixed. Kits may comprise one or more separate containers, such as vessels, bottles, tins, bags, sacks or canisters, each container comprising a separate component of the agrochemical composition. The components of the composition according to the invention can be packaged and used individually or as a ready-mix or as a kit of parts. In both forms, a component may be used separately or together with the other components or as a part of a kit of parts according to the invention for preparing the mixture according to the invention.

The user uses the composition according to the invention usually for use in a predosage device, a knapsack sprayer, a spray tank or a spray plane. Here, the agrochemical composition is diluted with water and/or buffer to the desired application concentration, with further auxiliaries being added, if appropriate, thus giving the ready-to-use spray liquor or the agrochemical composition according to the invention. Usually, from 50 to 500 liters of the ready-to-use spray liquor are applied per hectare of agricultural utilized area, preferably from 100 to 400 liters.

According to one embodiment, the user may himself mix individual components, such as, for example, parts of a kit or a two- or three-component mixture of the composition according to the invention in a spray tank and, if appropriate, add further auxiliaries (tank mix).

In a further embodiment, the user may mix both individual components of the composition according to the invention and partially pre-mixed components, for example components comprising compounds I and/or active compounds from groups A) to I), in a spray tank and, if appropriate, add further auxiliaries (tank mix).

In a further embodiment, the user may use both individual components of the composition according to the invention and partially pre-mixed components, for example components comprising compounds I and/or active compounds from groups A) to I), jointly (for example as a tank mix) or in succession.

The compounds of the formula I can be present in the “thiol” form of the formula Ia or in the “thiono” form of the formula Ib:

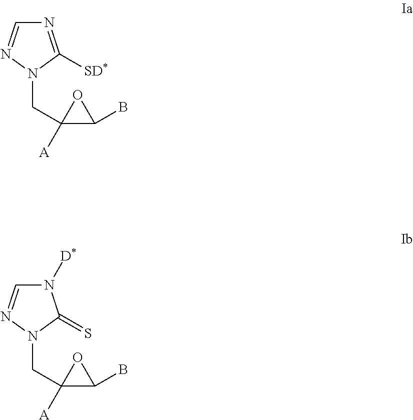

in which D* is:
R, where R has the meaning defined above;
a group DII*

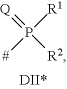

where # is the point of attachment to the sulfur atom in formula Ia or the azolyl ring in formula Ib and Q, R1 and R2 have the meaning defined above; or
a group M, where M has the meaning defined above,
   and in which the remaining substituents have the meaning defined above. This is particularly the case when D*=H.

Here, for the sake of simplicity, in each case only one of the two forms, in general the “thiol” form is shown.

Owing to the basic character of their nitrogen atoms, the compounds I are capable of forming salts or adducts with inorganic or organic acids or with metal ions. This also applies to most of the precursors described herein of compounds I, the salts and adducts of which are also provided by the present invention.

Examples of inorganic acids are hydrohalic acids, such as hydrogen fluoride, hydrogen chloride, hydrogen bromide and hydrogen iodide, carbonic acid, sulfuric acid, phosphoric acid and nitric acid.

Suitable organic acids are, for example, formic acid and alkanoic acids, such as acetic acid, trifluoroacetic acid, trichloroacetic acid and propionic acid, and also glycolic acid, thiocyanic acid, lactic acid, succinic acid, citric acid, benzoic acid and other arylcarboxylic acids, cinnamic acid, oxalic acid, alkylsulfonic acids (sulfonic acids having straight-chain or branched alkyl radicals with 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals with 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid etc.

Suitable metal ions are in particular the ions of the elements of the second main group, in particular calcium and magnesium, of the third and fourth main groups, in particular aluminum, tin and lead, and also of the elements of transition groups one to eight, in particular chromium, manganese, iron, cobalt, nickel, copper, zinc, and others. Particular preference is given to the metal ions of the elements of transition groups of the fourth period. The metals can be present in the various valencies that they can assume.

The compounds I contain centers of chirality, with the trans-configuration being preferred. The compounds I are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions.

Accordingly, the invention provides both mixtures in which compound I is the pure enantiomers or diastereomers and mixtures thereof. This applies to the mixtures according to the invention of the compounds of the formula I. The scope of the present invention includes in particular the (R) and (S) isomer mixtures and the racemates of the compounds I which have centers of chirality. Suitable compounds I also comprise all possible stereoisomers (cis/trans isomers) and mixtures thereof.

The compounds of the formula I according to the invention can be prepared as described in DE19520097, WO97/41107, WO97/42178, WO97/43269, WO97/44331, WO97/44332 or WO99/05149, or by various routes analogously to prior art processes known per se (see, for example, the prior art cited at the outset and Pflanzenschutz-Nachrichten Bayer 57/2004, 2, pages 145-162).

Preparation of the Compound I-A can be Carried Out as Described Below, Starting with compounds I-A.1 in which A and B are defined as described for I-A:

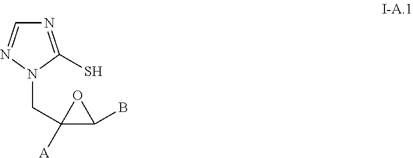

To this end, a compound I-A.1 is reacted with C₂H₅X, where X is a leaving group such as, for example, halogen, such as Cl, Br or I, or trifluoro-C₁-C₆-alkylsulfonate. In particular, a compound I-A.1 is reacted with an ethyl halide (see also WO 96/38440). To prepare a compound I-A.1, the corresponding unsulfurized triazole II-A

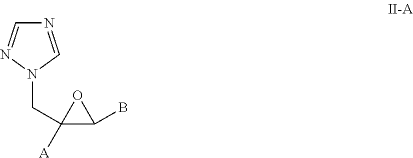

can be reacted with a strong base and sulfur powder. This gives compounds of the formula I-A.1. Suitable bases are all bases known to the person skilled in the art as being suitable for such reactions. Preference is given to using strong alkali metal bases, such as, for example, n-butyllithium, lithium diisopropylamide, sodium hydride, sodium amide or potassium tert-butoxide. It may be preferred to carry out the reaction in the presence of an additive, such as, for example, tetramethylethylenediamide (TMEDA). Suitable solvents are all inert organic solvents customary for such reactions, and preference is given to using ethers, such as tetrahydrofuran, dioxane, diethyl ether and 1,2-dimethoxyethane, or liquid ammonia, or strongly polar solvents, such as dimethyl sulfoxide. Sulfur is preferably used as a powder. Water, if appropriate in the presence of an organic or inorganic acid, such as, for example, acetic acid, dilute sulfuric acid or dilute hydrochloric acid, is used for the hydrolysis. The reaction temperature is preferably between −70° C. and +20° C., in particular between −70° C. and 0° C. The reaction is generally carried out under atmospheric pressure. In general, from 1 to 3 equivalents, preferably from 1 to 2.5 equivalents, of strong base and then an equivalent amount or an excess of sulfur are employed per mole of the compound of the formula II-A. The reaction can be carried out under an atmosphere of protective gas, such as, for example, under nitrogen or argon. Work-up is carried out by procedures known in a general manner to the person skilled in the art. Usually, the reaction mixture is extracted with a suitable organic solvent and the residue is, if appropriate, purified by recrystallization and/or chromatography.

It is also possible to prepare compounds I-A.1 by direct reaction with sulfur, preferably sulfur powder, without using a strong base such as butyllithium.

An alternative preparation of the compounds I-A is the reaction of the corresponding unsubstituted triazoles of the formula II-A with a base and the appropriate disulfide H₅C₂—S—S—C₂H₅. Suitable bases are all bases known to the person skilled in the art as being suitable for such reactions. Preference is given to using strong alkali metal bases, such as, for example, n-butyllithium, lithium diisopropylamide, sodium hydride, sodium amide or potassium tert-butoxide. It may be preferred to carry out the reaction in the presence of an additive, such as, for example, tetramethylethylenediamine (TMEDA). The disulfides are commercially available or can be synthesized by known preparation processes. Suitable solvents are all inert organic solvents customary for such reactions, and preferance is given to using ethers, such as tetrahydrofuran, dioxane, diethyl ether and 1,2-dimethoxyethane, or liquid ammonia, or strongly polar solvents, such as dimethyl sulfoxide. The reaction temperature is preferably between −70° C. and +20° C., in particular between −70° C. and 0° C. The reaction is generally carried out under atmospheric pressure. In general, from 1 to 3 equivalents, preferably from 1 to 2.5 equivalents, of strong base and then an equivalent amount or an excess of disulfide are employed per mole of the compound of the formula II-A. The reaction can be carried out under an atmosphere of protective gas, such as, for example, under nitrogen or argon. Work-up is carried out by procedures known in a general manner to the person skilled in the art. Usually, the reaction mixture is extracted with a suitable organic solvent and the residue is, if appropriate, purified by recrystallization and/or chromatography.

In some of the definitions of the symbols in the formulae given herein, collective terms are used which are generally representative of the following substituents:

halogen: fluorine, chlorine, bromine and iodine;
alkyl and the alkyl moieties of composite groups such as, for example, alkylamino: saturated straight-chain or branched hydrocarbon radicals having 1 to 4, 6, 8 or 12 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: alkyl as mentioned above, where some or all of the hydrogen atoms in these groups are replaced by halogen atoms as mentioned above; in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl and also the alkenyl moieties in composite groups, such as alkenyloxy: unsaturated straight-chain or branched hydrocarbon radicals having 2 to 4, 2 to 6 or 2 to 8 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: alkenyl as defined above, where some or all of the hydrogen atoms in these groups are replaced by halogen atoms as described above under haloalkyl, in particular by fluorine, chlorine or bromine;
alkynyl and the alkynyl moieties in composite groups: straight-chain or branched hydrocarbon groups having 2 to 4, 2 to 6 or 2 to 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butyryl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
haloalkynyl: alkoxy as defined above, where some or all of the hydrogen atoms in these groups are replaced by halogen atoms as described above under haloalkyl, in particular by fluorine, chlorine or bromine;
cycloalkyl and also the cycloalkyl moieties in composite groups: mono- or bicyclic saturated hydrocarbon groups having 3 to 8, in particular 3 to 6, carbon ring members, for example C₃-C₆-cycloalkyl, such as cyclopropyl, cyclobutyl, cyclopentyl, cyclohexyl;
halocycloalkyl: cycloalkyl as defined above, where some or all of the hydrogen atoms in these groups are replaced by halogen atoms as described above under haloalkyl, in particular by fluorine, chlorine or bromine;
alkoxy: an alkyl group as defined above which is attached via an oxygen, preferably having 1 to 8, more preferably 2 to 6, carbon atoms. Examples are: methoxy, ethoxy, npropoxy, 1-methylethoxy, butoxy, 1-methylpropoxy, 2-methylpropoxy or 1,1-dimethylethoxy, and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
haloalkoxy: alkoxy as defined above, where some or all of the hydrogen atoms in these groups are replaced by halogen atoms as described above under haloalkyl, in particular by fluorine, chlorine or bromine. Examples are OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;
alkylene: divalent unbranched chains of CH₂ groups. Preference is given to (C₁-C₆)-alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄CH₂;

In the compounds I (component 1 of the mixtures according to the invention), particular preference is given to the following meanings of the substituents, in each case on their own or in combination.

According to the invention, A is phenyl, 2-fluorophenyl, 2-chlorophenyl, 4-fluorophenyl, 4-chlorophenyl, 4-bromophenyl, 3-chlorophenyl, 2,4-dichlorophenyl, 3,4-dichlorophenyl, 3,5-dichlorophenyl, 4-methylphenyl or 4-tert-butylphenyl.

In a preferred embodiment, A is phenyl, 4-fluorophenyl, 2-chlorophenyl or 4-chlorophenyl.

In a further preferred embodiment, A is phenyl.

In a further preferred embodiment, A is 4-fluorophenyl.

In a further preferred embodiment, A is 2-chlorophenyl.

In a further preferred embodiment, A is 4-chlorophenyl.

According to the invention, B is 2-fluorophenyl, 2-chlorophenyl or 2-bromophenyl.

In a preferred embodiment, B is 2-chlorophenyl.

According to one embodiment of the invention, D is a group SR, where R is hydrogen (compounds I-SH). In a particularly preferred embodiment, the present invention therefore relates to mixtures of the compounds of the formula I in which D is SH.

According to a further embodiment, D is a group SR, where R is C₁-C₄-alkyl, in particular methyl or ethyl, preferably methyl.

According to a further embodiment of the invention, D is a group SR, where R is C(═O)R³ and R³ is NA³A⁴, where A³ and A⁴ independently of one another are hydrogen or C₁-C₈-alkyl.

According to a further embodiment of the invention, D is a group SR, where R is C(═O)R³ and R³ is hydrogen, C₁-C₄-alkyl, C₁-C₄-haloalkyl, C₁-C₄-alkoxy, C₁-C₄-haloalkoxy, phenyl or benzyl. According to a specific aspect thereof, R³ is here hydrogen. According to a further aspect thereof, R³ is C₁-C₄-alkyl, in particular methyl or ethyl, preferably methyl. According to yet a further aspect, R³ is C₁-C₄-haloalkyl, in particular trifluoromethyl. According to yet a further aspect, R³ is C₁-C₄-alkoxy, in particular methoxy or ethoxy.

According to a further embodiment of the invention, D is a group SR, where R is C(═O)R³ and R³ is (C₁-C₄)alkylamino, di(C₁-C₄)alkylamino or phenylamino. According to one aspect thereof, R³ is methylamino, dimethylamino, ethylamino, diethylamino or phenylamino.

According to a further embodiment of the invention, D is a group SR where R is CN.

According to a further embodiment of the invention, D is a group SR, where R is SO₂R⁴ and R⁴ is C₁-C₄-alkyl, phenyl-C₁-C₄-alkyl or phenyl, where the phenyl groups are in each case unsubstituted or substituted by one, two or three groups independently selected from the group consisting of halogen and C₁-C₄-alkyl.

According to a further embodiment of the invention, D is a group SM, where M is an alkali metal cation, an equivalent of an alkaline earth metal cation, an equivalent of a copper, zinc, iron or nickel cation or an ammonium cation of the formula (E)

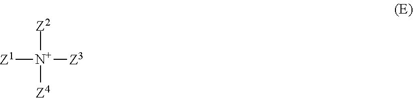

in which
Z¹ and Z² independently are hydrogen or C₁-C₄-alkyl; and
Z³ and Z⁴ independently are hydrogen, C₁-C₄-alkyl, benzyl or phenyl.
According to one embodiment, M is Na, ½ Cu, ⅓ Fe, HN(CH₃)₃, HN(C₂H₅)₃, N(CH₃)₄ or H₂N(C₃H₇)₂, in particular Na, ½ Cu, HN(CH₃)₃ or HN(C₂H₅)₃ especially Na, ½ Cu, HN(CH₃)₃ or HN(C₂H₅)₃.

According to a further embodiment of the invention, D is a group DI (compounds I-dimer) where A and B independently are as defined herein or as defined herein as preferred:

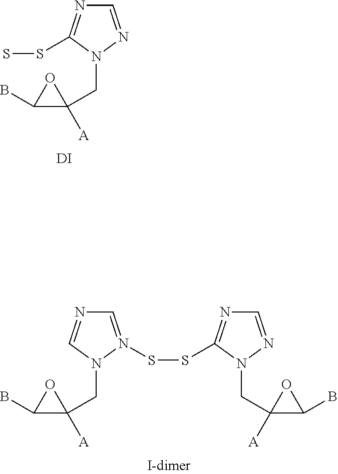

Preferably, both A and both B in the compounds I-dimer have the same meaning.

According to a further embodiment of the invention, D is a group DII, where # denotes the point of attachment to the triazolyl ring and Q, R¹ and R² are as defined herein or as defined as being preferred:

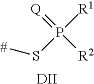

The present invention additionally provides the following compounds I-A-1 to I-A-36 individualized in table I-A:

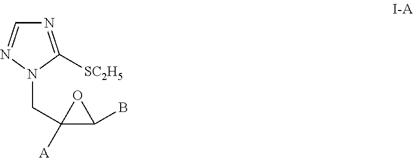

TABLE I-A

| Row | A | B |
|---|---|---|
| 1-A-1 | phenyl | 2-fluorophenyl |
| 1-A-2 | 2-fluorophenyl | 2-fluorophenyl |
| 1-A-3 | 2-chlorophenyl | 2-fluorophenyl |
| 1-A-4 | 4-fluorophenyl | 2-fluorophenyl |
| 1-A-5 | 4-chlorophenyl | 2-fluorophenyl |
| 1-A-6 | 4-bromophenyl | 2-fluorophenyl |
| 1-A-7 | 3-chlorophenyl | 2-fluorophenyl |
| 1-A-8 | 2,4-dichlorophenyl | 2-fluorophenyl |
| 1-A-9 | 3,4-dichlorophenyl | 2-fluorophenyl |
| 1-A-10 | 3,5-dichlorophenyl | 2-fluorophenyl |
| 1-A-11 | 4-methylphenyl | 2-fluorophenyl |
| 1-A-12 | 4-tert-butylphenyl | 2-fluorophenyl |
| 1-A-13 | phenyl | 2-chlorophenyl |
| 1-A-14 | 2-fluorophenyl | 2-chlorophenyl |
| 1-A-15 | 2-chlorophenyl | 2-chlorophenyl |
| 1-A-16 | 4-fluorophenyl | 2-chlorophenyl |
| 1-A-17 | 4-chlorophenyl | 2-chlorophenyl |
| 1-A-18 | 4-bromophenyl | 2-chlorophenyl |
| 1-A-19 | 3-chlorophenyl | 2-chlorophenyl |
| 1-A-20 | 2,4-dichlorophenyl | 2-chlorophenyl |
| 1-A-21 | 3,4-dichlorophenyl | 2-chlorophenyl |
| 1-A-22 | 3,5-dichlorophenyl | 2-chlorophenyl |
| 1-A-23 | 4-methylphenyl | 2-chlorophenyl |
| 1-A-24 | 4-tert-butylphenyl | 2-chlorophenyl |
| 1-A-25 | phenyl | 2-bromophenyl |
| 1-A-26 | 2-fluorophenyl | 2-bromophenyl |
| 1-A-27 | 2-chlorophenyl | 2-bromophenyl |
| 1-A-28 | 4-fluorophenyl | 2-bromophenyl |
| 1-A-29 | 4-chlorophenyl | 2-bromophenyl |
| 1-A-30 | 4-bromophenyl | 2-bromophenyl |
| 1-A-31 | 3-chlorophenyl | 2-bromophenyl |
| 1-A-32 | 2,4-dichlorophenyl | 2-bromophenyl |
| 1-A-33 | 3,4-dichlorophenyl | 2-bromophenyl |
| 1-A-34 | 3,5-dichlorophenyl | 2-bromophenyl |
| 1-A-35 | 4-methylphenyl | 2-bromophenyl |
| 1-A-36 | 4-tert-butylphenyl | 2-bromophenyl |

According to one specific embodiment, the invention relates to compound I-A-16 (A=4-fluorophenyl, B=2-chlorophenyl).

The invention furthermore also relates in particular to mixtures comprising, as component 1, at least one of the compounds of the formula I-A according to the invention as listed in the individual rows of table I-A. According to one specific embodiment, the invention relates to mixtures comprising compound 1-A-16 (A=4-fluorophenyl, B=2-chlorophenyl).

The invention also relates in particular to mixtures of the compounds of the formula I according to the invention as listed in the individual rows of Table C.

TABLE C
Individualized compounds I

| Row | A | B | D |
|---|---|---|---|
| C-1 | phenyl | 2-chlorophenyl | SH |
| C-2 | phenyl | 2-chlorophenyl | SMe |
| C-3 | phenyl | 2-chlorophenyl | SNa |
| C-4 | phenyl | 2-chlorophenyl | S—CO—Me |
| C-5 | phenyl | 2-chlorophenyl | S—CO—OMe |
| C-6 | 4-fluorophenyl | 2-chlorophenyl | SH |
| C-7 | 4-fluorophenyl | 2-chlorophenyl | SMe |
| C-8 | 4-fluorophenyl | 2-chlorophenyl | SNa |
| C-9 | 4-fluorophenyl | 2-chlorophenyl | S—CO—Me |
| C-10 | 4-fluorophenyl | 2-chlorophenyl | S—CO—OMe |
| C-11 | 2-chlorophenyl | 2-chlorophenyl | SH |
| C-12 | 2-chlorophenyl | 2-chlorophenyl | SMe |
| C-13 | 2-chlorophenyl | 2-chlorophenyl | SNa |
| C-14 | 2-chlorophenyl | 2-chlorophenyl | S—CO—Me |
| C-15 | 2-chlorophenyl | 2-chlorophenyl | S—CO—OMe |
| C-16 | 4-chlorophenyl | 2-chlorophenyl | SH |
| C-17 | 4-chlorophenyl | 2-chlorophenyl | SMe |
| C-18 | 4-chlorophenyl | 2-chlorophenyl | SNa |
| C-19 | 4-chlorophenyl | 2-chlorophenyl | S—CO—Me |
| C-20 | 4-chlorophenyl | 2-chlorophenyl | S—CO—OMe |

Particular preference is given to mixtures of the compounds of the formula I (component 1) in which the variables have the following meanings:

A is phenyl, 4-fluorophenyl, 2-chlorophenyl or 4-chlorophenyl.
B is 2-chlorophenyl or 2-fluorophenyl.

Particular preference is furthermore given to mixtures of the compounds of the formula I (component 1) in which the variables have the following meanings:

A is phenyl, 4-fluorophenyl, 2-chlorophenyl or 4-chlorophenyl.
B is 2-chlorophenyl.

Particular preference is given to mixtures of the compounds of the formula I (component 1) in which the variables have the following meanings:

D is —S—R, where
R is hydrogen, C₁-C₈-alkyl, C(═O)R³, SO₂R⁴ or CN; where
R³ is C₁-C₈-alkyl or C₁-C₈-alkoxy,
R⁴ is C₁-C₈-alkyl.

Particular preference is also given to mixtures of the compounds of the formula I (component 1) in which D is —S—C₂H₅ (compounds I-A).

Preference is likewise given to mixtures of the compounds of the formula I (component 1) in which the variables have the following meanings:

D is —S-M, where
where M is as defined below:
   M is an alkali metal cation, an equivalent of an alkaline earth metal cation, an equivalent of a copper, zinc, iron or nickel cation or an ammonium cation of the formula (E)

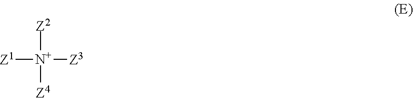

in which
   Z¹ and Z² independently are hydrogen or C₁-C₈-alkyl;
   Z³ and Z⁴ independently are hydrogen, C₁-C₈-alkyl, benzyl or phenyl;
   where the phenyl groups are in each case unsubstituted or substituted by one, two or three groups independently selected from the group consisting of halogen and C₁-C₄-alkyl.

Particular preference is given to mixtures of the compounds of the formula I (component 1) in which the variables have the following meanings:
A is phenyl, 4-fluorophenyl or 4-phenyl, 4-fluorophenyl, 2-chlorophenyl or 4-chlorophenyl,
B is 2-chlorophenyl.
D is —S—R, where
R is hydrogen, C₁-C₈-alkyl, C(═O)R³, SO₂R⁴ or CN; where
   R³ is C₁-C₈-alkyl or C₁-C₈-alkoxy,
   R⁴ is C₁-C₈-alkyl.
      Preference is also give to mixtures of the compounds of the formula I (or I-A) (component 1) in which the variables have the following meanings:
A is phenyl, 4-fluorophenyl or 4-phenyl, 4-fluorophenyl, 2-chlorophenyl or 4-chlorophenyl,
B is 2-chlorophenyl.
D is —S—C₂H₅.

Particular preference is given to mixtures of the compounds of the formula I (component 1) in which the variables have the following meanings:
A is phenyl, 4-fluorophenyl, 2-chlorophenyl or 4-chlorophenyl.
B is 2-chlorophenyl.
D is —S—R, where
R is hydrogen, methyl, C(═O)R³, SO₂R⁴ or CN; where
   R³ is methyl or methoxy,
   R⁴ is methyl.

Particular preference is given to mixtures of the compounds of the formula I (component 1) in which the variables have the following meanings:
A is phenyl, 4-fluorophenyl, 2-chlorophenyl or 4-chlorophenyl.
B is 2-chlorophenyl.
D is —S—R, where
R is hydrogen, methyl, C(═O)R³ or CN; where
   R³ is methyl or methoxy,
   R⁴ is methyl.

Preference is also given to mixtures of the compounds of the formula I (or I-A) (component 1) in which the variables have the following meanings:

A is phenyl, 4-fluorophenyl, 2-chlorophenyl or 4-chlorophenyl.
B is 2-chlorophenyl.

### D is —S—C₂H₅.

The most preferred compounds of the formula I are the compounds 1-1 to 1-18 below, particular preference being given, in each case, to pairs of enantiomers or enantiomers having a trans-configuration of ring A and ring B:
I-1 trans-2-[3-(2-chlorophenyl)-2-(4-fluorophenyl)oxiranylmethyl]-2,4-dihydro-[1,2,4]triazole-3-thione
I-2 trans-1-[3-(2-chlorophenyl)-2-(4-fluorophenyl)oxiranylmethyl]-5-methylsulfanyl-1H-[1,2,4]triazole
I-3 trans-S-{2-[3-(2-chlorophenyl)-2-(4-fluorophenyl)oxiranylmethyl]-2H-[1,2,4]triazol-3-yl}thioacetate
I-4 sodium trans-2-[3-(2-chlorophenyl)-2-(4-fluorophenyl)oxiranylmethyl]-2H-[1,2,4]triazole-3-thiolate
I-5 {2-[3-(2-chlorophenyl)-2-(4-fluorophenyl)oxiranylmethyl]-2H-[1,2,4]triazol-3-yl}methyl thiocarbonate
I-6 trans-1-[3-(2-chlorophenyl)-2-(4-fluorophenyl)oxiranylmethyl]-5-thiocyanato-1H-[1,2,4]triazole
I-7 trans-1-[2,3-bis-(2-chlorophenyl)oxiranylmethyl]-5-methylsulfanyl-1H-[1,2,4]triazole
I-8 trans-2-[2-(4-fluorophenyl)-3-(2-fluorophenyl)oxiranylmethyl]-2H-[1,2,4]triazole-3-thiole
I-9 trans-1-[2-(4-fluorophenyl)-3-(2-fluorophenyl)oxiranylmethyl]-5-methylsulfanyl-1H-[1,2,4]triazole
I-10 sodium 2-[trans-2,3-bis-(2-chlorophenyl)oxiranylmethyl]-2H-[1,2,4]triazole-3-thiolate
I-11 trans-1-[-3-(2-chlorophenyl)-2-phenyloxiranylmethyl]-5-methylsulfanyl-1H-[1,2,4]triazole
I-12 2-[trans-3-(2-chlorophenyl)-2-phenyloxiranylmethyl]-2,4-dihydro-[1,2,4]triazole-3-thione
I-13 1-[trans-3-(2-chlorophenyl)-2-(2-fluorophenyl)oxiranylmethyl]-5-methylsulfanyl-1H-[1,2,4]triazole
I-14 2-[trans-3-(2-chlorophenyl)-2-(2-fluorophenyl)oxiranylmethyl]-2,4-dihydro-[1,2,4]triazole-3-thione
I-15 bis-{2-[trans-3-(2-chlorophenyl)-2-phenyloxiranylmethyl]-2H-[1,2,4]triazol-3-yl}disulfane
I-16 S-{2-[trans-2,3-bis-(2-chlorophenyl)oxiranylmethyl]-2H-[1,2,4]triazol-3-yl}thioacetate
I-17 S-{2-[trans-3-(2-fluorophenyl)-2-(4-fluorophenyl)oxiranylmethyl]-2H-[1,2,4]triazol-3-yl}thioacetate
I-18 trans-1-[3-(2-chlorophenyl)-2-(4-fluorophenyl)oxiranylmethyl]-5-ethylsulfanyl-1H-[1,2,4]triazole

Alternative notations of the trans-enantiomer pairs:
I-1 2-rel-[(2S,3R)-3-(2-chlorophenyl)-2-(4-fluorophenyl)oxiranylmethyl]-2,4-dihydro-[1,2,4]triazole-3-thione
I-2 1-rel-[(2S,3R)-3-(2-chlorophenyl)-2-(4-fluorophenyl)oxiranylmethyl]-5-methylsulfanyl-1H-[1,2,4]triazole
I-3 S-{2-rel-[(2S,3R)-3-(2-chlorophenyl)-2-(4-fluorophenyl)oxiranylmethyl]-2H-[1,2,4]triazol-3-yl}thioacetate
I-4 sodium 2-rel-[(2S,3R)-3-(2-chlorophenyl)-2-(4-fluorophenyl)oxiranylmethyl]-2H-[1,2,4]triazole-3-thiolate
I-5 {2-[3-(2-chlorophenyl)-2-(4-fluorophenyl)oxiranylmethyl]-2H-[1,2,4]triazol-3-yl}methyl thiocarbonate
I-6 1-rel-[(2S,3R)-3-(2-chlorophenyl)-2-(4-fluorophenyl)oxiranylmethyl]-5-thiocyanato-1H-[1,2,4]triazole
I-7 1-rel-[(2S,3R)-2,3-bis-(2-chlorophenyl)oxiranylmethyl]-5-methylsulfanyl-1H-[1,2,4]triazole
I-8 2-rel-[(2S,3R)-2-(4-fluorophenyl)-3-(2-fluorophenyl)oxiranylmethyl]-2H-[1,2,4]triazole-3-thiole
I-9 1-rel-[(2S,3R)-2-(4-fluorophenyl)-3-(2-fluorophenyl)oxiranylmethyl]-5-methylsulfanyl-1H-[1,2,4]triazole
I-10 sodium 2-rel-[(2S,3R)-2,3-bis-(2-chlorophenyl)oxiranylmethyl]-2H-[1,2,4]triazole-3-thiolate
I-11 1-rel-[(2S,3R)-3-(2-chlorophenyl)-2-phenyloxiranylmethyl]-5-methylsulfanyl-1H-[1,2,4]triazole
I-12 2-[rel-(2S,3R)-3-(2-chlorophenyl)-2-phenyloxiranylmethyl]-2,4-dihydro-[1,2,4]triazole-3-thione
I-13 1-[rel-(2S,3R)-3-(2-chlorophenyl)-2-(2-fluorophenyl)oxiranylmethyl]-5-methylsulfanyl-1H-[1,2,4]triazole
I-14 2-[rel-(2S,3R)-3-(2-chlorophenyl)-2-(2-fluorophenyl)oxiranylmethyl]-2,4-dihydro-[1,2,4]triazole-3-thione
I-15 bis-{2-[rel-(2S,3R)-3-(2-chlorophenyl)-2-phenyloxiranylmethyl]-2H-[1,2,4]triazol-3-yl}disulfan
I-16 S-{2-[rel-(2S,3R)-2,3-bis-(2-chlorophenyl)oxiranylmethyl]-2H-[1,2,4]triazol-3-yl}thioacetate
I-17 S-{2-[rel-(2S,3R)-3-(2-fluorophenyl)-2-(4-fluorophenyl)oxiranylmethyl]-2H-[1,2,4]triazol-3-yl}thioacetate
I-18 1-rel-[(2S,3R)-3-(2-chlorophenyl)-2-(4-fluorophenyl)oxiranylmethyl]-5-ethylsulfanyl-1H-[1,2,4]triazole

| Example | | | |
|---|---|---|---|
| No. | A | B | D |
| I-1 | 4-fluorophenyl | 2-chlorophenyl | SH |
| I-2 | 4-fluorophenyl | 2-chlorophenyl | S—Me |
| I-3 | 4-fluorophenyl | 2-chlorophenyl | S—CO—Me |
| I-4 | 4-fluorophenyl | 2-chlorophenyl | S—Na |
| I-5 | 4-fluorophenyl | 2-chlorophenyl | S—CO—OMe |
| I-6 | 4-fluorophenyl | 2-chlorophenyl | S—CN |
| I-7 | 2-chlorophenyl | 2-chlorophenyl | S—Me |
| I-8 | 4-fluorophenyl | 2-fluorophenyl | SH |
| I-9 | 4-fluorophenyl | 2-fluorophenyl | S—Me |
| I-10 | 2-chlorophenyl | 2-chlorophenyl | S—Na |
| I-11 | phenyl | 2-chlorophenyl | S—Me |
| I-12 | phenyl | 2-chlorophenyl | SH |
| I-13 | 2-fluorophenyl | 2-chlorophenyl | S—Me |
| I-14 | 2-fluorophenyl | 2-chlorophenyl | SH |
| I-15 | phenyl | 2-chlorophenyl | dimer |
| I-16 | 2-chlorophenyl | 2-chlorophenyl | S—CO—Me |
| I-17 | 4-fluorophenyl | 2-fluorophenyl | S—CO—Me |
| I-18 | 4-fluorophenyl | 2-chlorophenyl | SC₂H₅ |

In a preferred embodiment, the invention relates to a compound of the formula I in which A is 4-fluorophenyl, B is 2-chlorophenyl and D is 5-CN. In particular, the invention relates to the compound I-6 trans-1-[3-(2-chlorophenyl)-2-(4-fluorophenyl)oxiranylmethyl]-5-thiocyanato-1H-[1,2,4]triazole, and also to its use for controlling phytopathogenic fungi and to the preparations and compositions comprising it. Furthermore, the invention relates to seed comprising the compound I-6. Furthermore, the invention relates to methods for controlling phytopathogenic fungi wherein the fungi or the materials, plants, the soil or seed to be protected from fungal attack are treated with an effective amount of the compound I-6. The invention furthermore also relates to processes for preparing the compound I-6.

In a further preferred embodiment, the invention relates to a compound I-A (i.e. a compound I in which D is SC₂H₅), in particular to the compound I-18, and to its use for controlling phytopathogenic fungi, and to preparations or compositions comprising the compound. Furthermore, the invention also relates to seed comprising the compound I-A, in particular I-18. Furthermore, the invention also relates to methods for controlling phytopathogenic fungi wherein the fungi or the materials, plants, the soil or seed to be protected from fungal attack are treated with an effective amount of a compound I-A, in particular I-18.

Independently of one another, components 2 and 3 are preferably selected as illustrated in the compositions below. Components 4 and further components in quaternary and higher mixtures, too, are preferably independently of one another selected as illustrated in the compositions below:

Preference is given to compositions of a compound I (component 1) with at least one active compound from group A) (component 2 and/or 3 and/or component 4) of the strobilurins and in particular selected from the group consisting of azoxystrobin, dimoxystrobin, fluoxastrobin, kresoxim-methyl, orysastrobin, picoxystrobin, pyraclostrobin and trifloxystrobin.

Preference is also given to compositions of a compound I (component 1) with at least one active compound selected from group B) (component 2 and/or 3 and/or component 4) of the carboxamides and in particular selected from the group consisting of bixafen, boscalid, isopyrazam, fluopyram, penflufen, penthiopyrad, sedaxane, fenhexamid, metalaxyl, mefenoxam, ofurace, dimethomorph, flumorph, fluopicolide (picobenzamid), zoxamide, carpropamid, mandipropamid and N-(3′,4′,5′-trifluorobiphenyl-2-yl)-3-difluoromethyl-1-methyl-1H-pyrazole-4-carboxamide.

Preference is also given to compositions of a compound I (component I) with at least one active compound selected from group C) (component 2 and/or 3 and/or component 4) of the azoles and in particular selected from the group consisting of cyproconazole, difenoconazole, epoxiconazole, fluquinconazole, flusilazole, flutriafol, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to compositions of a compound I (component I) with at least one active compound selected from group D) (component 2 and/or 3 and/or component 4) of the nitrogenous heterocyclyl compounds and in particular selected from the group consisting of fluazinam, cyprodinil, fenarimol, mepanipyrim, pyrimethanil, triforin, fludioxonil, fodemorph, fenpropimorph, tridemorph, fenpropidin, iprodione, vinclozolin, famoxadone, fenamidone, probenazole, proquinazid, acibenzolar-5-methyl, captafol, folpet, fenoxanil, quinoxyfen and 5-ethyl-6-octyl-[1,2,4]triazolo[1,5-a]pyrimidin-7-ylamine.

Preference is also given to compositions of a compound I (component I) with at least one active compound selected from group E) (component 2 and/or 3 and/or component 4) of the carbamates and in particular selected from the group consisting of mancozeb, metiram, propineb, thiram, iprovalicarb, benthiavalicarb and propamocarb.

Preference is also given to compositions of a compound I (component 1) with at least one active compound selected from the fungicides of group F) (component 2 and/or 3 and/or component 4) and in particular selected from the group consisting of dithianon, fentin salts, such as fentin acetate, fosetyl, fosetyl-aluminum, H₃PO₃ and salts thereof, chlorothalonil, dichlofluanid, thiophanatemethyl, copper acetate, copper hydroxide, copper oxychloride, copper sulfate, sulfur, cymoxanil, metrafenone, spiroxamine and N-methyl-2-{1-[(5-methyl-3-trifluoromethyl-1H-pyrazol-1-yl)acetyl]piperidin-4-yl}-M(1R)-1,2,3,4-tetrahydronaphthalen-1-yl]-4-thiazolecarboxamide.

According to one embodiment, the compositions according to the invention comprise a compound I (component 1) and a component 2, component 2 being an insecticide selected from group I). According to a preferred embodiment, the compositions are binary mixtures comprising, as active compounds, one component 1) and one component 2) selected from group I).

According to one aspect, the insecticide of component 2) is selected from the group of the organo(thio)phosphates, in particular from the group consisting of acephate, chlorpyrifos, diazinon, dichlorvos, dimethoate, fenitrothion, methamidophos, methidathion, methyl-parathion, monocrotophos, phorate, profenofos and terbufos.

According to a further aspect, the insecticide of component 2) is selected from the group of the carbamates, in particular selected from the group consisting of aldicarb, carbaryl, carbofuran, carbosulfan, methomyl and thiodicarb.

According to yet a further aspect, the insecticide of component 2) is selected from the group of the pyrethroids, in particular selected from the group consisting of: bifenthrin, cyfluthrin, cypermethrin, alpha-cypermethrin, zeta-cypermethrin, deltamethrin, esfenvalerate, lambda-cyhalothrin and tefluthrin.

According to yet a further aspect, the insecticide of component 2) is selected from the group of inhibitors of insect growth, in particular selected from the group consisting of lufenuron and spirotetramat.

According to yet a further aspect, the insecticide of component 2) is selected from the group of the nicotine receptor agonists/antagonists, in particular selected from the group consisting of: clothianidin, imidacloprid, thiamethoxam and thiacloprid.

According to yet a further aspect, the insecticide of component 2) is selected from the group of the GABA antagonists, in particular selected from the group consisting of: endosulfan and fipronil.

According to yet a further aspect, the insecticide of component 2) is selected from the group of the macrocyclic lactones, in particular selected from the group consisting of abamectin, emamectin, spinosad and spinetoram.

According to yet a further aspect, the insecticide of component 2) is hydramethylnon.

According to yet a further aspect, the insecticide of component 2) is fenbutatin oxide.

According to yet a further aspect, the insecticide of component 2) is selected from the group consisting of chlorfenapyr, cyazypyr (HGW86), cyflumetofen, flonicamid, flubendiamide, indoxacarb and metaflumizone.

According to a further embodiment, the compositions are ternary mixtures comprising, in addition to the components mentioned, a component 3) selected from the active compounds II of group I) mentioned above.

According to a further embodiment, the compositions are ternary mixtures comprising, in addition to the two components mentioned, a component 3) selected from the active compounds II of groups A) to G).

The active compounds II of group I) and their pesticidal action and processes for their preparation are known (see also http://www.hclrss.demon.co.uk/index.html). Commercially available active compounds can be found, for example, in The Pesticide Manual, 14th Edition, British Crop Protection Council (2006) and other publications. Compound BB) of group I)

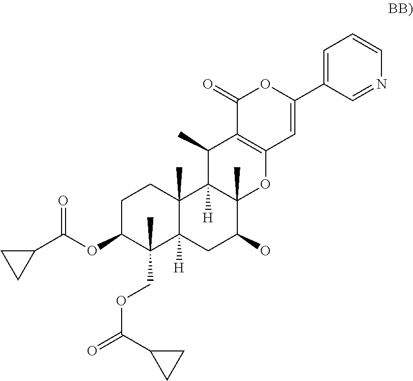

having the IUPAC name [(3S,4R,4aR,6S,6aS,12R,12aS,12bS)-3-(cyclopropanecarbonyloxy)-6,12-dihydroxy-4,6a,12b-trimethyl-[1-oxo-9-(pyridin-3-yl)-1,2,3,4,4a,5,6,6a,12a,12b-decahydro-11H,12H-benzo[f]pyrano[4,3-b]chromen-4-yl]methyl cyclopropanecarboxylate and its pesticidal action are disclosed in WO2006/129714 and WO2009/081851.

In a preferred embodiment, the component 2 is a fungicide selected from groups A to F.

If a component 3 is present, it is in a further preferred embodiment an independently selected fungicide selected from groups A to F. In a further preferred embodiment, components 2 and 3 are two fungicides selected from groups A to F.

If a component 4 is present, it is in a further preferred embodiment an independently selected fungicide selected from groups A to F. In a further preferred embodiment, components 2, 3 and 4 are three fungicides independently selected from groups A to F.

Accordingly, the present invention furthermore relates to compositions of a compound I (component 1) with a further active compound (component 2), the latter being selected from rows A-1 to A-359 in the column “component 2” of Table A.

A further embodiment of the invention relates to the compositions A-1 to A-359 listed in Table A, where a row of Table A corresponds in each case to an agrochemical composition comprising a compound of the formula I individualized in the present description (component 1) and the respective further active compound from the groups A) to I) (component 2) stated in the row in question. In the compositions described, the active compounds are in each case preferably present in synergistically effective amounts.

TABLE A
Active compound composition, comprising an individualized compound
I and a further active compound from the groups A) to I)

| Row | Component 1 | Component 2 |
|---|---|---|
| A-1 | an individualized compound I | azoxystrobin |
| A-2 | an individualized compound I | dimoxystrobin |
| A-3 | an individualized compound I | enestroburin |
| A-4 | an individualized compound I | fluoxastrobin |
| A-5 | an individualized compound I | kresoxim-methyl |
| A-6 | an individualized compound I | metominostrobin |
| A-7 | an individualized compound I | orysastrobin |
| A-8 | an individualized compound I | picoxystrobin |
| A-9 | an individualized compound I | pyraclostrobin |
| A-10 | an individualized compound I | pyribencarb |
| A-11 | an individualized compound I | trifloxystrobin |
| A-12 | an individualized compound I | 2-(2-(6-(3-chloro-2-methylphenoxy)- |
| | | 5-fluoropyrimidin-4-yloxy)phenyl)- |
| | | 2-methoxyimino-N-methylacetamide |
| A-13 | an individualized compound I | methyl 2-(o-((2,5-dimethylphenyloxy- |
| | | methylene)phenyl)-3-methoxyacrylate |
| A-14 | an individualized compound I | methyl 3-methoxy-2-(2-(N-(4-methoxy- |
| | | phenyl)cyclopropanecarboximidoylsulfanyl- |
| | | methyl)phenyl)acrylate |
| A-15 | an individualized compound I | 2-(2-(3-(2,6-dichlorophenyl)-1- |
| | | methylallylideneaminooxymethyl)phenyl)- |
| | | 2-methoxyimino-N-methylacetamide |
| A-16 | an individualized compound I | benalaxyl |
| A-17 | an individualized compound I | benalaxyl-M |
| A-18 | an individualized compound I | benodanil |
| A-19 | an individualized compound I | bixafen |
| A-20 | an individualized compound I | boscalid |
| A-21 | an individualized compound I | carboxin |
| A-22 | an individualized compound I | fenfuram |
| A-23 | an individualized compound I | fenhexamid |
| A-24 | an individualized compound I | flutolanil |
| A-25 | an individualized compound I | furametpyr |
| A-26 | an individualized compound I | isopyrazam |
| A-27 | an individualized compound I | isotianil |
| A-28 | an individualized compound I | kiralaxyl |
| A-29 | an individualized compound I | mepronil |
| A-30 | an individualized compound I | metalaxyl |
| A-31 | an individualized compound I | metalaxyl-M |
| A-32 | an individualized compound I | ofurace |
| A-33 | an individualized compound I | oxadixyl |
| A-34 | an individualized compound I | oxycarboxin |
| A-35 | an individualized compound I | penflufen |
| A-36 | an individualized compound I | penthiopyrad |
| A-37 | an individualized compound I | sedaxane |
| A-38 | an individualized compound I | tecloftalam |
| A-39 | an individualized compound I | thifluzamide |
| A-40 | an individualized compound I | tiadinil |
| A-41 | an individualized compound I | 2-amino-4-methylthiazole-5-carboxanilide |
| A-42 | an individualized compound I | 2-chloro-N-(1,1,3-trimethylindan-4-yl)- |
| | | nicotinamide |
| A-43 | an individualized compound I | N-(3′,4′,5′-trifluorobiphenyl-2-yl)-3- |
| | | difluoromethyl-1-methyl-1H-pyrazole-4- |
| | | carboxamide |
| A-44 | an individualized compound I | N-(4′-trifluoromethylthiobiphenyl-2-yl)-3- |
| | | difluoromethyl-1-methyl-1H-pyrazole-4- |
| | | carboxamide |
| A-45 | an individualized compound I | N-(2-(1,3-dimethylbutyl)phenyl)-1,3- |
| | | dimethyl-5-fluoro-1H-pyrazole-4- |
| | | carboxamide |
| A-46 | an individualized compound I | N-(2-(1,3,3-trimethylbutyl)phenyl)-1,3- |
| | | dimethyl-5-fluoro-1H-pyrazole-4- |
| | | carboxamide |
| A-47 | an individualized compound I | dimethomorph |
| A-48 | an individualized compound I | flumorph |
| A-49 | an individualized compound I | pyrimorph |
| A-50 | an individualized compound I | flumetover |
| A-51 | an individualized compound I | fluopicolide |
| A-52 | an individualized compound I | fluopyram |
| A-53 | an individualized compound I | zoxamide |
| A-54 | an individualized compound I | N-(3-ethyl-3,5,5-trimethylcyclohexyl)- |
| | | 3-formylamino-2-hydroxybenzamide |
| A-55 | an individualized compound I | carpropamid |
| A-56 | an individualized compound I | diclocymet |
| A-57 | an individualized compound I | mandipropamid |
| A-58 | an individualized compound I | oxytetracyclin |
| A-59 | an individualized compound I | silthiofam |
| A-60 | an individualized compound I | N-(6-methoxypyridin-3-yl)cyclopropane- |
| | | carboxamide |
| A-61 | an individualized compound I | azaconazole |
| A-62 | an individualized compound I | bitertanol |
| A-63 | an individualized compound I | bromuconazole |
| A-64 | an individualized compound I | cyproconazole |
| A-65 | an individualized compound I | difenoconazole |
| A-66 | an individualized compound I | diniconazole |
| A-67 | an individualized compound I | diniconazole-M |
| A-68 | an individualized compound I | epoxiconazole |
| A-69 | an individualized compound I | fenbuconazole |
| A-70 | an individualized compound I | fluquinconazole |
| A-71 | an individualized compound I | flusilazole |
| A-72 | an individualized compound I | flutriafol |
| A-73 | an individualized compound I | hexaconazole |
| A-74 | an individualized compound I | imibenconazole |
| A-75 | an individualized compound I | ipconazole |
| A-76 | an individualized compound I | metconazole |
| A-77 | an individualized compound I | myclobutanil |
| A-78 | an individualized compound I | oxpoconazole |
| A-79 | an individualized compound I | paclobutrazole |
| A-80 | an individualized compound I | penconazole |
| A-81 | an individualized compound I | propiconazole |
| A-82 | an individualized compound I | prothioconazole |
| A-83 | an individualized compound I | simeconazole |
| A-84 | an individualized compound I | tebuconazole |
| A-85 | an individualized compound I | tetraconazole |
| A-86 | an individualized compound I | triadimefon |
| A-87 | an individualized compound I | triadimenol |
| A-88 | an individualized compound I | triticonazole |
| A-89 | an individualized compound I | uniconazole |
| A-90 | an individualized compound I | 1-(4-chlorophenyl)-2-([1,2,4]triazol-1-yl)- |
| | | cycloheptanol |
| A-91 | an individualized compound I | cyazofamid |
| A-92 | an individualized compound I | imazalil |
| A-93 | an individualized compound I | imazalil-sulfate |
| A-94 | an individualized compound I | pefurazoate |
| A-95 | an individualized compound I | prochloraz |
| A-96 | an individualized compound I | triflumizole |
| A-97 | an individualized compound I | benomyl |
| A-98 | an individualized compound I | carbendazim |
| A-99 | an individualized compound I | fuberidazole |
| A-100 | an individualized compound I | thiabendazole |
| A-101 | an individualized compound I | ethaboxam |
| A-102 | an individualized compound I | etridiazole |
| A-103 | an individualized compound I | hymexazole |
| A-104 | an individualized compound I | 2-(4-chlorophenyl)-N-[4-(3,4- |
| | | dimethoxyphenyl)isoxazol-5-yl]-2-prop-2- |
| | | ynyloxyacetamide |
| A-105 | an individualized compound I | fluazinam |
| A-106 | an individualized compound I | pyrifenox |
| A-107 | an individualized compound I | 3-[5-(4-chlorophenyl)-2,3-dimethyl- |
| | | isoxazolidin-3-yl]pyridine |
| A-108 | an individualized compound I | 3-[5-(4-methylphenyl)-2,3- |
| | | dimethylisoxazolidin-3-yl]pyridine |
| A-109 | an individualized compound I | 2,3,5,6-tetrachloro-4-methanesulfonyl- |
| | | pyridine |
| A-110 | an individualized compound I | 3,4,5-trichloropyridine-2,6-dicarbonitrile |
| A-111 | an individualized compound I | N-(1-(5-bromo-3-chloropyridin-2-yl)ethyl)- |
| | | 2,4-dichloronicotinamide |
| A-112 | an individualized compound I | N-((5-bromo-3-chloropyridin-2-yl)methyl)- |
| | | 2,4-dichloronicotinamide |
| A-113 | an individualized compound I | bupirimate |
| A-114 | an individualized compound I | cyprodinil |
| A-115 | an individualized compound I | diflumetorim |
| A-116 | an individualized compound I | fenarimol |
| A-117 | an individualized compound I | ferimzone |
| A-118 | an individualized compound I | mepanipyrim |
| A-119 | an individualized compound I | nitrapyrin |
| A-120 | an individualized compound I | nuarimol |
| A-121 | an individualized compound I | pyrimethanil |
| A-122 | an individualized compound I | triforine |
| A-123 | an individualized compound I | fenpiclonil |
| A-124 | an individualized compound I | fludioxonil |
| A-125 | an individualized compound I | aldimorph |
| A-126 | an individualized compound I | dodemorph |
| A-127 | an individualized compound I | dodemorph acetate |
| A-128 | an individualized compound I | fenpropimorph |
| A-129 | an individualized compound I | tridemorph |
| A-130 | an individualized compound I | fenpropidin |
| A-131 | an individualized compound I | fluoroimide |
| A-132 | an individualized compound I | iprodione |
| A-133 | an individualized compound I | procymidone |
| A-134 | an individualized compound I | vinclozolin |
| A-135 | an individualized compound I | famoxadone |
| A-136 | an individualized compound I | fenamidone |
| A-137 | an individualized compound I | flutianil |
| A-138 | an individualized compound I | octhilinone |
| A-139 | an individualized compound I | probenazole |
| A-140 | an individualized compound I | S-allyl 5-amino-2-isopropyl-4- |
| | | orthotolylpyrazol-3-one-1-thiocarboxylate |
| A-141 | an individualized compound I | acibenzolar-S-methyl |
| A-142 | an individualized compound I | amisulbrom |
| A-143 | an individualized compound I | anilazine |
| A-144 | an individualized compound I | blasticidin-S |
| A-145 | an individualized compound I | captafol |
| A-146 | an individualized compound I | captan |
| A-147 | an individualized compound I | chinomethionate |
| A-148 | an individualized compound I | dazomet |
| A-149 | an individualized compound I | debacarb |
| A-150 | an individualized compound I | diclomezine |
| A-151 | an individualized compound I | difenzoquat |
| A-152 | an individualized compound I | difenzoquat methylsulfate |
| A-153 | an individualized compound I | fenoxanil |
| A-154 | an individualized compound I | folpet |
| A-155 | an individualized compound I | oxolinic acid |
| A-156 | an individualized compound I | piperalin |
| A-157 | an individualized compound I | proquinazid |
| A-158 | an individualized compound I | pyroquilon |
| A-159 | an individualized compound I | quinoxyfen |
| A-160 | an individualized compound I | triazoxide |
| A-161 | an individualized compound I | tricyclazole |
| A-162 | an individualized compound I | 2-butoxy-6-iodo-3-propylchromen-4-one |
| A-163 | an individualized compound I | 5-chloro-1-(4,6-dimethoxypyrimidin-2-yl)- |
| | | 2-methyl-1H-benzimidazole |
| A-164 | an individualized compound I | 5-chloro-7-(4-methylpiperidin-1-yl)- |
| | | 6-(2,4,6-trifluorophenyl)-[1,2,4]triazolo- |
| | | [1,5-a]pyrimidine |
| A-165 | an individualized compound I | 5-ethyl-6-octyl-[1,2,4]triazolo[1,5- |
| | | a]pyrimidin-7-ylamine |
| A-166 | an individualized compound I | ferbam |
| A-167 | an individualized compound I | mancozeb |
| A-168 | an individualized compound I | maneb |
| A-169 | an individualized compound I | metam |
| A-170 | an individualized compound I | methasulfocarb |
| A-171 | an individualized compound I | metiram |
| A-172 | an individualized compound I | propineb |
| A-173 | an individualized compound I | thiram |
| A-174 | an individualized compound I | zineb |
| A-175 | an individualized compound I | ziram |
| A-176 | an individualized compound I | diethofencarb |
| A-177 | an individualized compound I | benthiavalicarb |
| A-178 | an individualized compound I | iprovalicarb |
| A-179 | an individualized compound I | propamocarb |
| A-180 | an individualized compound I | propamocarb hydrochloride |
| A-181 | an individualized compound I | valiphenal |
| A-182 | an individualized compound I | 4-fluorophenyl N-(1-(1-(4-cyanophenyl)- |
| | | ethanesulfonyl)but-2-yl)carbamate |
| A-183 | an individualized compound I | dodine |
| A-184 | an individualized compound I | dodine free base |
| A-185 | an individualized compound I | guazatine |
| A-186 | an individualized compound I | guazatine acetate |
| A-187 | an individualized compound I | iminoctadine |
| A-188 | an individualized compound I | iminoctadine triacetate |
| A-189 | an individualized compound I | iminoctadine tris(albesilate) |
| A-190 | an individualized compound I | kasugamycin |
| A-191 | an individualized compound I | kasugamycin hydrochloride hydrate |
| A-192 | an individualized compound I | polyoxin |
| A-193 | an individualized compound I | streptomycin |
| A-194 | an individualized compound I | validamycin A |
| A-195 | an individualized compound I | binapacryl |
| A-196 | an individualized compound I | dicloran |
| A-197 | an individualized compound I | dinobuton |
| A-198 | an individualized compound I | dinocap |
| A-199 | an individualized compound I | nitrothal-isopropyl |
| A-200 | an individualized compound I | tecnazen |
| A-201 | an individualized compound I | fentin salts |
| A-202 | an individualized compound I | dithianon |
| A-203 | an individualized compound I | isoprothiolane |
| A-204 | an individualized compound I | edifenphos |
| A-205 | an individualized compound I | fosetyl, fosetyl aluminum |
| A-206 | an individualized compound I | iprobenfos |
| A-207 | an individualized compound I | phosphorous acid and derivatives |
| A-208 | an individualized compound I | pyrazophos |
| A-209 | an individualized compound I | tolclofos-methyl |
| A-210 | an individualized compound I | chlorthalonil |
| A-211 | an individualized compound I | dichlofluanid |
| A-212 | an individualized compound I | dichlorophene |
| A-213 | an individualized compound I | flusulfamide |
| A-214 | an individualized compound I | hexachlorobenzene |
| A-215 | an individualized compound I | pencycuron |
| A-216 | an individualized compound I | pentachlorophenol and salts |
| A-217 | an individualized compound I | phthalide |
| A-218 | an individualized compound I | quintozene |
| A-219 | an individualized compound I | thiophanate methyl |
| A-220 | an individualized compound I | tolylfluanid |
| A-221 | an individualized compound I | N-(4-chloro-2-nitrophenyl)-N-ethyl- |
| | | 4-methylbenzenesulfonamide |
| A-222 | an individualized compound I | Bordeaux mixture |
| A-223 | an individualized compound I | copper acetate |
| A-224 | an individualized compound I | copper hydroxide |
| A-225 | an individualized compound I | copper oxychloride |
| A-226 | an individualized compound I | basic copper sulfate |
| A-227 | an individualized compound I | sulfur |
| A-228 | an individualized compound I | biphenyl |
| A-229 | an individualized compound I | bronopol |
| A-230 | an individualized compound I | cyflufenamid |
| A-231 | an individualized compound I | cymoxanil |
| A-232 | an individualized compound I | diphenylamine |
| A-233 | an individualized compound I | metrafenone |
| A-234 | an individualized compound I | mildiomycin |
| A-235 | an individualized compound I | oxine-copper |
| A-236 | an individualized compound I | prohexadione-calcium |
| A-237 | an individualized compound I | spiroxamine |
| A-238 | an individualized compound I | tolylfluanid |
| A-239 | an individualized compound I | N-(cyclopropylmethoxyimino-(6-difluoro- |
| | | methoxy-2,3-difluorophenyl)methyl)- |
| | | 2-phenylacetamide |
| A-240 | an individualized compound I | N′-(4-(4-chloro-3-trifluoromethylphenoxy)- |
| | | 2,5-dimethylphenyl)-N-ethyl-N-methyl- |
| | | formamidine |
| A-241 | an individualized compound I | N′-(4-(4-fluoro-3-trifluoromethylphenoxy)- |
| | | 2,5-dimethylphenyl)-N-ethyl-N-methyl- |
| | | formamidine |
| A-242 | an individualized compound I | N′-(2-methyl-5-trifluoromethyl-4-(3-tri- |
| | | methylsilanylpropoxy)phenyl)-N-ethyl- |
| | | N-methylformamidine |
| A-243 | an individualized compound I | N′-(5-difluoromethyl-2-methyl-4-(3-tri- |
| | | methylsilanylpropoxy)phenyl)-N-ethyl- |
| | | N-methylformamidine |
| A-244 | an individualized compound I | methyl N-(1,2,3,4-tetrahydronaphthalen- |
| | | 1-yl)-2-{1-[2-(5-methyl-3-trifluoromethyl- |
| | | pyrazol-1-yl)acetyl]piperidin-4-yl}thiazole- |
| | | 4-carboxamide |
| A-245 | an individualized compound I | methyl (R)-N-(1,2,3,4- |
| | | tetrahydronaphthalen-1-yl)-2-{1-[2-(5- |
| | | methyl-3-trifluoromethyl-pyrazol-1 - |
| | | yl)acetyl]piperidin-4-yl}thiazole-4- |
| | | carboxamide |
| A-246 | an individualized compound I | 6-tert-butyl-8-fluoro-2,3-dimethylquinolin- |
| | | 4-yl acetate |
| A-247 | an individualized compound I | 6-tert-butyl-8-fluoro-2,3-dimethylquinolin- |
| | | 4-yl methoxyacetate |
| A-248 | an individualized compound I | N-methyl-2-{1-[(5-methyl-3-trifluoro- |
| | | methyl-1H-pyrazol-1-yl)acetyl]piperidin-4- |
| | | yl}-N-[(1 R)-1,2,3,4-tetrahydronaphthalen- |
| | | 1-yl]-4-thiazolecarboxamide |
| A-249 | an individualized compound I | carbaryl |
| A-250 | an individualized compound I | carbofuran |
| A-251 | an individualized compound I | carbosulfan |
| A-252 | an individualized compound I | methomylthiodicarb |
| A-253 | an individualized compound I | bifenthrin |
| A-254 | an individualized compound I | cyfluthrin |
| A-255 | an individualized compound I | cypermethrin |
| A-256 | an individualized compound I | alpha-cypermethrin |
| A-257 | an individualized compound I | zeta-cypermethrin |
| A-258 | an individualized compound I | deltamethrin |
| A-259 | an individualized compound I | esfenvalerate |
| A-260 | an individualized compound I | lambda-cyhalothrin |
| A-261 | an individualized compound I | permethrin |
| A-262 | an individualized compound I | tefluthrin |
| A-263 | an individualized compound I | diflubenzuron |
| A-264 | an individualized compound I | flufenoxuron |
| A-265 | an individualized compound I | lufenuron |
| A-266 | an individualized compound I | teflubenzuron |
| A-267 | an individualized compound I | spirotetramate |
| A-268 | an individualized compound I | clothianidin |
| A-269 | an individualized compound I | dinotefuran |
| A-270 | an individualized compound I | imidacloprid |
| A-271 | an individualized compound I | thiamethoxam |
| A-272 | an individualized compound I | acetamiprid |
| A-273 | an individualized compound I | thiacloprid |
| A-274 | an individualized compound I | endosulfan |
| A-275 | an individualized compound I | fipronil |
| A-276 | an individualized compound I | abamectin |
| A-277 | an individualized compound I | emamectin |
| A-278 | an individualized compound I | spinosad |
| A-279 | an individualized compound I | spinetoram |
| A-280 | an individualized compound I | hydramethylnon |
| A-281 | an individualized compound I | chlorfenapyr |
| A-282 | an individualized compound I | fenbutatin oxide |
| A-283 | an individualized compound I | indoxacarb |
| A-284 | an individualized compound I | metaflumizone |
| A-285 | an individualized compound I | flonicamid |
| A-286 | an individualized compound I | lubendiamid |
| A-287 | an individualized compound I | chlorantraniliprol |
| A-288 | an individualized compound I | cyazypyr (HGW86) |
| A-289 | an individualized compound I | cyflumetofen |
| A-290 | an individualized compound I | acetochlor |
| A-291 | an individualized compound I | dimethenamid |
| A-292 | an individualized compound I | metolachlor |
| A-293 | an individualized compound I | metazachlor |
| A-294 | an individualized compound I | glyphosate |
| A-295 | an individualized compound I | glufosinate |
| A-296 | an individualized compound I | sulfosate |
| A-297 | an individualized compound I | clodinafop |
| A-298 | an individualized compound I | fenoxaprop |
| A-299 | an individualized compound I | fluazifop |
| A-300 | an individualized compound I | haloxyfop |
| A-301 | an individualized compound I | paraquat |
| A-302 | an individualized compound I | phenmedipham |
| A-303 | an individualized compound I | clethodim |
| A-304 | an individualized compound I | cycloxydim |
| A-305 | an individualized compound I | profoxydim |
| A-306 | an individualized compound I | sethoxydim |
| A-307 | an individualized compound I | tepraloxydim |
| A-308 | an individualized compound I | pendimethalin |
| A-309 | an individualized compound I | prodiamine |
| A-310 | an individualized compound I | trifluralin |
| A-311 | an individualized compound I | acifluorfen |
| A-312 | an individualized compound I | bromoxynil |
| A-313 | an individualized compound I | imazamethabenz |
| A-314 | an individualized compound I | imazamox |
| A-315 | an individualized compound I | imazapic |
| A-316 | an individualized compound I | imazapyr |
| A-317 | an individualized compound I | imazaquin |
| A-318 | an individualized compound I | imazethapyr |
| A-319 | an individualized compound I | 2,4-dichlorophenoxyacetic acid (2,4-D) |
| A-320 | an individualized compound I | chloridazon |
| A-321 | an individualized compound I | clopyralid |
| A-322 | an individualized compound I | fluroxypyr |
| A-323 | an individualized compound I | picloram |
| A-324 | an individualized compound I | picolinafen |
| A-325 | an individualized compound I | bensulfuron |
| A-326 | an individualized compound I | chlorimuron-ethyl |
| A-327 | an individualized compound I | cyclosulfamuron |
| A-328 | an individualized compound I | iodosulfuron |
| A-329 | an individualized compound I | mesosulfuron |
| A-330 | an individualized compound I | metsulfuron-methyl |
| A-331 | an individualized compound I | nicosulfuron |
| A-332 | an individualized compound I | rimsulfuron |
| A-333 | an individualized compound I | triflusulfuron |
| A-334 | an individualized compound I | atrazine |
| A-335 | an individualized compound I | hexazinone |
| A-336 | an individualized compound I | diuron |
| A-337 | an individualized compound I | florasulam |
| A-338 | an individualized compound I | pyroxasulfon |
| A-339 | an individualized compound I | bentazone |
| A-340 | an individualized compound I | cinidon-ethyl |
| A-341 | an individualized compound I | cinmethylin |
| A-342 | an individualized compound I | dicamba |
| A-343 | an individualized compound I | diflufenzopyr |
| A-344 | an individualized compound I | quinclorac |
| A-345 | an individualized compound I | quinmerac |
| A-346 | an individualized compound I | mesotrione |
| A-347 | an individualized compound I | saflufenacil |
| A-348 | an individualized compound I | topramezone |
| A-349 | an individualized compound I | betacyfluthrin |
| A-350 | an individualized compound I | pirimicarb |
| A-351 | an individualized compound I | acephate |
| A-352 | an individualized compound I | chlorpyrifos |
| A-353 | an individualized compound I | dimethoate |
| A-354 | an individualized compound I | methamidophos |
| A-355 | an individualized compound I | terbufos |
| A-356 | an individualized compound I | aldicarb |
| A-357 | an individualized compound I | flubendiamide |
| A-358 | an individualized compound I | nitenpyram |
| A-359 | an individualized compound I | [(3S,4R,4aR,6S,6aS,12R,12aS,12bS)-3- |
| | | (cyclopropanecarbonyloxy)-6,12- |
| | | dihydroxy-4,6a,12b-trimethyl-11-oxo-9- |
| | | (pyridin-3-yl)-1,2,3,4,4a,5,6,6a,12a,12b- |
| | | decahydro-11H,12H-benzo[f]pyrano[4,3- |
| | | b]chromen-4-yl]methyl |
| | | cyclopropanecarboxylate |

Particularly preferred components 2 are compounds II selected from the group of the following compounds:
II-1 epoxiconazole
II-2 metconazole
II-3 tebuconazole
II-4 fluquinconazole
II-5 flutriafol
II-6 triticonazole
II-7 prothioconazole
II-8 kresoxim-methyl
II-9 pyraclostrobin
II-10 orysastrobin
II-11 dimethomorph
II-12 5-ethyl-6-octyl-[1,2,4]triazolo[1,5-a]pyrimidin-7-ylamine
II-13 pyrimethanil
II-14 metalaxyl
II-15 fenpropimorph
II-16 dodemorph
II-17 iprodione
II-18 mancozeb
II-19 metiram
II-20 thiophanate methyl
II-21 chlorothalonil
II-22 metrafenone
II-23 bixafen
II-24 boscalid
II-25 N-(3′,4′,5′-trifluorobiphenyl-2-yl)-3-difluoromethyl-1-methyl-1H-pyrazole-4-carboxamide
II-26 sedaxane
II-27 isopyrazam
II-28 fluopyram
II-29 penflufen

Particularly preferred mixtures are the binary mixtures of Table B, where each row corresponds to one aspect of the mixtures according to the invention.

TABLE B
Binary mixtures comprising one component 1
selected from compounds of the formula I and
one component 2 selected from groups A to I

Component

| Mixture | 1 | 2 |
|---|---|---|
| B-1 | I-1 | II-1 |
| B-2 | I-1 | II-2 |
| B-3 | I-1 | II-3 |
| B-4 | I-1 | II-4 |
| B-5 | I-1 | II-5 |
| B-6 | I-1 | II-6 |
| B-7 | I-1 | II-7 |
| B-8 | I-1 | II-8 |
| B-9 | I-1 | II-9 |
| B-10 | I-1 | II-10 |
| B-11 | I-1 | II-11 |
| B-12 | I-1 | II-12 |
| B-13 | I-1 | II-13 |
| B-14 | I-1 | II-14 |
| B-15 | I-1 | II-15 |
| B-16 | I-1 | II-16 |
| B-17 | I-1 | II-17 |
| B-18 | I-1 | II-18 |
| B-19 | I-1 | II-19 |
| B-20 | I-1 | II-20 |
| B-21 | I-1 | II-21 |
| B-22 | I-1 | II-22 |
| B-23 | I-1 | II-23 |
| B-24 | I-1 | II-24 |
| B-25 | I-1 | II-25 |
| B-26 | I-1 | II-26 |
| B-27 | I-1 | II-27 |
| B-28 | I-1 | II-28 |
| B-29 | I-1 | II-29 |
| B-30 | I-2 | II-1 |
| B-31 | I-2 | II-2 |
| B-32 | I-2 | II-3 |
| B-33 | I-2 | II-4 |
| B-34 | I-2 | II-5 |
| B-35 | I-2 | II-6 |
| B-36 | I-2 | II-7 |
| B-37 | I-2 | II-8 |
| B-38 | I-2 | II-9 |
| B-39 | I-2 | II-10 |
| B-40 | I-2 | II-11 |
| B-41 | I-2 | II-12 |
| B-42 | I-2 | II-13 |
| B-43 | I-2 | II-14 |
| B-44 | I-2 | II-15 |
| B-45 | I-2 | II-16 |
| B-46 | I-2 | II-17 |
| B-47 | I-2 | II-18 |
| B-48 | I-2 | II-19 |
| B-49 | I-2 | II-20 |
| B-50 | I-2 | II-21 |
| B-51 | I-2 | II-22 |
| B-52 | I-2 | II-23 |
| B-53 | I-2 | II-24 |
| B-54 | I-2 | II-25 |
| B-55 | I-2 | II-26 |
| B-56 | I-2 | II-27 |
| B-57 | I-2 | II-28 |
| B-58 | I-2 | II-29 |
| B-59 | I-3 | II-1 |
| B-60 | I-3 | II-2 |
| B-61 | I-3 | II-3 |
| B-62 | I-3 | II-4 |
| B-63 | I-3 | II-5 |
| B-64 | I-3 | II-6 |
| B-65 | I-3 | II-7 |
| B-66 | I-3 | II-8 |
| B-67 | I-3 | II-9 |
| B-68 | I-3 | II-10 |
| B-69 | I-3 | II-11 |
| B-70 | I-3 | II-12 |
| B-71 | I-3 | II-13 |
| B-72 | I-3 | II-14 |
| B-73 | I-3 | II-15 |
| B-74 | I-3 | II-16 |
| B-75 | I-3 | II-17 |
| B-76 | I-3 | II-18 |
| B-77 | I-3 | II-19 |
| B-78 | I-3 | II-20 |
| B-79 | I-3 | II-21 |
| B-80 | I-3 | II-22 |
| B-81 | I-3 | II-23 |
| B-82 | I-3 | II-24 |
| B-83 | I-3 | II-25 |
| B-84 | I-3 | II-26 |
| B-85 | I-3 | II-27 |
| B-86 | I-3 | II-28 |
| B-87 | I-3 | II-29 |
| B-88 | I-4 | II-1 |
| B-89 | I-4 | II-2 |
| B-90 | I-4 | II-3 |
| B-91 | I-4 | II-4 |
| B-92 | I-4 | II-5 |
| B-93 | I-4 | II-6 |
| B-94 | I-4 | II-7 |
| B-95 | I-4 | II-8 |
| B-96 | I-4 | II-9 |
| B-97 | I-4 | II-10 |
| B-98 | I-4 | II-11 |
| B-99 | I-4 | II-12 |
| B-100 | I-4 | II-13 |
| B-101 | I-4 | II-14 |
| B-102 | I-4 | II-15 |
| B-103 | I-4 | II-16 |
| B-104 | I-4 | II-17 |
| B-105 | I-4 | II-18 |
| B-106 | I-4 | II-19 |
| B-107 | I-4 | II-20 |
| B-108 | I-4 | II-21 |
| B-109 | I-4 | II-22 |
| B-110 | I-4 | II-23 |
| B-111 | I-4 | II-24 |
| B-112 | I-4 | II-25 |
| B-113 | I-4 | II-26 |
| B-114 | I-4 | II-27 |
| B-115 | I-4 | II-28 |
| B-116 | I-4 | II-29 |
| B-117 | I-5 | II-1 |
| B-118 | I-5 | II-2 |
| B-119 | I-5 | II-3 |
| B-120 | I-5 | II-4 |
| B-121 | I-5 | II-5 |
| B-122 | I-5 | II-6 |
| B-123 | I-5 | II-7 |
| B-124 | I-5 | II-8 |
| B-125 | I-5 | II-9 |
| B-126 | I-5 | II-10 |
| B-127 | I-5 | II-11 |
| B-128 | I-5 | II-12 |
| B-129 | I-5 | II-13 |
| B-130 | I-5 | II-14 |
| B-131 | I-5 | II-15 |
| B-132 | I-5 | II-16 |
| B-133 | I-5 | II-17 |
| B-134 | I-5 | II-18 |
| B-135 | I-5 | II-19 |
| B-136 | I-5 | II-20 |
| B-137 | I-5 | II-21 |
| B-138 | I-5 | II-22 |
| B-139 | I-5 | II-23 |
| B-140 | I-5 | II-24 |
| B-141 | I-5 | II-25 |
| B-142 | I-5 | II-26 |
| B-143 | I-5 | II-27 |
| B-144 | I-5 | II-28 |
| B-145 | I-5 | II-29 |
| B-146 | I-6 | II-1 |
| B-147 | I-6 | II-2 |
| B-148 | I-6 | II-3 |
| B-149 | I-6 | II-4 |
| B-150 | I-6 | II-5 |
| B-151 | I-6 | II-6 |
| B-152 | I-6 | II-7 |
| B-153 | I-6 | II-8 |
| B-154 | I-6 | II-9 |
| B-155 | I-6 | II-10 |
| B-156 | I-6 | II-11 |
| B-157 | I-6 | II-12 |
| B-158 | I-6 | II-13 |
| B-159 | I-6 | II-14 |
| B-160 | I-6 | II-15 |
| B-161 | I-6 | II-16 |
| B-162 | I-6 | II-17 |
| B-163 | I-6 | II-18 |
| B-164 | I-6 | II-19 |
| B-165 | I-6 | II-20 |
| B-166 | I-6 | II-21 |
| B-167 | I-6 | II-22 |
| B-168 | I-6 | II-23 |
| B-169 | I-6 | II-24 |
| B-170 | I-6 | II-25 |
| B-171 | I-6 | II-26 |
| B-172 | I-6 | II-27 |
| B-173 | I-6 | II-28 |
| B-174 | I-6 | II-29 |
| B-175 | I-7 | II-1 |
| B-176 | I-7 | II-2 |
| B-177 | I-7 | II-3 |
| B-178 | I-7 | II-4 |
| B-179 | I-7 | II-5 |
| B-180 | I-7 | II-6 |
| B-181 | I-7 | II-7 |
| B-182 | I-7 | II-8 |
| B-183 | I-7 | II-9 |
| B-184 | I-7 | II-10 |
| B-185 | I-7 | II-11 |
| B-186 | I-7 | II-12 |
| B-187 | I-7 | II-13 |
| B-188 | I-7 | II-14 |
| B-189 | I-7 | II-15 |
| B-190 | I-7 | II-16 |
| B-191 | I-7 | II-17 |
| B-192 | I-7 | II-18 |
| B-193 | I-7 | II-19 |
| B-194 | I-7 | II-20 |
| B-195 | I-7 | II-21 |
| B-196 | I-7 | II-22 |
| B-197 | I-7 | II-23 |
| B-198 | I-7 | II-24 |
| B-199 | I-7 | II-25 |
| B-200 | I-7 | II-26 |
| B-201 | I-7 | II-27 |
| B-202 | I-7 | II-28 |
| B-203 | I-7 | II-29 |
| B-204 | I-8 | II-1 |
| B-205 | I-8 | II-2 |
| B-206 | I-8 | II-3 |
| B-207 | I-8 | II-4 |
| B-208 | I-8 | II-5 |
| B-209 | I-8 | II-6 |
| B-210 | I-8 | II-7 |
| B-211 | I-8 | II-8 |
| B-212 | I-8 | II-9 |
| B-213 | I-8 | II-10 |
| B-214 | I-8 | II-11 |
| B-215 | I-8 | II-12 |
| B-216 | I-8 | II-13 |
| B-217 | I-8 | II-14 |
| B-218 | I-8 | II-15 |
| B-219 | I-8 | II-16 |
| B-220 | I-8 | II-17 |
| B-221 | I-8 | II-18 |
| B-222 | I-8 | II-19 |
| B-223 | I-8 | II-20 |
| B-224 | I-8 | II-21 |
| B-225 | I-8 | II-22 |
| B-226 | I-8 | II-23 |
| B-227 | I-8 | II-24 |
| B-228 | I-8 | II-25 |
| B-229 | I-8 | II-26 |
| B-230 | I-8 | II-27 |
| B-231 | I-8 | II-28 |
| B-232 | I-8 | II-29 |
| B-233 | I-9 | II-1 |
| B-234 | I-9 | II-2 |
| B-235 | I-9 | II-3 |
| B-236 | I-9 | II-4 |
| B-237 | I-9 | II-5 |
| B-238 | I-9 | II-6 |
| B-239 | I-9 | II-7 |
| B-240 | I-9 | II-8 |
| B-241 | I-9 | II-9 |
| B-242 | I-9 | II-10 |
| B-243 | I-9 | II-11 |
| B-244 | I-9 | II-12 |
| B-245 | I-9 | II-13 |
| B-246 | I-9 | II-14 |
| B-247 | I-9 | II-15 |
| B-248 | I-9 | II-16 |
| B-249 | I-9 | II-17 |
| B-250 | I-9 | II-18 |
| B-251 | I-9 | II-19 |
| B-252 | I-9 | II-20 |
| B-253 | I-9 | II-21 |
| B-254 | I-9 | II-22 |
| B-255 | I-9 | II-23 |
| B-256 | I-9 | II-24 |
| B-257 | I-9 | II-25 |
| B-258 | I-9 | II-26 |
| B-259 | I-9 | II-27 |
| B-260 | I-9 | II-28 |
| B-261 | I-9 | II-29 |
| B-262 | I-10 | II-1 |
| B-263 | I-10 | II-2 |
| B-264 | I-10 | II-3 |
| B-265 | I-10 | II-4 |
| B-266 | I-10 | II-5 |
| B-267 | I-10 | II-6 |
| B-268 | I-10 | II-7 |
| B-269 | I-10 | II-8 |
| B-270 | I-10 | II-9 |
| B-271 | I-10 | II-10 |
| B-272 | I-10 | II-11 |
| B-273 | I-10 | II-12 |
| B-274 | I-10 | II-13 |
| B-275 | I-10 | II-14 |
| B-276 | I-10 | II-15 |
| B-277 | I-10 | II-16 |
| B-278 | I-10 | II-17 |
| B-279 | I-10 | II-18 |
| B-280 | I-10 | II-19 |
| B-281 | I-10 | II-20 |
| B-282 | I-10 | II-21 |
| B-283 | I-10 | II-22 |
| B-284 | I-10 | II-23 |
| B-285 | I-10 | II-24 |
| B-286 | I-10 | II-25 |
| B-287 | I-10 | II-26 |
| B-288 | I-10 | II-27 |
| B-289 | I-10 | II-28 |
| B-290 | I-10 | II-29 |
| B-291 | I-11 | II-1 |
| B-292 | I-11 | II-2 |
| B-293 | I-11 | II-3 |
| B-294 | I-11 | II-4 |
| B-295 | I-11 | II-5 |
| B-296 | I-11 | II-6 |
| B-297 | I-11 | II-7 |
| B-298 | I-11 | II-8 |
| B-299 | I-11 | II-9 |
| B-300 | I-11 | II-10 |
| B-301 | I-11 | II-11 |
| B-302 | I-11 | II-12 |
| B-303 | I-11 | II-13 |
| B-304 | I-11 | II-14 |
| B-305 | I-11 | II-15 |
| B-306 | I-11 | II-16 |
| B-307 | I-11 | II-17 |
| B-308 | I-11 | II-18 |
| B-309 | I-11 | II-19 |
| B-310 | I-11 | II-20 |
| B-311 | I-11 | II-21 |
| B-312 | I-11 | II-22 |
| B-313 | I-11 | II-23 |
| B-314 | I-11 | II-24 |
| B-315 | I-11 | II-25 |
| B-316 | I-11 | II-26 |
| B-317 | I-11 | II-27 |
| B-318 | I-11 | II-28 |
| B-319 | I-11 | II-29 |
| B-320 | I-12 | II-1 |
| B-321 | I-12 | II-2 |
| B-322 | I-12 | II-3 |
| B-323 | I-12 | II-4 |
| B-324 | I-12 | II-5 |
| B-325 | I-12 | II-6 |
| B-326 | I-12 | II-7 |
| B-327 | I-12 | II-8 |
| B-328 | I-12 | II-9 |
| B-329 | I-12 | II-10 |
| B-330 | I-12 | II-11 |
| B-331 | I-12 | II-12 |
| B-332 | I-12 | II-13 |
| B-333 | I-12 | II-14 |
| B-334 | I-12 | II-15 |
| B-335 | I-12 | II-16 |
| B-336 | I-12 | II-17 |
| B-337 | I-12 | II-18 |
| B-338 | I-12 | II-19 |
| B-339 | I-12 | II-20 |
| B-340 | I-12 | II-21 |
| B-341 | I-12 | II-22 |
| B-342 | I-12 | II-23 |
| B-343 | I-12 | II-24 |
| B-344 | I-12 | II-25 |
| B-345 | I-12 | II-26 |
| B-346 | I-12 | II-27 |
| B-347 | I-12 | II-28 |
| B-348 | I-12 | II-29 |
| B-349 | I-13 | II-1 |
| B-350 | I-13 | II-2 |
| B-351 | I-13 | II-3 |
| B-352 | I-13 | II-4 |
| B-353 | I-13 | II-5 |
| B-354 | I-13 | II-6 |
| B-355 | I-13 | II-7 |
| B-356 | I-13 | II-8 |
| B-357 | I-13 | II-9 |
| B-358 | I-13 | II-10 |
| B-359 | I-13 | II-11 |
| B-360 | I-13 | II-12 |
| B-361 | I-13 | II-13 |
| B-362 | I-13 | II-14 |
| B-363 | I-13 | II-15 |
| B-364 | I-13 | II-16 |
| B-365 | I-13 | II-17 |
| B-366 | I-13 | II-18 |
| B-367 | I-13 | II-19 |
| B-368 | I-13 | II-20 |
| B-369 | I-13 | II-21 |
| B-370 | I-13 | II-22 |
| B-371 | I-13 | II-23 |
| B-372 | I-13 | II-24 |
| B-373 | I-13 | II-25 |
| B-374 | I-13 | II-26 |
| B-375 | I-13 | II-27 |
| B-376 | I-13 | II-28 |
| B-377 | I-13 | II-29 |
| B-378 | I-14 | II-1 |
| B-379 | I-14 | II-2 |
| B-380 | I-14 | II-3 |
| B-381 | I-14 | II-4 |
| B-382 | I-14 | II-5 |
| B-383 | I-14 | II-6 |
| B-384 | I-14 | II-7 |
| B-385 | I-14 | II-8 |
| B-386 | I-14 | II-9 |
| B-387 | I-14 | II-10 |
| B-388 | I-14 | II-11 |
| B-389 | I-14 | II-12 |
| B-390 | I-14 | II-13 |
| B-391 | I-14 | II-14 |
| B-392 | I-14 | II-15 |
| B-393 | I-14 | II-16 |
| B-394 | I-14 | II-17 |
| B-395 | I-14 | II-18 |
| B-396 | I-14 | II-19 |
| B-397 | I-14 | II-20 |
| B-398 | I-14 | II-21 |
| B-399 | I-14 | II-22 |
| B-400 | I-14 | II-23 |
| B-401 | I-14 | II-24 |
| B-402 | I-14 | II-25 |
| B-403 | I-14 | II-26 |
| B-404 | I-14 | II-27 |
| B-405 | I-14 | II-28 |
| B-406 | I-14 | II-29 |
| B-407 | I-15 | II-1 |
| B-408 | I-15 | II-2 |
| B-409 | I-15 | II-3 |
| B-410 | I-15 | II-4 |
| B-411 | I-15 | II-5 |
| B-412 | I-15 | II-6 |
| B-413 | I-15 | II-7 |
| B-414 | I-15 | II-8 |
| B-415 | I-15 | II-9 |
| B-416 | I-15 | II-10 |
| B-417 | I-15 | II-11 |
| B-418 | I-15 | II-12 |
| B-419 | I-15 | II-13 |
| B-420 | I-15 | II-14 |
| B-421 | I-15 | II-15 |
| B-422 | I-15 | II-16 |
| B-423 | I-15 | II-17 |
| B-424 | I-15 | II-18 |
| B-425 | I-15 | II-19 |
| B-426 | I-15 | II-20 |
| B-427 | I-15 | II-21 |
| B-428 | I-15 | II-22 |
| B-429 | I-15 | II-23 |
| B-430 | I-15 | II-24 |
| B-431 | I-15 | II-25 |
| B-432 | I-15 | II-26 |
| B-433 | I-15 | II-27 |
| B-434 | I-15 | II-28 |
| B-435 | I-15 | II-29 |
| B-436 | I-16 | II-1 |
| B-437 | I-16 | II-2 |
| B-438 | I-16 | II-3 |
| B-439 | I-16 | II-4 |
| B-440 | I-16 | II-5 |
| B-441 | I-16 | II-6 |
| B-442 | I-16 | II-7 |
| B-443 | I-16 | II-8 |
| B-444 | I-16 | II-9 |
| B-445 | I-16 | II-10 |
| B-446 | I-16 | II-11 |
| B-447 | I-16 | II-12 |
| B-448 | I-16 | II-13 |
| B-449 | I-16 | II-14 |
| B-450 | I-16 | II-15 |
| B-451 | I-16 | II-16 |
| B-452 | I-16 | II-17 |
| B-453 | I-16 | II-18 |
| B-454 | I-16 | II-19 |
| B-455 | I-16 | II-20 |
| B-456 | I-16 | II-21 |
| B-457 | I-16 | II-22 |
| B-458 | I-16 | II-23 |
| B-459 | I-16 | II-24 |
| B-460 | I-16 | II-25 |
| B-461 | I-16 | II-26 |
| B-462 | I-16 | II-27 |
| B-463 | I-16 | II-28 |
| B-464 | I-16 | II-29 |
| B-465 | I-17 | II-1 |
| B-466 | I-17 | II-2 |
| B-467 | I-17 | II-3 |
| B-468 | I-17 | II-4 |
| B-469 | I-17 | II-5 |
| B-470 | I-17 | II-6 |
| B-471 | I-17 | II-7 |
| B-472 | I-17 | II-8 |
| B-473 | I-17 | II-9 |
| B-474 | I-17 | II-10 |
| B-475 | I-17 | II-11 |
| B-476 | I-17 | II-12 |
| B-477 | I-17 | II-13 |
| B-478 | I-17 | II-14 |
| B-479 | I-17 | II-15 |
| B-480 | I-17 | II-16 |
| B-481 | I-17 | II-17 |
| B-482 | I-17 | II-18 |
| B-483 | I-17 | II-19 |
| B-484 | I-17 | II-20 |
| B-485 | I-17 | II-21 |
| B-486 | I-17 | II-22 |
| B-487 | I-17 | II-23 |
| B-488 | I-17 | II-24 |
| B-489 | I-17 | II-25 |
| B-490 | I-17 | II-26 |
| B-491 | I-17 | II-27 |
| B-492 | I-17 | II-28 |
| B-493 | I-17 | II-29 |
| B-494 | I-18 | II-1 |
| B-495 | I-18 | II-2 |
| B-496 | I-18 | II-3 |
| B-497 | I-18 | II-4 |
| B-498 | I-18 | II-5 |
| B-499 | I-18 | II-6 |
| B-500 | I-18 | II-7 |
| B-501 | I-18 | II-8 |
| B-502 | I-18 | II-9 |
| B-503 | I-18 | II-10 |
| B-504 | I-18 | II-11 |
| B-505 | I-18 | II-12 |
| B-506 | I-18 | II-13 |
| B-507 | I-18 | II-14 |
| B-508 | I-18 | II-15 |
| B-509 | I-18 | II-16 |
| B-510 | I-18 | II-17 |
| B-511 | I-18 | II-18 |
| B-512 | I-18 | II-19 |
| B-513 | I-18 | II-20 |
| B-514 | I-18 | II-21 |
| B-515 | I-18 | II-22 |
| B-516 | I-18 | II-23 |
| B-517 | I-18 | II-24 |
| B-518 | I-18 | II-25 |
| B-519 | I-18 | II-26 |
| B-520 | I-18 | II-27 |
| B-521 | I-18 | II-28 |
| B-522 | I-18 | II-29 |

Other preferred components 2 are compounds II selected from the group of the following compounds:

| | |
|---|---|
| II-30 | cyflufenamid |
| II-31 | spiroxamine |
| II-32 | fenpropidin |
| II-33 | proquinazid |
| II-34 | dimoxystrobin |
| II-35 | iprovalicarb |
| II-36 | cyprodinil |
| II-37 | folpet |
| II-38 | fludioxonil |
| II-39 | zoxamide |
| II-40 | fluazinam |
| II-41 | cyazofamid |
| II-42 | benthiavalicarb |
| II-43 | fluopicolid |
| II-44 | ethaboxam |
| II-45 | amisulbrom |

Other preferred mixtures are the binary mixtures of Table B1, where each row corresponds to one aspect of the mixtures according to the invention.

TABLE B1
Binary mixtures comprising one component 1 selected
from compounds of the formula I and one component 2
selected from groups A to F

Component

| Mixture | 1 | 2 |
|---|---|---|
| B1-1 | I-1 | II-30 |
| B1-2 | I-1 | II-31 |
| B1-3 | I-1 | II-32 |
| B1-4 | I-1 | II-33 |
| B1-5 | I-1 | II-34 |
| B1-6 | I-1 | II-35 |
| B1-7 | I-1 | II-36 |
| B1-8 | I-1 | II-37 |
| B1-9 | I-1 | II-38 |
| B1-10 | I-1 | II-39 |
| B1-11 | I-1 | II-40 |
| B1-12 | I-1 | II-41 |
| B1-13 | I-1 | II-42 |
| B1-14 | I-1 | II-43 |
| B1-15 | I-1 | II-44 |
| B1-16 | I-1 | II-45 |
| B1-17 | I-2 | II-30 |
| B1-18 | I-2 | II-31 |
| B1-19 | I-2 | II-32 |
| B1-20 | I-2 | II-33 |
| B1-21 | I-2 | II-34 |
| B1-22 | I-2 | II-35 |
| B1-23 | I-2 | II-36 |
| B1-24 | I-2 | II-37 |
| B1-25 | I-2 | II-38 |
| B1-26 | I-2 | II-39 |
| B1-27 | I-2 | II-40 |
| B1-28 | I-2 | II-41 |
| B1-29 | I-2 | II-42 |
| B1-30 | I-2 | II-43 |
| B1-31 | I-2 | II-44 |
| B1-32 | I-2 | II-45 |
| B1-33 | I-3 | II-30 |
| B1-34 | I-3 | II-31 |
| B1-35 | I-3 | II-32 |
| B1-36 | I-3 | II-33 |
| B1-37 | I-3 | II-34 |
| B1-38 | I-3 | II-35 |
| B1-39 | I-3 | II-36 |
| B1-40 | I-3 | II-37 |
| B1-41 | I-3 | II-38 |
| B1-42 | I-3 | II-39 |
| B1-43 | I-3 | II-40 |
| B1-44 | I-3 | II-41 |
| B1-45 | I-3 | II-42 |
| B1-46 | I-3 | II-43 |
| B1-47 | I-3 | II-44 |
| B1-48 | I-3 | II-45 |
| B1-49 | I-4 | II-30 |
| B1-50 | I-4 | II-31 |
| B1-51 | I-4 | II-32 |
| B1-52 | I-4 | II-33 |
| B1-53 | I-4 | II-34 |
| B1-54 | I-4 | II-35 |
| B1-55 | I-4 | II-36 |
| B1-56 | I-4 | II-37 |
| B1-57 | I-4 | II-38 |
| B1-58 | I-4 | II-39 |
| B1-59 | I-4 | II-40 |
| B1-60 | I-4 | II-41 |
| B1-61 | I-4 | II-42 |
| B1-62 | I-4 | II-43 |
| B1-63 | I-4 | II-44 |
| B1-64 | I-4 | II-45 |
| B1-65 | I-5 | II-30 |
| B1-66 | I-5 | II-31 |
| B1-67 | I-5 | II-32 |
| B1-68 | I-5 | II-33 |
| B1-69 | I-5 | II-34 |
| B1-70 | I-5 | II-35 |
| B1-71 | I-5 | II-36 |
| B1-72 | I-5 | II-37 |
| B1-73 | I-5 | II-38 |
| B1-74 | I-5 | II-39 |
| B1-75 | I-5 | II-40 |
| B1-76 | I-5 | II-41 |
| B1-77 | I-5 | II-42 |
| B1-78 | I-5 | II-43 |
| B1-79 | I-5 | II-44 |
| B1-80 | I-5 | II-45 |
| B1-81 | I-6 | II-30 |
| B1-82 | I-6 | II-31 |
| B1-83 | I-6 | II-32 |
| B1-84 | I-6 | II-33 |
| B1-85 | I-6 | II-34 |
| B1-86 | I-6 | II-35 |
| B1-87 | I-6 | II-36 |
| B1-88 | I-6 | II-37 |
| B1-89 | I-6 | II-38 |
| B1-90 | I-6 | II-39 |
| B1-91 | I-6 | II-40 |
| B1-92 | I-6 | II-41 |
| B1-93 | I-6 | II-42 |
| B1-94 | I-6 | II-43 |
| B1-95 | I-6 | II-44 |
| B1-96 | I-6 | II-45 |
| B1-97 | I-7 | II-30 |
| B1-98 | I-7 | II-31 |
| B1-99 | I-7 | II-32 |
| B1-100 | I-7 | II-33 |
| B1-101 | I-7 | II-34 |
| B1-102 | I-7 | II-35 |
| B1-103 | I-7 | II-36 |
| B1-104 | I-7 | II-37 |
| B1-105 | I-7 | II-38 |
| B1-106 | I-7 | II-39 |
| B1-107 | I-7 | II-40 |
| B1-108 | I-7 | II-41 |
| B1-109 | I-7 | II-42 |
| B1-110 | I-7 | II-43 |
| B1-111 | I-7 | II-44 |
| B1-112 | I-7 | II-45 |
| B1-113 | I-8 | II-30 |
| B1-114 | I-8 | II-31 |
| B1-115 | I-8 | II-32 |
| B1-116 | I-8 | II-33 |
| B1-117 | I-8 | II-34 |
| B1-118 | I-8 | II-35 |
| B1-119 | I-8 | II-36 |
| B1-120 | I-8 | II-37 |
| B1-121 | I-8 | II-38 |
| B1-122 | I-8 | II-39 |
| B1-123 | I-8 | II-40 |
| B1-124 | I-8 | II-41 |
| B1-125 | I-8 | II-42 |
| B1-126 | I-8 | II-43 |
| B1-127 | I-8 | II-44 |
| B1-128 | I-8 | II-45 |
| B1-129 | I-9 | II-30 |
| B1-130 | I-9 | II-31 |
| B1-131 | I-9 | II-32 |
| B1-132 | I-9 | II-33 |
| B1-133 | I-9 | II-34 |
| B1-134 | I-9 | II-35 |
| B1-135 | I-9 | II-36 |
| B1-136 | I-9 | II-37 |
| B1-137 | I-9 | II-38 |
| B1-138 | I-9 | II-39 |
| B1-139 | I-9 | II-40 |
| B1-140 | I-9 | II-41 |
| B1-141 | I-9 | II-42 |
| B1-142 | I-9 | II-43 |
| B1-143 | I-9 | II-44 |
| B1-144 | I-9 | II-45 |
| B1-145 | I-10 | II-30 |
| B1-146 | I-10 | II-31 |
| B1-147 | I-10 | II-32 |
| B1-148 | I-10 | II-33 |
| B1-149 | I-10 | II-34 |
| B1-150 | I-10 | II-35 |
| B1-151 | I-10 | II-36 |
| B1-152 | I-10 | II-37 |
| B1-153 | I-10 | II-38 |
| B1-154 | I-10 | II-39 |
| B1-155 | I-10 | II-40 |
| B1-156 | I-10 | II-41 |
| B1-157 | I-10 | II-42 |
| B1-158 | I-10 | II-43 |
| B1-159 | I-10 | II-44 |
| B1-160 | I-10 | II-45 |
| B1-161 | I-11 | II-30 |
| B1-162 | I-11 | II-31 |
| B1-163 | I-11 | II-32 |
| B1-164 | I-11 | II-33 |
| B1-165 | I-11 | II-34 |
| B1-166 | I-11 | II-35 |
| B1-167 | I-11 | II-36 |
| B1-168 | I-11 | II-37 |
| B1-169 | I-11 | II-38 |
| B1-170 | I-11 | II-39 |
| B1-171 | I-11 | II-40 |
| B1-172 | I-11 | II-41 |
| B1-173 | I-11 | II-42 |
| B1-174 | I-11 | II-43 |
| B1-175 | I-11 | II-44 |
| B1-176 | I-11 | II-45 |
| B1-177 | I-12 | II-30 |
| B1-178 | I-12 | II-31 |
| B1-179 | I-12 | II-32 |
| B1-180 | I-12 | II-33 |
| B1-181 | I-12 | II-34 |
| B1-182 | I-12 | II-35 |
| B1-183 | I-12 | II-36 |
| B1-184 | I-12 | II-37 |
| B1-185 | I-12 | II-38 |
| B1-186 | I-12 | II-39 |
| B1-187 | I-12 | II-40 |
| B1-188 | I-12 | II-41 |
| B1-189 | I-12 | II-42 |
| B1-190 | I-12 | II-43 |
| B1-191 | I-12 | II-44 |
| B1-192 | I-12 | II-45 |
| B1-193 | I-13 | II-30 |
| B1-194 | I-13 | II-31 |
| B1-195 | I-13 | II-32 |
| B1-196 | I-13 | II-33 |
| B1-197 | I-13 | II-34 |
| B1-198 | I-13 | II-35 |
| B1-199 | I-13 | II-36 |
| B1-200 | I-13 | II-37 |
| B1-201 | I-13 | II-38 |
| B1-202 | I-13 | II-39 |
| B1-203 | I-13 | II-40 |
| B1-204 | I-13 | II-41 |
| B1-205 | I-13 | II-42 |
| B1-206 | I-13 | II-43 |
| B1-207 | I-13 | II-44 |
| B1-208 | I-13 | II-45 |
| B1-209 | I-14 | II-30 |
| B1-210 | I-14 | II-31 |
| B1-211 | I-14 | II-32 |
| B1-212 | I-14 | II-33 |
| B1-213 | I-14 | II-34 |
| B1-214 | I-14 | II-35 |
| B1-215 | I-14 | II-36 |
| B1-216 | I-14 | II-37 |
| B1-217 | I-14 | II-38 |
| B1-218 | I-14 | II-39 |
| B1-219 | I-14 | II-40 |
| B1-220 | I-14 | II-41 |
| B1-221 | I-14 | II-42 |
| B1-222 | I-14 | II-43 |
| B1-223 | I-14 | II-44 |
| B1-224 | I-14 | II-45 |
| B1-225 | I-15 | II-30 |
| B1-226 | I-15 | II-31 |
| B1-227 | I-15 | II-32 |
| B1-228 | I-15 | II-33 |
| B1-229 | I-15 | II-34 |
| B1-230 | I-15 | II-35 |
| B1-231 | I-15 | II-36 |
| B1-232 | I-15 | II-37 |
| B1-233 | I-15 | II-38 |
| B1-234 | I-15 | II-39 |
| B1-235 | I-15 | II-40 |
| B1-236 | I-15 | II-41 |
| B1-237 | I-15 | II-42 |
| B1-238 | I-15 | II-43 |
| B1-239 | I-15 | II-44 |
| B1-240 | I-15 | II-45 |
| B1-241 | I-16 | II-30 |
| B1-242 | I-16 | II-31 |
| B1-243 | I-16 | II-32 |
| B1-244 | I-16 | II-33 |
| B1-245 | I-16 | II-34 |
| B1-246 | I-16 | II-35 |
| B1-247 | I-16 | II-36 |
| B1-248 | I-16 | II-37 |
| B1-249 | I-16 | II-38 |
| B1-250 | I-16 | II-39 |
| B1-251 | I-16 | II-40 |
| B1-252 | I-16 | II-41 |
| B1-253 | I-16 | II-42 |
| B1-254 | I-16 | II-43 |
| B1-255 | I-16 | II-44 |
| B1-256 | I-16 | II-45 |
| B1-257 | I-17 | II-30 |
| B1-258 | I-17 | II-31 |
| B1-259 | I-17 | II-32 |
| B1-260 | I-17 | II-33 |
| B1-261 | I-17 | II-34 |
| B1-262 | I-17 | II-35 |
| B1-263 | I-17 | II-36 |
| B1-264 | I-17 | II-37 |
| B1-265 | I-17 | II-38 |
| B1-266 | I-17 | II-39 |
| B1-267 | I-17 | II-40 |
| B1-268 | I-17 | II-41 |
| B1-269 | I-17 | II-42 |
| B1-270 | I-17 | II-43 |
| B1-271 | I-17 | II-44 |
| B1-272 | I-17 | II-45 |
| B1-273 | I-18 | II-30 |
| B1-274 | I-18 | II-31 |
| B1-275 | I-18 | II-32 |
| B1-276 | I-18 | II-33 |
| B1-277 | I-18 | II-34 |
| B1-278 | I-18 | II-35 |
| B1-279 | I-18 | II-36 |
| B1-280 | I-18 | II-37 |
| B1-281 | I-18 | II-38 |
| B1-282 | I-18 | II-39 |
| B1-283 | I-18 | II-40 |
| B1-284 | I-18 | II-41 |
| B1-285 | I-18 | II-42 |
| B1-286 | I-18 | II-43 |
| B1-287 | I-18 | II-44 |
| B1-288 | I-18 | II-45 |

Other preferred components 2 are compounds II selected from the group of the following compounds:

| | |
|---|---|
| II-46 | prochloraz |
| II-47 | dithianon |
| II-48 | difenoconazole |

Other preferred mixtures are the binary mixtures of Table B2, where each row corresponds to one aspect of the mixtures according to the invention.

TABLE B2
Binary mixtures comprising one component 1 selected
from compounds of the formula I and one component 2
selected from groups A to F

Component

| Mixture | 1 | 2 |
|---|---|---|
| B2-1 | I-1 | II-46 |
| B2-2 | I-1 | II-47 |
| B2-3 | I-1 | II-48 |
| B2-4 | I-2 | II-46 |
| B2-5 | I-2 | II-47 |
| B2-6 | I-2 | II-48 |
| B2-7 | I-3 | II-46 |
| B2-8 | I-3 | II-47 |
| B2-9 | I-3 | II-48 |
| B2-10 | I-4 | II-46 |
| B2-11 | I-4 | II-47 |
| B2-12 | I-4 | II-48 |
| B2-13 | I-5 | II-46 |
| B2-14 | I-5 | II-47 |
| B2-15 | I-5 | II-48 |
| B2-16 | I-6 | II-46 |
| B2-17 | I-6 | II-47 |
| B2-18 | I-6 | II-48 |
| B2-19 | I-7 | II-46 |
| B2-20 | I-7 | II-47 |
| B2-21 | I-7 | II-48 |
| B2-22 | I-8 | II-46 |
| B2-23 | I-8 | II-47 |
| B2-24 | I-8 | II-48 |
| B2-25 | I-9 | II-46 |
| B2-26 | I-9 | II-47 |
| B2-27 | I-9 | II-48 |
| B2-28 | I-10 | II-46 |
| B2-29 | I-10 | II-47 |
| B2-30 | I-10 | II-48 |
| B2-31 | I-11 | II-46 |
| B2-32 | I-11 | II-47 |
| B2-33 | I-11 | II-48 |
| B2-34 | I-12 | II-46 |
| B2-35 | I-12 | II-47 |
| B2-36 | I-12 | II-48 |
| B2-37 | I-13 | II-46 |
| B2-38 | I-13 | II-47 |
| B2-39 | I-13 | II-48 |
| B2-40 | I-14 | II-46 |
| B2-41 | I-14 | II-47 |
| B2-42 | I-14 | II-48 |
| B2-43 | I-15 | II-46 |
| B2-44 | I-15 | II-47 |
| B2-45 | I-15 | II-48 |
| B2-46 | I-16 | II-46 |
| B2-47 | I-16 | II-47 |
| B2-48 | I-16 | II-48 |
| B2-49 | I-17 | II-46 |
| B2-50 | I-17 | II-47 |
| B2-51 | I-17 | II-48 |
| B2-52 | I-18 | II-46 |
| B2-53 | I-18 | II-47 |
| B2-54 | I-18 | II-48 |

Other preferred components 2 are compounds II selected from the group of the following compounds:

| | |
|---|---|
| II-49 | azoxystrobin |
| II-50 | trifloxystrobin |
| II-51 | penthiopyrad |

Other preferred mixtures are the binary mixtures of the continuation of Table B2, where each row corresponds to one aspect of the mixtures according to the invention.

Continuation of Table B2: Binary mixtures comprising
one component 1 selected from compounds of the formula I
and one component 2 selected from groups A to F

Component

| Mixture | 1 | 2 |
|---|---|---|
| B2-55 | I-1 | II-49 |
| B2-56 | I-1 | II-50 |
| B2-57 | I-1 | II-51 |
| B2-58 | I-2 | II-49 |
| B2-59 | I-2 | II-50 |
| B2-60 | I-2 | II-51 |
| B2-61 | I-3 | II-49 |
| B2-62 | I-3 | II-50 |
| B2-63 | I-3 | II-51 |
| B2-64 | I-4 | II-49 |
| B2-65 | I-4 | II-50 |
| B2-66 | I-4 | II-51 |
| B2-67 | I-5 | II-49 |
| B2-68 | I-5 | II-50 |
| B2-69 | I-5 | II-51 |
| B2-70 | I-6 | II-49 |
| B2-71 | I-6 | II-50 |
| B2-72 | I-6 | II-51 |
| B2-73 | I-7 | II-49 |
| B2-74 | I-7 | II-50 |
| B2-75 | I-7 | II-51 |
| B2-76 | I-8 | II-49 |
| B2-77 | I-8 | II-50 |
| B2-78 | I-8 | II-51 |
| B2-79 | I-9 | II-49 |
| B2-80 | I-9 | II-50 |
| B2-81 | I-9 | II-51 |
| B2-82 | I-10 | II-49 |
| B2-83 | I-10 | II-50 |
| B2-84 | I-10 | II-51 |
| B2-85 | I-11 | II-49 |
| B2-86 | I-11 | II-50 |
| B2-87 | I-11 | II-51 |
| B2-88 | I-12 | II-49 |
| B2-89 | I-12 | II-50 |
| B2-90 | I-12 | II-51 |
| B2-91 | I-13 | II-49 |
| B2-92 | I-13 | II-50 |
| B2-93 | I-13 | II-51 |
| B2-94 | I-14 | II-49 |
| B2-95 | I-14 | II-50 |
| B2-96 | I-14 | II-51 |
| B2-97 | I-15 | II-49 |
| B2-98 | I-15 | II-50 |
| B2-99 | I-15 | II-51 |
| B2-100 | I-16 | II-49 |
| B2-101 | I-16 | II-50 |
| B2-102 | I-16 | II-51 |
| B2-103 | I-17 | II-49 |
| B2-104 | I-17 | II-50 |
| B2-105 | I-17 | II-51 |
| B2-106 | I-18 | II-49 |
| B2-107 | I-18 | II-50 |
| B2-108 | I-18 | II-51 |

In a preferred embodiment, the mixtures are the following binary mixtures:
I-3 and boscalid II-24
I-10 and pyraclostrobin II-9
I-14 and pyraclostrobin II-9
I-3 and epoxiconazole II-1
I-8 and orysastrobin II-10
I-1 and pyrimethanil II-13
I-3 and pyrimethanil II-13
I-10 and epoxiconazole II-1
I-10 and pyrimethanil II-13
I-12 and epoxiconazole II-1
I-12 and boscalid II-24
I-12 and pyrimethanil II-13
I-14 and pyrimethanil II-13
I-3 and chlorothalonil II-21
I-8 and epoxiconazole II-1
I-14 and epoxiconazole II-1
I-14 and chlorothalonil II-21

According to a further embodiment, particularly preferred components 2 are compounds II selected from the group of the following compounds:
II-1a acephate
II-2a chlorpyrifos
II-3a dimethoate
II-4a methamidophos
II-5a terbufos
II-6a aldicarb
II-7a carbofuran
II-8a bifenthrin
II-9a alpha-cypermethrin
II-10a deltamethrin
II-11a lambda-cyhalothrin
II-12a betacyfluthrin
II-13a zetacypermethrin
II-14a esfenvalerate
II-15a pirimicarb
II-16a tefluthrin
II-17a spirotetramat
II-18a endosulfan
II-19a abamectin
II-20a spinosad
II-21a spinetoram
II-22a hydramethylnon
II-23a fenbutatin oxide
II-24a chlorfenapyr
II-25a cyazypyr
II-26a cyflumetofen
II-27a flubendiamide
II-28a indoxacarb
II-29a metaflumizone
II-30a fipronil
II-31a [(3S,4R,4aR,6S,6aS,12R,12aS,12bS)-3-(cyclopropanecarbonyloxy)-6,12-dihydroxy-4,6a,12b-trimethyl-1′-oxo-9-(pyridin-3-yl)-1,2,3,4,4a,5,6,6a,12a,12b-decahydro-11H,12H-benzo[f]pyrano[4,3-b]chromen-4-yl]methyl cyclopropanecarboxylate.

Particularly preferred mixtures are the binary mixtures of Table B3, where each row corresponds to one aspect of the mixtures according to the invention.

TABLE B3
Binary mixtures comprising one component 1 selected
from preferred compounds of the formula I and one
component 2 selected from group I)

Component

| Mixture | 1 | 2 |
|---|---|---|
| B3-1 | I-1 | II-1a |
| B3-2 | I-1 | II-2a |
| B3-3 | I-1 | II-3a |
| B3-4 | I-1 | II-4a |
| B3-5 | I-1 | II-5a |
| B3-6 | I-1 | II-6a |
| B3-7 | I-1 | II-7a |
| B3-8 | I-1 | II-8a |
| B3-9 | I-1 | II-9a |
| B3-10 | I-1 | II-10a |
| B3-11 | I-1 | II-11a |
| B3-12 | I-1 | II-12a |
| B3-13 | I-1 | II-13a |
| B3-14 | I-1 | II-14a |
| B3-15 | I-1 | II-15a |
| B3-16 | I-1 | II-16a |
| B3-17 | I-1 | II-17a |
| B3-18 | I-1 | II-18a |
| B3-19 | I-1 | II-19a |
| B3-20 | I-1 | II-20a |
| B3-21 | I-1 | II-21a |
| B3-22 | I-1 | II-22a |
| B3-23 | I-1 | II-23a |
| B3-24 | I-1 | II-24a |
| B3-25 | I-1 | II-25a |
| B3-26 | I-1 | II-26a |
| B3-27 | I-1 | II-27a |
| B3-28 | I-1 | II-28a |
| B3-29 | I-1 | II-29a |
| B3-30 | I-1 | II-30a |
| B3-31 | I-1 | II-31a |
| B3-32 | I-2 | II-1a |
| B3-33 | I-2 | II-2a |
| B3-34 | I-2 | II-3a |
| B3-35 | I-2 | II-4a |
| B3-36 | I-2 | II-5a |
| B3-37 | I-2 | II-6a |
| B3-38 | I-2 | II-7a |
| B3-39 | I-2 | II-8a |
| B3-40 | I-2 | II-9a |
| B3-41 | I-2 | II-10a |
| B3-42 | I-2 | II-11a |
| B3-43 | I-2 | II-12a |
| B3-44 | I-2 | II-13a |
| B3-45 | I-2 | II-14a |
| B3-46 | I-2 | II-15a |
| B3-47 | I-2 | II-16a |
| B3-48 | I-2 | II-17a |
| B3-49 | I-2 | II-18a |
| B3-50 | I-2 | II-19a |
| B3-51 | I-2 | II-20a |
| B3-52 | I-2 | II-21a |
| B3-53 | I-2 | II-22a |
| B3-54 | I-2 | II-23a |
| B3-55 | I-2 | II-24a |
| B3-56 | I-2 | II-25a |
| B3-57 | I-2 | II-26a |
| B3-58 | I-2 | II-27a |
| B3-59 | I-2 | II-28a |
| B3-60 | I-2 | II-29a |
| B3-61 | I-2 | II-30a |
| B3-62 | I-2 | II-31a |
| B3-63 | I-3 | II-1a |
| B3-64 | I-3 | II-2a |
| B3-65 | I-3 | II-3a |
| B3-66 | I-3 | II-4a |
| B3-67 | I-3 | II-5a |
| B3-68 | I-3 | II-6a |
| B3-69 | I-3 | II-7a |
| B3-70 | I-3 | II-8a |
| B3-71 | I-3 | II-9a |
| B3-72 | I-3 | II-10a |
| B3-73 | I-3 | II-11a |
| B3-74 | I-3 | II-12a |
| B3-75 | I-3 | II-13a |
| B3-76 | I-3 | II-14a |
| B3-77 | I-3 | II-15a |
| B3-78 | I-3 | II-16a |
| B3-79 | I-3 | II-17a |
| B3-80 | I-3 | II-18a |
| B3-81 | I-3 | II-19a |
| B3-82 | I-3 | II-20a |
| B3-83 | I-3 | II-21a |
| B3-84 | I-3 | II-22a |
| B3-85 | I-3 | II-23a |
| B3-86 | I-3 | II-24a |
| B3-87 | I-3 | II-25a |
| B3-88 | I-3 | II-26a |
| B3-89 | I-3 | II-27a |
| B3-90 | I-3 | II-28a |
| B3-91 | I-3 | II-29a |
| B3-92 | I-3 | II-30a |
| B3-93 | I-3 | II-31a |
| B3-94 | I-4 | II-1a |
| B3-95 | I-4 | II-2a |
| B3-96 | I-4 | II-3a |
| B3-97 | I-4 | II-4a |
| B3-98 | I-4 | II-5a |
| B3-99 | I-4 | II-6a |
| B3-100 | I-4 | II-7a |
| B3-101 | I-4 | II-8a |
| B3-102 | I-4 | II-9a |
| B3-103 | I-4 | II-10a |
| B3-104 | I-4 | II-11a |
| B3-105 | I-4 | II-12a |
| B3-106 | I-4 | II-13a |
| B3-107 | I-4 | II-14a |
| B3-108 | I-4 | II-15a |
| B3-109 | I-4 | II-16a |
| B3-110 | I-4 | II-17a |
| B3-111 | I-4 | II-18a |
| B3-112 | I-4 | II-19a |
| B3-113 | I-4 | II-20a |
| B3-114 | I-4 | II-21a |
| B3-115 | I-4 | II-22a |
| B3-116 | I-4 | II-23a |
| B3-117 | I-4 | II-24a |
| B3-118 | I-4 | II-25a |
| B3-119 | I-4 | II-26a |
| B3-120 | I-4 | II-27a |
| B3-121 | I-4 | II-28a |
| B3-122 | I-4 | II-29a |
| B3-123 | I-4 | II-30a |
| B3-124 | I-4 | II-31a |
| B3-125 | I-5 | II-1a |
| B3-126 | I-5 | II-2a |
| B3-127 | I-5 | II-3a |
| B3-128 | I-5 | II-4a |
| B3-129 | I-5 | II-5a |
| B3-130 | I-5 | II-6a |
| B3-131 | I-5 | II-7a |
| B3-132 | I-5 | II-8a |
| B3-133 | I-5 | II-9a |
| B3-134 | I-5 | II-10a |
| B3-135 | I-5 | II-11a |
| B3-136 | I-5 | II-12a |
| B3-137 | I-5 | II-13a |
| B3-138 | I-5 | II-14a |
| B3-139 | I-5 | II-15a |
| B3-140 | I-5 | II-16a |
| B3-141 | I-5 | II-17a |
| B3-142 | I-5 | II-18a |
| B3-143 | I-5 | II-19a |
| B3-144 | I-5 | II-20a |
| B3-145 | I-5 | II-21a |
| B3-146 | I-5 | II-22a |
| B3-147 | I-5 | II-23a |
| B3-148 | I-5 | II-24a |
| B3-149 | I-5 | II-25a |
| B3-150 | I-5 | II-26a |
| B3-151 | I-5 | II-27a |
| B3-152 | I-5 | II-28a |
| B3-153 | I-5 | II-29a |
| B3-154 | I-5 | II-30a |
| B3-155 | I-5 | II-31a |
| B3-156 | I-6 | II-1a |
| B3-157 | I-6 | II-2a |
| B3-158 | I-6 | II-3a |
| B3-159 | I-6 | II-4a |
| B3-160 | I-6 | II-5a |
| B3-161 | I-6 | II-6a |
| B3-162 | I-6 | II-7a |
| B3-163 | I-6 | II-8a |
| B3-164 | I-6 | II-9a |
| B3-165 | I-6 | II-10a |
| B3-166 | I-6 | II-11a |
| B3-167 | I-6 | II-12a |
| B3-168 | I-6 | II-13a |
| B3-169 | I-6 | II-14a |
| B3-170 | I-6 | II-15a |
| B3-171 | I-6 | II-16a |
| B3-172 | I-6 | II-17a |
| B3-173 | I-6 | II-18a |
| B3-174 | I-6 | II-19a |
| B3-175 | I-6 | II-20a |
| B3-176 | I-6 | II-21a |
| B3-177 | I-6 | II-22a |
| B3-178 | I-6 | II-23a |
| B3-179 | I-6 | II-24a |
| B3-180 | I-6 | II-25a |
| B3-181 | I-6 | II-26a |
| B3-182 | I-6 | II-27a |
| B3-183 | I-6 | II-28a |
| B3-184 | I-6 | II-29a |
| B3-185 | I-6 | II-30a |
| B3-186 | I-6 | II-31a |
| B3-187 | I-7 | II-1a |
| B3-188 | I-7 | II-2a |
| B3-189 | I-7 | II-3a |
| B3-190 | I-7 | II-4a |
| B3-191 | I-7 | II-5a |
| B3-192 | I-7 | II-6a |
| B3-193 | I-7 | II-7a |
| B3-194 | I-7 | II-8a |
| B3-195 | I-7 | II-9a |
| B3-196 | I-7 | II-10a |
| B3-197 | I-7 | II-11a |
| B3-198 | I-7 | II-12a |
| B3-199 | I-7 | II-13a |
| B3-200 | I-7 | II-14a |
| B3-201 | I-7 | II-15a |
| B3-202 | I-7 | II-16a |
| B3-203 | I-7 | II-17a |
| B3-204 | I-7 | II-18a |
| B3-205 | I-7 | II-19a |
| B3-206 | I-7 | II-20a |
| B3-207 | I-7 | II-21a |
| B3-208 | I-7 | II-22a |
| B3-209 | I-7 | II-23a |
| B3-210 | I-7 | II-24a |
| B3-211 | I-7 | II-25a |
| B3-212 | I-7 | II-26a |
| B3-213 | I-7 | II-27a |
| B3-214 | I-7 | II-28a |
| B3-215 | I-7 | II-29a |
| B3-216 | I-7 | II-30a |
| B3-217 | I-7 | II-31a |
| B3-218 | I-8 | II-1a |
| B3-219 | I-8 | II-2a |
| B3-220 | I-8 | II-3a |
| B3-221 | I-8 | II-4a |
| B3-222 | I-8 | II-5a |
| B3-223 | I-8 | II-6a |
| B3-224 | I-8 | II-7a |
| B3-225 | I-8 | II-8a |
| B3-226 | I-8 | II-9a |
| B3-227 | I-8 | II-10a |
| B3-228 | I-8 | II-11a |
| B3-229 | I-8 | II-12a |
| B3-230 | I-8 | II-13a |
| B3-231 | I-8 | II-14a |
| B3-232 | I-8 | II-15a |
| B3-233 | I-8 | II-16a |
| B3-234 | I-8 | II-17a |
| B3-235 | I-8 | II-18a |
| B3-236 | I-8 | II-19a |
| B3-237 | I-8 | II-20a |
| B3-238 | I-8 | II-21a |
| B3-239 | I-8 | II-22a |
| B3-240 | I-8 | II-23a |
| B3-241 | I-8 | II-24a |
| B3-242 | I-8 | II-25a |
| B3-243 | I-8 | II-26a |
| B3-244 | I-8 | II-27a |
| B3-245 | I-8 | II-28a |
| B3-246 | I-8 | II-29a |
| B3-247 | I-8 | II-30a |
| B3-248 | I-8 | II-31a |
| B3-249 | I-9 | II-1a |
| B3-250 | I-9 | II-2a |
| B3-251 | I-9 | II-3a |
| B3-252 | I-9 | II-4a |
| B3-253 | I-9 | II-5a |
| B3-254 | I-9 | II-6a |
| B3-255 | I-9 | II-7a |
| B3-256 | I-9 | II-8a |
| B3-257 | I-9 | II-9a |
| B3-258 | I-9 | II-10a |
| B3-259 | I-9 | II-11a |
| B3-260 | I-9 | II-12a |
| B3-261 | I-9 | II-13a |
| B3-262 | I-9 | II-14a |
| B3-263 | I-9 | II-15a |
| B3-264 | I-9 | II-16a |
| B3-265 | I-9 | II-17a |
| B3-266 | I-9 | II-18a |
| B3-267 | I-9 | II-19a |
| B3-268 | I-9 | II-20a |
| B3-269 | I-9 | II-21a |
| B3-270 | I-9 | II-22a |
| B3-271 | I-9 | II-23a |
| B3-272 | I-9 | II-24a |
| B3-273 | I-9 | II-25a |
| B3-274 | I-9 | II-26a |
| B3-275 | I-9 | II-27a |
| B3-276 | I-9 | II-28a |
| B3-277 | I-9 | II-29a |
| B3-278 | I-9 | II-30a |
| B3-279 | I-9 | II-31a |
| B3-280 | I-10 | II-1a |
| B3-281 | I-10 | II-2a |
| B3-282 | I-10 | II-3a |
| B3-283 | I-10 | II-4a |
| B3-284 | I-10 | II-5a |
| B3-285 | I-10 | II-6a |
| B3-286 | I-10 | II-7a |
| B3-287 | I-10 | II-8a |
| B3-288 | I-10 | II-9a |
| B3-289 | I-10 | II-10a |
| B3-290 | I-10 | II-11a |
| B3-291 | I-10 | II-12a |
| B3-292 | I-10 | II-13a |
| B3-293 | I-10 | II-14a |
| B3-294 | I-10 | II-15a |
| B3-295 | I-10 | II-16a |
| B3-296 | I-10 | II-17a |
| B3-297 | I-10 | II-18a |
| B3-298 | I-10 | II-19a |
| B3-299 | I-10 | II-20a |
| B3-300 | I-10 | II-21a |
| B3-301 | I-10 | II-22a |
| B3-302 | I-10 | II-23a |
| B3-303 | I-10 | II-24a |
| B3-304 | I-10 | II-25a |
| B3-305 | I-10 | II-26a |
| B3-306 | I-10 | II-27a |
| B3-307 | I-10 | II-28a |
| B3-308 | I-10 | II-29a |
| B3-309 | I-10 | II-30a |
| B3-310 | I-10 | II-31a |
| B3-311 | I-11 | II-1a |
| B3-312 | I-11 | II-2a |
| B3-313 | I-11 | II-3a |
| B3-314 | I-11 | II-4a |
| B3-315 | I-11 | II-5a |
| B3-316 | I-11 | II-6a |
| B3-317 | I-11 | II-7a |
| B3-318 | I-11 | II-8a |
| B3-319 | I-11 | II-9a |
| B3-320 | I-11 | II-10a |
| B3-321 | I-11 | II-11a |
| B3-322 | I-11 | II-12a |
| B3-323 | I-11 | II-13a |
| B3-324 | I-11 | II-14a |
| B3-325 | I-11 | II-15a |
| B3-326 | I-11 | II-16a |
| B3-327 | I-11 | II-17a |
| B3-328 | I-11 | II-18a |
| B3-329 | I-11 | II-19a |
| B3-330 | I-11 | II-20a |
| B3-331 | I-11 | II-21a |
| B3-332 | I-11 | II-22a |
| B3-333 | I-11 | II-23a |
| B3-334 | I-11 | II-24a |
| B3-335 | I-11 | II-25a |
| B3-336 | I-11 | II-26a |
| B3-337 | I-11 | II-27a |
| B3-338 | I-11 | II-28a |
| B3-339 | I-11 | II-29a |
| B3-340 | I-11 | II-30a |
| B3-341 | I-11 | II-31a |
| B3-342 | I-12 | II-1a |
| B3-343 | I-12 | II-2a |
| B3-344 | I-12 | II-3a |
| B3-345 | I-12 | II-4a |
| B3-346 | I-12 | II-5a |
| B3-347 | I-12 | II-6a |
| B3-348 | I-12 | II-7a |
| B3-349 | I-12 | II-8a |
| B3-350 | I-12 | II-9a |
| B3-351 | I-12 | II-10a |
| B3-352 | I-12 | II-11a |
| B3-353 | I-12 | II-12a |
| B3-354 | I-12 | II-13a |
| B3-355 | I-12 | II-14a |
| B3-356 | I-12 | II-15a |
| B3-357 | I-12 | II-16a |
| B3-358 | I-12 | II-17a |
| B3-359 | I-12 | II-18a |
| B3-360 | I-12 | II-19a |
| B3-361 | I-12 | II-20a |
| B3-362 | I-12 | II-21a |
| B3-363 | I-12 | II-22a |
| B3-364 | I-12 | II-23a |
| B3-365 | I-12 | II-24a |
| B3-366 | I-12 | II-25a |
| B3-367 | I-12 | II-26a |
| B3-368 | I-12 | II-27a |
| B3-369 | I-12 | II-28a |
| B3-370 | I-12 | II-29a |
| B3-371 | I-12 | II-30a |
| B3-372 | I-12 | II-31a |
| B3-373 | I-13 | II-1a |
| B3-374 | I-13 | II-2a |
| B3-375 | I-13 | II-3a |
| B3-376 | I-13 | II-4a |
| B3-377 | I-13 | II-5a |
| B3-378 | I-13 | II-6a |
| B3-379 | I-13 | II-7a |
| B3-380 | I-13 | II-8a |
| B3-381 | I-13 | II-9a |
| B3-382 | I-13 | II-10a |
| B3-383 | I-13 | II-11a |
| B3-384 | I-13 | II-12a |
| B3-385 | I-13 | II-13a |
| B3-386 | I-13 | II-14a |
| B3-387 | I-13 | II-15a |
| B3-388 | I-13 | II-16a |
| B3-389 | I-13 | II-17a |
| B3-390 | I-13 | II-18a |
| B3-391 | I-13 | II-19a |
| B3-392 | I-13 | II-20a |
| B3-393 | I-13 | II-21a |
| B3-394 | I-13 | II-22a |
| B3-395 | I-13 | II-23a |
| B3-396 | I-13 | II-24a |
| B3-397 | I-13 | II-25a |
| B3-398 | I-13 | II-26a |
| B3-399 | I-13 | II-27a |
| B3-400 | I-13 | II-28a |
| B3-401 | I-13 | II-29a |
| B3-402 | I-13 | II-30a |
| B3-403 | I-13 | II-31a |
| B3-404 | I-14 | II-1a |
| B3-405 | I-14 | II-2a |
| B3-406 | I-14 | II-3a |
| B3-407 | I-14 | II-4a |
| B3-408 | I-14 | II-5a |
| B3-409 | I-14 | II-6a |
| B3-410 | I-14 | II-7a |
| B3-411 | I-14 | II-8a |
| B3-412 | I-14 | II-9a |
| B3-413 | I-14 | II-10a |
| B3-414 | I-14 | II-11a |
| B3-415 | I-14 | II-12a |
| B3-416 | I-14 | II-13a |
| B3-417 | I-14 | II-14a |
| B3-418 | I-14 | II-15a |
| B3-419 | I-14 | II-16a |
| B3-420 | I-14 | II-17a |
| B3-421 | I-14 | II-18a |
| B3-422 | I-14 | II-19a |
| B3-423 | I-14 | II-20a |
| B3-424 | I-14 | II-21a |
| B3-425 | I-14 | II-22a |
| B3-426 | I-14 | II-23a |
| B3-427 | I-14 | II-24a |
| B3-428 | I-14 | II-25a |
| B3-429 | I-14 | II-26a |
| B3-430 | I-14 | II-27a |
| B3-431 | I-14 | II-28a |
| B3-432 | I-14 | II-29a |
| B3-433 | I-14 | II-30a |
| B3-434 | I-14 | II-31a |
| B3-435 | I-15 | II-1a |
| B3-436 | I-15 | II-2a |
| B3-437 | I-15 | II-3a |
| B3-438 | I-15 | II-4a |
| B3-439 | I-15 | II-5a |
| B3-440 | I-15 | II-6a |
| B3-441 | I-15 | II-7a |
| B3-442 | I-15 | II-8a |
| B3-443 | I-15 | II-9a |
| B3-444 | I-15 | II-10a |
| B3-445 | I-15 | II-11a |
| B3-446 | I-15 | II-12a |
| B3-447 | I-15 | II-13a |
| B3-448 | I-15 | II-14a |
| B3-449 | I-15 | II-15a |
| B3-450 | I-15 | II-16a |
| B3-451 | I-15 | II-17a |
| B3-452 | I-15 | II-18a |
| B3-453 | I-15 | II-19a |
| B3-454 | I-15 | II-20a |
| B3-455 | I-15 | II-21a |
| B3-456 | I-15 | II-22a |
| B3-457 | I-15 | II-23a |
| B3-458 | I-15 | II-24a |
| B3-459 | I-15 | II-25a |
| B3-460 | I-15 | II-26a |
| B3-461 | I-15 | II-27a |
| B3-462 | I-15 | II-28a |
| B3-463 | I-15 | II-29a |
| B3-464 | I-15 | II-30a |
| B3-465 | I-15 | II-31a |
| B3-466 | I-16 | II-1a |
| B3-467 | I-16 | II-2a |
| B3-468 | I-16 | II-3a |
| B3-469 | I-16 | II-4a |
| B3-470 | I-16 | II-5a |
| B3-471 | I-16 | II-6a |
| B3-472 | I-16 | II-7a |
| B3-473 | I-16 | II-8a |
| B3-474 | I-16 | II-9a |
| B3-475 | I-16 | II-10a |
| B3-476 | I-16 | II-11a |
| B3-477 | I-16 | II-12a |
| B3-478 | I-16 | II-13a |
| B3-479 | I-16 | II-14a |
| B3-480 | I-16 | II-15a |
| B3-481 | I-16 | II-16a |
| B3-482 | I-16 | II-17a |
| B3-483 | I-16 | II-18a |
| B3-484 | I-16 | II-19a |
| B3-485 | I-16 | II-20a |
| B3-486 | I-16 | II-21a |
| B3-487 | I-16 | II-22a |
| B3-488 | I-16 | II-23a |
| B3-489 | I-16 | II-24a |
| B3-490 | I-16 | II-25a |
| B3-491 | I-16 | II-26a |
| B3-492 | I-16 | II-27a |
| B3-493 | I-16 | II-28a |
| B3-494 | I-16 | II-29a |
| B3-495 | I-16 | II-30a |
| B3-496 | I-16 | II-31a |
| B3-497 | I-17 | II-1a |
| B3-498 | I-17 | II-2a |
| B3-499 | I-17 | II-3a |
| B3-500 | I-17 | II-4a |
| B3-501 | I-17 | II-5a |
| B3-502 | I-17 | II-6a |
| B3-503 | I-17 | II-7a |
| B3-504 | I-17 | II-8a |
| B3-505 | I-17 | II-9a |
| B3-506 | I-17 | II-10a |
| B3-507 | I-17 | II-11a |
| B3-508 | I-17 | II-12a |
| B3-509 | I-17 | II-13a |
| B3-510 | I-17 | II-14a |
| B3-511 | I-17 | II-15a |
| B3-512 | I-17 | II-16a |
| B3-513 | I-17 | II-17a |
| B3-514 | I-17 | II-18a |
| B3-515 | I-17 | II-19a |
| B3-516 | I-17 | II-20a |
| B3-517 | I-17 | II-21a |
| B3-518 | I-17 | II-22a |
| B3-519 | I-17 | II-23a |
| B3-520 | I-17 | II-24a |
| B3-521 | I-17 | II-25a |
| B3-522 | I-17 | II-26a |
| B3-523 | I-17 | II-27a |
| B3-524 | I-17 | II-28a |
| B3-525 | I-17 | II-29a |
| B3-526 | I-17 | II-30a |
| B3-527 | I-17 | II-31a |
| B3-528 | I-18 | II-1a |
| B3-529 | I-18 | II-2a |
| B3-530 | I-18 | II-3a |
| B3-531 | I-18 | II-4a |
| B3-532 | I-18 | II-5a |
| B3-533 | I-18 | II-6a |
| B3-534 | I-18 | II-7a |
| B3-535 | I-18 | II-8a |
| B3-536 | I-18 | II-9a |
| B3-537 | I-18 | II-10a |
| B3-538 | I-18 | II-11a |
| B3-539 | I-18 | II-12a |
| B3-540 | I-18 | II-13a |
| B3-541 | I-18 | II-14a |
| B3-542 | I-18 | II-15a |
| B3-543 | I-18 | II-16a |
| B3-544 | I-18 | II-17a |
| B3-545 | I-18 | II-18a |
| B3-546 | I-18 | II-19a |
| B3-547 | I-18 | II-21a |
| B3-548 | I-18 | II-21a |
| B3-549 | I-18 | II-22a |
| B3-550 | I-18 | II-23a |
| B3-551 | I-18 | II-24a |
| B3-552 | I-18 | II-25a |
| B3-553 | I-18 | II-26a |
| B3-554 | I-18 | II-27a |
| B3-555 | I-18 | II-28a |
| B3-556 | I-18 | II-29a |
| B3-557 | I-18 | II-30a |
| B3-558 | I-18 | II-31a |

According to a further embodiment, particularly preferred components 2 are compounds II selected from the group of the following compounds:

| | |
|---|---|
| II-32a | clothianidin |
| II-33a | dinotefuran |
| II-34a | imidacloprid |
| II-35a | thiamethoxam |
| II-36a | nitenpyram |
| II-37a | acetamiprid |
| II-38a | thiacloprid |

Particularly preferred mixtures are the binary mixtures of Table B4, where each row corresponds to one aspect of the mixtures according to the invention.

TABLE B4
Binary mixtures comprising one component 1 selected from preferred
compounds of the formula I and one component 2 selected from group I

Component

| Mixture | 1 | 2 |
|---|---|---|
| B4-1 | I-1 | II-32a |
| B4-2 | I-1 | II-33a |
| B4-3 | I-1 | II-34a |
| B4-4 | I-1 | II-35a |
| B4-5 | I-1 | II-36a |
| B4-6 | I-1 | II-37a |
| B4-7 | I-1 | II-38a |
| B4-8 | I-2 | II-32a |
| B4-9 | I-2 | II-33a |
| B4-10 | I-2 | II-34a |
| B4-11 | I-2 | II-35a |
| B4-12 | I-2 | II-36a |
| B4-13 | I-2 | II-37a |
| B4-14 | I-2 | II-38a |
| B4-15 | I-3 | II-32a |
| B4-16 | I-3 | II-33a |
| B4-17 | I-3 | II-34a |
| B4-18 | I-3 | II-35a |
| B4-19 | I-3 | II-36a |
| B4-20 | I-3 | II-37a |
| B4-21 | I-3 | II-38a |
| B4-22 | I-4 | II-32a |
| B4-23 | I-4 | II-33a |
| B4-24 | I-4 | II-34a |
| B4-25 | I-4 | II-35a |
| B4-26 | I-4 | II-36a |
| B4-27 | I-4 | II-37a |
| B4-28 | I-4 | II-38a |
| B4-29 | I-5 | II-32a |
| B4-30 | I-5 | II-33a |
| B4-31 | I-5 | II-34a |
| B4-32 | I-5 | II-35a |
| B4-33 | I-5 | II-36a |
| B4-34 | I-5 | II-37a |
| B4-35 | I-5 | II-38a |
| B4-36 | I-6 | II-32a |
| B4-37 | I-6 | II-33a |
| B4-38 | I-6 | II-34a |
| B4-39 | I-6 | II-35a |
| B4-40 | I-6 | II-36a |
| B4-41 | I-6 | II-37a |
| B4-42 | I-6 | II-38a |
| B4-43 | I-7 | II-32a |
| B4-44 | I-7 | II-33a |
| B4-45 | I-7 | II-34a |
| B4-46 | I-7 | II-35a |
| B4-47 | I-7 | II-36a |
| B4-48 | I-7 | II-37a |
| B4-49 | I-7 | II-38a |
| B4-50 | I-8 | II-32a |
| B4-51 | I-8 | II-33a |
| B4-52 | I-8 | II-34a |
| B4-53 | I-8 | II-35a |
| B4-54 | I-8 | II-36a |
| B4-55 | I-8 | II-37a |
| B4-56 | I-8 | II-38a |
| B4-57 | I-9 | II-32a |
| B4-58 | I-9 | II-33a |
| B4-59 | I-9 | II-34a |
| B4-60 | I-9 | II-35a |
| B4-61 | I-9 | II-36a |
| B4-62 | I-9 | II-37a |
| B4-63 | I-9 | II-38a |
| B4-64 | I-10 | II-32a |
| B4-65 | I-10 | II-33a |
| B4-66 | I-10 | II-34a |
| B4-67 | I-10 | II-35a |
| B4-68 | I-10 | II-36a |
| B4-69 | I-10 | II-37a |
| B4-70 | I-10 | II-38a |
| B4-71 | I-11 | II-32a |
| B4-72 | I-11 | II-33a |
| B4-73 | I-11 | II-34a |
| B4-74 | I-11 | II-35a |
| B4-75 | I-11 | II-36a |
| B4-76 | I-11 | II-37a |
| B4-77 | I-11 | II-38a |
| B4-78 | I-12 | II-32a |
| B4-79 | I-12 | II-33a |
| B4-80 | I-12 | II-34a |
| B4-81 | I-12 | II-35a |
| B4-82 | I-12 | II-36a |
| B4-83 | I-12 | II-37a |
| B4-84 | I-12 | II-38a |
| B4-85 | I-13 | II-32a |
| B4-86 | I-13 | II-33a |
| B4-87 | I-13 | II-34a |
| B4-88 | I-13 | II-35a |
| B4-89 | I-13 | II-36a |
| B4-90 | I-13 | II-37a |
| B4-91 | I-13 | II-38a |
| B4-92 | I-14 | II-32a |
| B4-93 | I-14 | II-33a |
| B4-94 | I-14 | II-34a |
| B4-95 | I-14 | II-35a |
| B4-96 | I-14 | II-36a |
| B4-97 | I-14 | II-37a |
| B4-98 | I-14 | II-38a |
| B4-99 | I-15 | II-32a |
| B4-100 | I-15 | II-33a |
| B4-101 | I-15 | II-34a |
| B4-102 | I-15 | II-35a |
| B4-103 | I-15 | II-36a |
| B4-104 | I-15 | II-37a |
| B4-105 | I-15 | II-38a |
| B4-106 | I-16 | II-32a |
| B4-107 | I-16 | II-33a |
| B4-108 | I-16 | II-34a |
| B4-109 | I-16 | II-35a |
| B4-110 | I-16 | II-36a |
| B4-111 | I-16 | II-37a |
| B4-112 | I-16 | II-38a |
| B4-113 | I-17 | II-32a |
| B4-114 | I-17 | II-33a |
| B4-115 | I-17 | II-34a |
| B4-116 | I-17 | II-35a |
| B4-117 | I-17 | II-36a |
| B4-118 | I-17 | II-37a |
| B4-119 | I-17 | II-38a |
| B4-120 | I-18 | II-32a |
| B4-121 | I-18 | II-33a |
| B4-122 | I-18 | II-34a |
| B4-123 | I-18 | II-35a |
| B4-124 | I-18 | II-36a |
| B4-125 | I-18 | II-37a |
| B4-126 | I-18 | II-38a |

According to a further embodiment, particularly preferred components 2 are compounds II selected from the active compounds of group H).

Special preference is given to the active compounds according to this embodiment selected from the group of the following compounds of group H):

| | |
|---|---|
| II-1b | glyphosate |
| II-2b | imazamox |

Particularly preferred mixtures are the binary mixtures of Table B5, where each row corresponds to one aspect of the mixtures according to the invention.

TABLE B5
Binary mixtures comprising one component 1 selected from preferred
compounds of the formula I and one component 2 selected from group H

Component

| Mixture | 1 | 2 |
|---|---|---|
| B5-1 | I-1 | II-1b |
| B5-2 | I-1 | II-2b |
| B5-3 | I-2 | II-1b |
| B5-4 | I-2 | II-2b |
| B5-5 | I-3 | II-1b |
| B5-6 | I-3 | II-2b |
| B5-7 | I-4 | II-1b |
| B5-8 | I-4 | II-2b |
| B5-9 | I-5 | II-1b |
| B5-10 | I-5 | II-2b |
| B5-11 | I-6 | II-1b |
| B5-12 | I-6 | II-2b |
| B5-13 | I-7 | II-1b |
| B5-14 | I-7 | II-2b |
| B5-15 | I-8 | II-1b |
| B5-16 | I-8 | II-2b |
| B5-17 | I-9 | II-1b |
| B5-18 | I-9 | II-2b |
| B5-19 | I-10 | II-1b |
| B5-20 | I-10 | II-2b |
| B5-21 | I-11 | II-1b |
| B5-22 | I-11 | II-2b |
| B5-23 | I-12 | II-1b |
| B5-24 | I-12 | II-2b |
| B5-25 | I-13 | II-1b |
| B5-26 | I-13 | II-2b |
| B5-27 | I-14 | II-1b |
| B5-28 | I-14 | II-2b |
| B5-29 | I-15 | II-1b |
| B5-30 | I-15 | II-2b |
| B5-31 | I-16 | II-1b |
| B5-32 | I-16 | II-2b |
| B5-33 | I-17 | II-1b |
| B5-34 | I-17 | II-2b |
| B5-35 | I-18 | II-1b |
| B5-36 | I-18 | II-2b |

In a preferred embodiment, the invention relates to fungicidal mixtures comprising a component 1, and also component 2 (compound II) and a further component 3 (further component 3), with the proviso that component 2 and component 3 are not identical.

More preferred mixtures are the ternary mixtures of Table T, where each row corresponds to one aspect of the mixtures according to the invention.

Moreover, the present invention furthermore relates to compositions of a compound I (component 1) with two further active compounds selected from the compounds II (component 2 and component 3). The present invention relates in particular to compositions of the mixtures, described as being preferred, of the compounds I and II with a further active compound II. Particular preference is given here to ternary mixtures.

A further embodiment of the invention relates to the compositions T-1 to T-359 listed in Table T, where a row of Table T corresponds in each case to an agrochemical composition comprising one of the mixtures, individualized in the present description, of the compounds of the formula I and the compounds II (components 1 and 2) and the respective further active compound from the groups A) to I) (component 3) stated in the row in question. In the compositions described, the active compounds are in each case preferably present in synergistically effective amounts.

The invention relates in particular to the compositions of the mixtures B-1 to B-359 with a further compound II selected from rows T-1 to T-359 in the column “Component 3” of Table T, particularly preferably selected from the compounds II-1 to 11-29. In each case, components 2 and 3 must not be identical.

TABLE T
Ternary mixtures comprising an individualized mixture of component 1 and
component 2 (mixture B), and also a component 3 selected from groups A to I:

| Row | Components 1 and 2 | Component 3 |
|---|---|---|
| T-1 | an individualized mixture B | azoxystrobin |
| T-2 | an individualized mixture B | dimoxystrobin |
| T-3 | an individualized mixture B | enestroburin |
| T-4 | an individualized mixture B | fluoxastrobin |
| T-5 | an individualized mixture B | kresoxim-methyl |
| T-6 | an individualized mixture B | metominostrobin |
| T-7 | an individualized mixture B | orysastrobin |
| T-8 | an individualized mixture B | picoxystrobin |
| T-9 | an individualized mixture B | pyraclostrobin |
| T-10 | an individualized mixture B | pyribencarb |
| T-11 | an individualized mixture B | trifloxystrobin |
| T-12 | an individualized mixture B | 2-(2-(6-(3-chloro-2-methylphenoxy)- |
| | | 5-fluoropyrimidin-4-yloxy)phenyl)- |
| | | 2-methoxyimino-N-methylacetamide |
| T-13 | an individualized mixture B | methyl 2-(o-((2,5-dimethylphenyloxy- |
| | | methylene)phenyl)-3-methoxyacrylate |
| T-14 | an individualized mixture B | methyl 3-methoxy-2-(2-(N-(4- |
| | | methoxy- |
| | | phenyl)cyclopropanecarboximidoylsulfanylmethyl)phenyl)acrylate |
| T-15 | an individualized mixture B | 2-(2-(3-(2,6-dichlorophenyl)-1- |
| | | methylallylideneaminooxymethyl)phenyl)- |
| | | 2-methoxyimino-N- |
| | | methylacetamide |
| T-16 | an individualized mixture B | benalaxyl |
| T-17 | an individualized mixture B | benalaxyl-M |
| T-18 | an individualized mixture B | benodanil |
| T-19 | an individualized mixture B | bixafen |
| T-20 | an individualized mixture B | boscalid |
| T-21 | an individualized mixture B | carboxin |
| T-22 | an individualized mixture B | fenfuram |
| T-23 | an individualized mixture B | fenhexamid |
| T-24 | an individualized mixture B | flutolanil |
| T-25 | an individualized mixture B | furametpyr |
| T-26 | an individualized mixture B | isopyrazam |
| T-27 | an individualized mixture B | isotianil |
| T-28 | an individualized mixture B | kiralaxyl |
| T-29 | an individualized mixture B | mepronil |
| T-30 | an individualized mixture B | metalaxyl |
| T-31 | an individualized mixture B | metalaxyl-M |
| T-32 | an individualized mixture B | ofurace |
| T-33 | an individualized mixture B | oxadixyl |
| T-34 | an individualized mixture B | oxycarboxin |
| T-35 | an individualized mixture B | penflufen |
| T-36 | an individualized mixture B | penthiopyrad |
| T-37 | an individualized mixture B | sedaxane |
| T-38 | an individualized mixture B | tecloftalam |
| T-39 | an individualized mixture B | thifluzamide |
| T-40 | an individualized mixture B | tiadinil |
| T-41 | an individualized mixture B | 2-amino-4-methylthiazole-5- |
| | | carboxanilide |
| T-42 | an individualized mixture B | 2-chloro-N-(1,1,3-trimethylindan-4-yl)- |
| | | nicotinamide |
| T-43 | an individualized mixture B | N-(3′,4′,5′-trifluorobiphenyl-2-yl)-3- |
| | | difluoromethyl-1-methyl-1H-pyrazole- |
| | | 4-carboxamide |
| T-44 | an individualized mixture B | N-(4′-trifluoromethylthiobiphenyl-2-yl)- |
| | | 3-difluoromethyl-1-methyl-1H- |
| | | pyrazole-4-carboxamide |
| T-45 | an individualized mixture B | N-(2-(1,3-dimethylbutyl)phenyl)-1,3- |
| | | dimethyl-5-fluoro-1H-pyrazole-4- |
| | | carboxamide |
| T-46 | an individualized mixture B | N-(2-(1,3,3-trimethylbutyl)phenyl)-1,3- |
| | | dimethyl-5-fluoro-1H-pyrazole-4- |
| | | carboxamide |
| T-47 | an individualized mixture B | dimethomorph |
| T-48 | an individualized mixture B | flumorph |
| T-49 | an individualized mixture B | pyrimorph |
| T-50 | an individualized mixture B | flumetover |
| T-51 | an individualized mixture B | fluopicolide |
| T-52 | an individualized mixture B | fluopyram |
| T-53 | an individualized mixture B | zoxamide |
| T-54 | an individualized mixture B | N-(3-ethyl-3,5,5-trimethylcyclohexyl)- |
| | | 3-formylamino-2-hydroxybenzamide |
| T-55 | an individualized mixture B | carpropamid |
| T-56 | an individualized mixture B | diclocymet |
| T-57 | an individualized mixture B | mandipropamid |
| T-58 | an individualized mixture B | oxytetracyclin |
| T-59 | an individualized mixture B | silthiofam |
| T-60 | an individualized mixture B | N-(6-methoxypyridin-3- |
| | | yl)cyclopropanecarboxamide |
| T-61 | an individualized mixture B | azaconazole |
| T-62 | an individualized mixture B | bitertanol |
| T-63 | an individualized mixture B | bromuconazole |
| T-64 | an individualized mixture B | cyproconazole |
| T-65 | an individualized mixture B | difenoconazole |
| T-66 | an individualized mixture B | diniconazole |
| T-67 | an individualized mixture B | diniconazole-M |
| T-68 | an individualized mixture B | epoxiconazole |
| T-69 | an individualized mixture B | fenbuconazole |
| T-70 | an individualized mixture B | fluquinconazole |
| T-71 | an individualized mixture B | flusilazole |
| T-72 | an individualized mixture B | flutriafol |
| T-73 | an individualized mixture B | hexaconazole |
| T-74 | an individualized mixture B | imibenconazole |
| T-75 | an individualized mixture B | ipconazole |
| T-76 | an individualized mixture B | metconazole |
| T-77 | an individualized mixture B | myclobutanil |
| T-78 | an individualized mixture B | oxpoconazole |
| T-79 | an individualized mixture B | paclobutrazole |
| T-80 | an individualized mixture B | penconazole |
| T-81 | an individualized mixture B | propiconazole |
| T-82 | an individualized mixture B | prothioconazole |
| T-83 | an individualized mixture B | simeconazole |
| T-84 | an individualized mixture B | tebuconazole |
| T-85 | an individualized mixture B | tetraconazole |
| T-86 | an individualized mixture B | triadimefon |
| T-87 | an individualized mixture B | triadimenol |
| T-88 | an individualized mixture B | triticonazole |
| T-89 | an individualized mixture B | uniconazole |
| T-90 | an individualized mixture B | 1-(4-chlorophenyl)-2-([1,2,4]triazol-1- |
| | | yl)-cycloheptanol |
| T-91 | an individualized mixture B | cyazofamid |
| T-92 | an individualized mixture B | imazalil |
| T-93 | an individualized mixture B | imazalil-sulfate |
| T-94 | an individualized mixture B | pefurazoate |
| T-95 | an individualized mixture B | prochloraz |
| T-96 | an individualized mixture B | triflumizole |
| T-97 | an individualized mixture B | benomyl |
| T-98 | an individualized mixture B | carbendazim |
| T-99 | an individualized mixture B | fuberidazole |
| T-100 | an individualized mixture B | thiabendazole |
| T-101 | an individualized mixture B | ethaboxam |
| T-102 | an individualized mixture B | etridiazole |
| T-103 | an individualized mixture B | hymexazole |
| T-104 | an individualized mixture B | 2-(4-chlorophenyl)-N-[4-(3,4- |
| | | dimethoxyphenyl)isoxazol-5-yl]-2- |
| | | prop-2-ynyloxyacetamide |
| T-105 | an individualized mixture B | fluazinam |
| T-106 | an individualized mixture B | pyrifenox |
| T-107 | an individualized mixture B | 3-[5-(4-chlorophenyl)-2,3-dimethyl- |
| | | isoxazolidin-3-yl]pyridine |
| T-108 | an individualized mixture B | 3-[5-(4-methylphenyl)-2,3- |
| | | dimethylisoxazolidin-3-yl]pyridine |
| T-109 | an individualized mixture B | 2,3,5,6-tetrachloro-4-methanesulfonyl- |
| | | pyridine |
| T-110 | an individualized mixture B | 3,4,5-trichloropyridine-2,6- |
| | | dicarbonitrile |
| T-111 | an individualized mixture B | N-(1-(5-bromo-3-chloropyridin-2- |
| | | yl)ethyl)-2,4-dichloronicotinamide |
| T-112 | an individualized mixture B | N-((5-bromo-3-chloropyridin-2- |
| | | yl)methyl)-2,4-dichloronicotinamide |
| T-113 | an individualized mixture B | bupirimate |
| T-114 | an individualized mixture B | cyprodinil |
| T-115 | an individualized mixture B | diflumetorim |
| T-116 | an individualized mixture B | fenarimol |
| T-117 | an individualized mixture B | ferimzone |
| T-118 | an individualized mixture B | mepanipyrim |
| T-119 | an individualized mixture B | nitrapyrin |
| T-120 | an individualized mixture B | nuarimol |
| T-121 | an individualized mixture B | pyrimethanil |
| T-122 | an individualized mixture B | triforine |
| T-123 | an individualized mixture B | fenpiclonil |
| T-124 | an individualized mixture B | fludioxonil |
| T-125 | an individualized mixture B | aldimorph |
| T-126 | an individualized mixture B | dodemorph |
| T-127 | an individualized mixture B | dodemorph acetate |
| T-128 | an individualized mixture B | fenpropimorph |
| T-129 | an individualized mixture B | tridemorph |
| T-130 | an individualized mixture B | fenpropidin |
| T-131 | an individualized mixture B | fluoroimide |
| T-132 | an individualized mixture B | iprodione |
| T-133 | an individualized mixture B | procymidone |
| T-134 | an individualized mixture B | vinclozolin |
| T-135 | an individualized mixture B | famoxadone |
| T-136 | an individualized mixture B | fenamidone |
| T-137 | an individualized mixture B | flutianil |
| T-138 | an individualized mixture B | octhilinone |
| T-139 | an individualized mixture B | probenazole |
| T-140 | an individualized mixture B | S-allyl 5-amino-2-isopropyl-4- |
| | | orthotolylpyrazol-3-one-1- |
| | | thiocarboxylate |
| T-141 | an individualized mixture B | acibenzolar-S-methyl |
| T-142 | an individualized mixture B | amisulbrom |
| T-143 | an individualized mixture B | anilazine |
| T-144 | an individualized mixture B | blasticidin-S |
| T-145 | an individualized mixture B | captafol |
| T-146 | an individualized mixture B | captan |
| T-147 | an individualized mixture B | chinomethionate |
| T-148 | an individualized mixture B | dazomet |
| T-149 | an individualized mixture B | debacarb |
| T-150 | an individualized mixture B | diclomezine |
| T-151 | an individualized mixture B | difenzoquat |
| T-152 | an individualized mixture B | difenzoquat methylsulfate |
| T-153 | an individualized mixture B | fenoxanil |
| T-154 | an individualized mixture B | folpet |
| T-155 | an individualized mixture B | oxolinic acid |
| T-156 | an individualized mixture B | piperalin |
| T-157 | an individualized mixture B | proquinazid |
| T-158 | an individualized mixture B | pyroquilon |
| T-159 | an individualized mixture B | quinoxyfen |
| T-160 | an individualized mixture B | triazoxide |
| T-161 | an individualized mixture B | tricyclazole |
| T-162 | an individualized mixture B | 2-butoxy-6-iodo-3-propylchromen-4- |
| | | one |
| T-163 | an individualized mixture B | 5-chloro-1-(4,6-dimethoxypyrimidin-2- |
| | | yl)-2-methyl-1H-benzimidazole |
| T-164 | an individualized mixture B | 5-chloro-7-(4-methylpiperidin-1-yl)- |
| | | 6-(2,4,6-trifluorophenyl)- |
| | | [1,2,4]triazolo[1,5-a]pyrimidine |
| T-165 | an individualized mixture B | 5-ethyl-6-octyl-[1,2,4]triazolo[1,5- |
| | | a]pyrimidin-7-ylamine |
| T-166 | an individualized mixture B | ferbam |
| T-167 | an individualized mixture B | mancozeb |
| T-168 | an individualized mixture B | maneb |
| T-169 | an individualized mixture B | metam |
| T-170 | an individualized mixture B | methasulfocarb |
| T-171 | an individualized mixture B | metiram |
| T-172 | an individualized mixture B | propineb |
| T-173 | an individualized mixture B | thiram |
| T-174 | an individualized mixture B | zineb |
| T-175 | an individualized mixture B | ziram |
| T-176 | an individualized mixture B | diethofencarb |
| T-177 | an individualized mixture B | benthiavalicarb |
| T-178 | an individualized mixture B | iprovalicarb |
| T-179 | an individualized mixture B | propamocarb |
| T-180 | an individualized mixture B | propamocarb hydrochloride |
| T-181 | an individualized mixture B | valiphenal |
| T-182 | an individualized mixture B | 4-fluorophenyl N-(1-(1-(4- |
| | | cyanophenyl)-ethanesulfonyl)but-2- |
| | | yl)carbamate |
| T-183 | an individualized mixture B | dodine |
| T-184 | an individualized mixture B | dodine free base |
| T-185 | an individualized mixture B | guazatine |
| T-186 | an individualized mixture B | guazatine acetate |
| T-187 | an individualized mixture B | iminoctadine |
| T-188 | an individualized mixture B | iminoctadine triacetate |
| T-189 | an individualized mixture B | iminoctadine tris(albesilate) |
| T-190 | an individualized mixture B | kasugamycin |
| T-191 | an individualized mixture B | kasugamycin hydrochloride hydrate |
| T-192 | an individualized mixture B | polyoxin |
| T-193 | an individualized mixture B | streptomycin |
| T-194 | an individualized mixture B | validamycin A |
| T-195 | an individualized mixture B | binapacryl |
| T-196 | an individualized mixture B | dicloran |
| T-197 | an individualized mixture B | dinobuton |
| T-198 | an individualized mixture B | dinocap |
| T-199 | an individualized mixture B | nitrothal-isopropyl |
| T-200 | an individualized mixture B | tecnazen |
| T-201 | an individualized mixture B | fentin salts |
| T-202 | an individualized mixture B | dithianon |
| T-203 | an individualized mixture B | isoprothiolane |
| T-204 | an individualized mixture B | edifenphos |
| T-205 | an individualized mixture B | fosetyl, fosetyl aluminum |
| T-206 | an individualized mixture B | iprobenfos |
| T-207 | an individualized mixture B | phosphorous acid and derivatives |
| T-208 | an individualized mixture B | pyrazophos |
| T-209 | an individualized mixture B | tolclofos-methyl |
| T-210 | an individualized mixture B | chlorthalonil |
| T-211 | an individualized mixture B | dichlofluanid |
| T-212 | an individualized mixture B | dichlorophene |
| T-213 | an individualized mixture B | flusulfamide |
| T-214 | an individualized mixture B | hexachlorobenzene |
| T-215 | an individualized mixture B | pencycuron |
| T-216 | an individualized mixture B | pentachlorophenol and salts |
| T-217 | an individualized mixture B | phthalide |
| T-218 | an individualized mixture B | quintozene |
| T-219 | an individualized mixture B | thiophanate methyl |
| T-220 | an individualized mixture B | tolylfluanid |
| T-221 | an individualized mixture B | N-(4-chloro-2-nitrophenyl)-N-ethyl- |
| | | 4-methylbenzenesulfonamide |
| T-222 | an individualized mixture B | Bordeaux mixture |
| T-223 | an individualized mixture B | copper acetate |
| T-224 | an individualized mixture B | copper hydroxide |
| T-225 | an individualized mixture B | copper oxychloride |
| T-226 | an individualized mixture B | basic copper sulfate |
| T-227 | an individualized mixture B | sulfur |
| T-228 | an individualized mixture B | biphenyl |
| T-229 | an individualized mixture B | bronopol |
| T-230 | an individualized mixture B | cyflufenamid |
| T-231 | an individualized mixture B | cymoxanil |
| T-232 | an individualized mixture B | diphenylamine |
| T-233 | an individualized mixture B | metrafenone |
| T-234 | an individualized mixture B | mildiomycin |
| T-235 | an individualized mixture B | oxine-copper |
| T-236 | an individualized mixture B | prohexadione-calcium |
| T-237 | an individualized mixture B | spiroxamine |
| T-238 | an individualized mixture B | tolylfluanid |
| T-239 | an individualized mixture B | N-(cyclopropylmethoxyimino-(6- |
| | | difluoromethoxy-2,3- |
| | | difluorophenyl)methyl)- |
| | | 2-phenylacetamide |
| T-240 | an individualized mixture B | N′-(4-(4-chloro-3- |
| | | trifluoromethylphenoxy)- |
| | | 2,5-dimethylphenyl)-N-ethyl-N-methyl- |
| | | formamidine |
| T-241 | an individualized mixture B | N′-(4-(4-fluoro-3- |
| | | trifluoromethylphenoxy)- |
| | | 2,5-dimethylphenyl)-N-ethyl-N-methyl- |
| | | formamidine |
| T-242 | an individualized mixture B | N′-(2-methyl-5-trifluoromethyl-4-(3-tri- |
| | | methylsilanylpropoxy)phenyl)-N-ethyl- |
| | | N-methylformamidine |
| T-243 | an individualized mixture B | N′-(5-difluoromethyl-2-methyl-4-(3-tri- |
| | | methylsilanylpropoxy)phenyl)-N-ethyl- |
| | | N-methylformamidine |
| T-244 | an individualized mixture B | methyl N-(1,2,3,4- |
| | | tetrahydronaphthalen-1-yl)-2-{1-[2-(5- |
| | | methyl-3-trifluoromethyl-pyrazol-1- |
| | | yl)acetyl]piperidin-4-yl}thiazole-4- |
| | | carboxamide |
| T-245 | an individualized mixture B | methyl (R)-N-(1,2,3,4- |
| | | tetrahydronaphthalen-1-yl)-2-{1-[2-(5- |
| | | methyl-3-trifluoromethyl-pyrazol-1- |
| | | yl)acetyl]piperidin-4-yl}thiazole-4- |
| | | carboxamide |
| T-246 | an individualized mixture B | 6-tert-butyl-8-fluoro-2,3- |
| | | dimethylquinolin-4-yl acetate |
| T-247 | an individualized mixture B | 6-tert-butyl-8-fluoro-2,3- |
| | | dimethylquinolin-4-yl methoxyacetate |
| T-248 | an individualized mixture B | N-methyl-2-{1-[(5-methyl-3-trifluoro- |
| | | methyl-1H-pyrazol-1- |
| | | yl)acetyl]piperidin-4-yl}-N-[(1R)- |
| | | 1,2,3,4-tetrahydronaphthalen-1-yl]-4- |
| | | thiazolecarboxamide |
| T-249 | an individualized mixture B | carbaryl |
| T-250 | an individualized mixture B | carbofuran |
| T-251 | an individualized mixture B | carbosulfan |
| T-252 | an individualized mixture B | methomylthiodicarb |
| T-253 | an individualized mixture B | bifenthrin |
| T-254 | an individualized mixture B | cyfluthrin |
| T-255 | an individualized mixture B | cypermethrin |
| T-256 | an individualized mixture B | alpha-cypermethrin |
| T-257 | an individualized mixture B | zeta-cypermethrin |
| T-258 | an individualized mixture B | deltamethrin |
| T-259 | an individualized mixture B | esfenvalerate |
| T-260 | an individualized mixture B | lambda-cyhalothrin |
| T-261 | an individualized mixture B | permethrin |
| T-262 | an individualized mixture B | tefluthrin |
| T-263 | an individualized mixture B | diflubenzuron |
| T-264 | an individualized mixture B | flufenoxuron |
| T-265 | an individualized mixture B | lufenuron |
| T-266 | an individualized mixture B | teflubenzuron |
| T-267 | an individualized mixture B | spirotetramate |
| T-268 | an individualized mixture B | clothianidin |
| T-269 | an individualized mixture B | dinotefuran |
| T-270 | an individualized mixture B | imidacloprid |
| T-271 | an individualized mixture B | thiamethoxam |
| T-272 | an individualized mixture B | acetamiprid |
| T-273 | an individualized mixture B | thiacloprid |
| T-274 | an individualized mixture B | endosulfan |
| T-275 | an individualized mixture B | fipronil |
| T-276 | an individualized mixture B | abamectin |
| T-277 | an individualized mixture B | emamectin |
| T-278 | an individualized mixture B | spinosad |
| T-279 | an individualized mixture B | spinetoram |
| T-280 | an individualized mixture B | hydramethylnon |
| T-281 | an individualized mixture B | chlorfenapyr |
| T-282 | an individualized mixture B | fenbutatin oxide |
| T-283 | an individualized mixture B | indoxacarb |
| T-284 | an individualized mixture B | metaflumizone |
| T-285 | an individualized mixture B | flonicamid |
| T-286 | an individualized mixture B | lubendiamid |
| T-287 | an individualized mixture B | chlorantraniliprol |
| T-288 | an individualized mixture B | cyazypyr (HGW86) |
| T-289 | an individualized mixture B | cyflumetofen |
| T-290 | an individualized mixture B | acetochlor |
| T-291 | an individualized mixture B | dimethenamid |
| T-292 | an individualized mixture B | metolachlor |
| T-293 | an individualized mixture B | metazachlor |
| T-294 | an individualized mixture B | glyphosate |
| T-295 | an individualized mixture B | glufosinate |
| T-296 | an individualized mixture B | sulfosate |
| T-297 | an individualized mixture B | clodinafop |
| T-298 | an individualized mixture B | fenoxaprop |
| T-299 | an individualized mixture B | fluazifop |
| T-300 | an individualized mixture B | haloxyfop |
| T-301 | an individualized mixture B | paraquat |
| T-302 | an individualized mixture B | phenmedipham |
| T-303 | an individualized mixture B | clethodim |
| T-304 | an individualized mixture B | cycloxydim |
| T-305 | an individualized mixture B | profoxydim |
| T-306 | an individualized mixture B | sethoxydim |
| T-307 | an individualized mixture B | tepraloxydim |
| T-308 | an individualized mixture B | pendimethalin |
| T-309 | an individualized mixture B | prodiamine |
| T-310 | an individualized mixture B | trifluralin |
| T-311 | an individualized mixture B | acifluorfen |
| T-312 | an individualized mixture B | bromoxynil |
| T-313 | an individualized mixture B | imazamethabenz |
| T-314 | an individualized mixture B | imazamox |
| T-315 | an individualized mixture B | imazapic |
| T-316 | an individualized mixture B | imazapyr |
| T-317 | an individualized mixture B | imazaquin |
| T-318 | an individualized mixture B | imazethapyr |
| T-319 | an individualized mixture B | 2,4-dichlorophenoxyacetic acid (2,4- |
| | | D) |
| T-320 | an individualized mixture B | chloridazon |
| T-321 | an individualized mixture B | clopyralid |
| T-322 | an individualized mixture B | fluroxypyr |
| T-323 | an individualized mixture B | picloram |
| T-324 | an individualized mixture B | picolinafen |
| T-325 | an individualized mixture B | bensulfuron |
| T-326 | an individualized mixture B | chlorimuron-ethyl |
| T-327 | an individualized mixture B | cyclosulfamuron |
| T-328 | an individualized mixture B | iodosulfuron |
| T-329 | an individualized mixture B | mesosulfuron |
| T-330 | an individualized mixture B | metsulfuron-methyl |
| T-331 | an individualized mixture B | nicosulfuron |
| T-332 | an individualized mixture B | rimsulfuron |
| T-333 | an individualized mixture B | triflusulfuron |
| T-334 | an individualized mixture B | atrazine |
| T-335 | an individualized mixture B | hexazinone |
| T-336 | an individualized mixture B | diuron |
| T-337 | an individualized mixture B | florasulam |
| T-338 | an individualized mixture B | pyroxasulfon |
| T-339 | an individualized mixture B | bentazone |
| T-340 | an individualized mixture B | cinidon-ethyl |
| T-341 | an individualized mixture B | cinmethylin |
| T-342 | an individualized mixture B | dicamba |
| T-343 | an individualized mixture B | diflufenzopyr |
| T-344 | an individualized mixture B | quinclorac |
| T-345 | an individualized mixture B | quinmerac |
| T-346 | an individualized mixture B | mesotrione |
| T-347 | an individualized mixture B | saflufenacil |
| T-348 | an individualized mixture B | topramezone |

Particular preference is given to the mixtures in which component 2 and component 3 is a compound II selected from the group of the compounds below:
II-1 epoxiconazole
II-2 metconazole
II-3 tebuconazole
   II-6 triticonazole
   II-7 prothioconazole
   II-8 kresoxim-methyl
II-9 pyraclostrobin
II-11 dimethomorph
II-12 5-ethyl-6-octyl-[1,2,4]triazolo[1,5-a]pyrimidin-7-ylamine
II-13 pyrimethanil
II-14 metalaxyl
II-15 fenpropimorph
II-18 mancozeb
II-19 metiram
II-20 thiophanate methyl
II-21 chlorothalonil
II-22 metrafenone
II-23 bixafen
II-24 boscalid
II-25 N-(3′,4′,5′-trifluorobiphenyl-2-yl)-3-difluoromethyl-1-methyl-1H-pyrazole-4-carboxamide
II-26 sedaxane
II-27 isopyrazam
II-28 fluopyram
II-29 penflufen
   with the proviso that component 2 and component 3 are not identical.

TABLE T1

| | | | |
|---|---|---|---|
| M | C1 | C2 | C3 |

The ternary mixtures T1-1 to T1-1881 below comprise, as component 1,
the compound I, a component 2 selected from the active compounds
of groups A to I preferred for component 2 and a component 3 selected
from the active compounds of groups A to I preferred for component 3.
In the table below, each row corresponds to a ternary mixture according
to the invention having the mixture components 1 to 3 listed in the row
in question.

| | | | |
|---|---|---|---|
| T1-1 | I-1 | II-1 | II-2 |
| T1-2 | I-1 | II-1 | II-3 |
| T1-3 | I-1 | II-1 | II-7 |
| T1-4 | I-1 | II-1 | II-8 |
| T1-5 | I-1 | II-1 | II-9 |
| T1-6 | I-1 | II-1 | II-13 |
| T1-7 | I-1 | II-1 | II-14 |
| T1-8 | I-1 | II-1 | II-15 |
| T1-9 | I-1 | II-1 | II-20 |
| T1-10 | I-1 | II-1 | II-21 |
| T1-11 | I-1 | II-1 | II-22 |
| T1-12 | I-1 | II-1 | II-23 |
| T1-13 | I-1 | II-1 | II-24 |
| T1-14 | I-1 | II-1 | II-25 |
| T1-15 | I-1 | II-1 | II-26 |
| T1-16 | I-1 | II-1 | II-27 |
| T1-17 | I-1 | II-1 | II-28 |
| T1-18 | I-1 | II-1 | II-29 |
| T1-19 | I-1 | II-2 | II-3 |
| T1-20 | I-1 | II-2 | II-7 |
| T1-21 | I-1 | II-2 | II-8 |
| T1-22 | I-1 | II-2 | II-9 |
| T1-23 | I-1 | II-2 | II-13 |
| T1-24 | I-1 | II-2 | II-14 |
| T1-25 | I-1 | II-2 | II-15 |
| T1-26 | I-1 | II-2 | II-20 |
| T1-27 | I-1 | II-2 | II-21 |
| T1-28 | I-1 | II-2 | II-22 |
| T1-29 | I-1 | II-2 | II-23 |
| T1-30 | I-1 | II-2 | II-24 |
| T1-31 | I-1 | II-2 | II-25 |
| T1-32 | I-1 | II-2 | II-26 |
| T1-33 | I-1 | II-2 | II-27 |
| T1-34 | I-1 | II-2 | II-28 |
| T1-35 | I-1 | II-2 | II-29 |
| T1-36 | I-1 | II-3 | II-7 |
| T1-37 | I-1 | II-3 | II-8 |
| T1-38 | I-1 | II-3 | II-9 |
| T1-39 | I-1 | II-3 | II-13 |
| T1-40 | I-1 | II-3 | II-14 |
| T1-41 | I-1 | II-3 | II-15 |
| T1-42 | I-1 | II-3 | II-20 |
| T1-43 | I-1 | II-3 | II-21 |
| T1-44 | I-1 | II-3 | II-22 |
| T1-45 | I-1 | II-3 | II-23 |
| T1-46 | I-1 | II-3 | II-24 |
| T1-47 | I-1 | II-3 | II-25 |
| T1-48 | I-1 | II-3 | II-26 |
| T1-49 | I-1 | II-3 | II-27 |
| T1-50 | I-1 | II-3 | II-28 |
| T1-51 | I-1 | II-3 | II-29 |
| T1-52 | I-1 | II-7 | II-8 |
| T1-53 | I-1 | II-7 | II-9 |
| T1-54 | I-1 | II-7 | II-13 |
| T1-55 | I-1 | II-7 | II-14 |
| T1-56 | I-1 | II-7 | II-15 |
| T1-57 | I-1 | II-7 | II-20 |
| T1-58 | I-1 | II-7 | II-21 |
| T1-59 | I-1 | II-7 | II-22 |
| T1-60 | I-1 | II-7 | II-23 |
| T1-61 | I-1 | II-7 | II-24 |
| T1-62 | I-1 | II-7 | II-25 |
| T1-63 | I-1 | II-7 | II-26 |
| T1-64 | I-1 | II-7 | II-27 |
| T1-65 | I-1 | II-7 | II-28 |
| T1-66 | I-1 | II-7 | II-29 |
| T1-67 | I-1 | II-8 | II-9 |
| T1-68 | I-1 | II-8 | II-13 |
| T1-69 | I-1 | II-8 | II-14 |
| T1-70 | I-1 | II-8 | II-15 |
| T1-71 | I-1 | II-8 | II-20 |
| T1-72 | I-1 | II-8 | II-21 |
| T1-73 | I-1 | II-8 | II-22 |
| T1-74 | I-1 | II-8 | II-23 |
| T1-75 | I-1 | II-8 | II-24 |
| T1-76 | I-1 | II-8 | II-25 |
| T1-77 | I-1 | II-8 | II-26 |
| T1-78 | I-1 | II-8 | II-27 |
| T1-79 | I-1 | II-8 | II-28 |
| T1-80 | I-1 | II-8 | II-29 |
| T1-81 | I-1 | II-9 | II-13 |
| T1-82 | I-1 | II-9 | II-14 |
| T1-83 | I-1 | II-9 | II-15 |
| T1-84 | I-1 | II-9 | II-20 |
| T1-85 | I-1 | II-9 | II-21 |
| T1-86 | I-1 | II-9 | II-22 |
| T1-87 | I-1 | II-9 | II-23 |
| T1-88 | I-1 | II-9 | II-24 |
| T1-89 | I-1 | II-9 | II-25 |
| T1-90 | I-1 | II-9 | II-26 |
| T1-91 | I-1 | II-9 | II-27 |
| T1-92 | I-1 | II-9 | II-28 |
| T1-93 | I-1 | II-9 | II-29 |
| T1-94 | I-1 | II-13 | II-14 |
| T1-95 | I-1 | II-13 | II-15 |
| T1-96 | I-1 | II-13 | II-20 |
| T1-97 | I-1 | II-13 | II-21 |
| T1-98 | I-1 | II-13 | II-22 |
| T1-99 | I-1 | II-13 | II-23 |
| T1-100 | I-1 | II-13 | II-24 |
| T1-101 | I-1 | II-13 | II-25 |
| T1-102 | I-1 | II-13 | II-26 |
| T1-103 | I-1 | II-13 | II-27 |
| T1-104 | I-1 | II-13 | II-28 |
| T1-105 | I-1 | II-13 | II-29 |
| T1-106 | I-1 | II-14 | II-15 |
| T1-107 | I-1 | II-14 | II-20 |
| T1-108 | I-1 | II-14 | II-21 |
| T1-109 | I-1 | II-14 | II-22 |
| T1-110 | I-1 | II-14 | II-23 |
| T1-111 | I-1 | II-14 | II-24 |
| T1-112 | I-1 | II-14 | II-25 |
| T1-113 | I-1 | II-14 | II-26 |
| T1-114 | I-1 | II-14 | II-27 |
| T1-115 | I-1 | II-14 | II-28 |
| T1-116 | I-1 | II-14 | II-29 |
| T1-117 | I-1 | II-15 | II-20 |
| T1-118 | I-1 | II-15 | II-21 |
| T1-119 | I-1 | II-15 | II-22 |
| T1-120 | I-1 | II-15 | II-23 |
| T1-121 | I-1 | II-15 | II-24 |
| T1-122 | I-1 | II-15 | II-25 |
| T1-123 | I-1 | II-15 | II-26 |
| T1-124 | I-1 | II-15 | II-27 |
| T1-125 | I-1 | II-15 | II-28 |
| T1-126 | I-1 | II-15 | II-29 |
| T1-127 | I-1 | II-20 | II-21 |
| T1-128 | I-1 | II-20 | II-22 |
| T1-129 | I-1 | II-20 | II-23 |
| T1-130 | I-1 | II-20 | II-24 |
| T1-131 | I-1 | II-20 | II-25 |
| T1-132 | I-1 | II-20 | II-26 |
| T1-133 | I-1 | II-20 | II-27 |
| T1-134 | I-1 | II-20 | II-28 |
| T1-135 | I-1 | II-20 | II-29 |
| T1-136 | I-1 | II-21 | II-22 |
| T1-137 | I-1 | II-21 | II-23 |
| T1-138 | I-1 | II-21 | II-24 |
| T1-139 | I-1 | II-21 | II-25 |
| T1-140 | I-1 | II-21 | II-26 |
| T1-141 | I-1 | II-21 | II-27 |
| T1-142 | I-1 | II-21 | II-28 |
| T1-143 | I-1 | II-21 | II-29 |
| T1-144 | I-1 | II-22 | II-23 |
| T1-145 | I-1 | II-22 | II-24 |
| T1-146 | I-1 | II-22 | II-25 |
| T1-147 | I-1 | II-22 | II-26 |
| T1-148 | I-1 | II-22 | II-27 |
| T1-149 | I-1 | II-22 | II-28 |
| T1-150 | I-1 | II-22 | II-29 |
| T1-151 | I-1 | II-23 | II-24 |
| T1-152 | I-1 | II-23 | II-25 |
| T1-153 | I-1 | II-23 | II-26 |
| T1-154 | I-1 | II-23 | II-27 |
| T1-155 | I-1 | II-23 | II-28 |
| T1-156 | I-1 | II-23 | II-29 |
| T1-157 | I-1 | II-24 | II-25 |
| T1-158 | I-1 | II-24 | II-26 |
| T1-159 | I-1 | II-24 | II-27 |
| T1-160 | I-1 | II-24 | II-28 |
| T1-161 | I-1 | II-24 | II-29 |
| T1-162 | I-1 | II-25 | II-26 |
| T1-163 | I-1 | II-25 | II-27 |
| T1-164 | I-1 | II-25 | II-28 |
| T1-165 | I-1 | II-25 | II-29 |
| T1-166 | I-1 | II-26 | II-27 |
| T1-167 | I-1 | II-26 | II-28 |
| T1-168 | I-1 | II-26 | II-29 |
| T1-169 | I-1 | II-27 | II-28 |
| T1-170 | I-1 | II-27 | II-29 |
| T1-171 | I-1 | II-28 | II-29 |
| T1-172 | I-2 | II-1 | II-2 |
| T1-173 | I-2 | II-1 | II-3 |
| T1-174 | I-2 | II-1 | II-7 |
| T1-175 | I-2 | II-1 | II-8 |
| T1-176 | I-2 | II-1 | II-9 |
| T1-177 | I-2 | II-1 | II-13 |
| T1-178 | I-2 | II-1 | II-14 |
| T1-179 | I-2 | II-1 | II-15 |
| T1-180 | I-2 | II-1 | II-20 |
| T1-181 | I-2 | II-1 | II-21 |
| T1-182 | I-2 | II-1 | II-22 |
| T1-183 | I-2 | II-1 | II-23 |
| T1-184 | I-2 | II-1 | II-24 |
| T1-185 | I-2 | II-1 | II-25 |
| T1-186 | I-2 | II-1 | II-26 |
| T1-187 | I-2 | II-1 | II-27 |
| T1-188 | I-2 | II-1 | II-28 |
| T1-189 | I-2 | II-1 | II-29 |
| T1-190 | I-2 | II-2 | II-3 |
| T1-191 | I-2 | II-2 | II-7 |
| T1-192 | I-2 | II-2 | II-8 |
| T1-193 | I-2 | II-2 | II-9 |
| T1-194 | I-2 | II-2 | II-13 |
| T1-195 | I-2 | II-2 | II-14 |
| T1-196 | I-2 | II-2 | II-15 |
| T1-197 | I-2 | II-2 | II-20 |
| T1-198 | I-2 | II-2 | II-21 |
| T1-199 | I-2 | II-2 | II-22 |
| T1-200 | I-2 | II-2 | II-23 |
| T1-201 | I-2 | II-2 | II-24 |
| T1-202 | I-2 | II-2 | II-25 |
| T1-203 | I-2 | II-2 | II-26 |
| T1-204 | I-2 | II-2 | II-27 |
| T1-205 | I-2 | II-2 | II-28 |
| T1-206 | I-2 | II-2 | II-29 |
| T1-207 | I-2 | II-3 | II-7 |
| T1-208 | I-2 | II-3 | II-8 |
| T1-209 | I-2 | II-3 | II-9 |
| T1-210 | I-2 | II-3 | II-13 |
| T1-211 | I-2 | II-3 | II-14 |
| T1-212 | I-2 | II-3 | II-15 |
| T1-213 | I-2 | II-3 | II-20 |
| T1-214 | I-2 | II-3 | II-21 |
| T1-215 | I-2 | II-3 | II-22 |
| T1-216 | I-2 | II-3 | II-23 |
| T1-217 | I-2 | II-3 | II-24 |
| T1-218 | I-2 | II-3 | II-25 |
| T1-219 | I-2 | II-3 | II-26 |
| T1-220 | I-2 | II-3 | II-27 |
| T1-221 | I-2 | II-3 | II-28 |
| T1-222 | I-2 | II-3 | II-29 |
| T1-223 | I-2 | II-7 | II-8 |
| T1-224 | I-2 | II-7 | II-9 |
| T1-225 | I-2 | II-7 | II-13 |
| T1-226 | I-2 | II-7 | II-14 |
| T1-227 | I-2 | II-7 | II-15 |
| T1-228 | I-2 | II-7 | II-20 |
| T1-229 | I-2 | II-7 | II-21 |
| T1-230 | I-2 | II-7 | II-22 |
| T1-231 | I-2 | II-7 | II-23 |
| T1-232 | I-2 | II-7 | II-24 |
| T1-233 | I-2 | II-7 | II-25 |
| T1-234 | I-2 | II-7 | II-26 |
| T1-235 | I-2 | II-7 | II-27 |
| T1-236 | I-2 | II-7 | II-28 |
| T1-237 | I-2 | II-7 | II-29 |
| T1-238 | I-2 | II-8 | II-9 |
| T1-239 | I-2 | II-8 | II-13 |
| T1-240 | I-2 | II-8 | II-14 |
| T1-241 | I-2 | II-8 | II-15 |
| T1-242 | I-2 | II-8 | II-20 |
| T1-243 | I-2 | II-8 | II-21 |
| T1-244 | I-2 | II-8 | II-22 |
| T1-245 | I-2 | II-8 | II-23 |
| T1-246 | I-2 | II-8 | II-24 |
| T1-247 | I-2 | II-8 | II-25 |
| T1-248 | I-2 | II-8 | II-26 |
| T1-249 | I-2 | II-8 | II-27 |
| T1-250 | I-2 | II-8 | II-28 |
| T1-251 | I-2 | II-8 | II-29 |
| T1-252 | I-2 | II-9 | II-13 |
| T1-253 | I-2 | II-9 | II-14 |
| T1-254 | I-2 | II-9 | II-15 |
| T1-255 | I-2 | II-9 | II-20 |
| T1-256 | I-2 | II-9 | II-21 |
| T1-257 | I-2 | II-9 | II-22 |
| T1-258 | I-2 | II-9 | II-23 |
| T1-259 | I-2 | II-9 | II-24 |
| T1-260 | I-2 | II-9 | II-25 |
| T1-261 | I-2 | II-9 | II-26 |
| T1-262 | I-2 | II-9 | II-27 |
| T1-263 | I-2 | II-9 | II-28 |
| T1-264 | I-2 | II-9 | II-29 |
| T1-265 | I-2 | II-13 | II-14 |
| T1-266 | I-2 | II-13 | II-15 |
| T1-267 | I-2 | II-13 | II-20 |
| T1-268 | I-2 | II-13 | II-21 |
| T1-269 | I-2 | II-13 | II-22 |
| T1-270 | I-2 | II-13 | II-23 |
| T1-271 | I-2 | II-13 | II-24 |
| T1-272 | I-2 | II-13 | II-25 |
| T1-273 | I-2 | II-13 | II-26 |
| T1-274 | I-2 | II-13 | II-27 |
| T1-275 | I-2 | II-13 | II-28 |
| T1-276 | I-2 | II-13 | II-29 |
| T1-277 | I-2 | II-14 | II-15 |
| T1-278 | I-2 | II-14 | II-20 |
| T1-279 | I-2 | II-14 | II-21 |
| T1-280 | I-2 | II-14 | II-22 |
| T1-281 | I-2 | II-14 | II-23 |
| T1-282 | I-2 | II-14 | II-24 |
| T1-283 | I-2 | II-14 | II-25 |
| T1-284 | I-2 | II-14 | II-26 |
| T1-285 | I-2 | II-14 | II-27 |
| T1-286 | I-2 | II-14 | II-28 |
| T1-287 | I-2 | II-14 | II-29 |
| T1-288 | I-2 | II-15 | II-20 |
| T1-289 | I-2 | II-15 | II-21 |
| T1-290 | I-2 | II-15 | II-22 |
| T1-291 | I-2 | II-15 | II-23 |
| T1-292 | I-2 | II-15 | II-24 |
| T1-293 | I-2 | II-15 | II-25 |
| T1-294 | I-2 | II-15 | II-26 |
| T1-295 | I-2 | II-15 | II-27 |
| T1-296 | I-2 | II-15 | II-28 |
| T1-297 | I-2 | II-15 | II-29 |
| T1-298 | I-2 | II-20 | II-21 |
| T1-299 | I-2 | II-20 | II-22 |
| T1-300 | I-2 | II-20 | II-23 |
| T1-301 | I-2 | II-20 | II-24 |
| T1-302 | I-2 | II-20 | II-25 |
| T1-303 | I-2 | II-20 | II-26 |
| T1-304 | I-2 | II-20 | II-27 |
| T1-305 | I-2 | II-20 | II-28 |
| T1-306 | I-2 | II-20 | II-29 |
| T1-307 | I-2 | II-21 | II-22 |
| T1-308 | I-2 | II-21 | II-23 |
| T1-309 | I-2 | II-21 | II-24 |
| T1-310 | I-2 | II-21 | II-25 |
| T1-311 | I-2 | II-21 | II-26 |
| T1-312 | I-2 | II-21 | II-27 |
| T1-313 | I-2 | II-21 | II-28 |
| T1-314 | I-2 | II-21 | II-29 |
| T1-315 | I-2 | II-22 | II-23 |
| T1-316 | I-2 | II-22 | II-24 |
| T1-317 | I-2 | II-22 | II-25 |
| T1-318 | I-2 | II-22 | II-26 |
| T1-319 | I-2 | II-22 | II-27 |
| T1-320 | I-2 | II-22 | II-28 |
| T1-321 | I-2 | II-22 | II-29 |
| T1-322 | I-2 | II-23 | II-24 |
| T1-323 | I-2 | II-23 | II-25 |
| T1-324 | I-2 | II-23 | II-26 |
| T1-325 | I-2 | II-23 | II-27 |
| T1-326 | I-2 | II-23 | II-28 |
| T1-327 | I-2 | II-23 | II-29 |
| T1-328 | I-2 | II-24 | II-25 |
| T1-329 | I-2 | II-24 | II-26 |
| T1-330 | I-2 | II-24 | II-27 |
| T1-331 | I-2 | II-24 | II-28 |
| T1-332 | I-2 | II-24 | II-29 |
| T1-333 | I-2 | II-25 | II-26 |
| T1-334 | I-2 | II-25 | II-27 |
| T1-335 | I-2 | II-25 | II-28 |
| T1-336 | I-2 | II-25 | II-29 |
| T1-337 | I-2 | II-26 | II-27 |
| T1-338 | I-2 | II-26 | II-28 |
| T1-339 | I-2 | II-26 | II-29 |
| T1-340 | I-2 | II-27 | II-28 |
| T1-341 | I-2 | II-27 | II-29 |
| T1-342 | I-2 | II-28 | II-29 |
| T1-343 | I-6 | II-1 | II-2 |
| T1-344 | I-6 | II-1 | II-3 |
| T1-345 | I-6 | II-1 | II-7 |
| T1-346 | I-6 | II-1 | II-8 |
| T1-347 | I-6 | II-1 | II-9 |
| T1-348 | I-6 | II-1 | II-13 |
| T1-349 | I-6 | II-1 | II-14 |
| T1-350 | I-6 | II-1 | II-15 |
| T1-351 | I-6 | II-1 | II-20 |
| T1-352 | I-6 | II-1 | II-21 |
| T1-353 | I-6 | II-1 | II-22 |
| T1-354 | I-6 | II-1 | II-23 |
| T1-355 | I-6 | II-1 | II-24 |
| T1-356 | I-6 | II-1 | II-25 |
| T1-357 | I-6 | II-1 | II-26 |
| T1-358 | I-6 | II-1 | II-27 |
| T1-359 | I-6 | II-1 | II-28 |
| T1-360 | I-6 | II-1 | II-29 |
| T1-361 | I-6 | II-2 | II-3 |
| T1-362 | I-6 | II-2 | II-7 |
| T1-363 | I-6 | II-2 | II-8 |
| T1-364 | I-6 | II-2 | II-9 |
| T1-365 | I-6 | II-2 | II-13 |
| T1-366 | I-6 | II-2 | II-14 |
| T1-367 | I-6 | II-2 | II-15 |
| T1-368 | I-6 | II-2 | II-20 |
| T1-369 | I-6 | II-2 | II-21 |
| T1-370 | I-6 | II-2 | II-22 |
| T1-371 | I-6 | II-2 | II-23 |
| T1-372 | I-6 | II-2 | II-24 |
| T1-373 | I-6 | II-2 | II-25 |
| T1-374 | I-6 | II-2 | II-26 |
| T1-375 | I-6 | II-2 | II-27 |
| T1-376 | I-6 | II-2 | II-28 |
| T1-377 | I-6 | II-2 | II-29 |
| T1-378 | I-6 | II-3 | II-7 |
| T1-379 | I-6 | II-3 | II-8 |
| T1-380 | I-6 | II-3 | II-9 |
| T1-381 | I-6 | II-3 | II-13 |
| T1-382 | I-6 | II-3 | II-14 |
| T1-383 | I-6 | II-3 | II-15 |
| T1-384 | I-6 | II-3 | II-20 |
| T1-385 | I-6 | II-3 | II-21 |
| T1-386 | I-6 | II-3 | II-22 |
| T1-387 | I-6 | II-3 | II-23 |
| T1-388 | I-6 | II-3 | II-24 |
| T1-389 | I-6 | II-3 | II-25 |
| T1-390 | I-6 | II-3 | II-26 |
| T1-391 | I-6 | II-3 | II-27 |
| T1-392 | I-6 | II-3 | II-28 |
| T1-393 | I-6 | II-3 | II-29 |
| T1-394 | I-6 | II-7 | II-8 |
| T1-395 | I-6 | II-7 | II-9 |
| T1-396 | I-6 | II-7 | II-13 |
| T1-397 | I-6 | II-7 | II-14 |
| T1-398 | I-6 | II-7 | II-15 |
| T1-399 | I-6 | II-7 | II-20 |
| T1-400 | I-6 | II-7 | II-21 |
| T1-401 | I-6 | II-7 | II-22 |
| T1-402 | I-6 | II-7 | II-23 |
| T1-403 | I-6 | II-7 | II-24 |
| T1-404 | I-6 | II-7 | II-25 |
| T1-405 | I-6 | II-7 | II-26 |
| T1-406 | I-6 | II-7 | II-27 |
| T1-407 | I-6 | II-7 | II-28 |
| T1-408 | I-6 | II-7 | II-29 |
| T1-409 | I-6 | II-8 | II-9 |
| T1-410 | I-6 | II-8 | II-13 |
| T1-411 | I-6 | II-8 | II-14 |
| T1-412 | I-6 | II-8 | II-15 |
| T1-413 | I-6 | II-8 | II-20 |
| T1-414 | I-6 | II-8 | II-21 |
| T1-415 | I-6 | II-8 | II-22 |
| T1-416 | I-6 | II-8 | II-23 |
| T1-417 | I-6 | II-8 | II-24 |
| T1-418 | I-6 | II-8 | II-25 |
| T1-419 | I-6 | II-8 | II-26 |
| T1-420 | I-6 | II-8 | II-27 |
| T1-421 | I-6 | II-8 | II-28 |
| T1-422 | I-6 | II-8 | II-29 |
| T1-423 | I-6 | II-9 | II-13 |
| T1-424 | I-6 | II-9 | II-14 |
| T1-425 | I-6 | II-9 | II-15 |
| T1-426 | I-6 | II-9 | II-20 |
| T1-427 | I-6 | II-9 | II-21 |
| T1-428 | I-6 | II-9 | II-22 |
| T1-429 | I-6 | II-9 | II-23 |
| T1-430 | I-6 | II-9 | II-24 |
| T1-431 | I-6 | II-9 | II-25 |
| T1-432 | I-6 | II-9 | II-26 |
| T1-433 | I-6 | II-9 | II-27 |
| T1-434 | I-6 | II-9 | II-28 |
| T1-435 | I-6 | II-9 | II-29 |
| T1-436 | I-6 | II-13 | II-14 |
| T1-437 | I-6 | II-13 | II-15 |
| T1-438 | I-6 | II-13 | II-20 |
| T1-439 | I-6 | II-13 | II-21 |
| T1-440 | I-6 | II-13 | II-22 |
| T1-441 | I-6 | II-13 | II-23 |
| T1-442 | I-6 | II-13 | II-24 |
| T1-443 | I-6 | II-13 | II-25 |
| T1-444 | I-6 | II-13 | II-26 |
| T1-445 | I-6 | II-13 | II-27 |
| T1-446 | I-6 | II-13 | II-28 |
| T1-447 | I-6 | II-13 | II-29 |
| T1-448 | I-6 | II-14 | II-15 |
| T1-449 | I-6 | II-14 | II-20 |
| T1-450 | I-6 | II-14 | II-21 |
| T1-451 | I-6 | II-14 | II-22 |
| T1-452 | I-6 | II-14 | II-23 |
| T1-453 | I-6 | II-14 | II-24 |
| T1-454 | I-6 | II-14 | II-25 |
| T1-455 | I-6 | II-14 | II-26 |
| T1-456 | I-6 | II-14 | II-27 |
| T1-457 | I-6 | II-14 | II-28 |
| T1-458 | I-6 | II-14 | II-29 |
| T1-459 | I-6 | II-15 | II-20 |
| T1-460 | I-6 | II-15 | II-21 |
| T1-461 | I-6 | II-15 | II-22 |
| T1-462 | I-6 | II-15 | II-23 |
| T1-463 | I-6 | II-15 | II-24 |
| T1-464 | I-6 | II-15 | II-25 |
| T1-465 | I-6 | II-15 | II-26 |
| T1-466 | I-6 | II-15 | II-27 |
| T1-467 | I-6 | II-15 | II-28 |
| T1-468 | I-6 | II-15 | II-29 |
| T1-469 | I-6 | II-20 | II-21 |
| T1-470 | I-6 | II-20 | II-22 |
| T1-471 | I-6 | II-20 | II-23 |
| T1-472 | I-6 | II-20 | II-24 |
| T1-473 | I-6 | II-20 | II-25 |
| T1-474 | I-6 | II-20 | II-26 |
| T1-475 | I-6 | II-20 | II-27 |
| T1-476 | I-6 | II-20 | II-28 |
| T1-477 | I-6 | II-20 | II-29 |
| T1-478 | I-6 | II-21 | II-22 |
| T1-479 | I-6 | II-21 | II-23 |
| T1-480 | I-6 | II-21 | II-24 |
| T1-481 | I-6 | II-21 | II-25 |
| T1-482 | I-6 | II-21 | II-26 |
| T1-483 | I-6 | II-21 | II-27 |
| T1-484 | I-6 | II-21 | II-28 |
| T1-485 | I-6 | II-21 | II-29 |
| T1-486 | I-6 | II-22 | II-23 |
| T1-487 | I-6 | II-22 | II-24 |
| T1-488 | I-6 | II-22 | II-25 |
| T1-489 | I-6 | II-22 | II-26 |
| T1-490 | I-6 | II-22 | II-27 |
| T1-491 | I-6 | II-22 | II-28 |
| T1-492 | I-6 | II-22 | II-29 |
| T1-493 | I-6 | II-23 | II-24 |
| T1-494 | I-6 | II-23 | II-25 |
| T1-495 | I-6 | II-23 | II-26 |
| T1-496 | I-6 | II-23 | II-27 |
| T1-497 | I-6 | II-23 | II-28 |
| T1-498 | I-6 | II-23 | II-29 |
| T1-499 | I-6 | II-24 | II-25 |
| T1-500 | I-6 | II-24 | II-26 |
| T1-501 | I-6 | II-24 | II-27 |
| T1-502 | I-6 | II-24 | II-28 |
| T1-503 | I-6 | II-24 | II-29 |
| T1-504 | I-6 | II-25 | II-26 |
| T1-505 | I-6 | II-25 | II-27 |
| T1-506 | I-6 | II-25 | II-28 |
| T1-507 | I-6 | II-25 | II-29 |
| T1-508 | I-6 | II-26 | II-27 |
| T1-509 | I-6 | II-26 | II-28 |
| T1-510 | I-6 | II-26 | II-29 |
| T1-511 | I-6 | II-27 | II-28 |
| T1-512 | I-6 | II-27 | II-29 |
| T1-513 | I-6 | II-28 | II-29 |
| T1-514 | I-7 | II-1 | II-2 |
| T1-515 | I-7 | II-1 | II-3 |
| T1-516 | I-7 | II-1 | II-7 |
| T1-517 | I-7 | II-1 | II-8 |
| T1-518 | I-7 | II-1 | II-9 |
| T1-519 | I-7 | II-1 | II-13 |
| T1-520 | I-7 | II-1 | II-14 |
| T1-521 | I-7 | II-1 | II-15 |
| T1-522 | I-7 | II-1 | II-20 |
| T1-523 | I-7 | II-1 | II-21 |
| T1-524 | I-7 | II-1 | II-22 |
| T1-525 | I-7 | II-1 | II-23 |
| T1-526 | I-7 | II-1 | II-24 |
| T1-527 | I-7 | II-1 | II-25 |
| T1-528 | I-7 | II-1 | II-26 |
| T1-529 | I-7 | II-1 | II-27 |
| T1-530 | I-7 | II-1 | II-28 |
| T1-531 | I-7 | II-1 | II-29 |
| T1-532 | I-7 | II-2 | II-3 |
| T1-533 | I-7 | II-2 | II-7 |
| T1-534 | I-7 | II-2 | II-8 |
| T1-535 | I-7 | II-2 | II-9 |
| T1-536 | I-7 | II-2 | II-13 |
| T1-537 | I-7 | II-2 | II-14 |
| T1-538 | I-7 | II-2 | II-15 |
| T1-539 | I-7 | II-2 | II-20 |
| T1-540 | I-7 | II-2 | II-21 |
| T1-541 | I-7 | II-2 | II-22 |
| T1-542 | I-7 | II-2 | II-23 |
| T1-543 | I-7 | II-2 | II-24 |
| T1-544 | I-7 | II-2 | II-25 |
| T1-545 | I-7 | II-2 | II-26 |
| T1-546 | I-7 | II-2 | II-27 |
| T1-547 | I-7 | II-2 | II-28 |
| T1-548 | I-7 | II-2 | II-29 |
| T1-549 | I-7 | II-3 | II-7 |
| T1-550 | I-7 | II-3 | II-8 |
| T1-551 | I-7 | II-3 | II-9 |
| T1-552 | I-7 | II-3 | II-13 |
| T1-553 | I-7 | II-3 | II-14 |
| T1-554 | I-7 | II-3 | II-15 |
| T1-555 | I-7 | II-3 | II-20 |
| T1-556 | I-7 | II-3 | II-21 |
| T1-557 | I-7 | II-3 | II-22 |
| T1-558 | I-7 | II-3 | II-23 |
| T1-559 | I-7 | II-3 | II-24 |
| T1-560 | I-7 | II-3 | II-25 |
| T1-561 | I-7 | II-3 | II-26 |
| T1-562 | I-7 | II-3 | II-27 |
| T1-563 | I-7 | II-3 | II-28 |
| T1-564 | I-7 | II-3 | II-29 |
| T1-565 | I-7 | II-7 | II-8 |
| T1-566 | I-7 | II-7 | II-9 |
| T1-567 | I-7 | II-7 | II-13 |
| T1-568 | I-7 | II-7 | II-14 |
| T1-569 | I-7 | II-7 | II-15 |
| T1-570 | I-7 | II-7 | II-20 |
| T1-571 | I-7 | II-7 | II-21 |
| T1-572 | I-7 | II-7 | II-22 |
| T1-573 | I-7 | II-7 | II-23 |
| T1-574 | I-7 | II-7 | II-24 |
| T1-575 | I-7 | II-7 | II-25 |
| T1-576 | I-7 | II-7 | II-26 |
| T1-577 | I-7 | II-7 | II-27 |
| T1-578 | I-7 | II-7 | II-28 |
| T1-579 | I-7 | II-7 | II-29 |
| T1-580 | I-7 | II-8 | II-9 |
| T1-581 | I-7 | II-8 | II-13 |
| T1-582 | I-7 | II-8 | II-14 |
| T1-583 | I-7 | II-8 | II-15 |
| T1-584 | I-7 | II-8 | II-20 |
| T1-585 | I-7 | II-8 | II-21 |
| T1-586 | I-7 | II-8 | II-22 |
| T1-587 | I-7 | II-8 | II-23 |
| T1-588 | I-7 | II-8 | II-24 |
| T1-589 | I-7 | II-8 | II-25 |
| T1-590 | I-7 | II-8 | II-26 |
| T1-591 | I-7 | II-8 | II-27 |
| T1-592 | I-7 | II-8 | II-28 |
| T1-593 | I-7 | II-8 | II-29 |
| T1-594 | I-7 | II-9 | II-13 |
| T1-595 | I-7 | II-9 | II-14 |
| T1-596 | I-7 | II-9 | II-15 |
| T1-597 | I-7 | II-9 | II-20 |
| T1-598 | I-7 | II-9 | II-21 |
| T1-599 | I-7 | II-9 | II-22 |
| T1-600 | I-7 | II-9 | II-23 |
| T1-601 | I-7 | II-9 | II-24 |
| T1-602 | I-7 | II-9 | II-25 |
| T1-603 | I-7 | II-9 | II-26 |
| T1-604 | I-7 | II-9 | II-27 |
| T1-605 | I-7 | II-9 | II-28 |
| T1-606 | I-7 | II-9 | II-29 |
| T1-607 | I-7 | II-13 | II-14 |
| T1-608 | I-7 | II-13 | II-15 |
| T1-609 | I-7 | II-13 | II-20 |
| T1-610 | I-7 | II-13 | II-21 |
| T1-611 | I-7 | II-13 | II-22 |
| T1-612 | I-7 | II-13 | II-23 |
| T1-613 | I-7 | II-13 | II-24 |
| T1-614 | I-7 | II-13 | II-25 |
| T1-615 | I-7 | II-13 | II-26 |
| T1-616 | I-7 | II-13 | II-27 |
| T1-617 | I-7 | II-13 | II-28 |
| T1-618 | I-7 | II-13 | II-29 |
| T1-619 | I-7 | II-14 | II-15 |
| T1-620 | I-7 | II-14 | II-20 |
| T1-621 | I-7 | II-14 | II-21 |
| T1-622 | I-7 | II-14 | II-22 |
| T1-623 | I-7 | II-14 | II-23 |
| T1-624 | I-7 | II-14 | II-24 |
| T1-625 | I-7 | II-14 | II-25 |
| T1-626 | I-7 | II-14 | II-26 |
| T1-627 | I-7 | II-14 | II-27 |
| T1-628 | I-7 | II-14 | II-28 |
| T1-629 | I-7 | II-14 | II-29 |
| T1-630 | I-7 | II-15 | II-20 |
| T1-631 | I-7 | II-15 | II-21 |
| T1-632 | I-7 | II-15 | II-22 |
| T1-633 | I-7 | II-15 | II-23 |
| T1-634 | I-7 | II-15 | II-24 |
| T1-635 | I-7 | II-15 | II-25 |
| T1-636 | I-7 | II-15 | II-26 |
| T1-637 | I-7 | II-15 | II-27 |
| T1-638 | I-7 | II-15 | II-28 |
| T1-639 | I-7 | II-15 | II-29 |
| T1-640 | I-7 | II-20 | II-21 |
| T1-641 | I-7 | II-20 | II-22 |
| T1-642 | I-7 | II-20 | II-23 |
| T1-643 | I-7 | II-20 | II-24 |
| T1-644 | I-7 | II-20 | II-25 |
| T1-645 | I-7 | II-20 | II-26 |
| T1-646 | I-7 | II-20 | II-27 |
| T1-647 | I-7 | II-20 | II-28 |
| T1-648 | I-7 | II-20 | II-29 |
| T1-649 | I-7 | II-21 | II-22 |
| T1-650 | I-7 | II-21 | II-23 |
| T1-651 | I-7 | II-21 | II-24 |
| T1-652 | I-7 | II-21 | II-25 |
| T1-653 | I-7 | II-21 | II-26 |
| T1-654 | I-7 | II-21 | II-27 |
| T1-655 | I-7 | II-21 | II-28 |
| T1-656 | I-7 | II-21 | II-29 |
| T1-657 | I-7 | II-22 | II-23 |
| T1-658 | I-7 | II-22 | II-24 |
| T1-659 | I-7 | II-22 | II-25 |
| T1-660 | I-7 | II-22 | II-26 |
| T1-661 | I-7 | II-22 | II-27 |
| T1-662 | I-7 | II-22 | II-28 |
| T1-663 | I-7 | II-22 | II-29 |
| T1-664 | I-7 | II-23 | II-24 |
| T1-665 | I-7 | II-23 | II-25 |
| T1-666 | I-7 | II-23 | II-26 |
| T1-667 | I-7 | II-23 | II-27 |
| T1-668 | I-7 | II-23 | II-28 |
| T1-669 | I-7 | II-23 | II-29 |
| T1-670 | I-7 | II-24 | II-25 |
| T1-671 | I-7 | II-24 | II-26 |
| T1-672 | I-7 | II-24 | II-27 |
| T1-673 | I-7 | II-24 | II-28 |
| T1-674 | I-7 | II-24 | II-29 |
| T1-675 | I-7 | II-25 | II-26 |
| T1-676 | I-7 | II-25 | II-27 |
| T1-677 | I-7 | II-25 | II-28 |
| T1-678 | I-7 | II-25 | II-29 |
| T1-679 | I-7 | II-26 | II-27 |
| T1-680 | I-7 | II-26 | II-28 |
| T1-681 | I-7 | II-26 | II-29 |
| T1-682 | I-7 | II-27 | II-28 |
| T1-683 | I-7 | II-27 | II-29 |
| T1-684 | I-7 | II-28 | II-29 |
| T1-685 | I-8 | II-1 | II-2 |
| T1-686 | I-8 | II-1 | II-3 |
| T1-687 | I-8 | II-1 | II-7 |
| T1-688 | I-8 | II-1 | II-8 |
| T1-689 | I-8 | II-1 | II-9 |
| T1-690 | I-8 | II-1 | II-13 |
| T1-691 | I-8 | II-1 | II-14 |
| T1-692 | I-8 | II-1 | II-15 |
| T1-693 | I-8 | II-1 | II-20 |
| T1-694 | I-8 | II-1 | II-21 |
| T1-695 | I-8 | II-1 | II-22 |
| T1-696 | I-8 | II-1 | II-23 |
| T1-697 | I-8 | II-1 | II-24 |
| T1-698 | I-8 | II-1 | II-25 |
| T1-699 | I-8 | II-1 | II-26 |
| T1-700 | I-8 | II-1 | II-27 |
| T1-701 | I-8 | II-1 | II-28 |
| T1-702 | I-8 | II-1 | II-29 |
| T1-703 | I-8 | II-2 | II-3 |
| T1-704 | I-8 | II-2 | II-7 |
| T1-705 | I-8 | II-2 | II-8 |
| T1-706 | I-8 | II-2 | II-9 |
| T1-707 | I-8 | II-2 | II-13 |
| T1-708 | I-8 | II-2 | II-14 |
| T1-709 | I-8 | II-2 | II-15 |
| T1-710 | I-8 | II-2 | II-20 |
| T1-711 | I-8 | II-2 | II-21 |
| T1-712 | I-8 | II-2 | II-22 |
| T1-713 | I-8 | II-2 | II-23 |
| T1-714 | I-8 | II-2 | II-24 |
| T1-715 | I-8 | II-2 | II-25 |
| T1-716 | I-8 | II-2 | II-26 |
| T1-717 | I-8 | II-2 | II-27 |
| T1-718 | I-8 | II-2 | II-28 |
| T1-719 | I-8 | II-2 | II-29 |
| T1-720 | I-8 | II-3 | II-7 |
| T1-721 | I-8 | II-3 | II-8 |
| T1-722 | I-8 | II-3 | II-9 |
| T1-723 | I-8 | II-3 | II-13 |
| T1-724 | I-8 | II-3 | II-14 |
| T1-725 | I-8 | II-3 | II-15 |
| T1-726 | I-8 | II-3 | II-20 |
| T1-727 | I-8 | II-3 | II-21 |
| T1-728 | I-8 | II-3 | II-22 |
| T1-729 | I-8 | II-3 | II-23 |
| T1-730 | I-8 | II-3 | II-24 |
| T1-731 | I-8 | II-3 | II-25 |
| T1-732 | I-8 | II-3 | II-26 |
| T1-733 | I-8 | II-3 | II-27 |
| T1-734 | I-8 | II-3 | II-28 |
| T1-735 | I-8 | II-3 | II-29 |
| T1-736 | I-8 | II-7 | II-8 |
| T1-737 | I-8 | II-7 | II-9 |
| T1-738 | I-8 | II-7 | II-13 |
| T1-739 | I-8 | II-7 | II-14 |
| T1-740 | I-8 | II-7 | II-15 |
| T1-741 | I-8 | II-7 | II-20 |
| T1-742 | I-8 | II-7 | II-21 |
| T1-743 | I-8 | II-7 | II-22 |
| T1-744 | I-8 | II-7 | II-23 |
| T1-745 | I-8 | II-7 | II-24 |
| T1-746 | I-8 | II-7 | II-25 |
| T1-747 | I-8 | II-7 | II-26 |
| T1-748 | I-8 | II-7 | II-27 |
| T1-749 | I-8 | II-7 | II-28 |
| T1-750 | I-8 | II-7 | II-29 |
| T1-751 | I-8 | II-8 | II-9 |
| T1-752 | I-8 | II-8 | II-13 |
| T1-753 | I-8 | II-8 | II-14 |
| T1-754 | I-8 | II-8 | II-15 |
| T1-755 | I-8 | II-8 | II-20 |
| T1-756 | I-8 | II-8 | II-21 |
| T1-757 | I-8 | II-8 | II-22 |
| T1-758 | I-8 | II-8 | II-23 |
| T1-759 | I-8 | II-8 | II-24 |
| T1-760 | I-8 | II-8 | II-25 |
| T1-761 | I-8 | II-8 | II-26 |
| T1-762 | I-8 | II-8 | II-27 |
| T1-763 | I-8 | II-8 | II-28 |
| T1-764 | I-8 | II-8 | II-29 |
| T1-765 | I-8 | II-9 | II-13 |
| T1-766 | I-8 | II-9 | II-14 |
| T1-767 | I-8 | II-9 | II-15 |
| T1-768 | I-8 | II-9 | II-20 |
| T1-769 | I-8 | II-9 | II-21 |
| T1-770 | I-8 | II-9 | II-22 |
| T1-771 | I-8 | II-9 | II-23 |
| T1-772 | I-8 | II-9 | II-24 |
| T1-773 | I-8 | II-9 | II-25 |
| T1-774 | I-8 | II-9 | II-26 |
| T1-775 | I-8 | II-9 | II-27 |
| T1-776 | I-8 | II-9 | II-28 |
| T1-777 | I-8 | II-9 | II-29 |
| T1-778 | I-8 | II-13 | II-14 |
| T1-779 | I-8 | II-13 | II-15 |
| T1-780 | I-8 | II-13 | II-20 |
| T1-781 | I-8 | II-13 | II-21 |
| T1-782 | I-8 | II-13 | II-22 |
| T1-783 | I-8 | II-13 | II-23 |
| T1-784 | I-8 | II-13 | II-24 |
| T1-785 | I-8 | II-13 | II-25 |
| T1-786 | I-8 | II-13 | II-26 |
| T1-787 | I-8 | II-13 | II-27 |
| T1-788 | I-8 | II-13 | II-28 |
| T1-789 | I-8 | II-13 | II-29 |
| T1-790 | I-8 | II-14 | II-15 |
| T1-791 | I-8 | II-14 | II-20 |
| T1-792 | I-8 | II-14 | II-21 |
| T1-793 | I-8 | II-14 | II-22 |
| T1-794 | I-8 | II-14 | II-23 |
| T1-795 | I-8 | II-14 | II-24 |
| T1-796 | I-8 | II-14 | II-25 |
| T1-797 | I-8 | II-14 | II-26 |
| T1-798 | I-8 | II-14 | II-27 |
| T1-799 | I-8 | II-14 | II-28 |
| T1-800 | I-8 | II-14 | II-29 |
| T1-801 | I-8 | II-15 | II-20 |
| T1-802 | I-8 | II-15 | II-21 |
| T1-803 | I-8 | II-15 | II-22 |
| T1-804 | I-8 | II-15 | II-23 |
| T1-805 | I-8 | II-15 | II-24 |
| T1-806 | I-8 | II-15 | II-25 |
| T1-807 | I-8 | II-15 | II-26 |
| T1-808 | I-8 | II-15 | II-27 |
| T1-809 | I-8 | II-15 | II-28 |
| T1-810 | I-8 | II-15 | II-29 |
| T1-811 | I-8 | II-20 | II-21 |
| T1-812 | I-8 | II-20 | II-22 |
| T1-813 | I-8 | II-20 | II-23 |
| T1-814 | I-8 | II-20 | II-24 |
| T1-815 | I-8 | II-20 | II-25 |
| T1-816 | I-8 | II-20 | II-26 |
| T1-817 | I-8 | II-20 | II-27 |
| T1-818 | I-8 | II-20 | II-28 |
| T1-819 | I-8 | II-20 | II-29 |
| T1-820 | I-8 | II-21 | II-22 |
| T1-821 | I-8 | II-21 | II-23 |
| T1-822 | I-8 | II-21 | II-24 |
| T1-823 | I-8 | II-21 | II-25 |
| T1-824 | I-8 | II-21 | II-26 |
| T1-825 | I-8 | II-21 | II-27 |
| T1-826 | I-8 | II-21 | II-28 |
| T1-827 | I-8 | II-21 | II-29 |
| T1-828 | I-8 | II-22 | II-23 |
| T1-829 | I-8 | II-22 | II-24 |
| T1-830 | I-8 | II-22 | II-25 |
| T1-831 | I-8 | II-22 | II-26 |
| T1-832 | I-8 | II-22 | II-27 |
| T1-833 | I-8 | II-22 | II-28 |
| T1-834 | I-8 | II-22 | II-29 |
| T1-835 | I-8 | II-23 | II-24 |
| T1-836 | I-8 | II-23 | II-25 |
| T1-837 | I-8 | II-23 | II-26 |
| T1-838 | I-8 | II-23 | II-27 |
| T1-839 | I-8 | II-23 | II-28 |
| T1-840 | I-8 | II-23 | II-29 |
| T1-841 | I-8 | II-24 | II-25 |
| T1-842 | I-8 | II-24 | II-26 |
| T1-843 | I-8 | II-24 | II-27 |
| T1-844 | I-8 | II-24 | II-28 |
| T1-845 | I-8 | II-24 | II-29 |
| T1-846 | I-8 | II-25 | II-26 |
| T1-847 | I-8 | II-25 | II-27 |
| T1-848 | I-8 | II-25 | II-28 |
| T1-849 | I-8 | II-25 | II-29 |
| T1-850 | I-8 | II-26 | II-27 |
| T1-851 | I-8 | II-26 | II-28 |
| T1-852 | I-8 | II-26 | II-29 |
| T1-853 | I-8 | II-27 | II-28 |
| T1-854 | I-8 | II-27 | II-29 |
| T1-855 | I-8 | II-28 | II-29 |
| T1-856 | I-9 | II-1 | II-2 |
| T1-857 | I-9 | II-1 | II-3 |
| T1-858 | I-9 | II-1 | II-7 |
| T1-859 | I-9 | II-1 | II-8 |
| T1-860 | I-9 | II-1 | II-9 |
| T1-861 | I-9 | II-1 | II-13 |
| T1-862 | I-9 | II-1 | II-14 |
| T1-863 | I-9 | II-1 | II-15 |
| T1-864 | I-9 | II-1 | II-20 |
| T1-865 | I-9 | II-1 | II-21 |
| T1-866 | I-9 | II-1 | II-22 |
| T1-867 | I-9 | II-1 | II-23 |
| T1-868 | I-9 | II-1 | II-24 |
| T1-869 | I-9 | II-1 | II-25 |
| T1-870 | I-9 | II-1 | II-26 |
| T1-871 | I-9 | II-1 | II-27 |
| T1-872 | I-9 | II-1 | II-28 |
| T1-873 | I-9 | II-1 | II-29 |
| T1-874 | I-9 | II-2 | II-3 |
| T1-875 | I-9 | II-2 | II-7 |
| T1-876 | I-9 | II-2 | II-8 |
| T1-877 | I-9 | II-2 | II-9 |
| T1-878 | I-9 | II-2 | II-13 |
| T1-879 | I-9 | II-2 | II-14 |
| T1-880 | I-9 | II-2 | II-15 |
| T1-881 | I-9 | II-2 | II-20 |
| T1-882 | I-9 | II-2 | II-21 |
| T1-883 | I-9 | II-2 | II-22 |
| T1-884 | I-9 | II-2 | II-23 |
| T1-885 | I-9 | II-2 | II-24 |
| T1-886 | I-9 | II-2 | II-25 |
| T1-887 | I-9 | II-2 | II-26 |
| T1-888 | I-9 | II-2 | II-27 |
| T1-889 | I-9 | II-2 | II-28 |
| T1-890 | I-9 | II-2 | II-29 |
| T1-891 | I-9 | II-3 | II-7 |
| T1-892 | I-9 | II-3 | II-8 |
| T1-893 | I-9 | II-3 | II-9 |
| T1-894 | I-9 | II-3 | II-13 |
| T1-895 | I-9 | II-3 | II-14 |
| T1-896 | I-9 | II-3 | II-15 |
| T1-897 | I-9 | II-3 | II-20 |
| T1-898 | I-9 | II-3 | II-21 |
| T1-899 | I-9 | II-3 | II-22 |
| T1-900 | I-9 | II-3 | II-23 |
| T1-901 | I-9 | II-3 | II-24 |
| T1-902 | I-9 | II-3 | II-25 |
| T1-903 | I-9 | II-3 | II-26 |
| T1-904 | I-9 | II-3 | II-27 |
| T1-905 | I-9 | II-3 | II-28 |
| T1-906 | I-9 | II-3 | II-29 |
| T1-907 | I-9 | II-7 | II-8 |
| T1-908 | I-9 | II-7 | II-9 |
| T1-909 | I-9 | II-7 | II-13 |
| T1-910 | I-9 | II-7 | II-14 |
| T1-911 | I-9 | II-7 | II-15 |
| T1-912 | I-9 | II-7 | II-20 |
| T1-913 | I-9 | II-7 | II-21 |
| T1-914 | I-9 | II-7 | II-22 |
| T1-915 | I-9 | II-7 | II-23 |
| T1-916 | I-9 | II-7 | II-24 |
| T1-917 | I-9 | II-7 | II-25 |
| T1-918 | I-9 | II-7 | II-26 |
| T1-919 | I-9 | II-7 | II-27 |
| T1-920 | I-9 | II-7 | II-28 |
| T1-921 | I-9 | II-7 | II-29 |
| T1-922 | I-9 | II-8 | II-9 |
| T1-923 | I-9 | II-8 | II-13 |
| T1-924 | I-9 | II-8 | II-14 |
| T1-925 | I-9 | II-8 | II-15 |
| T1-926 | I-9 | II-8 | II-20 |
| T1-927 | I-9 | II-8 | II-21 |
| T1-928 | I-9 | II-8 | II-22 |
| T1-929 | I-9 | II-8 | II-23 |
| T1-930 | I-9 | II-8 | II-24 |
| T1-931 | I-9 | II-8 | II-25 |
| T1-932 | I-9 | II-8 | II-26 |
| T1-933 | I-9 | II-8 | II-27 |
| T1-934 | I-9 | II-8 | II-28 |
| T1-935 | I-9 | II-8 | II-29 |
| T1-936 | I-9 | II-9 | II-13 |
| T1-937 | I-9 | II-9 | II-14 |
| T1-938 | I-9 | II-9 | II-15 |
| T1-939 | I-9 | II-9 | II-20 |
| T1-940 | I-9 | II-9 | II-21 |
| T1-941 | I-9 | II-9 | II-22 |
| T1-942 | I-9 | II-9 | II-23 |
| T1-943 | I-9 | II-9 | II-24 |
| T1-944 | I-9 | II-9 | II-25 |
| T1-945 | I-9 | II-9 | II-26 |
| T1-946 | I-9 | II-9 | II-27 |
| T1-947 | I-9 | II-9 | II-28 |
| T1-948 | I-9 | II-9 | II-29 |
| T1-949 | I-9 | II-13 | II-14 |
| T1-950 | I-9 | II-13 | II-15 |
| T1-951 | I-9 | II-13 | II-20 |
| T1-952 | I-9 | II-13 | II-21 |
| T1-953 | I-9 | II-13 | II-22 |
| T1-954 | I-9 | II-13 | II-23 |
| T1-955 | I-9 | II-13 | II-24 |
| T1-956 | I-9 | II-13 | II-25 |
| T1-957 | I-9 | II-13 | II-26 |
| T1-958 | I-9 | II-13 | II-27 |
| T1-959 | I-9 | II-13 | II-28 |
| T1-960 | I-9 | II-13 | II-29 |
| T1-961 | I-9 | II-14 | II-15 |
| T1-962 | I-9 | II-14 | II-20 |
| T1-963 | I-9 | II-14 | II-21 |
| T1-964 | I-9 | II-14 | II-22 |
| T1-965 | I-9 | II-14 | II-23 |
| T1-966 | I-9 | II-14 | II-24 |
| T1-967 | I-9 | II-14 | II-25 |
| T1-968 | I-9 | II-14 | II-26 |
| T1-969 | I-9 | II-14 | II-27 |
| T1-970 | I-9 | II-14 | II-28 |
| T1-971 | I-9 | II-14 | II-29 |
| T1-972 | I-9 | II-15 | II-20 |
| T1-973 | I-9 | II-15 | II-21 |
| T1-974 | I-9 | II-15 | II-22 |
| T1-975 | I-9 | II-15 | II-23 |
| T1-976 | I-9 | II-15 | II-24 |
| T1-977 | I-9 | II-15 | II-25 |
| T1-978 | I-9 | II-15 | II-26 |
| T1-979 | I-9 | II-15 | II-27 |
| T1-980 | I-9 | II-15 | II-28 |
| T1-981 | I-9 | II-15 | II-29 |
| T1-982 | I-9 | II-20 | II-21 |
| T1-983 | I-9 | II-20 | II-22 |
| T1-984 | I-9 | II-20 | II-23 |
| T1-985 | I-9 | II-20 | II-24 |
| T1-986 | I-9 | II-20 | II-25 |
| T1-987 | I-9 | II-20 | II-26 |
| T1-988 | I-9 | II-20 | II-27 |
| T1-989 | I-9 | II-20 | II-28 |
| T1-990 | I-9 | II-20 | II-29 |
| T1-991 | I-9 | II-21 | II-22 |
| T1-992 | I-9 | II-21 | II-23 |
| T1-993 | I-9 | II-21 | II-24 |
| T1-994 | I-9 | II-21 | II-25 |
| T1-995 | I-9 | II-21 | II-26 |
| T1-996 | I-9 | II-21 | II-27 |
| T1-997 | I-9 | II-21 | II-28 |
| T1-998 | I-9 | II-21 | II-29 |
| T1-999 | I-9 | II-22 | II-23 |
| T1-1000 | I-9 | II-22 | II-24 |
| T1-1001 | I-9 | II-22 | II-25 |
| T1-1002 | I-9 | II-22 | II-26 |
| T1-1003 | I-9 | II-22 | II-27 |
| T1-1004 | I-9 | II-22 | II-28 |
| T1-1005 | I-9 | II-22 | II-29 |
| T1-1006 | I-9 | II-23 | II-24 |
| T1-1007 | I-9 | II-23 | II-25 |
| T1-1008 | I-9 | II-23 | II-26 |
| T1-1009 | I-9 | II-23 | II-27 |
| T1-1010 | I-9 | II-23 | II-28 |
| T1-1011 | I-9 | II-23 | II-29 |
| T1-1012 | I-9 | II-24 | II-25 |
| T1-1013 | I-9 | II-24 | II-26 |
| T1-1014 | I-9 | II-24 | II-27 |
| T1-1015 | I-9 | II-24 | II-28 |
| T1-1016 | I-9 | II-24 | II-29 |
| T1-1017 | I-9 | II-25 | II-26 |
| T1-1018 | I-9 | II-25 | II-27 |
| T1-1019 | I-9 | II-25 | II-28 |
| T1-1020 | I-9 | II-25 | II-29 |
| T1-1021 | I-9 | II-26 | II-27 |
| T1-1022 | I-9 | II-26 | II-28 |
| T1-1023 | I-9 | II-26 | II-29 |
| T1-1024 | I-9 | II-27 | II-28 |
| T1-1025 | I-9 | II-27 | II-29 |
| T1-1026 | I-9 | II-28 | II-29 |
| T1-1027 | I-11 | II-1 | II-2 |
| T1-1028 | I-11 | II-1 | II-3 |
| T1-1029 | I-11 | II-1 | II-7 |
| T1-1030 | I-11 | II-1 | II-8 |
| T1-1031 | I-11 | II-1 | II-9 |
| T1-1032 | I-11 | II-1 | II-13 |
| T1-1033 | I-11 | II-1 | II-14 |
| T1-1034 | I-11 | II-1 | II-15 |
| T1-1035 | I-11 | II-1 | II-20 |
| T1-1036 | I-11 | II-1 | II-21 |
| T1-1037 | I-11 | II-1 | II-22 |
| T1-1038 | I-11 | II-1 | II-23 |
| T1-1039 | I-11 | II-1 | II-24 |
| T1-1040 | I-11 | II-1 | II-25 |
| T1-1041 | I-11 | II-1 | II-26 |
| T1-1042 | I-11 | II-1 | II-27 |
| T1-1043 | I-11 | II-1 | II-28 |
| T1-1044 | I-11 | II-1 | II-29 |
| T1-1045 | I-11 | II-2 | II-3 |
| T1-1046 | I-11 | II-2 | II-7 |
| T1-1047 | I-11 | II-2 | II-8 |
| T1-1048 | I-11 | II-2 | II-9 |
| T1-1049 | I-11 | II-2 | II-13 |
| T1-1050 | I-11 | II-2 | II-14 |
| T1-1051 | I-11 | II-2 | II-15 |
| T1-1052 | I-11 | II-2 | II-20 |
| T1-1053 | I-11 | II-2 | II-21 |
| T1-1054 | I-11 | II-2 | II-22 |
| T1-1055 | I-11 | II-2 | II-23 |
| T1-1056 | I-11 | II-2 | II-24 |
| T1-1057 | I-11 | II-2 | II-25 |
| T1-1058 | I-11 | II-2 | II-26 |
| T1-1059 | I-11 | II-2 | II-27 |
| T1-1060 | I-11 | II-2 | II-28 |
| T1-1061 | I-11 | II-2 | II-29 |
| T1-1062 | I-11 | II-3 | II-7 |
| T1-1063 | I-11 | II-3 | II-8 |
| T1-1064 | I-11 | II-3 | II-9 |
| T1-1065 | I-11 | II-3 | II-13 |
| T1-1066 | I-11 | II-3 | II-14 |
| T1-1067 | I-11 | II-3 | II-15 |
| T1-1068 | I-11 | II-3 | II-20 |
| T1-1069 | I-11 | II-3 | II-21 |
| T1-1070 | I-11 | II-3 | II-22 |
| T1-1071 | I-11 | II-3 | II-23 |
| T1-1072 | I-11 | II-3 | II-24 |
| T1-1073 | I-11 | II-3 | II-25 |
| T1-1074 | I-11 | II-3 | II-26 |
| T1-1075 | I-11 | II-3 | II-27 |
| T1-1076 | I-11 | II-3 | II-28 |
| T1-1077 | I-11 | II-3 | II-29 |
| T1-1078 | I-11 | II-7 | II-8 |
| T1-1079 | I-11 | II-7 | II-9 |
| T1-1080 | I-11 | II-7 | II-13 |
| T1-1081 | I-11 | II-7 | II-14 |
| T1-1082 | I-11 | II-7 | II-15 |
| T1-1083 | I-11 | II-7 | II-20 |
| T1-1084 | I-11 | II-7 | II-21 |
| T1-1085 | I-11 | II-7 | II-22 |
| T1-1086 | I-11 | II-7 | II-23 |
| T1-1087 | I-11 | II-7 | II-24 |
| T1-1088 | I-11 | II-7 | II-25 |
| T1-1089 | I-11 | II-7 | II-26 |
| T1-1090 | I-11 | II-7 | II-27 |
| T1-1091 | I-11 | II-7 | II-28 |
| T1-1092 | I-11 | II-7 | II-29 |
| T1-1093 | I-11 | II-8 | II-9 |
| T1-1094 | I-11 | II-8 | II-13 |
| T1-1095 | I-11 | II-8 | II-14 |
| T1-1096 | I-11 | II-8 | II-15 |
| T1-1097 | I-11 | II-8 | II-20 |
| T1-1098 | I-11 | II-8 | II-21 |
| T1-1099 | I-11 | II-8 | II-22 |
| T1-1100 | I-11 | II-8 | II-23 |
| T1-1101 | I-11 | II-8 | II-24 |
| T1-1102 | I-11 | II-8 | II-25 |
| T1-1103 | I-11 | II-8 | II-26 |
| T1-1104 | I-11 | II-8 | II-27 |
| T1-1105 | I-11 | II-8 | II-28 |
| T1-1106 | I-11 | II-8 | II-29 |
| T1-1107 | I-11 | II-9 | II-13 |
| T1-1108 | I-11 | II-9 | II-14 |
| T1-1109 | I-11 | II-9 | II-15 |
| T1-1110 | I-11 | II-9 | II-20 |
| T1-1111 | I-11 | II-9 | II-21 |
| T1-1112 | I-11 | II-9 | II-22 |
| T1-1113 | I-11 | II-9 | II-23 |
| T1-1114 | I-11 | II-9 | II-24 |
| T1-1115 | I-11 | II-9 | II-25 |
| T1-1116 | I-11 | II-9 | II-26 |
| T1-1117 | I-11 | II-9 | II-27 |
| T1-1118 | I-11 | II-9 | II-28 |
| T1-1119 | I-11 | II-9 | II-29 |
| T1-1120 | I-11 | II-13 | II-14 |
| T1-1121 | I-11 | II-13 | II-15 |
| T1-1122 | I-11 | II-13 | II-20 |
| T1-1123 | I-11 | II-13 | II-21 |
| T1-1124 | I-11 | II-13 | II-22 |
| T1-1125 | I-11 | II-13 | II-23 |
| T1-1126 | I-11 | II-13 | II-24 |
| T1-1127 | I-11 | II-13 | II-25 |
| T1-1128 | I-11 | II-13 | II-26 |
| T1-1129 | I-11 | II-13 | II-27 |
| T1-1130 | I-11 | II-13 | II-28 |
| T1-1131 | I-11 | II-13 | II-29 |
| T1-1132 | I-11 | II-14 | II-15 |
| T1-1133 | I-11 | II-14 | II-20 |
| T1-1134 | I-11 | II-14 | II-21 |
| T1-1135 | I-11 | II-14 | II-22 |
| T1-1136 | I-11 | II-14 | II-23 |
| T1-1137 | I-11 | II-14 | II-24 |
| T1-1138 | I-11 | II-14 | II-25 |
| T1-1139 | I-11 | II-14 | II-26 |
| T1-1140 | I-11 | II-14 | II-27 |
| T1-1141 | I-11 | II-14 | II-28 |
| T1-1142 | I-11 | II-14 | II-29 |
| T1-1143 | I-11 | II-15 | II-20 |
| T1-1144 | I-11 | II-15 | II-21 |
| T1-1145 | I-11 | II-15 | II-22 |
| T1-1146 | I-11 | II-15 | II-23 |
| T1-1147 | I-11 | II-15 | II-24 |
| T1-1148 | I-11 | II-15 | II-25 |
| T1-1149 | I-11 | II-15 | II-26 |
| T1-1150 | I-11 | II-15 | II-27 |
| T1-1151 | I-11 | II-15 | II-28 |
| T1-1152 | I-11 | II-15 | II-29 |
| T1-1153 | I-11 | II-20 | II-21 |
| T1-1154 | I-11 | II-20 | II-22 |
| T1-1155 | I-11 | II-20 | II-23 |
| T1-1156 | I-11 | II-20 | II-24 |
| T1-1157 | I-11 | II-20 | II-25 |
| T1-1158 | I-11 | II-20 | II-26 |
| T1-1159 | I-11 | II-20 | II-27 |
| T1-1160 | I-11 | II-20 | II-28 |
| T1-1161 | I-11 | II-20 | II-29 |
| T1-1162 | I-11 | II-21 | II-22 |
| T1-1163 | I-11 | II-21 | II-23 |
| T1-1164 | I-11 | II-21 | II-24 |
| T1-1165 | I-11 | II-21 | II-25 |
| T1-1166 | I-11 | II-21 | II-26 |
| T1-1167 | I-11 | II-21 | II-27 |
| T1-1168 | I-11 | II-21 | II-28 |
| T1-1169 | I-11 | II-21 | II-29 |
| T1-1170 | I-11 | II-22 | II-23 |
| T1-1171 | I-11 | II-22 | II-24 |
| T1-1172 | I-11 | II-22 | II-25 |
| T1-1173 | I-11 | II-22 | II-26 |
| T1-1174 | I-11 | II-22 | II-27 |
| T1-1175 | I-11 | II-22 | II-28 |
| T1-1176 | I-11 | II-22 | II-29 |
| T1-1177 | I-11 | II-23 | II-24 |
| T1-1178 | I-11 | II-23 | II-25 |
| T1-1179 | I-11 | II-23 | II-26 |
| T1-1180 | I-11 | II-23 | II-27 |
| T1-1181 | I-11 | II-23 | II-28 |
| T1-1182 | I-11 | II-23 | II-29 |
| T1-1183 | I-11 | II-24 | II-25 |
| T1-1184 | I-11 | II-24 | II-26 |
| T1-1185 | I-11 | II-24 | II-27 |
| T1-1186 | I-11 | II-24 | II-28 |
| T1-1187 | I-11 | II-24 | II-29 |
| T1-1188 | I-11 | II-25 | II-26 |
| T1-1189 | I-11 | II-25 | II-27 |
| T1-1190 | I-11 | II-25 | II-28 |
| T1-1191 | I-11 | II-25 | II-29 |
| T1-1192 | I-11 | II-26 | II-27 |
| T1-1193 | I-11 | II-26 | II-28 |
| T1-1194 | I-11 | II-26 | II-29 |
| T1-1195 | I-11 | II-27 | II-28 |
| T1-1196 | I-11 | II-27 | II-29 |
| T1-1197 | I-11 | II-28 | II-29 |
| T1-1198 | I-12 | II-1 | II-2 |
| T1-1199 | I-12 | II-1 | II-3 |
| T1-1200 | I-12 | II-1 | II-7 |
| T1-1201 | I-12 | II-1 | II-8 |
| T1-1202 | I-12 | II-1 | II-9 |
| T1-1203 | I-12 | II-1 | II-13 |
| T1-1204 | I-12 | II-1 | II-14 |
| T1-1205 | I-12 | II-1 | II-15 |
| T1-1206 | I-12 | II-1 | II-20 |
| T1-1207 | I-12 | II-1 | II-21 |
| T1-1208 | I-12 | II-1 | II-22 |
| T1-1209 | I-12 | II-1 | II-23 |
| T1-1210 | I-12 | II-1 | II-24 |
| T1-1211 | I-12 | II-1 | II-25 |
| T1-1212 | I-12 | II-1 | II-26 |
| T1-1213 | I-12 | II-1 | II-27 |
| T1-1214 | I-12 | II-1 | II-28 |
| T1-1215 | I-12 | II-1 | II-29 |
| T1-1216 | I-12 | II-2 | II-3 |
| T1-1217 | I-12 | II-2 | II-7 |
| T1-1218 | I-12 | II-2 | II-8 |
| T1-1219 | I-12 | II-2 | II-9 |
| T1-1220 | I-12 | II-2 | II-13 |
| T1-1221 | I-12 | II-2 | II-14 |
| T1-1222 | I-12 | II-2 | II-15 |
| T1-1223 | I-12 | II-2 | II-20 |
| T1-1224 | I-12 | II-2 | II-21 |
| T1-1225 | I-12 | II-2 | II-22 |
| T1-1226 | I-12 | II-2 | II-23 |
| T1-1227 | I-12 | II-2 | II-24 |
| T1-1228 | I-12 | II-2 | II-25 |
| T1-1229 | I-12 | II-2 | II-26 |
| T1-1230 | I-12 | II-2 | II-27 |
| T1-1231 | I-12 | II-2 | II-28 |
| T1-1232 | I-12 | II-2 | II-29 |
| T1-1233 | I-12 | II-3 | II-7 |
| T1-1234 | I-12 | II-3 | II-8 |
| T1-1235 | I-12 | II-3 | II-9 |
| T1-1236 | I-12 | II-3 | II-13 |
| T1-1237 | I-12 | II-3 | II-14 |
| T1-1238 | I-12 | II-3 | II-15 |
| T1-1239 | I-12 | II-3 | II-20 |
| T1-1240 | I-12 | II-3 | II-21 |
| T1-1241 | I-12 | II-3 | II-22 |
| T1-1242 | I-12 | II-3 | II-23 |
| T1-1243 | I-12 | II-3 | II-24 |
| T1-1244 | I-12 | II-3 | II-25 |
| T1-1245 | I-12 | II-3 | II-26 |
| T1-1246 | I-12 | II-3 | II-27 |
| T1-1247 | I-12 | II-3 | II-28 |
| T1-1248 | I-12 | II-3 | II-29 |
| T1-1249 | I-12 | II-7 | II-8 |
| T1-1250 | I-12 | II-7 | II-9 |
| T1-1251 | I-12 | II-7 | II-13 |
| T1-1252 | I-12 | II-7 | II-14 |
| T1-1253 | I-12 | II-7 | II-15 |
| T1-1254 | I-12 | II-7 | II-20 |
| T1-1255 | I-12 | II-7 | II-21 |
| T1-1256 | I-12 | II-7 | II-22 |
| T1-1257 | I-12 | II-7 | II-23 |
| T1-1258 | I-12 | II-7 | II-24 |
| T1-1259 | I-12 | II-7 | II-25 |
| T1-1260 | I-12 | II-7 | II-26 |
| T1-1261 | I-12 | II-7 | II-27 |
| T1-1262 | I-12 | II-7 | II-28 |
| T1-1263 | I-12 | II-7 | II-29 |
| T1-1264 | I-12 | II-8 | II-9 |
| T1-1265 | I-12 | II-8 | II-13 |
| T1-1266 | I-12 | II-8 | II-14 |
| T1-1267 | I-12 | II-8 | II-15 |
| T1-1268 | I-12 | II-8 | II-20 |
| T1-1269 | I-12 | II-8 | II-21 |
| T1-1270 | I-12 | II-8 | II-22 |
| T1-1271 | I-12 | II-8 | II-23 |
| T1-1272 | I-12 | II-8 | II-24 |
| T1-1273 | I-12 | II-8 | II-25 |
| T1-1274 | I-12 | II-8 | II-26 |
| T1-1275 | I-12 | II-8 | II-27 |
| T1-1276 | I-12 | II-8 | II-28 |
| T1-1277 | I-12 | II-8 | II-29 |
| T1-1278 | I-12 | II-9 | II-13 |
| T1-1279 | I-12 | II-9 | II-14 |
| T1-1280 | I-12 | II-9 | II-15 |
| T1-1281 | I-12 | II-9 | II-20 |
| T1-1282 | I-12 | II-9 | II-21 |
| T1-1283 | I-12 | II-9 | II-22 |
| T1-1284 | I-12 | II-9 | II-23 |
| T1-1285 | I-12 | II-9 | II-24 |
| T1-1286 | I-12 | II-9 | II-25 |
| T1-1287 | I-12 | II-9 | II-26 |
| T1-1288 | I-12 | II-9 | II-27 |
| T1-1289 | I-12 | II-9 | II-28 |
| T1-1290 | I-12 | II-9 | II-29 |
| T1-1291 | I-12 | II-13 | II-14 |
| T1-1292 | I-12 | II-13 | II-15 |
| T1-1293 | I-12 | II-13 | II-20 |
| T1-1294 | I-12 | II-13 | II-21 |
| T1-1295 | I-12 | II-13 | II-22 |
| T1-1296 | I-12 | II-13 | II-23 |
| T1-1297 | I-12 | II-13 | II-24 |
| T1-1298 | I-12 | II-13 | II-25 |
| T1-1299 | I-12 | II-13 | II-26 |
| T1-1300 | I-12 | II-13 | II-27 |
| T1-1301 | I-12 | II-13 | II-28 |
| T1-1302 | I-12 | II-13 | II-29 |
| T1-1303 | I-12 | II-14 | II-15 |
| T1-1304 | I-12 | II-14 | II-20 |
| T1-1305 | I-12 | II-14 | II-21 |
| T1-1306 | I-12 | II-14 | II-22 |
| T1-1307 | I-12 | II-14 | II-23 |
| T1-1308 | I-12 | II-14 | II-24 |
| T1-1309 | I-12 | II-14 | II-25 |
| T1-1310 | I-12 | II-14 | II-26 |
| T1-1311 | I-12 | II-14 | II-27 |
| T1-1312 | I-12 | II-14 | II-28 |
| T1-1313 | I-12 | II-14 | II-29 |
| T1-1314 | I-12 | II-15 | II-20 |
| T1-1315 | I-12 | II-15 | II-21 |
| T1-1316 | I-12 | II-15 | II-22 |
| T1-1317 | I-12 | II-15 | II-23 |
| T1-1318 | I-12 | II-15 | II-24 |
| T1-1319 | I-12 | II-15 | II-25 |
| T1-1320 | I-12 | II-15 | II-26 |
| T1-1321 | I-12 | II-15 | II-27 |
| T1-1322 | I-12 | II-15 | II-28 |
| T1-1323 | I-12 | II-15 | II-29 |
| T1-1324 | I-12 | II-20 | II-21 |
| T1-1325 | I-12 | II-20 | II-22 |
| T1-1326 | I-12 | II-20 | II-23 |
| T1-1327 | I-12 | II-20 | II-24 |
| T1-1328 | I-12 | II-20 | II-25 |
| T1-1329 | I-12 | II-20 | II-26 |
| T1-1330 | I-12 | II-20 | II-27 |
| T1-1331 | I-12 | II-20 | II-28 |
| T1-1332 | I-12 | II-20 | II-29 |
| T1-1333 | I-12 | II-21 | II-22 |
| T1-1334 | I-12 | II-21 | II-23 |
| T1-1335 | I-12 | II-21 | II-24 |
| T1-1336 | I-12 | II-21 | II-25 |
| T1-1337 | I-12 | II-21 | II-26 |
| T1-1338 | I-12 | II-21 | II-27 |
| T1-1339 | I-12 | II-21 | II-28 |
| T1-1340 | I-12 | II-21 | II-29 |
| T1-1341 | I-12 | II-22 | II-23 |
| T1-1342 | I-12 | II-22 | II-24 |
| T1-1343 | I-12 | II-22 | II-25 |
| T1-1344 | I-12 | II-22 | II-26 |
| T1-1345 | I-12 | II-22 | II-27 |
| T1-1346 | I-12 | II-22 | II-28 |
| T1-1347 | I-12 | II-22 | II-29 |
| T1-1348 | I-12 | II-23 | II-24 |
| T1-1349 | I-12 | II-23 | II-25 |
| T1-1350 | I-12 | II-23 | II-26 |
| T1-1351 | I-12 | II-23 | II-27 |
| T1-1352 | I-12 | II-23 | II-28 |
| T1-1353 | I-12 | II-23 | II-29 |
| T1-1354 | I-12 | II-24 | II-25 |
| T1-1355 | I-12 | II-24 | II-26 |
| T1-1356 | I-12 | II-24 | II-27 |
| T1-1357 | I-12 | II-24 | II-28 |
| T1-1358 | I-12 | II-24 | II-29 |
| T1-1359 | I-12 | II-25 | II-26 |
| T1-1360 | I-12 | II-25 | II-27 |
| T1-1361 | I-12 | II-25 | II-28 |
| T1-1362 | I-12 | II-25 | II-29 |
| T1-1363 | I-12 | II-26 | II-27 |
| T1-1364 | I-12 | II-26 | II-28 |
| T1-1365 | I-12 | II-26 | II-29 |
| T1-1366 | I-12 | II-27 | II-28 |
| T1-1367 | I-12 | II-27 | II-29 |
| T1-1368 | I-12 | II-28 | II-29 |
| T1-1369 | I-13 | II-1 | II-2 |
| T1-1370 | I-13 | II-1 | II-3 |
| T1-1371 | I-13 | II-1 | II-7 |
| T1-1372 | I-13 | II-1 | II-8 |
| T1-1373 | I-13 | II-1 | II-9 |
| T1-1374 | I-13 | II-1 | II-13 |
| T1-1375 | I-13 | II-1 | II-14 |
| T1-1376 | I-13 | II-1 | II-15 |
| T1-1377 | I-13 | II-1 | II-20 |
| T1-1378 | I-13 | II-1 | II-21 |
| T1-1379 | I-13 | II-1 | II-22 |
| T1-1380 | I-13 | II-1 | II-23 |
| T1-1381 | I-13 | II-1 | II-24 |
| T1-1382 | I-13 | II-1 | II-25 |
| T1-1383 | I-13 | II-1 | II-26 |
| T1-1384 | I-13 | II-1 | II-27 |
| T1-1385 | I-13 | II-1 | II-28 |
| T1-1386 | I-13 | II-1 | II-29 |
| T1-1387 | I-13 | II-2 | II-3 |
| T1-1388 | I-13 | II-2 | II-7 |
| T1-1389 | I-13 | II-2 | II-8 |
| T1-1390 | I-13 | II-2 | II-9 |
| T1-1391 | I-13 | II-2 | II-13 |
| T1-1392 | I-13 | II-2 | II-14 |
| T1-1393 | I-13 | II-2 | II-15 |
| T1-1394 | I-13 | II-2 | II-20 |
| T1-1395 | I-13 | II-2 | II-21 |
| T1-1396 | I-13 | II-2 | II-22 |
| T1-1397 | I-13 | II-2 | II-23 |
| T1-1398 | I-13 | II-2 | II-24 |
| T1-1399 | I-13 | II-2 | II-25 |
| T1-1400 | I-13 | II-2 | II-26 |
| T1-1401 | I-13 | II-2 | II-27 |
| T1-1402 | I-13 | II-2 | II-28 |
| T1-1403 | I-13 | II-2 | II-29 |
| T1-1404 | I-13 | II-3 | II-7 |
| T1-1405 | I-13 | II-3 | II-8 |
| T1-1406 | I-13 | II-3 | II-9 |
| T1-1407 | I-13 | II-3 | II-13 |
| T1-1408 | I-13 | II-3 | II-14 |
| T1-1409 | I-13 | II-3 | II-15 |
| T1-1410 | I-13 | II-3 | II-20 |
| T1-1411 | I-13 | II-3 | II-21 |
| T1-1412 | I-13 | II-3 | II-22 |
| T1-1413 | I-13 | II-3 | II-23 |
| T1-1414 | I-13 | II-3 | II-24 |
| T1-1415 | I-13 | II-3 | II-25 |
| T1-1416 | I-13 | II-3 | II-26 |
| T1-1417 | I-13 | II-3 | II-27 |
| T1-1418 | I-13 | II-3 | II-28 |
| T1-1419 | I-13 | II-3 | II-29 |
| T1-1420 | I-13 | II-7 | II-8 |
| T1-1421 | I-13 | II-7 | II-9 |
| T1-1422 | I-13 | II-7 | II-13 |
| T1-1423 | I-13 | II-7 | II-14 |
| T1-1424 | I-13 | II-7 | II-15 |
| T1-1425 | I-13 | II-7 | II-20 |
| T1-1426 | I-13 | II-7 | II-21 |
| T1-1427 | I-13 | II-7 | II-22 |
| T1-1428 | I-13 | II-7 | II-23 |
| T1-1429 | I-13 | II-7 | II-24 |
| T1-1430 | I-13 | II-7 | II-25 |
| T1-1431 | I-13 | II-7 | II-26 |
| T1-1432 | I-13 | II-7 | II-27 |
| T1-1433 | I-13 | II-7 | II-28 |
| T1-1434 | I-13 | II-7 | II-29 |
| T1-1435 | I-13 | II-8 | II-9 |
| T1-1436 | I-13 | II-8 | II-13 |
| T1-1437 | I-13 | II-8 | II-14 |
| T1-1438 | I-13 | II-8 | II-15 |
| T1-1439 | I-13 | II-8 | II-20 |
| T1-1440 | I-13 | II-8 | II-21 |
| T1-1441 | I-13 | II-8 | II-22 |
| T1-1442 | I-13 | II-8 | II-23 |
| T1-1443 | I-13 | II-8 | II-24 |
| T1-1444 | I-13 | II-8 | II-25 |
| T1-1445 | I-13 | II-8 | II-26 |
| T1-1446 | I-13 | II-8 | II-27 |
| T1-1447 | I-13 | II-8 | II-28 |
| T1-1448 | I-13 | II-8 | II-29 |
| T1-1449 | I-13 | II-9 | II-13 |
| T1-1450 | I-13 | II-9 | II-14 |
| T1-1451 | I-13 | II-9 | II-15 |
| T1-1452 | I-13 | II-9 | II-20 |
| T1-1453 | I-13 | II-9 | II-21 |
| T1-1454 | I-13 | II-9 | II-22 |
| T1-1455 | I-13 | II-9 | II-23 |
| T1-1456 | I-13 | II-9 | II-24 |
| T1-1457 | I-13 | II-9 | II-25 |
| T1-1458 | I-13 | II-9 | II-26 |
| T1-1459 | I-13 | II-9 | II-27 |
| T1-1460 | I-13 | II-9 | II-28 |
| T1-1461 | I-13 | II-9 | II-29 |
| T1-1462 | I-13 | II-13 | II-14 |
| T1-1463 | I-13 | II-13 | II-15 |
| T1-1464 | I-13 | II-13 | II-20 |
| T1-1465 | I-13 | II-13 | II-21 |
| T1-1466 | I-13 | II-13 | II-22 |
| T1-1467 | I-13 | II-13 | II-23 |
| T1-1468 | I-13 | II-13 | II-24 |
| T1-1469 | I-13 | II-13 | II-25 |
| T1-1470 | I-13 | II-13 | II-26 |
| T1-1471 | I-13 | II-13 | II-27 |
| T1-1472 | I-13 | II-13 | II-28 |
| T1-1473 | I-13 | II-13 | II-29 |
| T1-1474 | I-13 | II-14 | II-15 |
| T1-1475 | I-13 | II-14 | II-20 |
| T1-1476 | I-13 | II-14 | II-21 |
| T1-1477 | I-13 | II-14 | II-22 |
| T1-1478 | I-13 | II-14 | II-23 |
| T1-1479 | I-13 | II-14 | II-24 |
| T1-1480 | I-13 | II-14 | II-25 |
| T1-1481 | I-13 | II-14 | II-26 |
| T1-1482 | I-13 | II-14 | II-27 |
| T1-1483 | I-13 | II-14 | II-28 |
| T1-1484 | I-13 | II-14 | II-29 |
| T1-1485 | I-13 | II-15 | II-20 |
| T1-1486 | I-13 | II-15 | II-21 |
| T1-1487 | I-13 | II-15 | II-22 |
| T1-1488 | I-13 | II-15 | II-23 |
| T1-1489 | I-13 | II-15 | II-24 |
| T1-1490 | I-13 | II-15 | II-25 |
| T1-1491 | I-13 | II-15 | II-26 |
| T1-1492 | I-13 | II-15 | II-27 |
| T1-1493 | I-13 | II-15 | II-28 |
| T1-1494 | I-13 | II-15 | II-29 |
| T1-1495 | I-13 | II-20 | II-21 |
| T1-1496 | I-13 | II-20 | II-22 |
| T1-1497 | I-13 | II-20 | II-23 |
| T1-1498 | I-13 | II-20 | II-24 |
| T1-1499 | I-13 | II-20 | II-25 |
| T1-1500 | I-13 | II-20 | II-26 |
| T1-1501 | I-13 | II-20 | II-27 |
| T1-1502 | I-13 | II-20 | II-28 |
| T1-1503 | I-13 | II-20 | II-29 |
| T1-1504 | I-13 | II-21 | II-22 |
| T1-1505 | I-13 | II-21 | II-23 |
| T1-1506 | I-13 | II-21 | II-24 |
| T1-1507 | I-13 | II-21 | II-25 |
| T1-1508 | I-13 | II-21 | II-26 |
| T1-1509 | I-13 | II-21 | II-27 |
| T1-1510 | I-13 | II-21 | II-28 |
| T1-1511 | I-13 | II-21 | II-29 |
| T1-1512 | I-13 | II-22 | II-23 |
| T1-1513 | I-13 | II-22 | II-24 |
| T1-1514 | I-13 | II-22 | II-25 |
| T1-1515 | I-13 | II-22 | II-26 |
| T1-1516 | I-13 | II-22 | II-27 |
| T1-1517 | I-13 | II-22 | II-28 |
| T1-1518 | I-13 | II-22 | II-29 |
| T1-1519 | I-13 | II-23 | II-24 |
| T1-1520 | I-13 | II-23 | II-25 |
| T1-1521 | I-13 | II-23 | II-26 |
| T1-1522 | I-13 | II-23 | II-27 |
| T1-1523 | I-13 | II-23 | II-28 |
| T1-1524 | I-13 | II-23 | II-29 |
| T1-1525 | I-13 | II-24 | II-25 |
| T1-1526 | I-13 | II-24 | II-26 |
| T1-1527 | I-13 | II-24 | II-27 |
| T1-1528 | I-13 | II-24 | II-28 |
| T1-1529 | I-13 | II-24 | II-29 |
| T1-1530 | I-13 | II-25 | II-26 |
| T1-1531 | I-13 | II-25 | II-27 |
| T1-1532 | I-13 | II-25 | II-28 |
| T1-1533 | I-13 | II-25 | II-29 |
| T1-1534 | I-13 | II-26 | II-27 |
| T1-1535 | I-13 | II-26 | II-28 |
| T1-1536 | I-13 | II-26 | II-29 |
| T1-1537 | I-13 | II-27 | II-28 |
| T1-1538 | I-13 | II-27 | II-29 |
| T1-1539 | I-13 | II-28 | II-29 |
| T1-1540 | I-14 | II-1 | II-2 |
| T1-1541 | I-14 | II-1 | II-3 |
| T1-1542 | I-14 | II-1 | II-7 |
| T1-1543 | I-14 | II-1 | II-8 |
| T1-1544 | I-14 | II-1 | II-9 |
| T1-1545 | I-14 | II-1 | II-13 |
| T1-1546 | I-14 | II-1 | II-14 |
| T1-1547 | I-14 | II-1 | II-15 |
| T1-1548 | I-14 | II-1 | II-20 |
| T1-1549 | I-14 | II-1 | II-21 |
| T1-1550 | I-14 | II-1 | II-22 |
| T1-1551 | I-14 | II-1 | II-23 |
| T1-1552 | I-14 | II-1 | II-24 |
| T1-1553 | I-14 | II-1 | II-25 |
| T1-1554 | I-14 | II-1 | II-26 |
| T1-1555 | I-14 | II-1 | II-27 |
| T1-1556 | I-14 | II-1 | II-28 |
| T1-1557 | I-14 | II-1 | II-29 |
| T1-1558 | I-14 | II-2 | II-3 |
| T1-1559 | I-14 | II-2 | II-7 |
| T1-1560 | I-14 | II-2 | II-8 |
| T1-1561 | I-14 | II-2 | II-9 |
| T1-1562 | I-14 | II-2 | II-13 |
| T1-1563 | I-14 | II-2 | II-14 |
| T1-1564 | I-14 | II-2 | II-15 |
| T1-1565 | I-14 | II-2 | II-20 |
| T1-1566 | I-14 | II-2 | II-21 |
| T1-1567 | I-14 | II-2 | II-22 |
| T1-1568 | I-14 | II-2 | II-23 |
| T1-1569 | I-14 | II-2 | II-24 |
| T1-1570 | I-14 | II-2 | II-25 |
| T1-1571 | I-14 | II-2 | II-26 |
| T1-1572 | I-14 | II-2 | II-27 |
| T1-1573 | I-14 | II-2 | II-28 |
| T1-1574 | I-14 | II-2 | II-29 |
| T1-1575 | I-14 | II-3 | II-7 |
| T1-1576 | I-14 | II-3 | II-8 |
| T1-1577 | I-14 | II-3 | II-9 |
| T1-1578 | I-14 | II-3 | II-13 |
| T1-1579 | I-14 | II-3 | II-14 |
| T1-1580 | I-14 | II-3 | II-15 |
| T1-1581 | I-14 | II-3 | II-20 |
| T1-1582 | I-14 | II-3 | II-21 |
| T1-1583 | I-14 | II-3 | II-22 |
| T1-1584 | I-14 | II-3 | II-23 |
| T1-1585 | I-14 | II-3 | II-24 |
| T1-1586 | I-14 | II-3 | II-25 |
| T1-1587 | I-14 | II-3 | II-26 |
| T1-1588 | I-14 | II-3 | II-27 |
| T1-1589 | I-14 | II-3 | II-28 |
| T1-1590 | I-14 | II-3 | II-29 |
| T1-1591 | I-14 | II-7 | II-8 |
| T1-1592 | I-14 | II-7 | II-9 |
| T1-1593 | I-14 | II-7 | II-13 |
| T1-1594 | I-14 | II-7 | II-14 |
| T1-1595 | I-14 | II-7 | II-15 |
| T1-1596 | I-14 | II-7 | II-20 |
| T1-1597 | I-14 | II-7 | II-21 |
| T1-1598 | I-14 | II-7 | II-22 |
| T1-1599 | I-14 | II-7 | II-23 |
| T1-1600 | I-14 | II-7 | II-24 |
| T1-1601 | I-14 | II-7 | II-25 |
| T1-1602 | I-14 | II-7 | II-26 |
| T1-1603 | I-14 | II-7 | II-27 |
| T1-1604 | I-14 | II-7 | II-28 |
| T1-1605 | I-14 | II-7 | II-29 |
| T1-1606 | I-14 | II-8 | II-9 |
| T1-1607 | I-14 | II-8 | II-13 |
| T1-1608 | I-14 | II-8 | II-14 |
| T1-1609 | I-14 | II-8 | II-15 |
| T1-1610 | I-14 | II-8 | II-20 |
| T1-1611 | I-14 | II-8 | II-21 |
| T1-1612 | I-14 | II-8 | II-22 |
| T1-1613 | I-14 | II-8 | II-23 |
| T1-1614 | I-14 | II-8 | II-24 |
| T1-1615 | I-14 | II-8 | II-25 |
| T1-1616 | I-14 | II-8 | II-26 |
| T1-1617 | I-14 | II-8 | II-27 |
| T1-1618 | I-14 | II-8 | II-28 |
| T1-1619 | I-14 | II-8 | II-29 |
| T1-1620 | I-14 | II-9 | II-13 |
| T1-1621 | I-14 | II-9 | II-14 |
| T1-1622 | I-14 | II-9 | II-15 |
| T1-1623 | I-14 | II-9 | II-20 |
| T1-1624 | I-14 | II-9 | II-21 |
| T1-1625 | I-14 | II-9 | II-22 |
| T1-1626 | I-14 | II-9 | II-23 |
| T1-1627 | I-14 | II-9 | II-24 |
| T1-1628 | I-14 | II-9 | II-25 |
| T1-1629 | I-14 | II-9 | II-26 |
| T1-1630 | I-14 | II-9 | II-27 |
| T1-1631 | I-14 | II-9 | II-28 |
| T1-1632 | I-14 | II-9 | II-29 |
| T1-1633 | I-14 | II-13 | II-14 |
| T1-1634 | I-14 | II-13 | II-15 |
| T1-1635 | I-14 | II-13 | II-20 |
| T1-1636 | I-14 | II-13 | II-21 |
| T1-1637 | I-14 | II-13 | II-22 |
| T1-1638 | I-14 | II-13 | II-23 |
| T1-1639 | I-14 | II-13 | II-24 |
| T1-1640 | I-14 | II-13 | II-25 |
| T1-1641 | I-14 | II-13 | II-26 |
| T1-1642 | I-14 | II-13 | II-27 |
| T1-1643 | I-14 | II-13 | II-28 |
| T1-1644 | I-14 | II-13 | II-29 |
| T1-1645 | I-14 | II-14 | II-15 |
| T1-1646 | I-14 | II-14 | II-20 |
| T1-1647 | I-14 | II-14 | II-21 |
| T1-1648 | I-14 | II-14 | II-22 |
| T1-1649 | I-14 | II-14 | II-23 |
| T1-1650 | I-14 | II-14 | II-24 |
| T1-1651 | I-14 | II-14 | II-25 |
| T1-1652 | I-14 | II-14 | II-26 |
| T1-1653 | I-14 | II-14 | II-27 |
| T1-1654 | I-14 | II-14 | II-28 |
| T1-1655 | I-14 | II-14 | II-29 |
| T1-1656 | I-14 | II-15 | II-20 |
| T1-1657 | I-14 | II-15 | II-21 |
| T1-1658 | I-14 | II-15 | II-22 |
| T1-1659 | I-14 | II-15 | II-23 |
| T1-1660 | I-14 | II-15 | II-24 |
| T1-1661 | I-14 | II-15 | II-25 |
| T1-1662 | I-14 | II-15 | II-26 |
| T1-1663 | I-14 | II-15 | II-27 |
| T1-1664 | I-14 | II-15 | II-28 |
| T1-1665 | I-14 | II-15 | II-29 |
| T1-1666 | I-14 | II-20 | II-21 |
| T1-1667 | I-14 | II-20 | II-22 |
| T1-1668 | I-14 | II-20 | II-23 |
| T1-1669 | I-14 | II-20 | II-24 |
| T1-1670 | I-14 | II-20 | II-25 |
| T1-1671 | I-14 | II-20 | II-26 |
| T1-1672 | I-14 | II-20 | II-27 |
| T1-1673 | I-14 | II-20 | II-28 |
| T1-1674 | I-14 | II-20 | II-29 |
| T1-1675 | I-14 | II-21 | II-22 |
| T1-1676 | I-14 | II-21 | II-23 |
| T1-1677 | I-14 | II-21 | II-24 |
| T1-1678 | I-14 | II-21 | II-25 |
| T1-1679 | I-14 | II-21 | II-26 |
| T1-1680 | I-14 | II-21 | II-27 |
| T1-1681 | I-14 | II-21 | II-28 |
| T1-1682 | I-14 | II-21 | II-29 |
| T1-1683 | I-14 | II-22 | II-23 |
| T1-1684 | I-14 | II-22 | II-24 |
| T1-1685 | I-14 | II-22 | II-25 |
| T1-1686 | I-14 | II-22 | II-26 |
| T1-1687 | I-14 | II-22 | II-27 |
| T1-1688 | I-14 | II-22 | II-28 |
| T1-1689 | I-14 | II-22 | II-29 |
| T1-1690 | I-14 | II-23 | II-24 |
| T1-1691 | I-14 | II-23 | II-25 |
| T1-1692 | I-14 | II-23 | II-26 |
| T1-1693 | I-14 | II-23 | II-27 |
| T1-1694 | I-14 | II-23 | II-28 |
| T1-1695 | I-14 | II-23 | II-29 |
| T1-1696 | I-14 | II-24 | II-25 |
| T1-1697 | I-14 | II-24 | II-26 |
| T1-1698 | I-14 | II-24 | II-27 |
| T1-1699 | I-14 | II-24 | II-28 |
| T1-1700 | I-14 | II-24 | II-29 |
| T1-1701 | I-14 | II-25 | II-26 |
| T1-1702 | I-14 | II-25 | II-27 |
| T1-1703 | I-14 | II-25 | II-28 |
| T1-1704 | I-14 | II-25 | II-29 |
| T1-1705 | I-14 | II-26 | II-27 |
| T1-1706 | I-14 | II-26 | II-28 |
| T1-1707 | I-14 | II-26 | II-29 |
| T1-1708 | I-14 | II-27 | II-28 |
| T1-1709 | I-14 | II-27 | II-29 |
| T1-1710 | I-14 | II-28 | II-29 |
| T1-1711 | I-18 | II-1 | II-2 |
| T1-1712 | I-18 | II-1 | II-3 |
| T1-1713 | I-18 | II-1 | II-7 |
| T1-1714 | I-18 | II-1 | II-8 |
| T1-1715 | I-18 | II-1 | II-9 |
| T1-1716 | I-18 | II-1 | II-13 |
| T1-1717 | I-18 | II-1 | II-14 |
| T1-1718 | I-18 | II-1 | II-15 |
| T1-1719 | I-18 | II-1 | II-20 |
| T1-1720 | I-18 | II-1 | II-21 |
| T1-1721 | I-18 | II-1 | II-22 |
| T1-1722 | I-18 | II-1 | II-23 |
| T1-1723 | I-18 | II-1 | II-24 |
| T1-1724 | I-18 | II-1 | II-25 |
| T1-1725 | I-18 | II-1 | II-26 |
| T1-1726 | I-18 | II-1 | II-27 |
| T1-1727 | I-18 | II-1 | II-28 |
| T1-1728 | I-18 | II-1 | II-29 |
| T1-1729 | I-18 | II-2 | II-3 |
| T1-1730 | I-18 | II-2 | II-7 |
| T1-1731 | I-18 | II-2 | II-8 |
| T1-1732 | I-18 | II-2 | II-9 |
| T1-1733 | I-18 | II-2 | II-13 |
| T1-1734 | I-18 | II-2 | II-14 |
| T1-1735 | I-18 | II-2 | II-15 |
| T1-1736 | I-18 | II-2 | II-20 |
| T1-1737 | I-18 | II-2 | II-21 |
| T1-1738 | I-18 | II-2 | II-22 |
| T1-1739 | I-18 | II-2 | II-23 |
| T1-1740 | I-18 | II-2 | II-24 |
| T1-1741 | I-18 | II-2 | II-25 |
| T1-1742 | I-18 | II-2 | II-26 |
| T1-1743 | I-18 | II-2 | II-27 |
| T1-1744 | I-18 | II-2 | II-28 |
| T1-1745 | I-18 | II-2 | II-29 |
| T1-1746 | I-18 | II-3 | II-7 |
| T1-1747 | I-18 | II-3 | II-8 |
| T1-1748 | I-18 | II-3 | II-9 |
| T1-1749 | I-18 | II-3 | II-13 |
| T1-1750 | I-18 | II-3 | II-14 |
| T1-1751 | I-18 | II-3 | II-15 |
| T1-1752 | I-18 | II-3 | II-20 |
| T1-1753 | I-18 | II-3 | II-21 |
| T1-1754 | I-18 | II-3 | II-22 |
| T1-1755 | I-18 | II-3 | II-23 |
| T1-1756 | I-18 | II-3 | II-24 |
| T1-1757 | I-18 | II-3 | II-25 |
| T1-1758 | I-18 | II-3 | II-26 |
| T1-1759 | I-18 | II-3 | II-27 |
| T1-1760 | I-18 | II-3 | II-28 |
| T1-1761 | I-18 | II-3 | II-29 |
| T1-1762 | I-18 | II-7 | II-8 |
| T1-1763 | I-18 | II-7 | II-9 |
| T1-1764 | I-18 | II-7 | II-13 |
| T1-1765 | I-18 | II-7 | II-14 |
| T1-1766 | I-18 | II-7 | II-15 |
| T1-1767 | I-18 | II-7 | II-20 |
| T1-1768 | I-18 | II-7 | II-21 |
| T1-1769 | I-18 | II-7 | II-22 |
| T1-1770 | I-18 | II-7 | II-23 |
| T1-1771 | I-18 | II-7 | II-24 |
| T1-1772 | I-18 | II-7 | II-25 |
| T1-1773 | I-18 | II-7 | II-26 |
| T1-1774 | I-18 | II-7 | II-27 |
| T1-1775 | I-18 | II-7 | II-28 |
| T1-1776 | I-18 | II-7 | II-29 |
| T1-1777 | I-18 | II-8 | II-9 |
| T1-1778 | I-18 | II-8 | II-13 |
| T1-1779 | I-18 | II-8 | II-14 |
| T1-1780 | I-18 | II-8 | II-15 |
| T1-1781 | I-18 | II-8 | II-20 |
| T1-1782 | I-18 | II-8 | II-21 |
| T1-1783 | I-18 | II-8 | II-22 |
| T1-1784 | I-18 | II-8 | II-23 |
| T1-1785 | I-18 | II-8 | II-24 |
| T1-1786 | I-18 | II-8 | II-25 |
| T1-1787 | I-18 | II-8 | II-26 |
| T1-1788 | I-18 | II-8 | II-27 |
| T1-1789 | I-18 | II-8 | II-28 |
| T1-1790 | I-18 | II-8 | II-29 |
| T1-1791 | I-18 | II-9 | II-13 |
| T1-1792 | I-18 | II-9 | II-14 |
| T1-1793 | I-18 | II-9 | II-15 |
| T1-1794 | I-18 | II-9 | II-20 |
| T1-1795 | I-18 | II-9 | II-21 |
| T1-1796 | I-18 | II-9 | II-22 |
| T1-1797 | I-18 | II-9 | II-23 |
| T1-1798 | I-18 | II-9 | II-24 |
| T1-1799 | I-18 | II-9 | II-25 |
| T1-1800 | I-18 | II-9 | II-26 |
| T1-1801 | I-18 | II-9 | II-27 |
| T1-1802 | I-18 | II-9 | II-28 |
| T1-1803 | I-18 | II-9 | II-29 |
| T1-1804 | I-18 | II-9 | II-14 |
| T1-1805 | I-18 | II-13 | II-15 |
| T1-1806 | I-18 | II-13 | II-20 |
| T1-1807 | I-18 | II-13 | II-21 |
| T1-1808 | I-18 | II-13 | II-22 |
| T1-1809 | I-18 | II-13 | II-23 |
| T1-1810 | I-18 | II-13 | II-24 |
| T1-1811 | I-18 | II-13 | II-25 |
| T1-1812 | I-18 | II-13 | II-26 |
| T1-1813 | I-18 | II-13 | II-27 |
| T1-1814 | I-18 | II-13 | II-28 |
| T1-1815 | I-18 | II-13 | II-29 |
| T1-1816 | I-18 | II-14 | II-15 |
| T1-1817 | I-18 | II-14 | II-20 |
| T1-1818 | I-18 | II-14 | II-21 |
| T1-1819 | I-18 | II-14 | II-22 |
| T1-1820 | I-18 | II-14 | II-23 |
| T1-1821 | I-18 | II-14 | II-24 |
| T1-1822 | I-18 | II-14 | II-25 |
| T1-1823 | I-18 | II-14 | II-26 |
| T1-1824 | I-18 | II-14 | II-27 |
| T1-1825 | I-18 | II-14 | II-28 |
| T1-1826 | I-18 | II-14 | II-29 |
| T1-1827 | I-18 | II-15 | II-20 |
| T1-1828 | I-18 | II-15 | II-21 |
| T1-1829 | I-18 | II-15 | II-22 |
| T1-1830 | I-18 | II-15 | II-23 |
| T1-1831 | I-18 | II-15 | II-24 |
| T1-1832 | I-18 | II-15 | II-25 |
| T1-1833 | I-18 | II-15 | II-26 |
| T1-1834 | I-18 | II-15 | II-27 |
| T1-1835 | I-18 | II-15 | II-28 |
| T1-1836 | I-18 | II-15 | II-29 |
| T1-1837 | I-18 | II-20 | II-21 |
| T1-1838 | I-18 | II-20 | II-22 |
| T1-1839 | I-18 | II-20 | II-23 |
| T1-1840 | I-18 | II-20 | II-24 |
| T1-1841 | I-18 | II-20 | II-25 |
| T1-1842 | I-18 | II-20 | II-26 |
| T1-1843 | I-18 | II-20 | II-27 |
| T1-1844 | I-18 | II-20 | II-28 |
| T1-1845 | I-18 | II-20 | II-29 |
| T1-1846 | I-18 | II-21 | II-22 |
| T1-1847 | I-18 | II-21 | II-23 |
| T1-1848 | I-18 | II-21 | II-24 |
| T1-1849 | I-18 | II-21 | II-25 |
| T1-1850 | I-18 | II-21 | II-26 |
| T1-1851 | I-18 | II-21 | II-27 |
| T1-1852 | I-18 | II-21 | II-28 |
| T1-1853 | I-18 | II-21 | II-29 |
| T1-1854 | I-18 | II-22 | II-23 |
| T1-1855 | I-18 | II-22 | II-24 |
| T1-1856 | I-18 | II-22 | II-25 |
| T1-1857 | I-18 | II-22 | II-26 |
| T1-1858 | I-18 | II-22 | II-27 |
| T1-1859 | I-18 | II-22 | II-28 |
| T1-1860 | I-18 | II-22 | II-29 |
| T1-1861 | I-18 | II-23 | II-24 |
| T1-1862 | I-18 | II-23 | II-25 |
| T1-1863 | I-18 | II-23 | II-26 |
| T1-1864 | I-18 | II-23 | II-27 |
| T1-1865 | I-18 | II-23 | II-28 |
| T1-1866 | I-18 | II-23 | II-29 |
| T1-1867 | I-18 | II-24 | II-25 |
| T1-1868 | I-18 | II-24 | II-26 |
| T1-1869 | I-18 | II-24 | II-27 |
| T1-1870 | I-18 | II-24 | II-28 |
| T1-1871 | I-18 | II-24 | II-29 |
| T1-1872 | I-18 | II-25 | II-26 |
| T1-1873 | I-18 | II-25 | II-27 |
| T1-1874 | I-18 | II-25 | II-28 |
| T1-1875 | I-18 | II-25 | II-29 |
| T1-1876 | I-18 | II-26 | II-27 |
| T1-1877 | I-18 | II-26 | II-28 |
| T1-1878 | I-18 | II-26 | II-29 |
| T1-1879 | I-18 | II-27 | II-28 |
| T1-1880 | I-18 | II-27 | II-29 |
| T1-1881 | I-18 | II-28 | II-29 |

The ternary mixtures T1-1882 to T1-3036 below comprise, as
component 1, a compound I, a component 2 selected from the active
compounds of groups A to I preferred for component 2 and a
component 3 selected from the active compounds of groups A to I
preferred for component 3. In the table below, each row corresponds to
a ternary mixture according to the invention having the mixture
components 1 to 3 listed in each case in the row in question.

| | | | |
|---|---|---|---|
| T1-1882 | I-1 | II-1 | II-6 |
| T1-1883 | I-1 | II-1 | II-11 |
| T1-1884 | I-1 | II-1 | II-12 |
| T1-1885 | I-1 | II-1 | II-18 |
| T1-1886 | I-1 | II-1 | II-19 |
| T1-1887 | I-1 | II-2 | II-6 |
| T1-1888 | I-1 | II-2 | II-11 |
| T1-1889 | I-1 | II-2 | II-12 |
| T1-1890 | I-1 | II-2 | II-18 |
| T1-1891 | I-1 | II-2 | II-19 |
| T1-1892 | I-1 | II-3 | II-6 |
| T1-1893 | I-1 | II-3 | II-11 |
| T1-1894 | I-1 | II-3 | II-12 |
| T1-1895 | I-1 | II-3 | II-18 |
| T1-1896 | I-1 | II-3 | II-19 |
| T1-1897 | I-1 | II-6 | II-7 |
| T1-1898 | I-1 | II-6 | II-8 |
| T1-1899 | I-1 | II-6 | II-9 |
| T1-1900 | I-1 | II-6 | II-11 |
| T1-1901 | I-1 | II-6 | II-12 |
| T1-1902 | I-1 | II-6 | II-13 |
| T1-1903 | I-1 | II-6 | II-14 |
| T1-1904 | I-1 | II-6 | II-15 |
| T1-1905 | I-1 | II-6 | II-18 |
| T1-1906 | I-1 | II-6 | II-19 |
| T1-1907 | I-1 | II-6 | II-20 |
| T1-1908 | I-1 | II-6 | II-21 |
| T1-1909 | I-1 | II-6 | II-22 |
| T1-1910 | I-1 | II-6 | II-23 |
| T1-1911 | I-1 | II-6 | II-24 |
| T1-1912 | I-1 | II-6 | II-25 |
| T1-1913 | I-1 | II-6 | II-26 |
| T1-1914 | I-1 | II-6 | II-27 |
| T1-1915 | I-1 | II-6 | II-28 |
| T1-1916 | I-1 | II-6 | II-29 |
| T1-1917 | I-1 | II-7 | II-11 |
| T1-1918 | I-1 | II-7 | II-12 |
| T1-1919 | I-1 | II-7 | II-18 |
| T1-1920 | I-1 | II-7 | II-19 |
| T1-1921 | I-1 | II-8 | II-11 |
| T1-1922 | I-1 | II-8 | II-12 |
| T1-1923 | I-1 | II-8 | II-18 |
| T1-1924 | I-1 | II-8 | II-19 |
| T1-1925 | I-1 | II-9 | II-11 |
| T1-1926 | I-1 | II-9 | II-12 |
| T1-1927 | I-1 | II-9 | II-18 |
| T1-1928 | I-1 | II-9 | II-19 |
| T1-1929 | I-1 | II-11 | II-12 |
| T1-1930 | I-1 | II-11 | II-13 |
| T1-1931 | I-1 | II-11 | II-14 |
| T1-1932 | I-1 | II-11 | II-15 |
| T1-1933 | I-1 | II-11 | II-18 |
| T1-1934 | I-1 | II-11 | II-19 |
| T1-1935 | I-1 | II-11 | II-20 |
| T1-1936 | I-1 | II-11 | II-21 |
| T1-1937 | I-1 | II-11 | II-22 |
| T1-1938 | I-1 | II-11 | II-23 |
| T1-1939 | I-1 | II-11 | II-24 |
| T1-1940 | I-1 | II-11 | II-25 |
| T1-1941 | I-1 | II-11 | II-26 |
| T1-1942 | I-1 | II-11 | II-27 |
| T1-1943 | I-1 | II-11 | II-28 |
| T1-1944 | I-1 | II-11 | II-29 |
| T1-1945 | I-1 | II-12 | II-13 |
| T1-1946 | I-1 | II-12 | II-14 |
| T1-1947 | I-1 | II-12 | II-15 |
| T1-1948 | I-1 | II-12 | II-18 |
| T1-1949 | I-1 | II-12 | II-19 |
| T1-1950 | I-1 | II-12 | II-20 |
| T1-1951 | I-1 | II-12 | II-21 |
| T1-1952 | I-1 | II-12 | II-22 |
| T1-1953 | I-1 | II-12 | II-23 |
| T1-1954 | I-1 | II-12 | II-24 |
| T1-1955 | I-1 | II-12 | II-25 |
| T1-1956 | I-1 | II-12 | II-26 |
| T1-1957 | I-1 | II-12 | II-27 |
| T1-1958 | I-1 | II-12 | II-28 |
| T1-1959 | I-1 | II-12 | II-29 |
| T1-1960 | I-1 | II-13 | II-18 |
| T1-1961 | I-1 | II-13 | II-19 |
| T1-1962 | I-1 | II-14 | II-18 |
| T1-1963 | I-1 | II-14 | II-19 |
| T1-1964 | I-1 | II-15 | II-18 |
| T1-1965 | I-1 | II-15 | II-19 |
| T1-1966 | I-1 | II-18 | II-19 |
| T1-1967 | I-1 | II-18 | II-20 |
| T1-1968 | I-1 | II-18 | II-21 |
| T1-1969 | I-1 | II-18 | II-22 |
| T1-1970 | I-1 | II-18 | II-23 |
| T1-1971 | I-1 | II-18 | II-24 |
| T1-1972 | I-1 | II-18 | II-25 |
| T1-1973 | I-1 | II-18 | II-26 |
| T1-1974 | I-1 | II-18 | II-27 |
| T1-1975 | I-1 | II-18 | II-28 |
| T1-1976 | I-1 | II-18 | II-29 |
| T1-1977 | I-1 | II-19 | II-20 |
| T1-1978 | I-1 | II-19 | II-21 |
| T1-1979 | I-1 | II-19 | II-22 |
| T1-1980 | I-1 | II-19 | II-23 |
| T1-1981 | I-1 | II-19 | II-24 |
| T1-1982 | I-1 | II-19 | II-25 |
| T1-1983 | I-1 | II-19 | II-26 |
| T1-1984 | I-1 | II-19 | II-27 |
| T1-1985 | I-1 | II-19 | II-28 |
| T1-1986 | I-1 | II-19 | II-29 |
| T1-1987 | I-2 | II-1 | II-6 |
| T1-1988 | I-2 | II-1 | II-11 |
| T1-1989 | I-2 | II-1 | II-12 |
| T1-1990 | I-2 | II-1 | II-18 |
| T1-1991 | I-2 | II-1 | II-19 |
| T1-1992 | I-2 | II-2 | II-6 |
| T1-1993 | I-2 | II-2 | II-11 |
| T1-1994 | I-2 | II-2 | II-12 |
| T1-1995 | I-2 | II-2 | II-18 |
| T1-1996 | I-2 | II-2 | II-19 |
| T1-1997 | I-2 | II-3 | II-6 |
| T1-1998 | I-2 | II-3 | II-11 |
| T1-1999 | I-2 | II-3 | II-12 |
| T1-2000 | I-2 | II-3 | II-18 |
| T1-2001 | I-2 | II-3 | II-19 |
| T1-2002 | I-2 | II-6 | II-7 |
| T1-2003 | I-2 | II-6 | II-8 |
| T1-2004 | I-2 | II-6 | II-9 |
| T1-2005 | I-2 | II-6 | II-11 |
| T1-2006 | I-2 | II-6 | II-12 |
| T1-2007 | I-2 | II-6 | II-13 |
| T1-2008 | I-2 | II-6 | II-14 |
| T1-2009 | I-2 | II-6 | II-15 |
| T1-2010 | I-2 | II-6 | II-18 |
| T1-2011 | I-2 | II-6 | II-19 |
| T1-2012 | I-2 | II-6 | II-20 |
| T1-2013 | I-2 | II-6 | II-21 |
| T1-2014 | I-2 | II-6 | II-22 |
| T1-2015 | I-2 | II-6 | II-23 |
| T1-2016 | I-2 | II-6 | II-24 |
| T1-2017 | I-2 | II-6 | II-25 |
| T1-2018 | I-2 | II-6 | II-26 |
| T1-2019 | I-2 | II-6 | II-27 |
| T1-2020 | I-2 | II-6 | II-28 |
| T1-2021 | I-2 | II- | II-29 |
| T1-2022 | I-2 | II-7 | II-11 |
| T1-2023 | I-2 | II-7 | II-12 |
| T1-2024 | I-2 | II-7 | II-18 |
| T1-2025 | I-2 | II-7 | II-19 |
| T1-2026 | I-2 | II-8 | II-11 |
| T1-2027 | I-2 | II-8 | II-12 |
| T1-2028 | I-2 | II-8 | II-18 |
| T1-2029 | I-2 | II-8 | II-19 |
| T1-2030 | I-2 | II-9 | II-11 |
| T1-2031 | I-2 | II-9 | II-12 |
| T1-2032 | I-2 | II-9 | II-18 |
| T1-2033 | I-2 | II-9 | II-19 |
| T1-2034 | I-2 | II-11 | II-12 |
| T1-2035 | I-2 | II-11 | II-13 |
| T1-2036 | I-2 | II-11 | II-14 |
| T1-2037 | I-2 | II-11 | II-15 |
| T1-2038 | I-2 | II-11 | II-18 |
| T1-2039 | I-2 | II-11 | II-19 |
| T1-2040 | I-2 | II-11 | II-20 |
| T1-2041 | I-2 | II-11 | II-21 |
| T1-2042 | I-2 | II-11 | II-22 |
| T1-2043 | I-2 | II-11 | II-23 |
| T1-2044 | I-2 | II-11 | II-24 |
| T1-2045 | I-2 | II-11 | II-25 |
| T1-2046 | I-2 | II-11 | II-26 |
| T1-2047 | I-2 | II-11 | II-27 |
| T1-2048 | I-2 | II-11 | II-28 |
| T1-2049 | I-2 | II-11 | II-29 |
| T1-2050 | I-2 | II-12 | II-13 |
| T1-2051 | I-2 | II-12 | II-14 |
| T1-2052 | I-2 | II-12 | II-15 |
| T1-2053 | I-2 | II-12 | II-18 |
| T1-2054 | I-2 | II-12 | II-19 |
| T1-2055 | I-2 | II-12 | II-20 |
| T1-2056 | I-2 | II-12 | II-21 |
| T1-2057 | I-2 | II-12 | II-22 |
| T1-2058 | I-2 | II-12 | II-23 |
| T1-2059 | I-2 | II-12 | II-24 |
| T1-2060 | I-2 | II-12 | II-25 |
| T1-2061 | I-2 | II-12 | II-26 |
| T1-2062 | I-2 | II-12 | II-27 |
| T1-2063 | I-2 | II-12 | II-28 |
| T1-2064 | I-2 | II-12 | II-29 |
| T1-2065 | I-2 | II-13 | II-18 |
| T1-2066 | I-2 | II-13 | II-19 |
| T1-2067 | I-2 | II-14 | II-18 |
| T1-2068 | I-2 | II-14 | II-19 |
| T1-2069 | I-2 | II-15 | II-18 |
| T1-2070 | I-2 | II-15 | II-19 |
| T1-2071 | I-2 | II-18 | II-19 |
| T1-2072 | I-2 | II-18 | II-20 |
| T1-2073 | I-2 | II-18 | II-21 |
| T1-2074 | I-2 | II-18 | II-22 |
| T1-2075 | I-2 | II-18 | II-23 |
| T1-2076 | I-2 | II-18 | II-24 |
| T1-2077 | I-2 | II-18 | II-25 |
| T1-2078 | I-2 | II-18 | II-26 |
| T1-2079 | I-2 | II-18 | II-27 |
| T1-2080 | I-2 | II-18 | II-28 |
| T1-2081 | I-2 | II-18 | II-29 |
| T1-2082 | I-2 | II-19 | II-20 |
| T1-2083 | I-2 | II-19 | II-21 |
| T1-2084 | I-2 | II-19 | II-22 |
| T1-2085 | I-2 | II-19 | II-23 |
| T1-2086 | I-2 | II-19 | II-24 |
| T1-2087 | I-2 | II-19 | II-25 |
| T1-2088 | I-2 | II-19 | II-26 |
| T1-2089 | I-2 | II-19 | II-27 |
| T1-2090 | I-2 | II-19 | II-28 |
| T1-2091 | I-2 | II-19 | II-29 |
| T1-2092 | I-6 | II-1 | II-6 |
| T1-2093 | I-6 | II-1 | II-11 |
| T1-2094 | I-6 | II-1 | II-12 |
| T1-2095 | I-6 | II-1 | II-18 |
| T1-2096 | I-6 | II-1 | II-19 |
| T1-2097 | I-6 | II-2 | II-6 |
| T1-2098 | I-6 | II-2 | II-11 |
| T1-2099 | I-6 | II-2 | II-12 |
| T1-2100 | I-6 | II-2 | II-18 |
| T1-2101 | I-6 | II-2 | II-19 |
| T1-2102 | I-6 | II-3 | II-6 |
| T1-2103 | I-6 | II-3 | II-11 |
| T1-2104 | I-6 | II-3 | II-12 |
| T1-2105 | I-6 | II-3 | II-18 |
| T1-2106 | I-6 | II-3 | II-19 |
| T1-2107 | I-6 | II-6 | II-7 |
| T1-2108 | I-6 | II-6 | II-8 |
| T1-2109 | I-6 | II-6 | II-9 |
| T1-2110 | I-6 | II-6 | II-11 |
| T1-2111 | I-6 | II-6 | II-12 |
| T1-2112 | I-6 | II-6 | II-13 |
| T1-2113 | I-6 | II-6 | II-14 |
| T1-2114 | I-6 | II-6 | II-15 |
| T1-2115 | I-6 | II-6 | II-18 |
| T1-2116 | I-6 | II-6 | II-19 |
| T1-2117 | I-6 | II-6 | II-20 |
| T1-2118 | I-6 | II-6 | II-21 |
| T1-2119 | I-6 | II-6 | II-22 |
| T1-2120 | I-6 | II-6 | II-23 |
| T1-2121 | I-6 | II-6 | II-24 |
| T1-2122 | I-6 | II-6 | II-25 |
| T1-2123 | I-6 | II-6 | II-26 |
| T1-2124 | I-6 | II-6 | II-27 |
| T1-2125 | I-6 | II-6 | II-28 |
| T1-2126 | I-6 | II-6 | II-29 |
| T1-2127 | I-6 | II-7 | II-11 |
| T1-2128 | I-6 | II-7 | II-12 |
| T1-2129 | I-6 | II-7 | II-18 |
| T1-2130 | I-6 | II-7 | II-19 |
| T1-2131 | I-6 | II-8 | II-11 |
| T1-2132 | I-6 | II-8 | II-12 |
| T1-2133 | I-6 | II-8 | II-18 |
| T1-2134 | I-6 | II-8 | II-19 |
| T1-2135 | I-6 | II-9 | II-11 |
| T1-2136 | I-6 | II-9 | II-12 |
| T1-2137 | I-6 | II-9 | II-18 |
| T1-2138 | I-6 | II-9 | II-19 |
| T1-2139 | I-6 | II-11 | II-12 |
| T1-2140 | I-6 | II-11 | II-13 |
| T1-2141 | I-6 | II-11 | II-14 |
| T1-2142 | I-6 | II-11 | II-15 |
| T1-2143 | I-6 | II-11 | II-18 |
| T1-2144 | I-6 | II-11 | II-19 |
| T1-2145 | I-6 | II-11 | II-20 |
| T1-2146 | I-6 | II-11 | II-21 |
| T1-2147 | I-6 | II-11 | II-22 |
| T1-2148 | I-6 | II-11 | II-23 |
| T1-2149 | I-6 | II-11 | II-24 |
| T1-2150 | I-6 | II-11 | II-25 |
| T1-2151 | I-6 | II-11 | II-26 |
| T1-2152 | I-6 | II-11 | II-27 |
| T1-2153 | I-6 | II-11 | II-28 |
| T1-2154 | I-6 | II-11 | II-29 |
| T1-2155 | I-6 | II-12 | II-13 |
| T1-2156 | I-6 | II-12 | II-14 |
| T1-2157 | I-6 | II-12 | II-15 |
| T1-2158 | I-6 | II-12 | II-18 |
| T1-2159 | I-6 | II-12 | II-19 |
| T1-2160 | I-6 | II-12 | II-20 |
| T1-2161 | I-6 | II-12 | II-21 |
| T1-2162 | I-6 | II-12 | II-22 |
| T1-2163 | I-6 | II-12 | II-23 |
| T1-2164 | I-6 | II-12 | II-24 |
| T1-2165 | I-6 | II-12 | II-25 |
| T1-2166 | I-6 | II-12 | II-26 |
| T1-2167 | I-6 | II-12 | II-27 |
| T1-2168 | I-6 | II-12 | II-28 |
| T1-2169 | I-6 | II-12 | II-29 |
| T1-2170 | I-6 | II-13 | II-18 |
| T1-2171 | I-6 | II-13 | II-19 |
| T1-2172 | I-6 | II-14 | II-18 |
| T1-2173 | I-6 | II-14 | II-19 |
| T1-2174 | I-6 | II-15 | II-18 |
| T1-2175 | I-6 | II-15 | II-19 |
| T1-2176 | I-6 | II-18 | II-19 |
| T1-2177 | I-6 | II-18 | II-20 |
| T1-2178 | I-6 | II-18 | II-21 |
| T1-2179 | I-6 | II-18 | II-22 |
| T1-2180 | I-6 | II-18 | II-23 |
| T1-2181 | I-6 | II-18 | II-24 |
| T1-2182 | I-6 | II-18 | II-25 |
| T1-2183 | I-6 | II-18 | II-26 |
| T1-2184 | I-6 | II-18 | II-27 |
| T1-2185 | I-6 | II-18 | II-28 |
| T1-2186 | I-6 | II-18 | II-29 |
| T1-2187 | I-6 | II-19 | II-20 |
| T1-2188 | I-6 | II-19 | II-21 |
| T1-2189 | I-6 | II-19 | II-22 |
| T1-2190 | I-6 | II-19 | II-23 |
| T1-2191 | I-6 | II-19 | II-24 |
| T1-2192 | I-6 | II-19 | II-25 |
| T1-2193 | I-6 | II-19 | II-26 |
| T1-2194 | I-6 | II-19 | II-27 |
| T1-2195 | I-6 | II-19 | II-28 |
| T1-2196 | I-6 | II-19 | II-29 |
| T1-2197 | I-7 | II-1 | II-6 |
| T1-2198 | I-7 | II-1 | II-11 |
| T1-2199 | I-7 | II-1 | II-12 |
| T1-2200 | I-7 | II-1 | II-18 |
| T1-2201 | I-7 | II-1 | II-19 |
| T1-2202 | I-7 | II-2 | II-6 |
| T1-2203 | I-7 | II-2 | II-11 |
| T1-2204 | I-7 | II-2 | II-12 |
| T1-2205 | I-7 | II-2 | II-18 |
| T1-2206 | I-7 | II-2 | II-19 |
| T1-2207 | I-7 | II-3 | II-6 |
| T1-2208 | I-7 | II-3 | II-11 |
| T1-2209 | I-7 | II-3 | II-12 |
| T1-2210 | I-7 | II-3 | II-18 |
| T1-2211 | I-7 | II-3 | II-19 |
| T1-2212 | I-7 | II-6 | II-7 |
| T1-2213 | I-7 | II-6 | II-8 |
| T1-2214 | I-7 | II-6 | II-9 |
| T1-2215 | I-7 | II-6 | II-11 |
| T1-2216 | I-7 | II-6 | II-12 |
| T1-2217 | I-7 | II-6 | II-13 |
| T1-2218 | I-7 | II-6 | II-14 |
| T1-2219 | I-7 | II-6 | II-15 |
| T1-2220 | I-7 | II-6 | II-18 |
| T1-2221 | I-7 | II-6 | II-19 |
| T1-2222 | I-7 | II-6 | II-20 |
| T1-2223 | I-7 | II-6 | II-21 |
| T1-2224 | I-7 | II-6 | II-22 |
| T1-2225 | I-7 | II-6 | II-23 |
| T1-2226 | I-7 | II-6 | II-24 |
| T1-2227 | I-7 | II-6 | II-25 |
| T1-2228 | I-7 | II-6 | II-26 |
| T1-2229 | I-7 | II-6 | II-27 |
| T1-2230 | I-7 | II-6 | II-28 |
| T1-2231 | I-7 | II-6 | II-29 |
| T1-2232 | I-7 | II-7 | II-11 |
| T1-2233 | I-7 | II-7 | II-12 |
| T1-2234 | I-7 | II-7 | II-18 |
| T1-2235 | I-7 | II-7 | II-19 |
| T1-2236 | I-7 | II-8 | II-11 |
| T1-2237 | I-7 | II-8 | II-12 |
| T1-2238 | I-7 | II-8 | II-18 |
| T1-2239 | I-7 | II-8 | II-19 |
| T1-2240 | I-7 | II-9 | II-11 |
| T1-2241 | I-7 | II-9 | II-12 |
| T1-2242 | I-7 | II-9 | II-18 |
| T1-2243 | I-7 | II-9 | II-19 |
| T1-2244 | I-7 | II-11 | II-12 |
| T1-2245 | I-7 | II-11 | II-13 |
| T1-2246 | I-7 | II-11 | II-14 |
| T1-2247 | I-7 | II-11 | II-15 |
| T1-2248 | I-7 | II-11 | II-18 |
| T1-2249 | I-7 | II-11 | II-19 |
| T1-2250 | I-7 | II-11 | II-20 |
| T1-2251 | I-7 | II-11 | II-21 |
| T1-2252 | I-7 | II-11 | II-22 |
| T1-2253 | I-7 | II-11 | II-23 |
| T1-2254 | I-7 | II-11 | II-24 |
| T1-2255 | I-7 | II-11 | II-25 |
| T1-2256 | I-7 | II-11 | II-26 |
| T1-2257 | I-7 | II-11 | II-27 |
| T1-2258 | I-7 | II-11 | II-28 |
| T1-2259 | I-7 | II-11 | II-29 |
| T1-2260 | I-7 | II-12 | II-13 |
| T1-2261 | I-7 | II-12 | II-14 |
| T1-2262 | I-7 | II-12 | II-15 |
| T1-2263 | I-7 | II-12 | II-18 |
| T1-2264 | I-7 | II-12 | II-19 |
| T1-2265 | I-7 | II-12 | II-20 |
| T1-2266 | I-7 | II-12 | II-21 |
| T1-2267 | I-7 | II-12 | II-22 |
| T1-2268 | I-7 | II-12 | II-23 |
| T1-2269 | I-7 | II-12 | II-24 |
| T1-2270 | I-7 | II-12 | II-25 |
| T1-2271 | I-7 | II-12 | II-26 |
| T1-2272 | I-7 | II-12 | II-27 |
| T1-2273 | I-7 | II-12 | II-28 |
| T1-2274 | I-7 | II-12 | II-29 |
| T1-2275 | I-7 | II-13 | II-18 |
| T1-2276 | I-7 | II-13 | II-19 |
| T1-2277 | I-7 | II-14 | II-18 |
| T1-2278 | I-7 | II-14 | II-19 |
| T1-2279 | I-7 | II-15 | II-18 |
| T1-2280 | I-7 | II-15 | II-19 |
| T1-2281 | I-7 | II-18 | II-19 |
| T1-2282 | I-7 | II-18 | II-20 |
| T1-2283 | I-7 | II-18 | II-21 |
| T1-2284 | I-7 | II-18 | II-22 |
| T1-2285 | I-7 | II-18 | II-23 |
| T1-2286 | I-7 | II-18 | II-24 |
| T1-2287 | I-7 | II-18 | II-25 |
| T1-2288 | I-7 | II-18 | II-26 |
| T1-2289 | I-7 | II-18 | II-27 |
| T1-2290 | I-7 | II-18 | II-28 |
| T1-2291 | I-7 | II-18 | II-29 |
| T1-2292 | I-7 | II-19 | II-20 |
| T1-2293 | I-7 | II-19 | II-21 |
| T1-2294 | I-7 | II-19 | II-22 |
| T1-2295 | I-7 | II-19 | II-23 |
| T1-2296 | I-7 | II-19 | II-24 |
| T1-2297 | I-7 | II-19 | II-25 |
| T1-2298 | I-7 | II-19 | II-26 |
| T1-2299 | I-7 | II-19 | II-27 |
| T1-2300 | I-7 | II-19 | II-28 |
| T1-2301 | I-7 | II-19 | II-29 |
| T1-2302 | I-8 | II-1 | II-6 |
| T1-2303 | I-8 | II-1 | II-11 |
| T1-2304 | I-8 | II-1 | II-12 |
| T1-2305 | I-8 | II-1 | II-18 |
| T1-2306 | I-8 | II-1 | II-19 |
| T1-2307 | I-8 | II-2 | II-6 |
| T1-2308 | I-8 | II-2 | II-11 |
| T1-2309 | I-8 | II-2 | II-12 |
| T1-2310 | I-8 | II-2 | II-18 |
| T1-2311 | I-8 | II-2 | II-19 |
| T1-2312 | I-8 | II-3 | II-6 |
| T1-2313 | I-8 | II-3 | II-11 |
| T1-2314 | I-8 | II-3 | II-12 |
| T1-2315 | I-8 | II-3 | II-18 |
| T1-2316 | I-8 | II-3 | II-19 |
| T1-2317 | I-8 | II-6 | II-7 |
| T1-2318 | I-8 | II-6 | II-8 |
| T1-2319 | I-8 | II-6 | II-9 |
| T1-2320 | I-8 | II-6 | II-11 |
| T1-2321 | I-8 | II-6 | II-12 |
| T1-2322 | I-8 | II-6 | II-13 |
| T1-2323 | I-8 | II-6 | II-14 |
| T1-2324 | I-8 | II-6 | II-15 |
| T1-2325 | I-8 | II-6 | II-18 |
| T1-2326 | I-8 | II-6 | II-19 |
| T1-2327 | I-8 | II-6 | II-20 |
| T1-2328 | I-8 | II-6 | II-21 |
| T1-2329 | I-8 | II-6 | II-22 |
| T1-2330 | I-8 | II-6 | II-23 |
| T1-2331 | I-8 | II-6 | II-24 |
| T1-2332 | I-8 | II-6 | II-25 |
| T1-2333 | I-8 | II-6 | II-26 |
| T1-2334 | I-8 | II-6 | II-27 |
| T1-2335 | I-8 | II-6 | II-28 |
| T1-2336 | I-8 | II-6 | II-29 |
| T1-2337 | I-8 | II-7 | II-11 |
| T1-2338 | I-8 | II-7 | II-12 |
| T1-2339 | I-8 | II-7 | II-18 |
| T1-2340 | I-8 | II-7 | II-19 |
| T1-2341 | I-8 | II-8 | II-11 |
| T1-2342 | I-8 | II-8 | II-12 |
| T1-2343 | I-8 | II-8 | II-18 |
| T1-2344 | I-8 | II-8 | II-19 |
| T1-2345 | I-8 | II-9 | II-11 |
| T1-2346 | I-8 | II-9 | II-12 |
| T1-2347 | I-8 | II-9 | II-18 |
| T1-2348 | I-8 | II-9 | II-19 |
| T1-2349 | I-8 | II-11 | II-12 |
| T1-2350 | I-8 | II-11 | II-13 |
| T1-2351 | I-8 | II-11 | II-14 |
| T1-2352 | I-8 | II-11 | II-15 |
| T1-2353 | I-8 | II-11 | II-18 |
| T1-2354 | I-8 | II-11 | II-19 |
| T1-2355 | I-8 | II-11 | II-20 |
| T1-2356 | I-8 | II-11 | II-21 |
| T1-2357 | I-8 | II-11 | II-22 |
| T1-2358 | I-8 | II-11 | II-23 |
| T1-2359 | I-8 | II-11 | II-24 |
| T1-2360 | I-8 | II-11 | II-25 |
| T1-2361 | I-8 | II-11 | II-26 |
| T1-2362 | I-8 | II-11 | II-27 |
| T1-2363 | I-8 | II-11 | II-28 |
| T1-2364 | I-8 | II-11 | II-29 |
| T1-2365 | I-8 | II-12 | II-13 |
| T1-2366 | I-8 | II-12 | II-14 |
| T1-2367 | I-8 | II-12 | II-15 |
| T1-2368 | I-8 | II-12 | II-18 |
| T1-2369 | I-8 | II-12 | II-19 |
| T1-2370 | I-8 | II-12 | II-20 |
| T1-2371 | I-8 | II-12 | II-21 |
| T1-2372 | I-8 | II-12 | II-22 |
| T1-2373 | I-8 | II-12 | II-23 |
| T1-2374 | I-8 | II-12 | II-24 |
| T1-2375 | I-8 | II-12 | II-25 |
| T1-2376 | I-8 | II-12 | II-26 |
| T1-2377 | I-8 | II-12 | II-27 |
| T1-2378 | I-8 | II-12 | II-28 |
| T1-2379 | I-8 | II-12 | II-29 |
| T1-2380 | I-8 | II-13 | II-18 |
| T1-2381 | I-8 | II-13 | II-19 |
| T1-2382 | I-8 | II-14 | II-18 |
| T1-2383 | I-8 | II-14 | II-19 |
| T1-2384 | I-8 | II-15 | II-18 |
| T1-2385 | I-8 | II-15 | II-19 |
| T1-2386 | I-8 | II-18 | II-19 |
| T1-2387 | I-8 | II-18 | II-20 |
| T1-2388 | I-8 | II-18 | II-21 |
| T1-2389 | I-8 | II-18 | II-22 |
| T1-2390 | I-8 | II-18 | II-23 |
| T1-2391 | I-8 | II-18 | II-24 |
| T1-2392 | I-8 | II-18 | II-25 |
| T1-2393 | I-8 | II-18 | II-26 |
| T1-2394 | I-8 | II-18 | II-27 |
| T1-2395 | I-8 | II-18 | II-28 |
| T1-2396 | I-8 | II-18 | II-29 |
| T1-2397 | I-8 | II-19 | II-20 |
| T1-2398 | I-8 | II-19 | II-21 |
| T1-2399 | I-8 | II-19 | II-22 |
| T1-2400 | I-8 | II-19 | II-23 |
| T1-2401 | I-8 | II-19 | II-24 |
| T1-2402 | I-8 | II-19 | II-25 |
| T1-2403 | I-8 | II-19 | II-26 |
| T1-2404 | I-8 | II-19 | II-27 |
| T1-2405 | I-8 | II-19 | II-28 |
| T1-2406 | I-8 | II-19 | II-29 |
| T1-2407 | I-9 | II-1 | II-6 |
| T1-2408 | I-9 | II-1 | II-11 |
| T1-2409 | I-9 | II-1 | II-12 |
| T1-2410 | I-9 | II-1 | II-18 |
| T1-2411 | I-9 | II-1 | II-19 |
| T1-2412 | I-9 | II-2 | II-6 |
| T1-2413 | I-9 | II-2 | II-11 |
| T1-2414 | I-9 | II-2 | II-12 |
| T1-2415 | I-9 | II-2 | II-18 |
| T1-2416 | I-9 | II-2 | II-19 |
| T1-2417 | I-9 | II-3 | II-6 |
| T1-2418 | I-9 | II-3 | II-11 |
| T1-2419 | I-9 | II-3 | II-12 |
| T1-2420 | I-9 | II-3 | II-18 |
| T1-2421 | I-9 | II-3 | II-19 |
| T1-2422 | I-9 | II-6 | II-7 |
| T1-2423 | I-9 | II-6 | II-8 |
| T1-2424 | I-9 | II-6 | II-9 |
| T1-2425 | I-9 | II-6 | II-11 |
| T1-2426 | I-9 | II-6 | II-12 |
| T1-2427 | I-9 | II-6 | II-13 |
| T1-2428 | I-9 | II-6 | II-14 |
| T1-2429 | I-9 | II-6 | II-15 |
| T1-2430 | I-9 | II-6 | II-18 |
| T1-2431 | I-9 | II-6 | II-19 |
| T1-2432 | I-9 | II-6 | II-20 |
| T1-2433 | I-9 | II-6 | II-21 |
| T1-2434 | I-9 | II-6 | II-22 |
| T1-2435 | I-9 | II-6 | II-23 |
| T1-2436 | I-9 | II-6 | II-24 |
| T1-2437 | I-9 | II-6 | II-25 |
| T1-2438 | I-9 | II-6 | II-26 |
| T1-2439 | I-9 | II-6 | II-27 |
| T1-2440 | I-9 | II-6 | II-28 |
| T1-2441 | I-9 | II-6 | II-29 |
| T1-2442 | I-9 | II-7 | II-11 |
| T1-2443 | I-9 | II-7 | II-12 |
| T1-2444 | I-9 | II-7 | II-18 |
| T1-2445 | I-9 | II-7 | II-19 |
| T1-2446 | I-9 | II-8 | II-11 |
| T1-2447 | I-9 | II-8 | II-12 |
| T1-2448 | I-9 | II-8 | II-18 |
| T1-2449 | I-9 | II-8 | II-19 |
| T1-2450 | I-9 | II-9 | II-11 |
| T1-2451 | I-9 | II-9 | II-12 |
| T1-2452 | I-9 | II-9 | II-18 |
| T1-2453 | I-9 | II-9 | II-19 |
| T1-2454 | I-9 | II-11 | II-12 |
| T1-2455 | I-9 | II-11 | II-13 |
| T1-2456 | I-9 | II-11 | II-14 |
| T1-2457 | I-9 | II-11 | II-15 |
| T1-2458 | I-9 | II-11 | II-18 |
| T1-2459 | I-9 | II-11 | II-19 |
| T1-2460 | I-9 | II-11 | II-20 |
| T1-2461 | I-9 | II-11 | II-21 |
| T1-2462 | I-9 | II-11 | II-22 |
| T1-2463 | I-9 | II-11 | II-23 |
| T1-2464 | I-9 | II-11 | II-24 |
| T1-2465 | I-9 | II-11 | II-25 |
| T1-2466 | I-9 | II-11 | II-26 |
| T1-2467 | I-9 | II-11 | II-27 |
| T1-2468 | I-9 | II-11 | II-28 |
| T1-2469 | I-9 | II-11 | II-29 |
| T1-2470 | I-9 | II-12 | II-13 |
| T1-2471 | I-9 | II-12 | II-14 |
| T1-2472 | I-9 | II-12 | II-15 |
| T1-2473 | I-9 | II-12 | II-18 |
| T1-2474 | I-9 | II-12 | II-19 |
| T1-2475 | I-9 | II-12 | II-20 |
| T1-2476 | I-9 | II-12 | II-21 |
| T1-2477 | I-9 | II-12 | II-22 |
| T1-2478 | I-9 | II-12 | II-23 |
| T1-2479 | I-9 | II-12 | II-24 |
| T1-2480 | I-9 | II-12 | II-25 |
| T1-2481 | I-9 | II-12 | II-26 |
| T1-2482 | I-9 | II-12 | II-27 |
| T1-2483 | I-9 | II-12 | II-28 |
| T1-2484 | I-9 | II-12 | II-29 |
| T1-2485 | I-9 | II-13 | II-18 |
| T1-2486 | I-9 | II-13 | II-19 |
| T1-2487 | I-9 | II-14 | II-18 |
| T1-2488 | I-9 | II-14 | II-19 |
| T1-2489 | I-9 | II-15 | II-18 |
| T1-2490 | I-9 | II-15 | II-19 |
| T1-2491 | I-9 | II-18 | II-19 |
| T1-2492 | I-9 | II-18 | II-20 |
| T1-2493 | I-9 | II-18 | II-21 |
| T1-2494 | I-9 | II-18 | II-22 |
| T1-2495 | I-9 | II-18 | II-23 |
| T1-2496 | I-9 | II-18 | II-24 |
| T1-2497 | I-9 | II-18 | II-25 |
| T1-2498 | I-9 | II-18 | II-26 |
| T1-2499 | I-9 | II-18 | II-27 |
| T1-2500 | I-9 | II-18 | II-28 |
| T1-2501 | I-9 | II-18 | II-29 |
| T1-2502 | I-9 | II-19 | II-20 |
| T1-2503 | I-9 | II-19 | II-21 |
| T1-2504 | I-9 | II-19 | II-22 |
| T1-2505 | I-9 | II-19 | II-23 |
| T1-2506 | I-9 | II-19 | II-24 |
| T1-2507 | I-9 | II-19 | II-25 |
| T1-2508 | I-9 | II-19 | II-26 |
| T1-2509 | I-9 | II-19 | II-27 |
| T1-2510 | I-9 | II-19 | II-28 |
| T1-2511 | I-9 | II-19 | II-29 |
| T1-2512 | I-11 | II-1 | II-6 |
| T1-2513 | I-11 | II-1 | II-11 |
| T1-2514 | I-11 | II-1 | II-12 |
| T1-2515 | I-11 | II-1 | II-18 |
| T1-2516 | I-11 | II-1 | II-19 |
| T1-2517 | I-11 | II-2 | II-6 |
| T1-2518 | I-11 | II-2 | II-11 |
| T1-2519 | I-11 | II-2 | II-12 |
| T1-2520 | I-11 | II-2 | II-18 |
| T1-2521 | I-11 | II-2 | II-19 |
| T1-2522 | I-11 | II-3 | II-6 |
| T1-2523 | I-11 | II-3 | II-11 |
| T1-2524 | I-11 | II-3 | II-12 |
| T1-2525 | I-11 | II-3 | II-18 |
| T1-2526 | I-11 | II-3 | II-19 |
| T1-2527 | I-11 | II-6 | II-7 |
| T1-2528 | I-11 | II-6 | II-8 |
| T1-2529 | I-11 | II-6 | II-9 |
| T1-2530 | I-11 | II-6 | II-11 |
| T1-2531 | I-11 | II-6 | II-12 |
| T1-2532 | I-11 | II-6 | II-13 |
| T1-2533 | I-11 | II-6 | II-14 |
| T1-2534 | I-11 | II-6 | II-15 |
| T1-2535 | I-11 | II-6 | II-18 |
| T1-2536 | I-11 | II-6 | II-19 |
| T1-2537 | I-11 | II-6 | II-20 |
| T1-2538 | I-11 | II-6 | II-21 |
| T1-2539 | I-11 | II-6 | II-22 |
| T1-2540 | I-11 | II-6 | II-23 |
| T1-2541 | I-11 | II-6 | II-24 |
| T1-2542 | I-11 | II-6 | II-25 |
| T1-2543 | I-11 | II-6 | II-26 |
| T1-2544 | I-11 | II-6 | II-27 |
| T1-2545 | I-11 | II-6 | II-28 |
| T1-2546 | I-11 | II-6 | II-29 |
| T1-2547 | I-11 | II-7 | II-11 |
| T1-2548 | I-11 | II-7 | II-12 |
| T1-2549 | I-11 | II-7 | II-18 |
| T1-2550 | I-11 | II-7 | II-19 |
| T1-2551 | I-11 | II-8 | II-11 |
| T1-2552 | I-11 | II-8 | II-12 |
| T1-2553 | I-11 | II-8 | II-18 |
| T1-2554 | I-11 | II-8 | II-19 |
| T1-2555 | I-11 | II-9 | II-11 |
| T1-2556 | I-11 | II-9 | II-12 |
| T1-2557 | I-11 | II-9 | II-18 |
| T1-2558 | I-11 | II-9 | II-19 |
| T1-2559 | I-11 | II-11 | II-12 |
| T1-2560 | I-11 | II-11 | II-13 |
| T1-2561 | I-11 | II-11 | II-14 |
| T1-2562 | I-11 | II-11 | II-15 |
| T1-2563 | I-11 | II-11 | II-18 |
| T1-2564 | I-11 | II-11 | II-19 |
| T1-2565 | I-11 | II-11 | II-20 |
| T1-2566 | I-11 | II-11 | II-21 |
| T1-2567 | I-11 | II-11 | II-22 |
| T1-2568 | I-11 | II-11 | II-23 |
| T1-2569 | I-11 | II-11 | II-24 |
| T1-2570 | I-11 | II-11 | II-25 |
| T1-2571 | I-11 | II-11 | II-26 |
| T1-2572 | I-11 | II-11 | II-27 |
| T1-2573 | I-11 | II-11 | II-28 |
| T1-2574 | I-11 | II-11 | II-29 |
| T1-2575 | I-11 | II-12 | II-13 |
| T1-2576 | I-11 | II-12 | II-14 |
| T1-2577 | I-11 | II-12 | II-15 |
| T1-2578 | I-11 | II-12 | II-18 |
| T1-2579 | I-11 | II-12 | II-19 |
| T1-2580 | I-11 | II-12 | II-20 |
| T1-2581 | I-11 | II-12 | II-21 |
| T1-2582 | I-11 | II-12 | II-22 |
| T1-2583 | I-11 | II-12 | II-23 |
| T1-2584 | I-11 | II-12 | II-24 |
| T1-2585 | I-11 | II-12 | II-25 |
| T1-2586 | I-11 | II-12 | II-26 |
| T1-2587 | I-11 | II-12 | II-27 |
| T1-2588 | I-11 | II-12 | II-28 |
| T1-2589 | I-11 | II-12 | II-29 |
| T1-2590 | I-11 | II-13 | II-18 |
| T1-2591 | I-11 | II-13 | II-19 |
| T1-2592 | I-11 | II-14 | II-18 |
| T1-2593 | I-11 | II-14 | II-19 |
| T1-2594 | I-11 | II-15 | II-18 |
| T1-2595 | I-11 | II-15 | II-19 |
| T1-2596 | I-11 | II-18 | II-19 |
| T1-2597 | I-11 | II-18 | II-20 |
| T1-2598 | I-11 | II-18 | II-21 |
| T1-2599 | I-11 | II-18 | II-22 |
| T1-2600 | I-11 | II-18 | II-23 |
| T1-2601 | I-11 | II-18 | II-24 |
| T1-2602 | I-11 | II-18 | II-25 |
| T1-2603 | I-11 | II-18 | II-26 |
| T1-2604 | I-11 | II-18 | II-27 |
| T1-2605 | I-11 | II-18 | II-28 |
| T1-2606 | I-11 | II-18 | II-29 |
| T1-2607 | I-11 | II-19 | II-20 |
| T1-2608 | I-11 | II-19 | II-21 |
| T1-2609 | I-11 | II-19 | II-22 |
| T1-2610 | I-11 | II-19 | II-23 |
| T1-2611 | I-11 | II-19 | II-24 |
| T1-2612 | I-11 | II-19 | II-25 |
| T1-2613 | I-11 | II-19 | II-26 |
| T1-2614 | I-11 | II-19 | II-27 |
| T1-2615 | I-11 | II-19 | II-28 |
| T1-2616 | I-11 | II-19 | II-29 |
| T1-2617 | I-12 | II-1 | II-6 |
| T1-2618 | I-12 | II-1 | II-11 |
| T1-2619 | I-12 | II-1 | II-12 |
| T1-2620 | I-12 | II-1 | II-18 |
| T1-2621 | I-12 | II-1 | II-19 |
| T1-2622 | I-12 | II-2 | II-6 |
| T1-2623 | I-12 | II-2 | II-11 |
| T1-2624 | I-12 | II-2 | II-12 |
| T1-2625 | I-12 | II-2 | II-18 |
| T1-2626 | I-12 | II-2 | II-19 |
| T1-2627 | I-12 | II-3 | II-6 |
| T1-2628 | I-12 | II-3 | II-11 |
| T1-2629 | I-12 | II-3 | II-12 |
| T1-2630 | I-12 | II-3 | II-18 |
| T1-2631 | I-12 | II-3 | II-19 |
| T1-2632 | I-12 | II-6 | II-7 |
| T1-2633 | I-12 | II-6 | II-8 |
| T1-2634 | I-12 | II-6 | II-9 |
| T1-2635 | I-12 | II-6 | II-11 |
| T1-2636 | I-12 | II-6 | II-12 |
| T1-2637 | I-12 | II-6 | II-13 |
| T1-2638 | I-12 | II-6 | II-14 |
| T1-2639 | I-12 | II-6 | II-15 |
| T1-2640 | I-12 | II-6 | II-18 |
| T1-2641 | I-12 | II-6 | II-19 |
| T1-2642 | I-12 | II-6 | II-20 |
| T1-2643 | I-12 | II-6 | II-21 |
| T1-2644 | I-12 | II-6 | II-22 |
| T1-2645 | I-12 | II-6 | II-23 |
| T1-2646 | I-12 | II-6 | II-24 |
| T1-2647 | I-12 | II-6 | II-25 |
| T1-2648 | I-12 | II-6 | II-26 |
| T1-2649 | I-12 | II-6 | II-27 |
| T1-2650 | I-12 | II-6 | II-28 |
| T1-2651 | I-12 | II-6 | II-29 |
| T1-2652 | I-12 | II-7 | II-11 |
| T1-2653 | I-12 | II-7 | II-12 |
| T1-2654 | I-12 | II-7 | II-18 |
| T1-2655 | I-12 | II-7 | II-19 |
| T1-2656 | I-12 | II-8 | II-11 |
| T1-2657 | I-12 | II-8 | II-12 |
| T1-2658 | I-12 | II-8 | II-18 |
| T1-2659 | I-12 | II-8 | II-19 |
| T1-2660 | I-12 | II-9 | II-11 |
| T1-2661 | I-12 | II-9 | II-12 |
| T1-2662 | I-12 | II-9 | II-18 |
| T1-2663 | I-12 | II-9 | II-19 |
| T1-2664 | I-12 | II-11 | II-12 |
| T1-2665 | I-12 | II-11 | II-13 |
| T1-2666 | I-12 | II-11 | II-14 |
| T1-2667 | I-12 | II-11 | II-15 |
| T1-2668 | I-12 | II-11 | II-18 |
| T1-2669 | I-12 | II-11 | II-19 |
| T1-2670 | I-12 | II-11 | II-20 |
| T1-2671 | I-12 | II-11 | II-21 |
| T1-2672 | I-12 | II-11 | II-22 |
| T1-2673 | I-12 | II-11 | II-23 |
| T1-2674 | I-12 | II-11 | II-24 |
| T1-2675 | I-12 | II-11 | II-25 |
| T1-2676 | I-12 | II-11 | II-26 |
| T1-2677 | I-12 | II-11 | II-27 |
| T1-2678 | I-12 | II-11 | II-28 |
| T1-2679 | I-12 | II-11 | II-29 |
| T1-2680 | I-12 | II-12 | II-13 |
| T1-2681 | I-12 | II-12 | II-14 |
| T1-2682 | I-12 | II-12 | II-15 |
| T1-2683 | I-12 | II-12 | II-18 |
| T1-2684 | I-12 | II-12 | II-19 |
| T1-2685 | I-12 | II-12 | II-20 |
| T1-2686 | I-12 | II-12 | II-21 |
| T1-2687 | I-12 | II-12 | II-22 |
| T1-2688 | I-12 | II-12 | II-23 |
| T1-2689 | I-12 | II-12 | II-24 |
| T1-2690 | I-12 | II-12 | II-25 |
| T1-2691 | I-12 | II-12 | II-26 |
| T1-2692 | I-12 | II-12 | II-27 |
| T1-2693 | I-12 | II-12 | II-28 |
| T1-2694 | I-12 | II-12 | II-29 |
| T1-2695 | I-12 | II-13 | II-18 |
| T1-2696 | I-12 | II-13 | II-19 |
| T1-2697 | I-12 | II-14 | II-18 |
| T1-2698 | I-12 | II-14 | II-19 |
| T1-2699 | I-12 | II-15 | II-18 |
| T1-2700 | I-12 | II-15 | II-19 |
| T1-2701 | I-12 | II-18 | II-19 |
| T1-2702 | I-12 | II-18 | II-20 |
| T1-2703 | I-12 | II-18 | II-21 |
| T1-2704 | I-12 | II-18 | II-22 |
| T1-2705 | I-12 | II-18 | II-23 |
| T1-2706 | I-12 | II-18 | II-24 |
| T1-2707 | I-12 | II-18 | II-25 |
| T1-2708 | I-12 | II-18 | II-26 |
| T1-2709 | I-12 | II-18 | II-27 |
| T1-2710 | I-12 | II-18 | II-28 |
| T1-2711 | I-12 | II-18 | II-29 |
| T1-2712 | I-12 | II-19 | II-20 |
| T1-2713 | I-12 | II-19 | II-21 |
| T1-2714 | I-12 | II-19 | II-22 |
| T1-2715 | I-12 | II-19 | II-23 |
| T1-2716 | I-12 | II-19 | II-24 |
| T1-2717 | I-12 | II-19 | II-25 |
| T1-2718 | I-12 | II-19 | II-26 |
| T1-2719 | I-12 | II-19 | II-27 |
| T1-2720 | I-12 | II-19 | II-28 |
| T1-2721 | I-12 | II-19 | II-29 |
| T1-2722 | I-13 | II-1 | II-6 |
| T1-2723 | I-13 | II-1 | II-11 |
| T1-2724 | I-13 | II-1 | II-12 |
| T1-2725 | I-13 | II-1 | II-18 |
| T1-2726 | I-13 | II-1 | II-19 |
| T1-2727 | I-13 | II-2 | II-6 |
| T1-2728 | I-13 | II-2 | II-11 |
| T1-2729 | I-13 | II-2 | II-12 |
| T1-2730 | I-13 | II-2 | II-18 |
| T1-2731 | I-13 | II-2 | II-19 |
| T1-2732 | I-13 | II-3 | II-6 |
| T1-2733 | I-13 | II-3 | II-11 |
| T1-2734 | I-13 | II-3 | II-12 |
| T1-2735 | I-13 | II-3 | II-18 |
| T1-2736 | I-13 | II-3 | II-19 |
| T1-2737 | I-13 | II-6 | II-7 |
| T1-2738 | I-13 | II-6 | II-8 |
| T1-2739 | I-13 | II-6 | II-9 |
| T1-2740 | I-13 | II-6 | II-11 |
| T1-2741 | I-13 | II-6 | II-12 |
| T1-2742 | I-13 | II-6 | II-13 |
| T1-2743 | I-13 | II-6 | II-14 |
| T1-2744 | I-13 | II-6 | II-15 |
| T1-2745 | I-13 | II-6 | II-18 |
| T1-2746 | I-13 | II-6 | II-19 |
| T1-2747 | I-13 | II-6 | II-20 |
| T1-2748 | I-13 | II-6 | II-21 |
| T1-2749 | I-13 | II-6 | II-22 |
| T1-2750 | I-13 | II-6 | II-23 |
| T1-2751 | I-13 | II-6 | II-24 |
| T1-2752 | I-13 | II-6 | II-25 |
| T1-2753 | I-13 | II-6 | II-26 |
| T1-2754 | I-13 | II-6 | II-27 |
| T1-2755 | I-13 | II-6 | II-28 |
| T1-2756 | I-13 | II-6 | II-29 |
| T1-2757 | I-13 | II-7 | II-11 |
| T1-2758 | I-13 | II-7 | II-12 |
| T1-2759 | I-13 | II-7 | II-18 |
| T1-2760 | I-13 | II-7 | II-19 |
| T1-2761 | I-13 | II-8 | II-11 |
| T1-2762 | I-13 | II-8 | II-12 |
| T1-2763 | I-13 | II-8 | II-18 |
| T1-2764 | I-13 | II-8 | II-19 |
| T1-2765 | I-13 | II-9 | II-11 |
| T1-2766 | I-13 | II-9 | II-12 |
| T1-2767 | I-13 | II-9 | II-18 |
| T1-2768 | I-13 | II-9 | II-19 |
| T1-2769 | I-13 | II-11 | II-12 |
| T1-2770 | I-13 | II-11 | II-13 |
| T1-2771 | I-13 | II-11 | II-14 |
| T1-2772 | I-13 | II-11 | II-15 |
| T1-2773 | I-13 | II-11 | II-18 |
| T1-2774 | I-13 | II-11 | II-19 |
| T1-2775 | I-13 | II-11 | II-20 |
| T1-2776 | I-13 | II-11 | II-21 |
| T1-2777 | I-13 | II-11 | II-22 |
| T1-2778 | I-13 | II-11 | II-23 |
| T1-2779 | I-13 | II-11 | II-24 |
| T1-2780 | I-13 | II-11 | II-25 |
| T1-2781 | I-13 | II-11 | II-26 |
| T1-2782 | I-13 | II-11 | II-27 |
| T1-2783 | I-13 | II-11 | II-28 |
| T1-2784 | I-13 | II-11 | II-29 |
| T1-2785 | I-13 | II-12 | II-13 |
| T1-2786 | I-13 | II-12 | II-14 |
| T1-2787 | I-13 | II-12 | II-15 |
| T1-2788 | I-13 | II-12 | II-18 |
| T1-2789 | I-13 | II-12 | II-19 |
| T1-2790 | I-13 | II-12 | II-20 |
| T1-2791 | I-13 | II-12 | II-21 |
| T1-2792 | I-13 | II-12 | II-22 |
| T1-2793 | I-13 | II-12 | II-23 |
| T1-2794 | I-13 | II-12 | II-24 |
| T1-2795 | I-13 | II-12 | II-25 |
| T1-2796 | I-13 | II-12 | II-26 |
| T1-2797 | I-13 | II-12 | II-27 |
| T1-2798 | I-13 | II-12 | II-28 |
| T1-2799 | I-13 | II-12 | II-29 |
| T1-2800 | I-13 | II-13 | II-18 |
| T1-2801 | I-13 | II-13 | II-19 |
| T1-2802 | I-13 | II-14 | II-18 |
| T1-2803 | I-13 | II-14 | II-19 |
| T1-2804 | I-13 | II-15 | II-18 |
| T1-2805 | I-13 | II-15 | II-19 |
| T1-2806 | I-13 | II-18 | II-19 |
| T1-2807 | I-13 | II-18 | II-20 |
| T1-2808 | I-13 | II-18 | II-21 |
| T1-2809 | I-13 | II-18 | II-22 |
| T1-2810 | I-13 | II-18 | II-23 |
| T1-2811 | I-13 | II-18 | II-24 |
| T1-2812 | I-13 | II-18 | II-25 |
| T1-2813 | I-13 | II-18 | II-26 |
| T1-2814 | I-13 | II-18 | II-27 |
| T1-2815 | I-13 | II-18 | II-28 |
| T1-2816 | I-13 | II-18 | II-29 |
| T1-2817 | I-13 | II-19 | II-20 |
| T1-2818 | I-13 | II-19 | II-21 |
| T1-2819 | I-13 | II-19 | II-22 |
| T1-2820 | I-13 | II-19 | II-23 |
| T1-2821 | I-13 | II-19 | II-24 |
| T1-2822 | I-13 | II-19 | II-25 |
| T1-2823 | I-13 | II-19 | II-26 |
| T1-2824 | I-13 | II-19 | II-27 |
| T1-2825 | I-13 | II-19 | II-28 |
| T1-2826 | I-13 | II-19 | II-29 |
| T1-2827 | I-14 | II-1 | II-6 |
| T1-2828 | I-14 | II-1 | II-11 |
| T1-2829 | I-14 | II-1 | II-12 |
| T1-2830 | I-14 | II-1 | II-18 |
| T1-2831 | I-14 | II-1 | II-19 |
| T1-2832 | I-14 | II-2 | II-6 |
| T1-2833 | I-14 | II-2 | II-11 |
| T1-2834 | I-14 | II-2 | II-12 |
| T1-2835 | I-14 | II-2 | II-18 |
| T1-2836 | I-14 | II-2 | II-19 |
| T1-2837 | I-14 | II-3 | II-6 |
| T1-2838 | I-14 | II-3 | II-11 |
| T1-2839 | I-14 | II-3 | II-12 |
| T1-2840 | I-14 | II-3 | II-18 |
| T1-2841 | I-14 | II-3 | II-19 |
| T1-2842 | I-14 | II-6 | II-7 |
| T1-2843 | I-14 | II-6 | II-8 |
| T1-2844 | I-14 | II-6 | II-9 |
| T1-2845 | I-14 | II-6 | II-11 |
| T1-2846 | I-14 | II-6 | II-12 |
| T1-2847 | I-14 | II-6 | II-13 |
| T1-2848 | I-14 | II-6 | II-14 |
| T1-2849 | I-14 | II-6 | II-15 |
| T1-2850 | I-14 | II-6 | II-18 |
| T1-2851 | I-14 | II-6 | II-19 |
| T1-2852 | I-14 | II-6 | II-20 |
| T1-2853 | I-14 | II-6 | II-21 |
| T1-2854 | I-14 | II-6 | II-22 |
| T1-2855 | I-14 | II-6 | II-23 |
| T1-2856 | I-14 | II-6 | II-24 |
| T1-2857 | I-14 | II-6 | II-25 |
| T1-2858 | I-14 | II-6 | II-26 |
| T1-2859 | I-14 | II-6 | II-27 |
| T1-2860 | I-14 | II-6 | II-28 |
| T1-2861 | I-14 | II-6 | II-29 |
| T1-2862 | I-14 | II-7 | II-11 |
| T1-2863 | I-14 | II-7 | II-12 |
| T1-2864 | I-14 | II-7 | II-18 |
| T1-2865 | I-14 | II-7 | II-19 |
| T1-2866 | I-14 | II-8 | II-11 |
| T1-2867 | I-14 | II-8 | II-12 |
| T1-2868 | I-14 | II-8 | II-18 |
| T1-2869 | I-14 | II-8 | II-19 |
| T1-2870 | I-14 | II-9 | II-11 |
| T1-2871 | I-14 | II-9 | II-12 |
| T1-2872 | I-14 | II-9 | II-18 |
| T1-2873 | I-14 | II-9 | II-19 |
| T1-2874 | I-14 | II-11 | II-12 |
| T1-2875 | I-14 | II-11 | II-13 |
| T1-2876 | I-14 | II-11 | II-14 |
| T1-2877 | I-14 | II-11 | II-15 |
| T1-2878 | I-14 | II-11 | II-18 |
| T1-2879 | I-14 | II-11 | II-19 |
| T1-2880 | I-14 | II-11 | II-20 |
| T1-2881 | I-14 | II-11 | II-21 |
| T1-2882 | I-14 | II-11 | II-22 |
| T1-2883 | I-14 | II-11 | II-23 |
| T1-2884 | I-14 | II-11 | II-24 |
| T1-2885 | I-14 | II-11 | II-25 |
| T1-2886 | I-14 | II-11 | II-26 |
| T1-2887 | I-14 | II-11 | II-27 |
| T1-2888 | I-14 | II-11 | II-28 |
| T1-2889 | I-14 | II-11 | II-29 |
| T1-2890 | I-14 | II-12 | II-13 |
| T1-2891 | I-14 | II-12 | II-14 |
| T1-2892 | I-14 | II-12 | II-15 |
| T1-2893 | I-14 | II-12 | II-18 |
| T1-2894 | I-14 | II-12 | II-19 |
| T1-2895 | I-14 | II-12 | II-20 |
| T1-2896 | I-14 | II-12 | II-21 |
| T1-2897 | I-14 | II-12 | II-22 |
| T1-2898 | I-14 | II-12 | II-23 |
| T1-2899 | I-14 | II-12 | II-24 |
| T1-2900 | I-14 | II-12 | II-25 |
| T1-2901 | I-14 | II-12 | II-26 |
| T1-2902 | I-14 | II-12 | II-27 |
| T1-2903 | I-14 | II-12 | II-28 |
| T1-2904 | I-14 | II-12 | II-29 |
| T1-2905 | I-14 | II-13 | II-18 |
| T1-2906 | I-14 | II-13 | II-19 |
| T1-2907 | I-14 | II-14 | II-18 |
| T1-2908 | I-14 | II-14 | II-19 |
| T1-2909 | I-14 | II-15 | II-18 |
| T1-2910 | I-14 | II-15 | II-19 |
| T1-2911 | I-14 | II-18 | II-19 |
| T1-2912 | I-14 | II-18 | II-20 |
| T1-2913 | I-14 | II-18 | II-21 |
| T1-2914 | I-14 | II-18 | II-22 |
| T1-2915 | I-14 | II-18 | II-23 |
| T1-2916 | I-14 | II-18 | II-24 |
| T1-2917 | I-14 | II-18 | II-25 |
| T1-2918 | I-14 | II-18 | II-26 |
| T1-2919 | I-14 | II-18 | II-27 |
| T1-2920 | I-14 | II-18 | II-28 |
| T1-2921 | I-14 | II-18 | II-29 |
| T1-2922 | I-14 | II-19 | II-20 |
| T1-2923 | I-14 | II-19 | II-21 |
| T1-2924 | I-14 | II-19 | II-22 |
| T1-2925 | I-14 | II-19 | II-23 |
| T1-2926 | I-14 | II-19 | II-24 |
| T1-2927 | I-14 | II-19 | II-25 |
| T1-2928 | I-14 | II-19 | II-26 |
| T1-2929 | I-14 | II-19 | II-27 |
| T1-2930 | I-14 | II-19 | II-28 |
| T1-2931 | I-14 | II-19 | II-29 |
| T1-2932 | I-18 | II-1 | II-6 |
| T1-2933 | I-18 | II-1 | II-11 |
| T1-2934 | I-18 | II-1 | II-12 |
| T1-2935 | I-18 | II-1 | II-18 |
| T1-2936 | I-18 | II-1 | II-19 |
| T1-2937 | I-18 | II-2 | II-6 |
| T1-2938 | I-18 | II-2 | II-11 |
| T1-2939 | I-18 | II-2 | II-12 |
| T1-2940 | I-18 | II-2 | II-18 |
| T1-2941 | I-18 | II-2 | II-19 |
| T1-2942 | I-18 | II-3 | II-6 |
| T1-2943 | I-18 | II-3 | II-11 |
| T1-2944 | I-18 | II-3 | II-12 |
| T1-2945 | I-18 | II-3 | II-18 |
| T1-2946 | I-18 | II-3 | II-19 |
| T1-2947 | I-18 | II-6 | II-7 |
| T1-2948 | I-18 | II-6 | II-8 |
| T1-2949 | I-18 | II-6 | II-9 |
| T1-2950 | I-18 | II-6 | II-11 |
| T1-2951 | I-18 | II-6 | II-12 |
| T1-2952 | I-18 | II-6 | II-13 |
| T1-2953 | I-18 | II-6 | II-14 |
| T1-2954 | I-18 | II-6 | II-15 |
| T1-2955 | I-18 | II-6 | II-18 |
| T1-2956 | I-18 | II-6 | II-19 |
| T1-2957 | I-18 | II-6 | II-20 |
| T1-2958 | I-18 | II-6 | II-21 |
| T1-2959 | I-18 | II-6 | II-22 |
| T1-2960 | I-18 | II-6 | II-23 |
| T1-2961 | I-18 | II-6 | II-24 |
| T1-2962 | I-18 | II-6 | II-25 |
| T1-2963 | I-18 | II-6 | II-26 |
| T1-2964 | I-18 | II-6 | II-27 |
| T1-2965 | I-18 | II-6 | II-28 |
| T1-2966 | I-18 | II-6 | II-29 |
| T1-2967 | I-18 | II-7 | II-11 |
| T1-2968 | I-18 | II-7 | II-12 |
| T1-2969 | I-18 | II-7 | II-18 |
| T1-2970 | I-18 | II-7 | II-19 |
| T1-2971 | I-18 | II-8 | II-11 |
| T1-2972 | I-18 | II-8 | II-12 |
| T1-2973 | I-18 | II-8 | II-18 |
| T1-2974 | I-18 | II-8 | II-19 |
| T1-2975 | I-18 | II-9 | II-11 |
| T1-2976 | I-18 | II-9 | II-12 |
| T1-2977 | I-18 | II-9 | II-18 |
| T1-2978 | I-18 | II-9 | II-19 |
| T1-2979 | I-18 | II-11 | II-12 |
| T1-2980 | I-18 | II-11 | II-13 |
| T1-2981 | I-18 | II-11 | II-14 |
| T1-2982 | I-18 | II-11 | II-15 |
| T1-2983 | I-18 | II-11 | II-18 |
| T1-2984 | I-18 | II-11 | II-19 |
| T1-2985 | I-18 | II-11 | II-20 |
| T1-2986 | I-18 | II-11 | II-21 |
| T1-2987 | I-18 | II-11 | II-22 |
| T1-2988 | I-18 | II-11 | II-23 |
| T1-2989 | I-18 | II-11 | II-24 |
| T1-2990 | I-18 | II-11 | II-25 |
| T1-2991 | I-18 | II-11 | II-26 |
| T1-2992 | I-18 | II-11 | II-27 |
| T1-2993 | I-18 | II-11 | II-28 |
| T1-2994 | I-18 | II-11 | II-29 |
| T1-2995 | I-18 | II-12 | II-13 |
| T1-2996 | I-18 | II-12 | II-14 |
| T1-2997 | I-18 | II-12 | II-15 |
| T1-2998 | I-18 | II-12 | II-18 |
| T1-2999 | I-18 | II-12 | II-19 |
| T1-3000 | I-18 | II-12 | II-20 |
| T1-3001 | I-18 | II-12 | II-21 |
| T1-3002 | I-18 | II-12 | II-22 |
| T1-3003 | I-18 | II-12 | II-23 |
| T1-3004 | I-18 | II-12 | II-24 |
| T1-3005 | I-18 | II-12 | II-25 |
| T1-3006 | I-18 | II-12 | II-26 |
| T1-3007 | I-18 | II-12 | II-27 |
| T1-3008 | I-18 | II-12 | II-28 |
| T1-3009 | I-18 | II-12 | II-29 |
| T1-3010 | I-18 | II-13 | II-18 |
| T1-3011 | I-18 | II-13 | II-19 |
| T1-3012 | I-18 | II-14 | II-18 |
| T1-3013 | I-18 | II-14 | II-19 |
| T1-3014 | I-18 | II-15 | II-18 |
| T1-3015 | I-18 | II-15 | II-19 |
| T1-3016 | I-18 | II-18 | II-19 |
| T1-3017 | I-18 | II-18 | II-20 |
| T1-3018 | I-18 | II-18 | II-21 |
| T1-3019 | I-18 | II-18 | II-22 |
| T1-3020 | I-18 | II-18 | II-23 |
| T1-3021 | I-18 | II-18 | II-24 |
| T1-3022 | I-18 | II-18 | II-25 |
| T1-3023 | I-18 | II-18 | II-26 |
| T1-3024 | I-18 | II-18 | II-27 |
| T1-3025 | I-18 | II-18 | II-28 |
| T1-3026 | I-18 | II-18 | II-29 |
| T1-3027 | I-18 | II-19 | II-20 |
| T1-3028 | I-18 | II-19 | II-21 |
| T1-3029 | I-18 | II-19 | II-22 |
| T1-3030 | I-18 | II-19 | II-23 |
| T1-3031 | I-18 | II-19 | II-24 |
| T1-3032 | I-18 | II-19 | II-25 |
| T1-3033 | I-18 | II-19 | II-26 |
| T1-3034 | I-18 | II-19 | II-27 |
| T1-3035 | I-18 | II-19 | II-28 |
| T1-3036 | I-18 | II-19 | II-29 |

“C” denotes “component”,
“M” denotes “mixture”.

According to a further embodiment, preference is given to mixtures in which component 3 is a compound II selected from the group of the following compounds:

| | |
|---|---|
| II-46 | prochloraz |
| II-47 | dithianon |
| II-48 | difenoconazole |

with the proviso that component 2 and component 3 are not identical.

TABLE T2
The ternary mixtures T2-1 to T2-825 below comprise, as component 1,
the compound I, a component 2 selected from the active compounds
preferred for component 2 and a component 3 selected from active
compounds preferred for component 3. In the table below, each row
corresponds to a ternary mixture according to the invention having the
mixture components 1 to 3 listed in the row in question.

| M | C1 | C2 | C3 |
|---|---|---|---|
| T2-1 | I-1 | II-1 | II-46 |
| T2-2 | I-1 | II-1 | II-47 |
| T2-3 | I-1 | II-1 | II-48 |
| T2-4 | I-1 | II-2 | II-46 |
| T2-5 | I-1 | II-2 | II-47 |
| T2-6 | I-1 | II-2 | II-48 |
| T2-7 | I-1 | II-3 | II-46 |
| T2-8 | I-1 | II-3 | II-47 |
| T2-9 | I-1 | II-3 | II-48 |
| T2-10 | I-1 | II-6 | II-46 |
| T2-11 | I-1 | II-6 | II-47 |
| T2-12 | I-1 | II-6 | II-48 |
| T2-13 | I-1 | II-7 | II-46 |
| T2-14 | I-1 | II-7 | II-47 |
| T2-15 | I-1 | II-7 | II-48 |
| T2-16 | I-1 | II-8 | II-46 |
| T2-17 | I-1 | II-8 | II-47 |
| T2-18 | I-1 | II-8 | II-48 |
| T2-19 | I-1 | II-9 | II-46 |
| T2-20 | I-1 | II-9 | II-47 |
| T2-21 | I-1 | II-9 | II-48 |
| T2-22 | I-1 | II-11 | II-46 |
| T2-23 | I-1 | II-11 | II-47 |
| T2-24 | I-1 | II-11 | II-48 |
| T2-25 | I-1 | II-12 | II-46 |
| T2-26 | I-1 | II-12 | II-47 |
| T2-27 | I-1 | II-12 | II-48 |
| T2-28 | I-1 | II-13 | II-46 |
| T2-29 | I-1 | II-13 | II-47 |
| T2-30 | I-1 | II-13 | II-48 |
| T2-31 | I-1 | II-14 | II-46 |
| T2-32 | I-1 | II-14 | II-47 |
| T2-33 | I-1 | II-14 | II-48 |
| T2-34 | I-1 | II-15 | II-46 |
| T2-35 | I-1 | II-15 | II-47 |
| T2-36 | I-1 | II-15 | II-48 |
| T2-37 | I-1 | II-18 | II-46 |
| T2-38 | I-1 | II-18 | II-47 |
| T2-39 | I-1 | II-18 | II-48 |
| T2-40 | I-1 | II-19 | II-46 |
| T2-41 | I-1 | II-19 | II-47 |
| T2-42 | I-1 | II-19 | II-48 |
| T2-43 | I-1 | II-20 | II-46 |
| T2-44 | I-1 | II-20 | II-47 |
| T2-45 | I-1 | II-20 | II-48 |
| T2-46 | I-1 | II-21 | II-46 |
| T2-47 | I-1 | II-21 | II-47 |
| T2-48 | I-1 | II-21 | II-48 |
| T2-49 | I-1 | II-22 | II-46 |
| T2-50 | I-1 | II-22 | II-47 |
| T2-51 | I-1 | II-22 | II-48 |
| T2-52 | I-1 | II-23 | II-46 |
| T2-53 | I-1 | II-23 | II-47 |
| T2-54 | I-1 | II-23 | II-48 |
| T2-55 | I-1 | II-24 | II-46 |
| T2-56 | I-1 | II-24 | II-47 |
| T2-57 | I-1 | II-24 | II-48 |
| T2-58 | I-1 | II-25 | II-46 |
| T2-59 | I-1 | II-25 | II-47 |
| T2-60 | I-1 | II-25 | II-48 |
| T2-61 | I-1 | II-26 | II-46 |
| T2-62 | I-1 | II-26 | II-47 |
| T2-63 | I-1 | II-26 | II-48 |
| T2-64 | I-1 | II-27 | II-46 |
| T2-65 | I-1 | II-27 | II-47 |
| T2-66 | I-1 | II-27 | II-48 |
| T2-67 | I-1 | II-28 | II-46 |
| T2-68 | I-1 | II-28 | II-47 |
| T2-69 | I-1 | II-28 | II-48 |
| T2-70 | I-1 | II-29 | II-46 |
| T2-71 | I-1 | II-29 | II-47 |
| T2-72 | I-1 | II-29 | II-48 |
| T2-73 | I-1 | II-46 | II-47 |
| T2-74 | I-1 | II-46 | II-48 |
| T2-75 | I-1 | II-47 | II-48 |
| T2-76 | I-2 | II-1 | II-46 |
| T2-77 | I-2 | II-1 | II-47 |
| T2-78 | I-2 | II-1 | II-48 |
| T2-79 | I-2 | II-2 | II-46 |
| T2-80 | I-2 | II-2 | II-47 |
| T2-81 | I-2 | II-2 | II-48 |
| T2-82 | I-2 | II-3 | II-46 |
| T2-83 | I-2 | II-3 | II-47 |
| T2-84 | I-2 | II-3 | II-48 |
| T2-85 | I-2 | II-6 | II-46 |
| T2-86 | I-2 | II-6 | II-47 |
| T2-87 | I-2 | II-6 | II-48 |
| T2-88 | I-2 | II-7 | II-46 |
| T2-89 | I-2 | II-7 | II-47 |
| T2-90 | I-2 | II-7 | II-48 |
| T2-91 | I-2 | II-8 | II-46 |
| T2-92 | I-2 | II-8 | II-47 |
| T2-93 | I-2 | II-8 | II-48 |
| T2-94 | I-2 | II-9 | II-46 |
| T2-95 | I-2 | II-9 | II-47 |
| T2-96 | I-2 | II-9 | II-48 |
| T2-97 | I-2 | II-11 | II-46 |
| T2-98 | I-2 | II-11 | II-47 |
| T2-99 | I-2 | II-11 | II-48 |
| T2-100 | I-2 | II-12 | II-46 |
| T2-101 | I-2 | II-12 | II-47 |
| T2-102 | I-2 | II-12 | II-48 |
| T2-103 | I-2 | II-13 | II-46 |
| T2-104 | I-2 | II-13 | II-47 |
| T2-105 | I-2 | II-13 | II-48 |
| T2-106 | I-2 | II-14 | II-46 |
| T2-107 | I-2 | II-14 | II-47 |
| T2-108 | I-2 | II-14 | II-48 |
| T2-109 | I-2 | II-15 | II-46 |
| T2-110 | I-2 | II-15 | II-47 |
| T2-111 | I-2 | II-15 | II-48 |
| T2-112 | I-2 | II-18 | II-46 |
| T2-113 | I-2 | II-18 | II-47 |
| T2-114 | I-2 | II-18 | II-48 |
| T2-115 | I-2 | II-19 | II-46 |
| T2-116 | I-2 | II-19 | II-47 |
| T2-117 | I-2 | II-19 | II-48 |
| T2-118 | I-2 | II-20 | II-46 |
| T2-119 | I-2 | II-20 | II-47 |
| T2-120 | I-2 | II-20 | II-48 |
| T2-121 | I-2 | II-21 | II-46 |
| T2-122 | I-2 | II-21 | II-47 |
| T2-123 | I-2 | II-21 | II-48 |
| T2-124 | I-2 | II-22 | II-46 |
| T2-125 | I-2 | II-22 | II-47 |
| T2-126 | I-2 | II-22 | II-48 |
| T2-127 | I-2 | II-23 | II-46 |
| T2-128 | I-2 | II-23 | II-47 |
| T2-129 | I-2 | II-23 | II-48 |
| T2-130 | I-2 | II-24 | II-46 |
| T2-131 | I-2 | II-24 | II-47 |
| T2-132 | I-2 | II-24 | II-48 |
| T2-133 | I-2 | II-25 | II-46 |
| T2-134 | I-2 | II-25 | II-47 |
| T2-135 | I-2 | II-25 | II-48 |
| T2-136 | I-2 | II-26 | II-46 |
| T2-137 | I-2 | II-26 | II-47 |
| T2-138 | I-2 | II-26 | II-48 |
| T2-139 | I-2 | II-27 | II-46 |
| T2-140 | I-2 | II-27 | II-47 |
| T2-141 | I-2 | II-27 | II-48 |
| T2-142 | I-2 | II-28 | II-46 |
| T2-143 | I-2 | II-28 | II-47 |
| T2-144 | I-2 | II-28 | II-48 |
| T2-145 | I-2 | II-29 | II-46 |
| T2-146 | I-2 | II-29 | II-47 |
| T2-147 | I-2 | II-29 | II-48 |
| T2-148 | I-2 | II-46 | II-47 |
| T2-149 | I-2 | II-46 | II-48 |
| T2-150 | I-2 | II-47 | II-48 |
| T2-151 | I-6 | II-1 | II-46 |
| T2-152 | I-6 | II-1 | II-47 |
| T2-153 | I-6 | II-1 | II-48 |
| T2-154 | I-6 | II-2 | II-46 |
| T2-155 | I-6 | II-2 | II-47 |
| T2-156 | I-6 | II-2 | II-48 |
| T2-157 | I-6 | II-3 | II-46 |
| T2-158 | I-6 | II-3 | II-47 |
| T2-159 | I-6 | II-3 | II-48 |
| T2-160 | I-6 | II-6 | II-46 |
| T2-161 | I-6 | II-6 | II-47 |
| T2-162 | I-6 | II-6 | II-48 |
| T2-163 | I-6 | II-7 | II-46 |
| T2-164 | I-6 | II-7 | II-47 |
| T2-165 | I-6 | II-7 | II-48 |
| T2-166 | I-6 | II-8 | II-46 |
| T2-167 | I-6 | II-8 | II-47 |
| T2-168 | I-6 | II-8 | II-48 |
| T2-169 | I-6 | II-9 | II-46 |
| T2-170 | I-6 | II-9 | II-47 |
| T2-171 | I-6 | II-9 | II-48 |
| T2-172 | I-6 | II-11 | II-46 |
| T2-173 | I-6 | II-11 | II-47 |
| T2-174 | I-6 | II-11 | II-48 |
| T2-175 | I-6 | II-12 | II-46 |
| T2-176 | I-6 | II-12 | II-47 |
| T2-177 | I-6 | II-12 | II-48 |
| T2-178 | I-6 | II-13 | II-46 |
| T2-179 | I-6 | II-13 | II-47 |
| T2-180 | I-6 | II-13 | II-48 |
| T2-181 | I-6 | II-14 | II-46 |
| T2-182 | I-6 | II-14 | II-47 |
| T2-183 | I-6 | II-14 | II-48 |
| T2-184 | I-6 | II-15 | II-46 |
| T2-185 | I-6 | II-15 | II-47 |
| T2-186 | I-6 | II-15 | II-48 |
| T2-187 | I-6 | II-18 | II-46 |
| T2-188 | I-6 | II-18 | II-47 |
| T2-189 | I-6 | II-18 | II-48 |
| T2-190 | I-6 | II-19 | II-46 |
| T2-191 | I-6 | II-19 | II-47 |
| T2-192 | I-6 | II-19 | II-48 |
| T2-193 | I-6 | II-20 | II-46 |
| T2-194 | I-6 | II-20 | II-47 |
| T2-195 | I-6 | II-20 | II-48 |
| T2-196 | I-6 | II-21 | II-46 |
| T2-197 | I-6 | II-21 | II-47 |
| T2-198 | I-6 | II-21 | II-48 |
| T2-199 | I-6 | II-22 | II-46 |
| T2-200 | I-6 | II-22 | II-47 |
| T2-201 | I-6 | II-22 | II-48 |
| T2-202 | I-6 | II-23 | II-46 |
| T2-203 | I-6 | II-23 | II-47 |
| T2-204 | I-6 | II-23 | II-48 |
| T2-205 | I-6 | II-24 | II-46 |
| T2-206 | I-6 | II-24 | II-47 |
| T2-207 | I-6 | II-24 | II-48 |
| T2-208 | I-6 | II-25 | II-46 |
| T2-209 | I-6 | II-25 | II-47 |
| T2-210 | I-6 | II-25 | II-48 |
| T2-211 | I-6 | II-26 | II-46 |
| T2-212 | I-6 | II-26 | II-47 |
| T2-213 | I-6 | II-26 | II-48 |
| T2-214 | I-6 | II-27 | II-46 |
| T2-215 | I-6 | II-27 | II-47 |
| T2-216 | I-6 | II-27 | II-48 |
| T2-217 | I-6 | II-28 | II-46 |
| T2-218 | I-6 | II-28 | II-47 |
| T2-219 | I-6 | II-28 | II-48 |
| T2-220 | I-6 | II-29 | II-46 |
| T2-221 | I-6 | II-29 | II-47 |
| T2-222 | I-6 | II-29 | II-48 |
| T2-223 | I-6 | II-46 | II-47 |
| T2-224 | I-6 | II-46 | II-48 |
| T2-225 | I-6 | II-47 | II-48 |
| T2-226 | I-7 | II-1 | II-46 |
| T2-227 | I-7 | II-1 | II-47 |
| T2-228 | I-7 | II-1 | II-48 |
| T2-229 | I-7 | II-2 | II-46 |
| T2-230 | I-7 | II-2 | II-47 |
| T2-231 | I-7 | II-2 | II-48 |
| T2-232 | I-7 | II-3 | II-46 |
| T2-233 | I-7 | II-3 | II-47 |
| T2-234 | I-7 | II-3 | II-48 |
| T2-235 | I-7 | II-6 | II-46 |
| T2-236 | I-7 | II-6 | II-47 |
| T2-237 | I-7 | II-6 | II-48 |
| T2-238 | I-7 | II-7 | II-46 |
| T2-239 | I-7 | II-7 | II-47 |
| T2-240 | I-7 | II-7 | II-48 |
| T2-241 | I-7 | II-8 | II-46 |
| T2-242 | I-7 | II-8 | II-47 |
| T2-243 | I-7 | II-8 | II-48 |
| T2-244 | I-7 | II-9 | II-46 |
| T2-245 | I-7 | II-9 | II-47 |
| T2-246 | I-7 | II-9 | II-48 |
| T2-247 | I-7 | II-11 | II-46 |
| T2-248 | I-7 | II-11 | II-47 |
| T2-249 | I-7 | II-11 | II-48 |
| T2-250 | I-7 | II-12 | II-46 |
| T2-251 | I-7 | II-12 | II-47 |
| T2-252 | I-7 | II-12 | II-48 |
| T2-253 | I-7 | II-13 | II-46 |
| T2-254 | I-7 | II-13 | II-47 |
| T2-255 | I-7 | II-13 | II-48 |
| T2-256 | I-7 | II-14 | II-46 |
| T2-257 | I-7 | II-14 | II-47 |
| T2-258 | I-7 | II-14 | II-48 |
| T2-259 | I-7 | II-15 | II-46 |
| T2-260 | I-7 | II-15 | II-47 |
| T2-261 | I-7 | II-15 | II-48 |
| T2-262 | I-7 | II-18 | II-46 |
| T2-263 | I-7 | II-18 | II-47 |
| T2-264 | I-7 | II-18 | II-48 |
| T2-265 | I-7 | II-19 | II-46 |
| T2-266 | I-7 | II-19 | II-47 |
| T2-267 | I-7 | II-19 | II-48 |
| T2-268 | I-7 | II-20 | II-46 |
| T2-269 | I-7 | II-20 | II-47 |
| T2-270 | I-7 | II-20 | II-48 |
| T2-271 | I-7 | II-21 | II-46 |
| T2-272 | I-7 | II-21 | II-47 |
| T2-273 | I-7 | II-21 | II-48 |
| T2-274 | I-7 | II-22 | II-46 |
| T2-275 | I-7 | II-22 | II-47 |
| T2-276 | I-7 | II-22 | II-48 |
| T2-277 | I-7 | II-23 | II-46 |
| T2-278 | I-7 | II-23 | II-47 |
| T2-279 | I-7 | II-23 | II-48 |
| T2-280 | I-7 | II-24 | II-46 |
| T2-281 | I-7 | II-24 | II-47 |
| T2-282 | I-7 | II-24 | II-48 |
| T2-283 | I-7 | II-25 | II-46 |
| T2-284 | I-7 | II-25 | II-47 |
| T2-285 | I-7 | II-25 | II-48 |
| T2-286 | I-7 | II-26 | II-46 |
| T2-287 | I-7 | II-26 | II-47 |
| T2-288 | I-7 | II-26 | II-48 |
| T2-289 | I-7 | II-27 | II-46 |
| T2-290 | I-7 | II-27 | II-47 |
| T2-291 | I-7 | II-27 | II-48 |
| T2-292 | I-7 | II-28 | II-46 |
| T2-293 | I-7 | II-28 | II-47 |
| T2-294 | I-7 | II-28 | II-48 |
| T2-295 | I-7 | II-29 | II-46 |
| T2-296 | I-7 | II-29 | II-47 |
| T2-297 | I-7 | II-29 | II-48 |
| T2-298 | I-7 | II-46 | II-47 |
| T2-299 | I-7 | II-46 | II-48 |
| T2-300 | I-7 | II-47 | II-48 |
| T2-301 | I-8 | II-1 | II-46 |
| T2-302 | I-8 | II-1 | II-47 |
| T2-303 | I-8 | II-1 | II-48 |
| T2-304 | I-8 | II-2 | II-46 |
| T2-305 | I-8 | II-2 | II-47 |
| T2-306 | I-8 | II-2 | II-48 |
| T2-307 | I-8 | II-3 | II-46 |
| T2-308 | I-8 | II-3 | II-47 |
| T2-309 | I-8 | II-3 | II-48 |
| T2-310 | I-8 | II-6 | II-46 |
| T2-311 | I-8 | II-6 | II-47 |
| T2-312 | I-8 | II-6 | II-48 |
| T2-313 | I-8 | II-7 | II-46 |
| T2-314 | I-8 | II-7 | II-47 |
| T2-315 | I-8 | II-7 | II-48 |
| T2-316 | I-8 | II-8 | II-46 |
| T2-317 | I-8 | II-8 | II-47 |
| T2-318 | I-8 | II-8 | II-48 |
| T2-319 | I-8 | II-9 | II-46 |
| T2-320 | I-8 | II-9 | II-47 |
| T2-321 | I-8 | II-9 | II-48 |
| T2-322 | I-8 | II-11 | II-46 |
| T2-323 | I-8 | II-11 | II-47 |
| T2-324 | I-8 | II-11 | II-48 |
| T2-325 | I-8 | II-12 | II-46 |
| T2-326 | I-8 | II-12 | II-47 |
| T2-327 | I-8 | II-12 | II-48 |
| T2-328 | I-8 | II-13 | II-46 |
| T2-329 | I-8 | II-13 | II-47 |
| T2-330 | I-8 | II-13 | II-48 |
| T2-331 | I-8 | II-14 | II-46 |
| T2-332 | I-8 | II-14 | II-47 |
| T2-333 | I-8 | II-14 | II-48 |
| T2-334 | I-8 | II-15 | II-46 |
| T2-335 | I-8 | II-15 | II-47 |
| T2-336 | I-8 | II-15 | II-48 |
| T2-337 | I-8 | II-18 | II-46 |
| T2-338 | I-8 | II-18 | II-47 |
| T2-339 | I-8 | II-18 | II-48 |
| T2-340 | I-8 | II-19 | II-46 |
| T2-341 | I-8 | II-19 | II-47 |
| T2-342 | I-8 | II-19 | II-48 |
| T2-343 | I-8 | II-20 | II-46 |
| T2-344 | I-8 | II-20 | II-47 |
| T2-345 | I-8 | II-20 | II-48 |
| T2-346 | I-8 | II-21 | II-46 |
| T2-347 | I-8 | II-21 | II-47 |
| T2-348 | I-8 | II-21 | II-48 |
| T2-349 | I-8 | II-22 | II-46 |
| T2-350 | I-8 | II-22 | II-47 |
| T2-351 | I-8 | II-22 | II-48 |
| T2-352 | I-8 | II-23 | II-46 |
| T2-353 | I-8 | II-23 | II-47 |
| T2-354 | I-8 | II-23 | II-48 |
| T2-355 | I-8 | II-24 | II-46 |
| T2-356 | I-8 | II-24 | II-47 |
| T2-357 | I-8 | II-24 | II-48 |
| T2-358 | I-8 | II-25 | II-46 |
| T2-359 | I-8 | II-25 | II-47 |
| T2-360 | I-8 | II-25 | II-48 |
| T2-361 | I-8 | II-26 | II-46 |
| T2-362 | I-8 | II-26 | II-47 |
| T2-363 | I-8 | II-26 | II-48 |
| T2-364 | I-8 | II-27 | II-46 |
| T2-365 | I-8 | II-27 | II-47 |
| T2-366 | I-8 | II-27 | II-48 |
| T2-367 | I-8 | II-28 | II-46 |
| T2-368 | I-8 | II-28 | II-47 |
| T2-369 | I-8 | II-28 | II-48 |
| T2-370 | I-8 | II-29 | II-46 |
| T2-371 | I-8 | II-29 | II-47 |
| T2-372 | I-8 | II-29 | II-48 |
| T2-373 | I-8 | II-46 | II-47 |
| T2-374 | I-8 | II-46 | II-48 |
| T2-375 | I-8 | II-47 | II-48 |
| T2-376 | I-9 | II-1 | II-46 |
| T2-377 | I-9 | II-1 | II-47 |
| T2-378 | I-9 | II-1 | II-48 |
| T2-379 | I-9 | II-2 | II-46 |
| T2-380 | I-9 | II-2 | II-47 |
| T2-381 | I-9 | II-2 | II-48 |
| T2-382 | I-9 | II-3 | II-46 |
| T2-383 | I-9 | II-3 | II-47 |
| T2-384 | I-9 | II-3 | II-48 |
| T2-385 | I-9 | II-6 | II-46 |
| T2-386 | I-9 | II-6 | II-47 |
| T2-387 | I-9 | II-6 | II-48 |
| T2-388 | I-9 | II-7 | II-46 |
| T2-389 | I-9 | II-7 | II-47 |
| T2-390 | I-9 | II-7 | II-48 |
| T2-391 | I-9 | II-8 | II-46 |
| T2-392 | I-9 | II-8 | II-47 |
| T2-393 | I-9 | II-8 | II-48 |
| T2-394 | I-9 | II-9 | II-46 |
| T2-395 | I-9 | II-9 | II-47 |
| T2-396 | I-9 | II-9 | II-48 |
| T2-397 | I-9 | II-11 | II-46 |
| T2-398 | I-9 | II-11 | II-47 |
| T2-399 | I-9 | II-11 | II-48 |
| T2-400 | I-9 | II-12 | II-46 |
| T2-401 | I-9 | II-12 | II-47 |
| T2-402 | I-9 | II-12 | II-48 |
| T2-403 | I-9 | II-13 | II-46 |
| T2-404 | I-9 | II-13 | II-47 |
| T2-405 | I-9 | II-13 | II-48 |
| T2-406 | I-9 | II-14 | II-46 |
| T2-407 | I-9 | II-14 | II-47 |
| T2-408 | I-9 | II-14 | II-48 |
| T2-409 | I-9 | II-15 | II-46 |
| T2-410 | I-9 | II-15 | II-47 |
| T2-411 | I-9 | II-15 | II-48 |
| T2-412 | I-9 | II-18 | II-46 |
| T2-413 | I-9 | II-18 | II-47 |
| T2-414 | I-9 | II-18 | II-48 |
| T2-415 | I-9 | II-19 | II-46 |
| T2-416 | I-9 | II-19 | II-47 |
| T2-417 | I-9 | II-19 | II-48 |
| T2-418 | I-9 | II-20 | II-46 |
| T2-419 | I-9 | II-20 | II-47 |
| T2-420 | I-9 | II-20 | II-48 |
| T2-421 | I-9 | II-21 | II-46 |
| T2-422 | I-9 | II-21 | II-47 |
| T2-423 | I-9 | II-21 | II-48 |
| T2-424 | I-9 | II-22 | II-46 |
| T2-425 | I-9 | II-22 | II-47 |
| T2-426 | I-9 | II-22 | II-48 |
| T2-427 | I-9 | II-23 | II-46 |
| T2-428 | I-9 | II-23 | II-47 |
| T2-429 | I-9 | II-23 | II-48 |
| T2-430 | I-9 | II-24 | II-46 |
| T2-431 | I-9 | II-24 | II-47 |
| T2-432 | I-9 | II-24 | II-48 |
| T2-433 | I-9 | II-25 | II-46 |
| T2-434 | I-9 | II-25 | II-47 |
| T2-435 | I-9 | II-25 | II-48 |
| T2-436 | I-9 | II-26 | II-46 |
| T2-437 | I-9 | II-26 | II-47 |
| T2-438 | I-9 | II-26 | II-48 |
| T2-439 | I-9 | II-27 | II-46 |
| T2-440 | I-9 | II-27 | II-47 |
| T2-441 | I-9 | II-27 | II-48 |
| T2-442 | I-9 | II-28 | II-46 |
| T2-443 | I-9 | II-28 | II-47 |
| T2-444 | I-9 | II-28 | II-48 |
| T2-445 | I-9 | II-29 | II-46 |
| T2-446 | I-9 | II-29 | II-47 |
| T2-447 | I-9 | II-29 | II-48 |
| T2-448 | I-9 | II-46 | II-47 |
| T2-449 | I-9 | II-46 | II-48 |
| T2-450 | I-9 | II-47 | II-48 |
| T2-451 | I-11 | II-1 | II-46 |
| T2-452 | I-11 | II-1 | II-47 |
| T2-453 | I-11 | II-1 | II-48 |
| T2-454 | I-11 | II-2 | II-46 |
| T2-455 | I-11 | II-2 | II-47 |
| T2-456 | I-11 | II-2 | II-48 |
| T2-457 | I-11 | II-3 | II-46 |
| T2-458 | I-11 | II-3 | II-47 |
| T2-459 | I-11 | II-3 | II-48 |
| T2-460 | I-11 | II-6 | II-46 |
| T2-461 | I-11 | II-6 | II-47 |
| T2-462 | I-11 | II-6 | II-48 |
| T2-463 | I-11 | II-7 | II-46 |
| T2-464 | I-11 | II-7 | II-47 |
| T2-465 | I-11 | II-7 | II-48 |
| T2-466 | I-11 | II-8 | II-46 |
| T2-467 | I-11 | II-8 | II-47 |
| T2-468 | I-11 | II-8 | II-48 |
| T2-469 | I-11 | II-9 | II-46 |
| T2-470 | I-11 | II-9 | II-47 |
| T2-471 | I-11 | II-9 | II-48 |
| T2-472 | I-11 | II-11 | II-46 |
| T2-473 | I-11 | II-11 | II-47 |
| T2-474 | I-11 | II-11 | II-48 |
| T2-475 | I-11 | II-12 | II-46 |
| T2-476 | I-11 | II-12 | II-47 |
| T2-477 | I-11 | II-12 | II-48 |
| T2-478 | I-11 | II-13 | II-46 |
| T2-479 | I-11 | II-13 | II-47 |
| T2-480 | I-11 | II-13 | II-48 |
| T2-481 | I-11 | II-14 | II-46 |
| T2-482 | I-11 | II-14 | II-47 |
| T2-483 | I-11 | II-14 | II-48 |
| T2-484 | I-11 | II-15 | II-46 |
| T2-485 | I-11 | II-15 | II-47 |
| T2-486 | I-11 | II-15 | II-48 |
| T2-487 | I-11 | II-18 | II-46 |
| T2-488 | I-11 | II-18 | II-47 |
| T2-489 | I-11 | II-18 | II-48 |
| T2-490 | I-11 | II-19 | II-46 |
| T2-491 | I-11 | II-19 | II-47 |
| T2-492 | I-11 | II-19 | II-48 |
| T2-493 | I-11 | II-20 | II-46 |
| T2-494 | I-11 | II-20 | II-47 |
| T2-495 | I-11 | II-20 | II-48 |
| T2-496 | I-11 | II-21 | II-46 |
| T2-497 | I-11 | II-21 | II-47 |
| T2-498 | I-11 | II-21 | II-48 |
| T2-499 | I-11 | II-22 | II-46 |
| T2-500 | I-11 | II-22 | II-47 |
| T2-501 | I-11 | II-22 | II-48 |
| T2-502 | I-11 | II-23 | II-46 |
| T2-503 | I-11 | II-23 | II-47 |
| T2-504 | I-11 | II-23 | II-48 |
| T2-505 | I-11 | II-24 | II-46 |
| T2-506 | I-11 | II-24 | II-47 |
| T2-507 | I-11 | II-24 | II-48 |
| T2-508 | I-11 | II-25 | II-46 |
| T2-509 | I-11 | II-25 | II-47 |
| T2-510 | I-11 | II-25 | II-48 |
| T2-511 | I-11 | II-26 | II-46 |
| T2-512 | I-11 | II-26 | II-47 |
| T2-513 | I-11 | II-26 | II-48 |
| T2-514 | I-11 | II-27 | II-46 |
| T2-515 | I-11 | II-27 | II-47 |
| T2-516 | I-11 | II-27 | II-48 |
| T2-517 | I-11 | II-28 | II-46 |
| T2-518 | I-11 | II-28 | II-47 |
| T2-519 | I-11 | II-28 | II-48 |
| T2-520 | I-11 | II-29 | II-46 |
| T2-521 | I-11 | II-29 | II-47 |
| T2-522 | I-11 | II-29 | II-48 |
| T2-523 | I-11 | II-46 | II-47 |
| T2-524 | I-11 | II-46 | II-48 |
| T2-525 | I-11 | II-47 | II-48 |
| T2-526 | I-12 | II-1 | II-46 |
| T2-527 | I-12 | II-1 | II-47 |
| T2-528 | I-12 | II-1 | II-48 |
| T2-529 | I-12 | II-2 | II-46 |
| T2-530 | I-12 | II-2 | II-47 |
| T2-531 | I-12 | II-2 | II-48 |
| T2-532 | I-12 | II-3 | II-46 |
| T2-533 | I-12 | II-3 | II-47 |
| T2-534 | I-12 | II-3 | II-48 |
| T2-535 | I-12 | II-6 | II-46 |
| T2-536 | I-12 | II-6 | II-47 |
| T2-537 | I-12 | II-6 | II-48 |
| T2-538 | I-12 | II-7 | II-46 |
| T2-539 | I-12 | II-7 | II-47 |
| T2-540 | I-12 | II-7 | II-48 |
| T2-541 | I-12 | II-8 | II-46 |
| T2-542 | I-12 | II-8 | II-47 |
| T2-543 | I-12 | II-8 | II-48 |
| T2-544 | I-12 | II-9 | II-46 |
| T2-545 | I-12 | II-9 | II-47 |
| T2-546 | I-12 | II-9 | II-48 |
| T2-547 | I-12 | II-11 | II-46 |
| T2-548 | I-12 | II-11 | II-47 |
| T2-549 | I-12 | II-11 | II-48 |
| T2-550 | I-12 | II-12 | II-46 |
| T2-551 | I-12 | II-12 | II-47 |
| T2-552 | I-12 | II-12 | II-48 |
| T2-553 | I-12 | II-13 | II-46 |
| T2-554 | I-12 | II-13 | II-47 |
| T2-555 | I-12 | II-13 | II-48 |
| T2-556 | I-12 | II-14 | II-46 |
| T2-557 | I-12 | II-14 | II-47 |
| T2-558 | I-12 | II-14 | II-48 |
| T2-559 | I-12 | II-15 | II-46 |
| T2-560 | I-12 | II-15 | II-47 |
| T2-561 | I-12 | II-15 | II-48 |
| T2-562 | I-12 | II-18 | II-46 |
| T2-563 | I-12 | II-18 | II-47 |
| T2-564 | I-12 | II-18 | II-48 |
| T2-565 | I-12 | II-19 | II-46 |
| T2-566 | I-12 | II-19 | II-47 |
| T2-567 | I-12 | II-19 | II-48 |
| T2-568 | I-12 | II-20 | II-46 |
| T2-569 | I-12 | II-20 | II-47 |
| T2-570 | I-12 | II-20 | II-48 |
| T2-571 | I-12 | II-21 | II-46 |
| T2-572 | I-12 | II-21 | II-47 |
| T2-573 | I-12 | II-21 | II-48 |
| T2-574 | I-12 | II-22 | II-46 |
| T2-575 | I-12 | II-22 | II-47 |
| T2-576 | I-12 | II-22 | II-48 |
| T2-577 | I-12 | II-23 | II-46 |
| T2-578 | I-12 | II-23 | II-47 |
| T2-579 | I-12 | II-23 | II-48 |
| T2-580 | I-12 | II-24 | II-46 |
| T2-581 | I-12 | II-24 | II-47 |
| T2-582 | I-12 | II-24 | II-48 |
| T2-583 | I-12 | II-25 | II-46 |
| T2-584 | I-12 | II-25 | II-47 |
| T2-585 | I-12 | II-25 | II-48 |
| T2-586 | I-12 | II-26 | II-46 |
| T2-587 | I-12 | II-26 | II-47 |
| T2-588 | I-12 | II-26 | II-48 |
| T2-589 | I-12 | II-27 | II-46 |
| T2-590 | I-12 | II-27 | II-47 |
| T2-591 | I-12 | II-27 | II-48 |
| T2-592 | I-12 | II-28 | II-46 |
| T2-593 | I-12 | II-28 | II-47 |
| T2-594 | I-12 | II-28 | II-48 |
| T2-595 | I-12 | II-29 | II-46 |
| T2-596 | I-12 | II-29 | II-47 |
| T2-597 | I-12 | II-29 | II-48 |
| T2-598 | I-12 | II-46 | II-47 |
| T2-599 | I-12 | II-46 | II-48 |
| T2-600 | I-12 | II-47 | II-48 |
| T2-601 | I-13 | II-1 | II-46 |
| T2-602 | I-13 | II-1 | II-47 |
| T2-603 | I-13 | II-1 | II-48 |
| T2-604 | I-13 | II-2 | II-46 |
| T2-605 | I-13 | II-2 | II-47 |
| T2-606 | I-13 | II-2 | II-48 |
| T2-607 | I-13 | II-3 | II-46 |
| T2-608 | I-13 | II-3 | II-47 |
| T2-609 | I-13 | II-3 | II-48 |
| T2-610 | I-13 | II-6 | II-46 |
| T2-611 | I-13 | II-6 | II-47 |
| T2-612 | I-13 | II-6 | II-48 |
| T2-613 | I-13 | II-7 | II-46 |
| T2-614 | I-13 | II-7 | II-47 |
| T2-615 | I-13 | II-7 | II-48 |
| T2-616 | I-13 | II-8 | II-46 |
| T2-617 | I-13 | II-8 | II-47 |
| T2-618 | I-13 | II-8 | II-48 |
| T2-619 | I-13 | II-9 | II-46 |
| T2-620 | I-13 | II-9 | II-47 |
| T2-621 | I-13 | II-9 | II-48 |
| T2-622 | I-13 | II-11 | II-46 |
| T2-623 | I-13 | II-11 | II-47 |
| T2-624 | I-13 | II-11 | II-48 |
| T2-625 | I-13 | II-12 | II-46 |
| T2-626 | I-13 | II-12 | II-47 |
| T2-627 | I-13 | II-12 | II-48 |
| T2-628 | I-13 | II-13 | II-46 |
| T2-629 | I-13 | II-13 | II-47 |
| T2-630 | I-13 | II-13 | II-48 |
| T2-631 | I-13 | II-14 | II-46 |
| T2-632 | I-13 | II-14 | II-47 |
| T2-633 | I-13 | II-14 | II-48 |
| T2-634 | I-13 | II-15 | II-46 |
| T2-635 | I-13 | II-15 | II-47 |
| T2-636 | I-13 | II-15 | II-48 |
| T2-637 | I-13 | II-18 | II-46 |
| T2-638 | I-13 | II-18 | II-47 |
| T2-639 | I-13 | II-18 | II-48 |
| T2-640 | I-13 | II-19 | II-46 |
| T2-641 | I-13 | II-19 | II-47 |
| T2-642 | I-13 | II-19 | II-48 |
| T2-643 | I-13 | II-20 | II-46 |
| T2-644 | I-13 | II-20 | II-47 |
| T2-645 | I-13 | II-20 | II-48 |
| T2-646 | I-13 | II-21 | II-46 |
| T2-647 | I-13 | II-21 | II-47 |
| T2-648 | I-13 | II-21 | II-48 |
| T2-649 | I-13 | II-22 | II-46 |
| T2-650 | I-13 | II-22 | II-47 |
| T2-651 | I-13 | II-22 | II-48 |
| T2-652 | I-13 | II-23 | II-46 |
| T2-653 | I-13 | II-23 | II-47 |
| T2-654 | I-13 | II-23 | II-48 |
| T2-655 | I-13 | II-24 | II-46 |
| T2-656 | I-13 | II-24 | II-47 |
| T2-657 | I-13 | II-24 | II-48 |
| T2-658 | I-13 | II-25 | II-46 |
| T2-659 | I-13 | II-25 | II-47 |
| T2-660 | I-13 | II-25 | II-48 |
| T2-661 | I-13 | II-26 | II-46 |
| T2-662 | I-13 | II-26 | II-47 |
| T2-663 | I-13 | II-26 | II-48 |
| T2-664 | I-13 | II-27 | II-46 |
| T2-665 | I-13 | II-27 | II-47 |
| T2-666 | I-13 | II-27 | II-48 |
| T2-667 | I-13 | II-28 | II-46 |
| T2-668 | I-13 | II-28 | II-47 |
| T2-669 | I-13 | II-28 | II-48 |
| T2-670 | I-13 | II-29 | II-46 |
| T2-671 | I-13 | II-29 | II-47 |
| T2-672 | I-13 | II-29 | II-48 |
| T2-673 | I-13 | II-46 | II-47 |
| T2-674 | I-13 | II-46 | II-48 |
| T2-675 | I-13 | II-47 | II-48 |
| T2-676 | I-14 | II-1 | II-46 |
| T2-677 | I-14 | II-1 | II-47 |
| T2-678 | I-14 | II-1 | II-48 |
| T2-679 | I-14 | II-2 | II-46 |
| T2-680 | I-14 | II-2 | II-47 |
| T2-681 | I-14 | II-2 | II-48 |
| T2-682 | I-14 | II-3 | II-46 |
| T2-683 | I-14 | II-3 | II-47 |
| T2-684 | I-14 | II-3 | II-48 |
| T2-685 | I-14 | II-6 | II-46 |
| T2-686 | I-14 | II-6 | II-47 |
| T2-687 | I-14 | II-6 | II-48 |
| T2-688 | I-14 | II-7 | II-46 |
| T2-689 | I-14 | II-7 | II-47 |
| T2-690 | I-14 | II-7 | II-48 |
| T2-691 | I-14 | II-8 | II-46 |
| T2-692 | I-14 | II-8 | II-47 |
| T2-693 | I-14 | II-8 | II-48 |
| T2-694 | I-14 | II-9 | II-46 |
| T2-695 | I-14 | II-9 | II-47 |
| T2-696 | I-14 | II-9 | II-48 |
| T2-697 | I-14 | II-11 | II-46 |
| T2-698 | I-14 | II-11 | II-47 |
| T2-699 | I-14 | II-11 | II-48 |
| T2-700 | I-14 | II-12 | II-46 |
| T2-701 | I-14 | II-12 | II-47 |
| T2-702 | I-14 | II-12 | II-48 |
| T2-703 | I-14 | II-13 | II-46 |
| T2-704 | I-14 | II-13 | II-47 |
| T2-705 | I-14 | II-13 | II-48 |
| T2-706 | I-14 | II-14 | II-46 |
| T2-707 | I-14 | II-14 | II-47 |
| T2-708 | I-14 | II-14 | II-48 |
| T2-709 | I-14 | II-15 | II-46 |
| T2-710 | I-14 | II-15 | II-47 |
| T2-711 | I-14 | II-15 | II-48 |
| T2-712 | I-14 | II-18 | II-46 |
| T2-713 | I-14 | II-18 | II-47 |
| T2-714 | I-14 | II-18 | II-48 |
| T2-715 | I-14 | II-19 | II-46 |
| T2-716 | I-14 | II-19 | II-47 |
| T2-717 | I-14 | II-19 | II-48 |
| T2-718 | I-14 | II-20 | II-46 |
| T2-719 | I-14 | II-20 | II-47 |
| T2-720 | I-14 | II-20 | II-48 |
| T2-721 | I-14 | II-21 | II-46 |
| T2-722 | I-14 | II-21 | II-47 |
| T2-723 | I-14 | II-21 | II-48 |
| T2-724 | I-14 | II-22 | II-46 |
| T2-725 | I-14 | II-22 | II-47 |
| T2-726 | I-14 | II-22 | II-48 |
| T2-727 | I-14 | II-23 | II-46 |
| T2-728 | I-14 | II-23 | II-47 |
| T2-729 | I-14 | II-23 | II-48 |
| T2-730 | I-14 | II-24 | II-46 |
| T2-731 | I-14 | II-24 | II-47 |
| T2-732 | I-14 | II-24 | II-48 |
| T2-733 | I-14 | II-25 | II-46 |
| T2-734 | I-14 | II-25 | II-47 |
| T2-735 | I-14 | II-25 | II-48 |
| T2-736 | I-14 | II-26 | II-46 |
| T2-737 | I-14 | II-26 | II-47 |
| T2-738 | I-14 | II-26 | II-48 |
| T2-739 | I-14 | II-27 | II-46 |
| T2-740 | I-14 | II-27 | II-47 |
| T2-741 | I-14 | II-27 | II-48 |
| T2-742 | I-14 | II-28 | II-46 |
| T2-743 | I-14 | II-28 | II-47 |
| T2-744 | I-14 | II-28 | II-48 |
| T2-745 | I-14 | II-29 | II-46 |
| T2-746 | I-14 | II-29 | II-47 |
| T2-747 | I-14 | II-29 | II-48 |
| T2-748 | I-14 | II-46 | II-47 |
| T2-749 | I-14 | II-46 | II-48 |
| T2-750 | I-14 | II-47 | II-48 |
| T2-751 | I-18 | II-1 | II-46 |
| T2-752 | I-18 | II-1 | II-47 |
| T2-753 | I-18 | II-1 | II-48 |
| T2-754 | I-18 | II-2 | II-46 |
| T2-755 | I-18 | II-2 | II-47 |
| T2-756 | I-18 | II-2 | II-48 |
| T2-757 | I-18 | II-3 | II-46 |
| T2-758 | I-18 | II-3 | II-47 |
| T2-759 | I-18 | II-3 | II-48 |
| T2-760 | I-18 | II-6 | II-46 |
| T2-761 | I-18 | II-6 | II-47 |
| T2-762 | I-18 | II-6 | II-48 |
| T2-763 | I-18 | II-7 | II-46 |
| T2-764 | I-18 | II-7 | II-47 |
| T2-765 | I-18 | II-7 | II-48 |
| T2-766 | I-18 | II-8 | II-46 |
| T2-767 | I-18 | II-8 | II-47 |
| T2-768 | I-18 | II-8 | II-48 |
| T2-769 | I-18 | II-9 | II-46 |
| T2-770 | I-18 | II-9 | II-47 |
| T2-771 | I-18 | II-9 | II-48 |
| T2-772 | I-18 | II-11 | II-46 |
| T2-773 | I-18 | II-11 | II-47 |
| T2-774 | I-18 | II-11 | II-48 |
| T2-775 | I-18 | II-12 | II-46 |
| T2-776 | I-18 | II-12 | II-47 |
| T2-777 | I-18 | II-12 | II-48 |
| T2-778 | I-18 | II-13 | II-46 |
| T2-779 | I-18 | II-13 | II-47 |
| T2-780 | I-18 | II-13 | II-48 |
| T2-781 | I-18 | II-14 | II-46 |
| T2-782 | I-18 | II-14 | II-47 |
| T2-783 | I-18 | II-14 | II-48 |
| T2-784 | I-18 | II-15 | II-46 |
| T2-785 | I-18 | II-15 | II-47 |
| T2-786 | I-18 | II-15 | II-48 |
| T2-787 | I-18 | II-18 | II-46 |
| T2-788 | I-18 | II-18 | II-47 |
| T2-789 | I-18 | II-18 | II-48 |
| T2-790 | I-18 | II-19 | II-46 |
| T2-791 | I-18 | II-19 | II-47 |
| T2-792 | I-18 | II-19 | II-48 |
| T2-793 | I-18 | II-20 | II-46 |
| T2-794 | I-18 | II-20 | II-47 |
| T2-795 | I-18 | II-20 | II-48 |
| T2-796 | I-18 | II-21 | II-46 |
| T2-797 | I-18 | II-21 | II-47 |
| T2-798 | I-18 | II-21 | II-48 |
| T2-799 | I-18 | II-22 | II-46 |
| T2-800 | I-18 | II-22 | II-47 |
| T2-801 | I-18 | II-22 | II-48 |
| T2-802 | I-18 | II-23 | II-46 |
| T2-803 | I-18 | II-23 | II-47 |
| T2-804 | I-18 | II-23 | II-48 |
| T2-805 | I-18 | II-24 | II-46 |
| T2-806 | I-18 | II-24 | II-47 |
| T2-807 | I-18 | II-24 | II-48 |
| T2-808 | I-18 | II-25 | II-46 |
| T2-809 | I-18 | II-25 | II-47 |
| T2-810 | I-18 | II-25 | II-48 |
| T2-811 | I-18 | II-26 | II-46 |
| T2-812 | I-18 | II-26 | II-47 |
| T2-813 | I-18 | II-26 | II-48 |
| T2-814 | I-18 | II-27 | II-46 |
| T2-815 | I-18 | II-27 | II-47 |
| T2-816 | I-18 | II-27 | II-48 |
| T2-817 | I-18 | II-28 | II-46 |
| T2-818 | I-18 | II-28 | II-47 |
| T2-819 | I-18 | II-28 | II-48 |
| T2-820 | I-18 | II-29 | II-46 |
| T2-821 | I-18 | II-29 | II-47 |
| T2-822 | I-18 | II-29 | II-48 |
| T2-823 | I-18 | II-46 | II-47 |
| T2-824 | I-18 | II-46 | II-48 |
| T2-825 | I-18 | II-47 | II-48 |

“C” denotes “component,
“M” denotes “mixture”.

According to a further embodiment, preference is given to ternary mixtures in which component 2 is a compound II selected from the group of the following compounds:

| | |
|---|---|
| II-8 | kresoxim-methyl |
| II-9 | pyraclostrobin |
| II-25 | N-(3′,4′,5′-trifluorobiphenyl-2-yl)-3-difluoromethyl-1-methyl-1H- |
| | pyrazole-4-carboxamide |
| II-26 | sedaxane |
| II-28 | fluopyram |
| II-29 | penflufen |
| II-49 | azoxystrobin |
| II-50 | trifloxystrobin |
| II-51 | penthiopyrad |

and in which component 3 is a compound II selected from the group of the following compounds:

| | |
|---|---|
| II-30a | fipronil |
| II-32a | clothianidin |
| II-34a | imidacloprid |
| II-35a | thiamethoxam |

TABLE T3
The ternary mixtures T3-1 to T3-396 below comprise, as component 1,
a preferred compound I, a component 2 selected from the active
compounds preferred for component 2 and a component 3 selected from
the active compounds preferred for component 3. In the table below, each
row corresponds to a ternary mixture according to the invention having the
mixture component 1 to 3 listed in the row in question.

| M | C1 | C2 | C3 |
|---|---|---|---|
| T3-1 | I-1 | II-8 | II-30a |
| T3-2 | I-1 | II-8 | II-32a |
| T3-3 | I-1 | II-8 | II-34a |
| T3-4 | I-1 | II-8 | II-35a |
| T3-5 | I-1 | II-9 | II-30a |
| T3-6 | I-1 | II-9 | II-32a |
| T3-7 | I-1 | II-9 | II-34a |
| T3-8 | I-1 | II-9 | II-35a |
| T3-9 | I-1 | II-25 | II-30a |
| T3-10 | I-1 | II-25 | II-32a |
| T3-11 | I-1 | II-25 | II-34a |
| T3-12 | I-1 | II-25 | II-35a |
| T3-13 | I-1 | II-26 | II-30a |
| T3-14 | I-1 | II-26 | II-32a |
| T3-15 | I-1 | II-26 | II-34a |
| T3-16 | I-1 | II-26 | II-35a |
| T3-17 | I-1 | II-28 | II-30a |
| T3-18 | I-1 | II-28 | II-32a |
| T3-19 | I-1 | II-28 | II-34a |
| T3-20 | I-1 | II-28 | II-35a |
| T3-21 | I-1 | II-29 | II-30a |
| T3-22 | I-1 | II-29 | II-32a |
| T3-23 | I-1 | II-29 | II-34a |
| T3-24 | I-1 | II-29 | II-35a |
| T3-25 | I-1 | II-49 | II-30a |
| T3-26 | I-1 | II-49 | II-32a |
| T3-27 | I-1 | II-49 | II-34a |
| T3-28 | I-1 | II-49 | II-35a |
| T3-29 | I-1 | II-50 | II-30a |
| T3-30 | I-1 | II-50 | II-32a |
| T3-31 | I-1 | II-50 | II-34a |
| T3-32 | I-1 | II-50 | II-35a |
| T3-33 | I-1 | II-51 | II-30a |
| T3-34 | I-1 | II-51 | II-32a |
| T3-35 | I-1 | II-51 | II-34a |
| T3-36 | I-1 | II-51 | II-35a |
| T3-37 | I-2 | II-8 | II-30a |
| T3-38 | I-2 | II-8 | II-32a |
| T3-39 | I-2 | II-8 | II-34a |
| T3-40 | I-2 | II-8 | II-35a |
| T3-41 | I-2 | II-9 | II-30a |
| T3-42 | I-2 | II-9 | II-32a |
| T3-43 | I-2 | II-9 | II-34a |
| T3-44 | I-2 | II-9 | II-35a |
| T3-45 | I-2 | II-25 | II-30a |
| T3-46 | I-2 | II-25 | II-32a |
| T3-47 | I-2 | II-25 | II-34a |
| T3-48 | I-2 | II-25 | II-35a |
| T3-49 | I-2 | II-26 | II-30a |
| T3-50 | I-2 | II-26 | II-32a |
| T3-51 | I-2 | II-26 | II-34a |
| T3-52 | I-2 | II-26 | II-35a |
| T3-53 | I-2 | II-28 | II-30a |
| T3-54 | I-2 | II-28 | II-32a |
| T3-55 | I-2 | II-28 | II-34a |
| T3-56 | I-2 | II-28 | II-35a |
| T3-57 | I-2 | II-29 | II-30a |
| T3-58 | I-2 | II-29 | II-32a |
| T3-59 | I-2 | II-29 | II-34a |
| T3-60 | I-2 | II-29 | II-35a |
| T3-61 | I-2 | II-49 | II-30a |
| T3-62 | I-2 | II-49 | II-32a |
| T3-63 | I-2 | II-49 | II-34a |
| T3-64 | I-2 | II-49 | II-35a |
| T3-65 | I-2 | II-50 | II-30a |
| T3-66 | I-2 | II-50 | II-32a |
| T3-67 | I-2 | II-50 | II-34a |
| T3-68 | I-2 | II-50 | II-35a |
| T3-69 | I-2 | II-51 | II-30a |
| T3-70 | I-2 | II-51 | II-32a |
| T3-71 | I-2 | II-51 | II-34a |
| T3-72 | I-2 | II-51 | II-35a |
| T3-73 | I-6 | II-8 | II-30a |
| T3-74 | I-6 | II-8 | II-32a |
| T3-75 | I-6 | II-8 | II-34a |
| T3-76 | I-6 | II-8 | II-35a |
| T3-77 | I-6 | II-9 | II-30a |
| T3-78 | I-6 | II-9 | II-32a |
| T3-79 | I-6 | II-9 | II-34a |
| T3-80 | I-6 | II-9 | II-35a |
| T3-81 | I-6 | II-25 | II-30a |
| T3-82 | I-6 | II-25 | II-32a |
| T3-83 | I-6 | II-25 | II-34a |
| T3-84 | I-6 | II-25 | II-35a |
| T3-85 | I-6 | II-26 | II-30a |
| T3-86 | I-6 | II-26 | II-32a |
| T3-87 | I-6 | II-26 | II-34a |
| T3-88 | I-6 | II-26 | II-35a |
| T3-89 | I-6 | II-28 | II-30a |
| T3-90 | I-6 | II-28 | II-32a |
| T3-91 | I-6 | II-28 | II-34a |
| T3-92 | I-6 | II-28 | II-35a |
| T3-93 | I-6 | II-29 | II-30a |
| T3-94 | I-6 | II-29 | II-32a |
| T3-95 | I-6 | II-29 | II-34a |
| T3-96 | I-6 | II-29 | II-35a |
| T3-97 | I-6 | II-49 | II-30a |
| T3-98 | I-6 | II-49 | II-32a |
| T3-99 | I-6 | II-49 | II-34a |
| T3-100 | I-6 | II-49 | II-35a |
| T3-101 | I-6 | II-50 | II-30a |
| T3-102 | I-6 | II-50 | II-32a |
| T3-103 | I-6 | II-50 | II-34a |
| T3-104 | I-6 | II-50 | II-35a |
| T3-105 | I-6 | II-51 | II-30a |
| T3-106 | I-6 | II-51 | II-32a |
| T3-107 | I-6 | II-51 | II-34a |
| T3-108 | I-6 | II-51 | II-35a |
| T3-109 | I-7 | II-8 | II-30a |
| T3-110 | I-7 | II-8 | II-32a |
| T3-111 | I-7 | II-8 | II-34a |
| T3-112 | I-7 | II-8 | II-35a |
| T3-113 | I-7 | II-9 | II-30a |
| T3-114 | I-7 | II-9 | II-32a |
| T3-115 | I-7 | II-9 | II-34a |
| T3-116 | I-7 | II-9 | II-35a |
| T3-117 | I-7 | II-25 | II-30a |
| T3-118 | I-7 | II-25 | II-32a |
| T3-119 | I-7 | II-25 | II-34a |
| T3-120 | I-7 | II-25 | II-35a |
| T3-121 | I-7 | II-26 | II-30a |
| T3-122 | I-7 | II-26 | II-32a |
| T3-123 | I-7 | II-26 | II-34a |
| T3-124 | I-7 | II-26 | II-35a |
| T3-125 | I-7 | II-28 | II-30a |
| T3-126 | I-7 | II-28 | II-32a |
| T3-127 | I-7 | II-28 | II-34a |
| T3-128 | I-7 | II-28 | II-35a |
| T3-129 | I-7 | II-29 | II-30a |
| T3-130 | I-7 | II-29 | II-32a |
| T3-131 | I-7 | II-29 | II-34a |
| T3-132 | I-7 | II-29 | II-35a |
| T3-133 | I-7 | II-49 | II-30a |
| T3-134 | I-7 | II-49 | II-32a |
| T3-135 | I-7 | II-49 | II-34a |
| T3-136 | I-7 | II-49 | II-35a |
| T3-137 | I-7 | II-50 | II-30a |
| T3-138 | I-7 | II-50 | II-32a |
| T3-139 | I-7 | II-50 | II-34a |
| T3-140 | I-7 | II-50 | II-35a |
| T3-141 | I-7 | II-51 | II-30a |
| T3-142 | I-7 | II-51 | II-32a |
| T3-143 | I-7 | II-51 | II-34a |
| T3-144 | I-7 | II-51 | II-35a |
| T3-145 | I-8 | II-8 | II-30a |
| T3-146 | I-8 | II-8 | II-32a |
| T3-147 | I-8 | II-8 | II-34a |
| T3-148 | I-8 | II-8 | II-35a |
| T3-149 | I-8 | II-9 | II-30a |
| T3-150 | I-8 | II-9 | II-32a |
| T3-151 | I-8 | II-9 | II-34a |
| T3-152 | I-8 | II-9 | II-35a |
| T3-153 | I-8 | II-25 | II-30a |
| T3-154 | I-8 | II-25 | II-32a |
| T3-155 | I-8 | II-25 | II-34a |
| T3-156 | I-8 | II-25 | II-35a |
| T3-157 | I-8 | II-26 | II-30a |
| T3-158 | I-8 | II-26 | II-32a |
| T3-159 | I-8 | II-26 | II-34a |
| T3-160 | I-8 | II-26 | II-35a |
| T3-161 | I-8 | II-28 | II-30a |
| T3-162 | I-8 | II-28 | II-32a |
| T3-163 | I-8 | II-28 | II-34a |
| T3-164 | I-8 | II-28 | II-35a |
| T3-165 | I-8 | II-29 | II-30a |
| T3-166 | I-8 | II-29 | II-32a |
| T3-167 | I-8 | II-29 | II-34a |
| T3-168 | I-8 | II-29 | II-35a |
| T3-169 | I-8 | II-49 | II-30a |
| T3-170 | I-8 | II-49 | II-32a |
| T3-171 | I-8 | II-49 | II-34a |
| T3-172 | I-8 | II-49 | II-35a |
| T3-173 | I-8 | II-50 | II-30a |
| T3-174 | I-8 | II-50 | II-32a |
| T3-175 | I-8 | II-50 | II-34a |
| T3-176 | I-8 | II-50 | II-35a |
| T3-177 | I-8 | II-51 | II-30a |
| T3-178 | I-8 | II-51 | II-32a |
| T3-179 | I-8 | II-51 | II-34a |
| T3-180 | I-8 | II-51 | II-35a |
| T3-181 | I-9 | II-8 | II-30a |
| T3-182 | I-9 | II-8 | II-32a |
| T3-183 | I-9 | II-8 | II-34a |
| T3-184 | I-9 | II-8 | II-35a |
| T3-185 | I-9 | II-9 | II-30a |
| T3-186 | I-9 | II-9 | II-32a |
| T3-187 | I-9 | II-9 | II-34a |
| T3-188 | I-9 | II-9 | II-35a |
| T3-189 | I-9 | II-25 | II-30a |
| T3-190 | I-9 | II-25 | II-32a |
| T3-191 | I-9 | II-25 | II-34a |
| T3-192 | I-9 | II-25 | II-35a |
| T3-193 | I-9 | II-26 | II-30a |
| T3-194 | I-9 | II-26 | II-32a |
| T3-195 | I-9 | II-26 | II-34a |
| T3-196 | I-9 | II-26 | II-35a |
| T3-197 | I-9 | II-28 | II-30a |
| T3-198 | I-9 | II-28 | II-32a |
| T3-199 | I-9 | II-28 | II-34a |
| T3-200 | I-9 | II-28 | II-35a |
| T3-201 | I-9 | II-29 | II-30a |
| T3-202 | I-9 | II-29 | II-32a |
| T3-203 | I-9 | II-29 | II-34a |
| T3-204 | I-9 | II-29 | II-35a |
| T3-205 | I-9 | II-49 | II-30a |
| T3-206 | I-9 | II-49 | II-32a |
| T3-207 | I-9 | II-49 | II-34a |
| T3-208 | I-9 | II-49 | II-35a |
| T3-209 | I-9 | II-50 | II-30a |
| T3-210 | I-9 | II-50 | II-32a |
| T3-211 | I-9 | II-50 | II-34a |
| T3-212 | I-9 | II-50 | II-35a |
| T3-213 | I-9 | II-51 | II-30a |
| T3-214 | I-9 | II-51 | II-32a |
| T3-215 | I-9 | II-51 | II-34a |
| T3-216 | I-9 | II-51 | II-35a |
| T3-217 | I-11 | II-8 | II-30a |
| T3-218 | I-11 | II-8 | II-32a |
| T3-219 | I-11 | II-8 | II-34a |
| T3-220 | I-11 | II-8 | II-35a |
| T3-221 | I-11 | II-9 | II-30a |
| T3-222 | I-11 | II-9 | II-32a |
| T3-223 | I-11 | II-9 | II-34a |
| T3-224 | I-11 | II-9 | II-35a |
| T3-225 | I-11 | II-25 | II-30a |
| T3-226 | I-11 | II-25 | II-32a |
| T3-227 | I-11 | II-25 | II-34a |
| T3-228 | I-11 | II-25 | II-35a |
| T3-229 | I-11 | II-26 | II-30a |
| T3-230 | I-11 | II-26 | II-32a |
| T3-231 | I-11 | II-26 | II-34a |
| T3-232 | I-11 | II-26 | II-35a |
| T3-233 | I-11 | II-28 | II-30a |
| T3-234 | I-11 | II-28 | II-32a |
| T3-235 | I-11 | II-28 | II-34a |
| T3-236 | I-11 | II-28 | II-35a |
| T3-237 | I-11 | II-29 | II-30a |
| T3-238 | I-11 | II-29 | II-32a |
| T3-239 | I-11 | II-29 | II-34a |
| T3-240 | I-11 | II-29 | II-35a |
| T3-241 | I-11 | II-49 | II-30a |
| T3-242 | I-11 | II-49 | II-32a |
| T3-243 | I-11 | II-49 | II-34a |
| T3-244 | I-11 | II-49 | II-35a |
| T3-245 | I-11 | II-50 | II-30a |
| T3-246 | I-11 | II-50 | II-32a |
| T3-247 | I-11 | II-50 | II-34a |
| T3-248 | I-11 | II-50 | II-35a |
| T3-249 | I-11 | II-51 | II-30a |
| T3-250 | I-11 | II-51 | II-32a |
| T3-251 | I-11 | II-51 | II-34a |
| T3-252 | I-11 | II-51 | II-35a |
| T3-253 | I-12 | II-8 | II-30a |
| T3-254 | I-12 | II-8 | II-32a |
| T3-255 | I-12 | II-8 | II-34a |
| T3-256 | I-12 | II-8 | II-35a |
| T3-257 | I-12 | II-9 | II-30a |
| T3-258 | I-12 | II-9 | II-32a |
| T3-259 | I-12 | II-9 | II-34a |
| T3-260 | I-12 | II-9 | II-35a |
| T3-261 | I-12 | II-25 | II-30a |
| T3-262 | I-12 | II-25 | II-32a |
| T3-263 | I-12 | II-25 | II-34a |
| T3-264 | I-12 | II-25 | II-35a |
| T3-265 | I-12 | II-26 | II-30a |
| T3-266 | I-12 | II-26 | II-32a |
| T3-267 | I-12 | II-26 | II-34a |
| T3-268 | I-12 | II-26 | II-35a |
| T3-269 | I-12 | II-28 | II-30a |
| T3-270 | I-12 | II-28 | II-32a |
| T3-271 | I-12 | II-28 | II-34a |
| T3-272 | I-12 | II-28 | II-35a |
| T3-273 | I-12 | II-29 | II-30a |
| T3-274 | I-12 | II-29 | II-32a |
| T3-275 | I-12 | II-29 | II-34a |
| T3-276 | I-12 | II-29 | II-35a |
| T3-277 | I-12 | II-49 | II-30a |
| T3-278 | I-12 | II-49 | II-32a |
| T3-279 | I-12 | II-49 | II-34a |
| T3-280 | I-12 | II-49 | II-35a |
| T3-281 | I-12 | II-50 | II-30a |
| T3-282 | I-12 | II-50 | II-32a |
| T3-283 | I-12 | II-50 | II-34a |
| T3-284 | I-12 | II-50 | II-35a |
| T3-285 | I-12 | II-51 | II-30a |
| T3-286 | I-12 | II-51 | II-32a |
| T3-287 | I-12 | II-51 | II-34a |
| T3-288 | I-12 | II-51 | II-35a |
| T3-289 | I-13 | II-8 | II-30a |
| T3-290 | I-13 | II-8 | II-32a |
| T3-291 | I-13 | II-8 | II-34a |
| T3-292 | I-13 | II-8 | II-35a |
| T3-293 | I-13 | II-9 | II-30a |
| T3-294 | I-13 | II-9 | II-32a |
| T3-295 | I-13 | II-9 | II-34a |
| T3-296 | I-13 | II-9 | II-35a |
| T3-297 | I-13 | II-25 | II-30a |
| T3-298 | I-13 | II-25 | II-32a |
| T3-299 | I-13 | II-25 | II-34a |
| T3-300 | I-13 | II-25 | II-35a |
| T3-301 | I-13 | II-26 | II-30a |
| T3-302 | I-13 | II-26 | II-32a |
| T3-303 | I-13 | II-26 | II-34a |
| T3-304 | I-13 | II-26 | II-35a |
| T3-305 | I-13 | II-28 | II-30a |
| T3-306 | I-13 | II-28 | II-32a |
| T3-307 | I-13 | II-28 | II-34a |
| T3-308 | I-13 | II-28 | II-35a |
| T3-309 | I-13 | II-29 | II-30a |
| T3-310 | I-13 | II-29 | II-32a |
| T3-311 | I-13 | II-29 | II-34a |
| T3-312 | I-13 | II-29 | II-35a |
| T3-313 | I-13 | II-49 | II-30a |
| T3-314 | I-13 | II-49 | II-32a |
| T3-315 | I-13 | II-49 | II-34a |
| T3-316 | I-13 | II-49 | II-35a |
| T3-317 | I-13 | II-50 | II-30a |
| T3-318 | I-13 | II-50 | II-32a |
| T3-319 | I-13 | II-50 | II-34a |
| T3-320 | I-13 | II-50 | II-35a |
| T3-321 | I-13 | II-51 | II-30a |
| T3-322 | I-13 | II-51 | II-32a |
| T3-323 | I-13 | II-51 | II-34a |
| T3-324 | I-13 | II-51 | II-35a |
| T3-325 | I-14 | II-8 | II-30a |
| T3-326 | I-14 | II-8 | II-32a |
| T3-327 | I-14 | II-8 | II-34a |
| T3-328 | I-14 | II-8 | II-35a |
| T3-329 | I-14 | II-9 | II-30a |
| T3-330 | I-14 | II-9 | II-32a |
| T3-331 | I-14 | II-9 | II-34a |
| T3-332 | I-14 | II-9 | II-35a |
| T3-333 | I-14 | II-25 | II-30a |
| T3-334 | I-14 | II-25 | II-32a |
| T3-335 | I-14 | II-25 | II-34a |
| T3-336 | I-14 | II-25 | II-35a |
| T3-337 | I-14 | II-26 | II-30a |
| T3-338 | I-14 | II-26 | II-32a |
| T3-339 | I-14 | II-26 | II-34a |
| T3-340 | I-14 | II-26 | II-35a |
| T3-341 | I-14 | II-28 | II-30a |
| T3-342 | I-14 | II-28 | II-32a |
| T3-343 | I-14 | II-28 | II-34a |
| T3-344 | I-14 | II-28 | II-35a |
| T3-345 | I-14 | II-29 | II-30a |
| T3-346 | I-14 | II-29 | II-32a |
| T3-347 | I-14 | II-29 | II-34a |
| T3-348 | I-14 | II-29 | II-35a |
| T3-349 | I-14 | II-49 | II-30a |
| T3-350 | I-14 | II-49 | II-32a |
| T3-351 | I-14 | II-49 | II-34a |
| T3-352 | I-14 | II-49 | II-35a |
| T3-353 | I-14 | II-50 | II-30a |
| T3-354 | I-14 | II-50 | II-32a |
| T3-355 | I-14 | II-50 | II-34a |
| T3-356 | I-14 | II-50 | II-35a |
| T3-357 | I-14 | II-51 | II-30a |
| T3-358 | I-14 | II-51 | II-32a |
| T3-359 | I-14 | II-51 | II-34a |
| T3-360 | I-14 | II-51 | II-35a |
| T3-361 | I-18 | II-8 | II-30a |
| T3-362 | I-18 | II-8 | II-32a |
| T3-363 | I-18 | II-8 | II-34a |
| T3-364 | I-18 | II-8 | II-35a |
| T3-365 | I-18 | II-9 | II-30a |
| T3-366 | I-18 | II-9 | II-32a |
| T3-367 | I-18 | II-9 | II-34a |
| T3-368 | I-18 | II-9 | II-35a |
| T3-369 | I-18 | II-25 | II-30a |
| T3-370 | I-18 | II-25 | II-32a |
| T3-371 | I-18 | II-25 | II-34a |
| T3-372 | I-18 | II-25 | II-35a |
| T3-373 | I-18 | II-26 | II-30a |
| T3-374 | I-18 | II-26 | II-32a |
| T3-375 | I-18 | II-26 | II-34a |
| T3-376 | I-18 | II-26 | II-35a |
| T3-377 | I-18 | II-28 | II-30a |
| T3-378 | I-18 | II-28 | II-32a |
| T3-379 | I-18 | II-28 | II-34a |
| T3-380 | I-18 | II-28 | II-35a |
| T3-381 | I-18 | II-29 | II-30a |
| T3-382 | I-18 | II-29 | II-32a |
| T3-383 | I-18 | II-29 | II-34a |
| T3-384 | I-18 | II-29 | II-35a |
| T3-385 | I-18 | II-49 | II-30a |
| T3-386 | I-18 | II-49 | II-32a |
| T3-387 | I-18 | II-49 | II-34a |
| T3-388 | I-18 | II-49 | II-35a |
| T3-389 | I-18 | II-50 | II-30a |
| T3-390 | I-18 | II-50 | II-32a |
| T3-391 | I-18 | II-50 | II-34a |
| T3-392 | I-18 | II-50 | II-35a |
| T3-393 | I-18 | II-51 | II-30a |
| T3-394 | I-18 | II-51 | II-32a |
| T3-395 | I-18 | II-51 | II-34a |
| T3-396 | I-18 | II-51 | II-35a |

“C” denotes “component”,
“M” denotes “mixture”.

According to a further embodiment, preference is given to ternary mixtures in which component 2 is a compound II selected from the group of the following compounds:

| | |
|---|---|
| II-32a | clothianidin |
| II-34a | imidacloprid |
| II-35a | thiamethoxam, |

and component 3 is fipronil (compound II-30a).

TABLE T4
The ternary mixtures T4-1 to T4-33 below comprise, as component 1,
a preferred compound I, and components selected from preferred active
compounds II of group I) and, as 3rd component, fipronil. In the table
below, each row corresponds to a ternary mixture according to the
invention having the mixture components 1 to 3 listed in the row in
question.

| M | C1 | C2 | C3 |
|---|---|---|---|
| T4-1 | I-1 | II-32a | II-30a |
| T4-2 | I-1 | II-34a | II-30a |
| T4-3 | I-1 | II-35a | II-30a |
| T4-4 | I-2 | II-32a | II-30a |
| T4-5 | I-2 | II-34a | II-30a |
| T4-6 | I-2 | II-35a | II-30a |
| T4-7 | I-6 | II-32a | II-30a |
| T4-8 | I-6 | II-34a | II-30a |
| T4-9 | I-6 | II-35a | II-30a |
| T4-10 | I-7 | II-32a | II-30a |
| T4-11 | I-7 | II-34a | II-30a |
| T4-12 | I-7 | II-35a | II-30a |
| T4-13 | I-8 | II-32a | II-30a |
| T4-14 | I-8 | II-34a | II-30a |
| T4-15 | I-8 | II-35a | II-30a |
| T4-16 | I-9 | II-32a | II-30a |
| T4-17 | I-9 | II-34a | II-30a |
| T4-18 | I-9 | II-35a | II-30a |
| T4-19 | I-11 | II-32a | II-30a |
| T4-20 | I-11 | II-34a | II-30a |
| T4-21 | I-11 | II-35a | II-30a |
| T4-22 | I-12 | II-32a | II-30a |
| T4-23 | I-12 | II-34a | II-30a |
| T4-24 | I-12 | II-35a | II-30a |
| T4-25 | I-13 | II-32a | II-30a |
| T4-26 | I-13 | II-34a | II-30a |
| T4-27 | I-13 | II-35a | II-30a |
| T4-28 | I-14 | II-32a | II-30a |
| T4-29 | I-14 | II-34a | II-30a |
| T4-30 | I-14 | II-35a | II-30a |
| T4-31 | I-18 | II-32a | II-30a |
| T4-32 | I-18 | II-34a | II-30a |
| T4-33 | I-18 | II-35a | II-30a |

“C” denotes “component”,
“M” denotes “mixture”.

According to a further embodiment, preference is given to ternary mixtures in which component 2 is a compound II selected from the group of the following compounds:

| | |
|---|---|
| II-8 | kresoxim-methyl |
| II-9 | pyraclostrobin |
| II-25 | N-(3′,4′,5′-trifluorobiphenyl-2-yl)-3-difluoromethyl-1-methyl-1H- |
| | pyrazole-4-carboxamide |
| II-26 | sedaxane |
| II-28 | fluopyram |
| II-29 | penflufen |
| II-49 | azoxystrobin |
| II-50 | trifloxystrobin |
| II-51 | penthiopyrad |

and component 3 is fipronil (compound II-30a).

TABLE T5
The ternary mixtures T5-1 to T5-99 below comprise, as component 1,
a preferred compound I, and components selected from preferred active
compounds II of group I) and, as 3rd component, fipronil. In the table
below, each row corresponds to a ternary mixture according to the
invention having the mixture components 1 to 3 listed in the row in
question.

| M | C1 | C2 | C3 |
|---|---|---|---|
| T5-1 | I-1 | II-8 | II-30a |
| T5-2 | I-1 | II-9 | II-30a |
| T5-3 | I-1 | II-25 | II-30a |
| T5-4 | I-1 | II-26 | II-30a |
| T5-5 | I-1 | II-28 | II-30a |
| T5-6 | I-1 | II-29 | II-30a |
| T5-7 | I-1 | II-49 | II-30a |
| T5-8 | I-1 | II-50 | II-30a |
| T5-9 | I-1 | II-51 | II-30a |
| T5-10 | I-2 | II-8 | II-30a |
| T5-11 | I-2 | II-9 | II-30a |
| T5-12 | I-2 | II-25 | II-30a |
| T5-13 | I-2 | II-26 | II-30a |
| T5-14 | I-2 | II-28 | II-30a |
| T5-15 | I-2 | II-29 | II-30a |
| T5-16 | I-2 | II-49 | II-30a |
| T5-17 | I-2 | II-50 | II-30a |
| T5-18 | I-2 | II-51 | II-30a |
| T5-19 | I-6 | II-8 | II-30a |
| T5-20 | I-6 | II-9 | II-30a |
| T5-21 | I-6 | II-25 | II-30a |
| T5-22 | I-6 | II-26 | II-30a |
| T5-23 | I-6 | II-28 | II-30a |
| T5-24 | I-6 | II-29 | II-30a |
| T5-25 | I-6 | II-49 | II-30a |
| T5-26 | I-6 | II-50 | II-30a |
| T5-27 | I-6 | II-51 | II-30a |
| T5-28 | I-7 | II-8 | II-30a |
| T5-29 | I-7 | II-9 | II-30a |
| T5-30 | I-7 | II-25 | II-30a |
| T5-31 | I-7 | II-26 | II-30a |
| T5-32 | I-7 | II-28 | II-30a |
| T5-33 | I-7 | II-29 | II-30a |
| T5-34 | I-7 | II-49 | II-30a |
| T5-35 | I-7 | II-50 | II-30a |
| T5-36 | I-7 | II-51 | II-30a |
| T5-37 | I-8 | II-8 | II-30a |
| T5-38 | I-8 | II-9 | II-30a |
| T5-39 | I-8 | II-25 | II-30a |
| T5-40 | I-8 | II-26 | II-30a |
| T5-41 | I-8 | II-28 | II-30a |
| T5-42 | I-8 | II-29 | II-30a |
| T5-43 | I-8 | II-49 | II-30a |
| T5-44 | I-8 | II-50 | II-30a |
| T5-45 | I-8 | II-51 | II-30a |
| T5-46 | I-9 | II-8 | II-30a |
| T5-47 | I-9 | II-9 | II-30a |
| T5-48 | I-9 | II-25 | II-30a |
| T5-49 | I-9 | II-26 | II-30a |
| T5-50 | I-9 | II-28 | II-30a |
| T5-51 | I-9 | II-29 | II-30a |
| T5-52 | I-9 | II-49 | II-30a |
| T5-53 | I-9 | II-50 | II-30a |
| T5-54 | I-9 | II-51 | II-30a |
| T5-55 | I-11 | II-8 | II-30a |
| T5-56 | I-11 | II-9 | II-30a |
| T5-57 | I-11 | II-25 | II-30a |
| T5-58 | I-11 | II-26 | II-30a |
| T5-59 | I-11 | II-28 | II-30a |
| T5-60 | I-11 | II-29 | II-30a |
| T5-61 | I-11 | II-49 | II-30a |
| T5-62 | I-11 | II-50 | II-30a |
| T5-63 | I-11 | II-51 | II-30a |
| T5-64 | I-12 | II-8 | II-30a |
| T5-65 | I-12 | II-9 | II-30a |
| T5-66 | I-12 | II-25 | II-30a |
| T5-67 | I-12 | II-26 | II-30a |
| T5-68 | I-12 | II-28 | II-30a |
| T5-69 | I-12 | II-29 | II-30a |
| T5-70 | I-12 | II-49 | II-30a |
| T5-71 | I-12 | II-50 | II-30a |
| T5-72 | I-12 | II-51 | II-30a |
| T5-73 | I-13 | II-8 | II-30a |
| T5-74 | I-13 | II-9 | II-30a |
| T5-75 | I-13 | II-25 | II-30a |
| T5-76 | I-13 | II-26 | II-30a |
| T5-77 | I-13 | II-28 | II-30a |
| T5-78 | I-13 | II-29 | II-30a |
| T5-79 | I-13 | II-49 | II-30a |
| T5-80 | I-13 | II-50 | II-30a |
| T5-81 | I-13 | II-51 | II-30a |
| T5-82 | I-14 | II-8 | II-30a |
| T5-83 | I-14 | II-9 | II-30a |
| T5-84 | I-14 | II-25 | II-30a |
| T5-85 | I-14 | II-26 | II-30a |
| T5-86 | I-14 | II-28 | II-30a |
| T5-87 | I-14 | II-29 | II-30a |
| T5-88 | I-14 | II-49 | II-30a |
| T5-89 | I-14 | II-50 | II-30a |
| T5-90 | I-14 | II-51 | II-30a |
| T5-91 | I-18 | II-8 | II-30a |
| T5-92 | I-18 | II-9 | II-30a |
| T5-93 | I-18 | II-25 | II-30a |
| T5-94 | I-18 | II-26 | II-30a |
| T5-95 | I-18 | II-28 | II-30a |
| T5-96 | I-18 | II-29 | II-30a |
| T5-97 | I-18 | II-49 | II-30a |
| T5-98 | I-18 | II-50 | II-30a |
| T5-99 | I-18 | II-51 | II-30a |

“C” denotes “component”,
“M” denotes “mixture”.

Particularly preferred components 4 are compounds II selected from the group of the compounds below:

| | |
|---|---|
| II-1 | epoxiconazole |
| II-2 | metconazole |
| II-3 | tebuconazole |
| II-7 | prothioconazole |
| II-8 | kresoxim-methyl |
| II-9 | pyraclostrobin |
| II-15 | fenpropimorph |
| II-21 | chlorothalonil |
| II-22 | metrafenone |
| II-23 | bixafen |
| II-24 | boscalid |
| II-25 | N-(3′,4′,5′-trifluorobiphenyl-2-yl)-3-difluoromethyl-1-methyl- |
| | 1H-pyrazole-4-carboxamide |
| II-26 | sedaxane |
| II-27 | isopyrazam |
| II-28 | fluopyram |
| II-29 | penflufen |

with the proviso that components 2, 3 and 4 are not identical active compounds.

Particularly preferred quaternary mixtures of the present invention are the mixtures listed in the table below:

TABLE Q
The quaternary mixtures Q-1 to Q1-2244 below comprise, as component
1, a compound I, a component 2 selected from the active compounds of
groups A to I preferred for component 2, a component 3 selected from the
active compounds of groups A to I preferred for component 3 and a
component 4 selected from the active compounds of groups A to I
preferred for component 4. In the table below, each row corresponds to a
quaternary mixture according to the invention having the mixture
components 1 to 4 listed in the row in question.

| M | C1 | C2 | C3 | C4 |
|---|---|---|---|---|
| Q-1 | I-1 | II-1 | II-2 | II-3 |
| Q-2 | I-1 | II-1 | II-2 | II-7 |
| Q-3 | I-1 | II-1 | II-2 | II-8 |
| Q-4 | I-1 | II-1 | II-2 | II-9 |
| Q-5 | I-1 | II-1 | II-2 | II-15 |
| Q-6 | I-1 | II-1 | II-2 | II-21 |
| Q-7 | I-1 | II-1 | II-2 | II-22 |
| Q-8 | I-1 | II-1 | II-2 | II-23 |
| Q-9 | I-1 | II-1 | II-2 | II-24 |
| Q-10 | I-1 | II-1 | II-2 | II-25 |
| Q-11 | I-1 | II-1 | II-2 | II-26 |
| Q-12 | I-1 | II-1 | II-2 | II-27 |
| Q-13 | I-1 | II-1 | II-2 | II-28 |
| Q-14 | I-1 | II-1 | II-2 | II-29 |
| Q-15 | I-1 | II-1 | II-3 | II-7 |
| Q-16 | I-1 | II-1 | II-3 | II-8 |
| Q-17 | I-1 | II-1 | II-3 | II-9 |
| Q-18 | I-1 | II-1 | II-3 | II-15 |
| Q-19 | I-1 | II-1 | II-3 | II-21 |
| Q-20 | I-1 | II-1 | II-3 | II-22 |
| Q-21 | I-1 | II-1 | II-3 | II-23 |
| Q-22 | I-1 | II-1 | II-3 | II-24 |
| Q-23 | I-1 | II-1 | II-3 | II-25 |
| Q-24 | I-1 | II-1 | II-3 | II-26 |
| Q-25 | I-1 | II-1 | II-3 | II-27 |
| Q-26 | I-1 | II-1 | II-3 | II-28 |
| Q-27 | I-1 | II-1 | II-3 | II-29 |
| Q-28 | I-1 | II-1 | II-7 | II-8 |
| Q-29 | I-1 | II-1 | II-7 | II-9 |
| Q-30 | I-1 | II-1 | II-7 | II-15 |
| Q-31 | I-1 | II-1 | II-7 | II-21 |
| Q-32 | I-1 | II-1 | II-7 | II-22 |
| Q-33 | I-1 | II-1 | II-7 | II-23 |
| Q-34 | I-1 | II-1 | II-7 | II-24 |
| Q-35 | I-1 | II-1 | II-7 | II-25 |
| Q-36 | I-1 | II-1 | II-7 | II-26 |
| Q-37 | I-1 | II-1 | II-7 | II-27 |
| Q-38 | I-1 | II-1 | II-7 | II-28 |
| Q-39 | I-1 | II-1 | II-7 | II-29 |
| Q-40 | I-1 | II-1 | II-8 | II-9 |
| Q-41 | I-1 | II-1 | II-8 | II-15 |
| Q-42 | I-1 | II-1 | II-8 | II-21 |
| Q-43 | I-1 | II-1 | II-8 | II-22 |
| Q-44 | I-1 | II-1 | II-8 | II-23 |
| Q-45 | I-1 | II-1 | II-8 | II-24 |
| Q-46 | I-1 | II-1 | II-8 | II-25 |
| Q-47 | I-1 | II-1 | II-8 | II-26 |
| Q-48 | I-1 | II-1 | II-8 | II-27 |
| Q-49 | I-1 | II-1 | II-8 | II-28 |
| Q-50 | I-1 | II-1 | II-8 | II-29 |
| Q-51 | I-1 | II-1 | II-9 | II-15 |
| Q-52 | I-1 | II-1 | II-9 | II-21 |
| Q-53 | I-1 | II-1 | II-9 | II-22 |
| Q-54 | I-1 | II-1 | II-9 | II-23 |
| Q-55 | I-1 | II-1 | II-9 | II-24 |
| Q-56 | I-1 | II-1 | II-9 | II-25 |
| Q-57 | I-1 | II-1 | II-9 | II-26 |
| Q-58 | I-1 | II-1 | II-9 | II-27 |
| Q-59 | I-1 | II-1 | II-9 | II-28 |
| Q-60 | I-1 | II-1 | II-9 | II-29 |
| Q-61 | I-1 | II-1 | II-15 | II-21 |
| Q-62 | I-1 | II-1 | II-15 | II-22 |
| Q-63 | I-1 | II-1 | II-15 | II-23 |
| Q-64 | I-1 | II-1 | II-15 | II-24 |
| Q-65 | I-1 | II-1 | II-15 | II-25 |
| Q-66 | I-1 | II-1 | II-15 | II-26 |
| Q-67 | I-1 | II-1 | II-15 | II-27 |
| Q-68 | I-1 | II-1 | II-15 | II-28 |
| Q-69 | I-1 | II-1 | II-15 | II-29 |
| Q-70 | I-1 | II-1 | II-21 | II-22 |
| Q-71 | I-1 | II-1 | II-21 | II-23 |
| Q-72 | I-1 | II-1 | II-21 | II-24 |
| Q-73 | I-1 | II-1 | II-21 | II-25 |
| Q-74 | I-1 | II-1 | II-21 | II-26 |
| Q-75 | I-1 | II-1 | II-21 | II-27 |
| Q-76 | I-1 | II-1 | II-21 | II-28 |
| Q-77 | I-1 | II-1 | II-21 | II-29 |
| Q-78 | I-1 | II-1 | II-22 | II-23 |
| Q-79 | I-1 | II-1 | II-22 | II-24 |
| Q-80 | I-1 | II-1 | II-22 | II-25 |
| Q-81 | I-1 | II-1 | II-22 | II-26 |
| Q-82 | I-1 | II-1 | II-22 | II-27 |
| Q-83 | I-1 | II-1 | II-22 | II-28 |
| Q-84 | I-1 | II-1 | II-22 | II-29 |
| Q-85 | I-1 | II-1 | II-23 | II-24 |
| Q-86 | I-1 | II-1 | II-23 | II-25 |
| Q-87 | I-1 | II-1 | II-23 | II-26 |
| Q-88 | I-1 | II-1 | II-23 | II-27 |
| Q-89 | I-1 | II-1 | II-23 | II-28 |
| Q-90 | I-1 | II-1 | II-23 | II-29 |
| Q-91 | I-1 | II-1 | II-24 | II-25 |
| Q-92 | I-1 | II-1 | II-24 | II-26 |
| Q-93 | I-1 | II-1 | II-24 | II-27 |
| Q-94 | I-1 | II-1 | II-24 | II-28 |
| Q-95 | I-1 | II-1 | II-24 | II-29 |
| Q-96 | I-1 | II-1 | II-25 | II-26 |
| Q-97 | I-1 | II-1 | II-25 | II-27 |
| Q-98 | I-1 | II-1 | II-25 | II-28 |
| Q-99 | I-1 | II-1 | II-25 | II-29 |
| Q-100 | I-1 | II-1 | II-26 | II-27 |
| Q-101 | I-1 | II-1 | II-26 | II-28 |
| Q-102 | I-1 | II-1 | II-26 | II-29 |
| Q-103 | I-1 | II-1 | II-27 | II-28 |
| Q-104 | I-1 | II-1 | II-27 | II-29 |
| Q-105 | I-1 | II-1 | II-28 | II-29 |
| Q-106 | I-1 | II-2 | II-3 | II-7 |
| Q-107 | I-1 | II-2 | II-3 | II-8 |
| Q-108 | I-1 | II-2 | II-3 | II-9 |
| Q-109 | I-1 | II-2 | II-3 | II-15 |
| Q-110 | I-1 | II-2 | II-3 | II-21 |
| Q-111 | I-1 | II-2 | II-3 | II-22 |
| Q-112 | I-1 | II-2 | II-3 | II-23 |
| Q-113 | I-1 | II-2 | II-3 | II-24 |
| Q-114 | I-1 | II-2 | II-3 | II-25 |
| Q-115 | I-1 | II-2 | II-3 | II-26 |
| Q-116 | I-1 | II-2 | II-3 | II-27 |
| Q-117 | I-1 | II-2 | II-3 | II-28 |
| Q-118 | I-1 | II-2 | II-3 | II-29 |
| Q-119 | I-1 | II-2 | II-7 | II-8 |
| Q-120 | I-1 | II-2 | II-7 | II-9 |
| Q-121 | I-1 | II-2 | II-7 | II-15 |
| Q-122 | I-1 | II-2 | II-7 | II-21 |
| Q-123 | I-1 | II-2 | II-7 | II-22 |
| Q-124 | I-1 | II-2 | II-7 | II-23 |
| Q-125 | I-1 | II-2 | II-7 | II-24 |
| Q-126 | I-1 | II-2 | II-7 | II-25 |
| Q-127 | I-1 | II-2 | II-7 | II-26 |
| Q-128 | I-1 | II-2 | II-7 | II-27 |
| Q-129 | I-1 | II-2 | II-7 | II-28 |
| Q-130 | I-1 | II-2 | II-7 | II-29 |
| Q-131 | I-1 | II-2 | II-8 | II-9 |
| Q-132 | I-1 | II-2 | II-8 | II-15 |
| Q-133 | I-1 | II-2 | II-8 | II-21 |
| Q-134 | I-1 | II-2 | II-8 | II-22 |
| Q-135 | I-1 | II-2 | II-8 | II-23 |
| Q-136 | I-1 | II-2 | II-8 | II-24 |
| Q-137 | I-1 | II-2 | II-8 | II-25 |
| Q-138 | I-1 | II-2 | II-8 | II-26 |
| Q-139 | I-1 | II-2 | II-8 | II-27 |
| Q-140 | I-1 | II-2 | II-8 | II-28 |
| Q-141 | I-1 | II-2 | II-8 | II-29 |
| Q-142 | I-1 | II-2 | II-9 | II-15 |
| Q-143 | I-1 | II-2 | II-9 | II-21 |
| Q-144 | I-1 | II-2 | II-9 | II-22 |
| Q-145 | I-1 | II-2 | II-9 | II-23 |
| Q-146 | I-1 | II-2 | II-9 | II-24 |
| Q-147 | I-1 | II-2 | II-9 | II-25 |
| Q-148 | I-1 | II-2 | II-9 | II-26 |
| Q-149 | I-1 | II-2 | II-9 | II-27 |
| Q-150 | I-1 | II-2 | II-9 | II-28 |
| Q-151 | I-1 | II-2 | II-9 | II-29 |
| Q-152 | I-1 | II-2 | II-15 | II-21 |
| Q-153 | I-1 | II-2 | II-15 | II-22 |
| Q-154 | I-1 | II-2 | II-15 | II-23 |
| Q-155 | I-1 | II-2 | II-15 | II-24 |
| Q-156 | I-1 | II-2 | II-15 | II-25 |
| Q-157 | I-1 | II-2 | II-15 | II-26 |
| Q-158 | I-1 | II-2 | II-15 | II-27 |
| Q-159 | I-1 | II-2 | II-15 | II-28 |
| Q-160 | I-1 | II-2 | II-15 | II-29 |
| Q-161 | I-1 | II-2 | II-21 | II-22 |
| Q-162 | I-1 | II-2 | II-21 | II-23 |
| Q-163 | I-1 | II-2 | II-21 | II-24 |
| Q-164 | I-1 | II-2 | II-21 | II-25 |
| Q-165 | I-1 | II-2 | II-21 | II-26 |
| Q-166 | I-1 | II-2 | II-21 | II-27 |
| Q-167 | I-1 | II-2 | II-21 | II-28 |
| Q-168 | I-1 | II-2 | II-21 | II-29 |
| Q-169 | I-1 | II-2 | II-22 | II-23 |
| Q-170 | I-1 | II-2 | II-22 | II-24 |
| Q-171 | I-1 | II-2 | II-22 | II-25 |
| Q-172 | I-1 | II-2 | II-22 | II-26 |
| Q-173 | I-1 | II-2 | II-22 | II-27 |
| Q-174 | I-1 | II-2 | II-22 | II-28 |
| Q-175 | I-1 | II-2 | II-22 | II-29 |
| Q-176 | I-1 | II-2 | II-23 | II-24 |
| Q-177 | I-1 | II-2 | II-23 | II-25 |
| Q-178 | I-1 | II-2 | II-23 | II-26 |
| Q-179 | I-1 | II-2 | II-23 | II-27 |
| Q-180 | I-1 | II-2 | II-23 | II-28 |
| Q-181 | I-1 | II-2 | II-23 | II-29 |
| Q-182 | I-1 | II-2 | II-24 | II-25 |
| Q-183 | I-1 | II-2 | II-24 | II-26 |
| Q-184 | I-1 | II-2 | II-24 | II-27 |
| Q-185 | I-1 | II-2 | II-24 | II-28 |
| Q-186 | I-1 | II-2 | II-24 | II-29 |
| Q-187 | I-1 | II-2 | II-25 | II-26 |
| Q-188 | I-1 | II-2 | II-25 | II-27 |
| Q-189 | I-1 | II-2 | II-25 | II-28 |
| Q-190 | I-1 | II-2 | II-25 | II-29 |
| Q-191 | I-1 | II-2 | II-26 | II-27 |
| Q-192 | I-1 | II-2 | II-26 | II-28 |
| Q-193 | I-1 | II-2 | II-26 | II-29 |
| Q-194 | I-1 | II-2 | II-27 | II-28 |
| Q-195 | I-1 | II-2 | II-27 | II-29 |
| Q-196 | I-1 | II-2 | II-28 | II-29 |
| Q-197 | I-1 | II-3 | II-7 | II-8 |
| Q-198 | I-1 | II-3 | II-7 | II-9 |
| Q-199 | I-1 | II-3 | II-7 | II-15 |
| Q-200 | I-1 | II-3 | II-7 | II-21 |
| Q-201 | I-1 | II-3 | II-7 | II-22 |
| Q-202 | I-1 | II-3 | II-7 | II-23 |
| Q-203 | I-1 | II-3 | II-7 | II-24 |
| Q-204 | I-1 | II-3 | II-7 | II-25 |
| Q-205 | I-1 | II-3 | II-7 | II-26 |
| Q-206 | I-1 | II-3 | II-7 | II-27 |
| Q-207 | I-1 | II-3 | II-7 | II-28 |
| Q-208 | I-1 | II-3 | II-7 | II-29 |
| Q-209 | I-1 | II-3 | II-8 | II-9 |
| Q-210 | I-1 | II-3 | II-8 | II-15 |
| Q-211 | I-1 | II-3 | II-8 | II-21 |
| Q-212 | I-1 | II-3 | II-8 | II-22 |
| Q-213 | I-1 | II-3 | II-8 | II-23 |
| Q-214 | I-1 | II-3 | II-8 | II-24 |
| Q-215 | I-1 | II-3 | II-8 | II-25 |
| Q-216 | I-1 | II-3 | II-8 | II-26 |
| Q-217 | I-1 | II-3 | II-8 | II-27 |
| Q-218 | I-1 | II-3 | II-8 | II-28 |
| Q-219 | I-1 | II-3 | II-8 | II-29 |
| Q-220 | I-1 | II-3 | II-9 | II-15 |
| Q-221 | I-1 | II-3 | II-9 | II-21 |
| Q-222 | I-1 | II-3 | II-9 | II-22 |
| Q-223 | I-1 | II-3 | II-9 | II-23 |
| Q-224 | I-1 | II-3 | II-9 | II-24 |
| Q-225 | I-1 | II-3 | II-9 | II-25 |
| Q-226 | I-1 | II-3 | II-9 | II-26 |
| Q-227 | I-1 | II-3 | II-9 | II-27 |
| Q-228 | I-1 | II-3 | II-9 | II-28 |
| Q-229 | I-1 | II-3 | II-9 | II-29 |
| Q-230 | I-1 | II-3 | II-15 | II-21 |
| Q-231 | I-1 | II-3 | II-15 | II-22 |
| Q-232 | I-1 | II-3 | II-15 | II-23 |
| Q-233 | I-1 | II-3 | II-15 | II-24 |
| Q-234 | I-1 | II-3 | II-15 | II-25 |
| Q-235 | I-1 | II-3 | II-15 | II-26 |
| Q-236 | I-1 | II-3 | II-15 | II-27 |
| Q-237 | I-1 | II-3 | II-15 | II-28 |
| Q-238 | I-1 | II-3 | II-15 | II-29 |
| Q-239 | I-1 | II-3 | II-21 | II-22 |
| Q-240 | I-1 | II-3 | II-21 | II-23 |
| Q-241 | I-1 | II-3 | II-21 | II-24 |
| Q-242 | I-1 | II-3 | II-21 | II-25 |
| Q-243 | I-1 | II-3 | II-21 | II-26 |
| Q-244 | I-1 | II-3 | II-21 | II-27 |
| Q-245 | I-1 | II-3 | II-21 | II-28 |
| Q-246 | I-1 | II-3 | II-21 | II-29 |
| Q-247 | I-1 | II-3 | II-22 | II-23 |
| Q-248 | I-1 | II-3 | II-22 | II-24 |
| Q-249 | I-1 | II-3 | II-22 | II-25 |
| Q-250 | I-1 | II-3 | II-22 | II-26 |
| Q-251 | I-1 | II-3 | II-22 | II-27 |
| Q-252 | I-1 | II-3 | II-22 | II-28 |
| Q-253 | I-1 | II-3 | II-22 | II-29 |
| Q-254 | I-1 | II-3 | II-23 | II-24 |
| Q-255 | I-1 | II-3 | II-23 | II-25 |
| Q-256 | I-1 | II-3 | II-23 | II-26 |
| Q-257 | I-1 | II-3 | II-23 | II-27 |
| Q-258 | I-1 | II-3 | II-23 | II-28 |
| Q-259 | I-1 | II-3 | II-23 | II-29 |
| Q-260 | I-1 | II-3 | II-24 | II-25 |
| Q-261 | I-1 | II-3 | II-24 | II-26 |
| Q-262 | I-1 | II-3 | II-24 | II-27 |
| Q-263 | I-1 | II-3 | II-24 | II-28 |
| Q-264 | I-1 | II-3 | II-24 | II-29 |
| Q-265 | I-1 | II-3 | II-25 | II-26 |
| Q-266 | I-1 | II-3 | II-25 | II-27 |
| Q-267 | I-1 | II-3 | II-25 | II-28 |
| Q-268 | I-1 | II-3 | II-25 | II-29 |
| Q-269 | I-1 | II-3 | II-26 | II-27 |
| Q-270 | I-1 | II-3 | II-26 | II-28 |
| Q-271 | I-1 | II-3 | II-26 | II-29 |
| Q-272 | I-1 | II-3 | II-27 | II-28 |
| Q-273 | I-1 | II-3 | II-27 | II-29 |
| Q-274 | I-1 | II-3 | II-28 | II-29 |
| Q-275 | I-1 | II-7 | II-8 | II-9 |
| Q-276 | I-1 | II-7 | II-8 | II-15 |
| Q-277 | I-1 | II-7 | II-8 | II-21 |
| Q-278 | I-1 | II-7 | II-8 | II-22 |
| Q-279 | I-1 | II-7 | II-8 | II-23 |
| Q-280 | I-1 | II-7 | II-8 | II-24 |
| Q-281 | I-1 | II-7 | II-8 | II-25 |
| Q-282 | I-1 | II-7 | II-8 | II-26 |
| Q-283 | I-1 | II-7 | II-8 | II-27 |
| Q-284 | I-1 | II-7 | II-8 | II-28 |
| Q-285 | I-1 | II-7 | II-8 | II-29 |
| Q-286 | I-1 | II-7 | II-9 | II-15 |
| Q-287 | I-1 | II-7 | II-9 | II-21 |
| Q-288 | I-1 | II-7 | II-9 | II-22 |
| Q-289 | I-1 | II-7 | II-9 | II-23 |
| Q-290 | I-1 | II-7 | II-9 | II-24 |
| Q-291 | I-1 | II-7 | II-9 | II-25 |
| Q-292 | I-1 | II-7 | II-9 | II-26 |
| Q-293 | I-1 | II-7 | II-9 | II-27 |
| Q-294 | I-1 | II-7 | II-9 | II-28 |
| Q-295 | I-1 | II-7 | II-9 | II-29 |
| Q-296 | I-1 | II-7 | II-15 | II-21 |
| Q-297 | I-1 | II-7 | II-15 | II-22 |
| Q-298 | I-1 | II-7 | II-15 | II-23 |
| Q-299 | I-1 | II-7 | II-15 | II-24 |
| Q-300 | I-1 | II-7 | II-15 | II-25 |
| Q-301 | I-1 | II-7 | II-15 | II-26 |
| Q-302 | I-1 | II-7 | II-15 | II-27 |
| Q-303 | I-1 | II-7 | II-15 | II-28 |
| Q-304 | I-1 | II-7 | II-15 | II-29 |
| Q-305 | I-1 | II-7 | II-21 | II-22 |
| Q-306 | I-1 | II-7 | II-21 | II-23 |
| Q-307 | I-1 | II-7 | II-21 | II-24 |
| Q-308 | I-1 | II-7 | II-21 | II-25 |
| Q-309 | I-1 | II-7 | II-21 | II-26 |
| Q-310 | I-1 | II-7 | II-21 | II-27 |
| Q-311 | I-1 | II-7 | II-21 | II-28 |
| Q-312 | I-1 | II-7 | II-21 | II-29 |
| Q-313 | I-1 | II-7 | II-22 | II-23 |
| Q-314 | I-1 | II-7 | II-22 | II-24 |
| Q-315 | I-1 | II-7 | II-22 | II-25 |
| Q-316 | I-1 | II-7 | II-22 | II-26 |
| Q-317 | I-1 | II-7 | II-22 | II-27 |
| Q-318 | I-1 | II-7 | II-22 | II-28 |
| Q-319 | I-1 | II-7 | II-22 | II-29 |
| Q-320 | I-1 | II-7 | II-23 | II-24 |
| Q-321 | I-1 | II-7 | II-23 | II-25 |
| Q-322 | I-1 | II-7 | II-23 | II-26 |
| Q-323 | I-1 | II-7 | II-23 | II-27 |
| Q-324 | I-1 | II-7 | II-23 | II-28 |
| Q-325 | I-1 | II-7 | II-23 | II-29 |
| Q-326 | I-1 | II-7 | II-24 | II-24 |
| Q-327 | I-1 | II-7 | II-24 | II-25 |
| Q-328 | I-1 | II-7 | II-24 | II-26 |
| Q-329 | I-1 | II-7 | II-24 | II-27 |
| Q-330 | I-1 | II-7 | II-24 | II-28 |
| Q-331 | I-1 | II-7 | II-24 | II-29 |
| Q-332 | I-1 | II-7 | II-25 | II-26 |
| Q-333 | I-1 | II-7 | II-25 | II-27 |
| Q-334 | I-1 | II-7 | II-25 | II-28 |
| Q-335 | I-1 | II-7 | II-25 | II-29 |
| Q-336 | I-1 | II-7 | II-26 | II-27 |
| Q-337 | I-1 | II-7 | II-26 | II-28 |
| Q-338 | I-1 | II-7 | II-26 | II-29 |
| Q-339 | I-1 | II-7 | II-27 | II-28 |
| Q-340 | I-1 | II-7 | II-27 | II-29 |
| Q-341 | I-1 | II-7 | II-28 | II-29 |
| Q-342 | I-1 | II-8 | II-9 | II-15 |
| Q-343 | I-1 | II-8 | II-9 | II-21 |
| Q-344 | I-1 | II-8 | II-9 | II-22 |
| Q-345 | I-1 | II-8 | II-9 | II-23 |
| Q-346 | I-1 | II-8 | II-9 | II-24 |
| Q-347 | I-1 | II-8 | II-9 | II-25 |
| Q-348 | I-1 | II-8 | II-9 | II-26 |
| Q-349 | I-1 | II-8 | II-9 | II-27 |
| Q-350 | I-1 | II-8 | II-9 | II-28 |
| Q-351 | I-1 | II-8 | II-9 | II-29 |
| Q-352 | I-1 | II-8 | II-15 | II-21 |
| Q-353 | I-1 | II-8 | II-15 | II-22 |
| Q-354 | I-1 | II-8 | II-15 | II-23 |
| Q-355 | I-1 | II-8 | II-15 | II-24 |
| Q-356 | I-1 | II-8 | II-15 | II-25 |
| Q-357 | I-1 | II-8 | II-15 | II-26 |
| Q-358 | I-1 | II-8 | II-15 | II-27 |
| Q-359 | I-1 | II-8 | II-15 | II-28 |
| Q-360 | I-1 | II-8 | II-15 | II-29 |
| Q-361 | I-1 | II-8 | II-21 | II-22 |
| Q-362 | I-1 | II-8 | II-21 | II-23 |
| Q-363 | I-1 | II-8 | II-21 | II-24 |
| Q-364 | I-1 | II-8 | II-21 | II-25 |
| Q-365 | I-1 | II-8 | II-21 | II-26 |
| Q-366 | I-1 | II-8 | II-21 | II-27 |
| Q-367 | I-1 | II-8 | II-21 | II-28 |
| Q-368 | I-1 | II-8 | II-21 | II-29 |
| Q-369 | I-1 | II-8 | II-22 | II-23 |
| Q-370 | I-1 | II-8 | II-22 | II-24 |
| Q-371 | I-1 | II-8 | II-22 | II-25 |
| Q-372 | I-1 | II-8 | II-22 | II-26 |
| Q-373 | I-1 | II-8 | II-22 | II-27 |
| Q-374 | I-1 | II-8 | II-22 | II-28 |
| Q-375 | I-1 | II-8 | II-22 | II-29 |
| Q-376 | I-1 | II-8 | II-23 | II-24 |
| Q-377 | I-1 | II-8 | II-23 | II-25 |
| Q-378 | I-1 | II-8 | II-23 | II-26 |
| Q-379 | I-1 | II-8 | II-23 | II-27 |
| Q-380 | I-1 | II-8 | II-23 | II-28 |
| Q-381 | I-1 | II-8 | II-23 | II-29 |
| Q-382 | I-1 | II-8 | II-24 | II-25 |
| Q-383 | I-1 | II-8 | II-24 | II-26 |
| Q-384 | I-1 | II-8 | II-24 | II-27 |
| Q-385 | I-1 | II-8 | II-24 | II-28 |
| Q-386 | I-1 | II-8 | II-24 | II-29 |
| Q-387 | I-1 | II-8 | II-25 | II-26 |
| Q-388 | I-1 | II-8 | II-25 | II-27 |
| Q-389 | I-1 | II-8 | II-25 | II-28 |
| Q-390 | I-1 | II-8 | II-25 | II-29 |
| Q-391 | I-1 | II-8 | II-26 | II-27 |
| Q-392 | I-1 | II-8 | II-26 | II-28 |
| Q-393 | I-1 | II-8 | II-26 | II-29 |
| Q-394 | I-1 | II-8 | II-27 | II-28 |
| Q-395 | I-1 | II-8 | II-27 | II-29 |
| Q-396 | I-1 | II-8 | II-28 | II-29 |
| Q-397 | I-1 | II-9 | II-15 | II-21 |
| Q-398 | I-1 | II-9 | II-15 | II-22 |
| Q-399 | I-1 | II-9 | II-15 | II-23 |
| Q-400 | I-1 | II-9 | II-15 | II-24 |
| Q-401 | I-1 | II-9 | II-15 | II-25 |
| Q-402 | I-1 | II-9 | II-15 | II-26 |
| Q-403 | I-1 | II-9 | II-15 | II-27 |
| Q-404 | I-1 | II-9 | II-15 | II-28 |
| Q-405 | I-1 | II-9 | II-15 | II-29 |
| Q-406 | I-1 | II-9 | II-21 | II-22 |
| Q-407 | I-1 | II-9 | II-21 | II-23 |
| Q-408 | I-1 | II-9 | II-21 | II-24 |
| Q-409 | I-1 | II-9 | II-21 | II-25 |
| Q-410 | I-1 | II-9 | II-21 | II-26 |
| Q-411 | I-1 | II-9 | II-21 | II-27 |
| Q-412 | I-1 | II-9 | II-21 | II-28 |
| Q-413 | I-1 | II-9 | II-21 | II-29 |
| Q-414 | I-1 | II-9 | II-22 | II-23 |
| Q-415 | I-1 | II-9 | II-22 | II-24 |
| Q-416 | I-1 | II-9 | II-22 | II-25 |
| Q-417 | I-1 | II-9 | II-22 | II-26 |
| Q-418 | I-1 | II-9 | II-22 | II-27 |
| Q-419 | I-1 | II-9 | II-22 | II-28 |
| Q-420 | I-1 | II-9 | II-22 | II-29 |
| Q-421 | I-1 | II-9 | II-23 | II-24 |
| Q-422 | I-1 | II-9 | II-23 | II-25 |
| Q-423 | I-1 | II-9 | II-23 | II-26 |
| Q-424 | I-1 | II-9 | II-23 | II-27 |
| Q-425 | I-1 | II-9 | II-23 | II-28 |
| Q-426 | I-1 | II-9 | II-23 | II-29 |
| Q-427 | I-1 | II-9 | II-24 | II-25 |
| Q-428 | I-1 | II-9 | II-24 | II-26 |
| Q-429 | I-1 | II-9 | II-24 | II-27 |
| Q-430 | I-1 | II-9 | II-24 | II-28 |
| Q-431 | I-1 | II-9 | II-24 | II-29 |
| Q-432 | I-1 | II-9 | II-25 | II-26 |
| Q-433 | I-1 | II-9 | II-25 | II-27 |
| Q-434 | I-1 | II-9 | II-25 | II-28 |
| Q-435 | I-1 | II-9 | II-25 | II-29 |
| Q-436 | I-1 | II-9 | II-26 | II-27 |
| Q-437 | I-1 | II-9 | II-26 | II-28 |
| Q-438 | I-1 | II-9 | II-26 | II-29 |
| Q-439 | I-1 | II-9 | II-27 | II-28 |
| Q-440 | I-1 | II-9 | II-27 | II-29 |
| Q-441 | I-1 | II-9 | II-28 | II-29 |
| Q-442 | I-1 | II-15 | II-21 | II-22 |
| Q-443 | I-1 | II-15 | II-21 | II-23 |
| Q-444 | I-1 | II-15 | II-21 | II-24 |
| Q-445 | I-1 | II-15 | II-21 | II-25 |
| Q-446 | I-1 | II-15 | II-21 | II-26 |
| Q-447 | I-1 | II-15 | II-21 | II-27 |
| Q-448 | I-1 | II-15 | II-21 | II-28 |
| Q-449 | I-1 | II-15 | II-21 | II-29 |
| Q-450 | I-1 | II-15 | II-22 | II-23 |
| Q-451 | I-1 | II-15 | II-22 | II-24 |
| Q-452 | I-1 | II-15 | II-22 | II-25 |
| Q-453 | I-1 | II-15 | II-22 | II-26 |
| Q-454 | I-1 | II-15 | II-22 | II-27 |
| Q-455 | I-1 | II-15 | II-22 | II-28 |
| Q-456 | I-1 | II-15 | II-22 | II-29 |
| Q-457 | I-1 | II-15 | II-23 | II-24 |
| Q-458 | I-1 | II-15 | II-23 | II-25 |
| Q-459 | I-1 | II-15 | II-23 | II-26 |
| Q-460 | I-1 | II-15 | II-23 | II-27 |
| Q-461 | I-1 | II-15 | II-23 | II-28 |
| Q-462 | I-1 | II-15 | II-23 | II-29 |
| Q-463 | I-1 | II-15 | II-24 | II-25 |
| Q-464 | I-1 | II-15 | II-24 | II-26 |
| Q-465 | I-1 | II-15 | II-24 | II-27 |
| Q-466 | I-1 | II-15 | II-24 | II-28 |
| Q-467 | I-1 | II-15 | II-24 | II-29 |
| Q-468 | I-1 | II-15 | II-25 | II-26 |
| Q-469 | I-1 | II-15 | II-25 | II-27 |
| Q-470 | I-1 | II-15 | II-25 | II-28 |
| Q-471 | I-1 | II-15 | II-25 | II-29 |
| Q-472 | I-1 | II-15 | II-26 | II-27 |
| Q-473 | I-1 | II-15 | II-26 | II-28 |
| Q-474 | I-1 | II-15 | II-26 | II-29 |
| Q-475 | I-1 | II-15 | II-27 | II-28 |
| Q-476 | I-1 | II-15 | II-27 | II-29 |
| Q-477 | I-1 | II-15 | II-28 | II-29 |
| Q-478 | I-1 | II-21 | II-22 | II-23 |
| Q-479 | I-1 | II-21 | II-22 | II-24 |
| Q-480 | I-1 | II-21 | II-22 | II-25 |
| Q-481 | I-1 | II-21 | II-22 | II-26 |
| Q-482 | I-1 | II-21 | II-22 | II-27 |
| Q-483 | I-1 | II-21 | II-22 | II-28 |
| Q-484 | I-1 | II-21 | II-22 | II-29 |
| Q-485 | I-1 | II-21 | II-23 | II-24 |
| Q-486 | I-1 | II-21 | II-23 | II-25 |
| Q-487 | I-1 | II-21 | II-23 | II-26 |
| Q-488 | I-1 | II-21 | II-23 | II-27 |
| Q-489 | I-1 | II-21 | II-23 | II-28 |
| Q-490 | I-1 | II-21 | II-23 | II-29 |
| Q-491 | I-1 | II-21 | II-24 | II-25 |
| Q-492 | I-1 | II-21 | II-24 | II-26 |
| Q-493 | I-1 | II-21 | II-24 | II-27 |
| Q-494 | I-1 | II-21 | II-24 | II-28 |
| Q-495 | I-1 | II-21 | II-24 | II-29 |
| Q-496 | I-1 | II-21 | II-25 | II-26 |
| Q-497 | I-1 | II-21 | II-25 | II-27 |
| Q-498 | I-1 | II-21 | II-25 | II-28 |
| Q-499 | I-1 | II-21 | II-25 | II-29 |
| Q-500 | I-1 | II-21 | II-26 | II-27 |
| Q-501 | I-1 | II-21 | II-26 | II-28 |
| Q-502 | I-1 | II-21 | II-26 | II-29 |
| Q-503 | I-1 | II-21 | II-27 | II-28 |
| Q-504 | I-1 | II-21 | II-27 | II-29 |
| Q-505 | I-1 | II-21 | II-28 | II-29 |
| Q-506 | I-1 | II-22 | II-23 | II-24 |
| Q-507 | I-1 | II-22 | II-23 | II-25 |
| Q-508 | I-1 | II-22 | II-23 | II-26 |
| Q-509 | I-1 | II-22 | II-23 | II-27 |
| Q-510 | I-1 | II-22 | II-23 | II-28 |
| Q-511 | I-1 | II-22 | II-23 | II-29 |
| Q-512 | I-1 | II-22 | II-24 | II-25 |
| Q-513 | I-1 | II-22 | II-24 | II-26 |
| Q-514 | I-1 | II-22 | II-24 | II-27 |
| Q-515 | I-1 | II-22 | II-24 | II-28 |
| Q-516 | I-1 | II-22 | II-24 | II-29 |
| Q-517 | I-1 | II-22 | II-25 | II-26 |
| Q-518 | I-1 | II-22 | II-25 | II-27 |
| Q-519 | I-1 | II-22 | II-25 | II-28 |
| Q-520 | I-1 | II-22 | II-25 | II-29 |
| Q-521 | I-1 | II-22 | II-26 | II-27 |
| Q-522 | I-1 | II-22 | II-26 | II-28 |
| Q-523 | I-1 | II-22 | II-26 | II-29 |
| Q-524 | I-1 | II-22 | II-27 | II-28 |
| Q-525 | I-1 | II-22 | II-27 | II-29 |
| Q-526 | I-1 | II-22 | II-28 | II-29 |
| Q-527 | I-1 | II-23 | II-24 | II-25 |
| Q-528 | I-1 | II-23 | II-24 | II-26 |
| Q-529 | I-1 | II-23 | II-24 | II-27 |
| Q-530 | I-1 | II-23 | II-24 | II-28 |
| Q-531 | I-1 | II-23 | II-24 | II-29 |
| Q-532 | I-1 | II-23 | II-25 | II-26 |
| Q-533 | I-1 | II-23 | II-25 | II-27 |
| Q-534 | I-1 | II-23 | II-25 | II-28 |
| Q-535 | I-1 | II-23 | II-25 | II-29 |
| Q-536 | I-1 | II-23 | II-26 | II-27 |
| Q-537 | I-1 | II-23 | II-26 | II-28 |
| Q-538 | I-1 | II-23 | II-26 | II-29 |
| Q-539 | I-1 | II-23 | II-27 | II-28 |
| Q-540 | I-1 | II-23 | II-27 | II-29 |
| Q-541 | I-1 | II-23 | II-28 | II-29 |
| Q-542 | I-1 | II-24 | II-25 | II-26 |
| Q-543 | I-1 | II-24 | II-25 | II-27 |
| Q-544 | I-1 | II-24 | II-25 | II-28 |
| Q-545 | I-1 | II-24 | II-25 | II-29 |
| Q-546 | I-1 | II-24 | II-26 | II-27 |
| Q-547 | I-1 | II-24 | II-26 | II-28 |
| Q-548 | I-1 | II-24 | II-26 | II-29 |
| Q-549 | I-1 | II-24 | II-27 | II-28 |
| Q-550 | I-1 | II-24 | II-27 | II-29 |
| Q-551 | I-1 | II-24 | II-28 | II-29 |
| Q-552 | I-1 | II-25 | II-26 | II-27 |
| Q-553 | I-1 | II-25 | II-26 | II-28 |
| Q-554 | I-1 | II-25 | II-26 | II-29 |
| Q-555 | I-1 | II-25 | II-27 | II-28 |
| Q-556 | I-1 | II-25 | II-27 | II-29 |
| Q-557 | I-1 | II-25 | II-28 | II-29 |
| Q-558 | I-1 | II-26 | II-27 | II-28 |
| Q-559 | I-1 | II-26 | II-27 | II-29 |
| Q-560 | I-1 | II-26 | II-28 | II-29 |
| Q-561 | I-1 | II-27 | II-28 | II-29 |
| Q-562 | I-2 | II-1 | II-2 | II-3 |
| Q-563 | I-2 | II-1 | II-2 | II-7 |
| Q-564 | I-2 | II-1 | II-2 | II-8 |
| Q-565 | I-2 | II-1 | II-2 | II-9 |
| Q-566 | I-2 | II-1 | II-2 | II-15 |
| Q-567 | I-2 | II-1 | II-2 | II-21 |
| Q-568 | I-2 | II-1 | II-2 | II-22 |
| Q-569 | I-2 | II-1 | II-2 | II-23 |
| Q-570 | I-2 | II-1 | II-2 | II-24 |
| Q-571 | I-2 | II-1 | II-2 | II-25 |
| Q-572 | I-2 | II-1 | II-2 | II-26 |
| Q-573 | I-2 | II-1 | II-2 | II-27 |
| Q-574 | I-2 | II-1 | II-2 | II-28 |
| Q-575 | I-2 | II-1 | II-2 | II-29 |
| Q-576 | I-2 | II-1 | II-3 | II-7 |
| Q-577 | I-2 | II-1 | II-3 | II-8 |
| Q-578 | I-2 | II-1 | II-3 | II-9 |
| Q-579 | I-2 | II-1 | II-3 | II-15 |
| Q-580 | I-2 | II-1 | II-3 | II-21 |
| Q-581 | I-2 | II-1 | II-3 | II-22 |
| Q-582 | I-2 | II-1 | II-3 | II-23 |
| Q-583 | I-2 | II-1 | II-3 | II-24 |
| Q-584 | I-2 | II-1 | II-3 | II-25 |
| Q-585 | I-2 | II-1 | II-3 | II-26 |
| Q-586 | I-2 | II-1 | II-3 | II-27 |
| Q-587 | I-2 | II-1 | II-3 | II-28 |
| Q-588 | I-2 | II-1 | II-3 | II-29 |
| Q-589 | I-2 | II-1 | II-7 | II-8 |
| Q-590 | I-2 | II-1 | II-7 | II-9 |
| Q-591 | I-2 | II-1 | II-7 | II-15 |
| Q-592 | I-2 | II-1 | II-7 | II-21 |
| Q-593 | I-2 | II-1 | II-7 | II-22 |
| Q-594 | I-2 | II-1 | II-7 | II-23 |
| Q-595 | I-2 | II-1 | II-7 | II-24 |
| Q-596 | I-2 | II-1 | II-7 | II-25 |
| Q-597 | I-2 | II-1 | II-7 | II-26 |
| Q-598 | I-2 | II-1 | II-7 | II-27 |
| Q-599 | I-2 | II-1 | II-7 | II-28 |
| Q-600 | I-2 | II-1 | II-7 | II-29 |
| Q-601 | I-2 | II-1 | II-8 | II-9 |
| Q-602 | I-2 | II-1 | II-8 | II-15 |
| Q-603 | I-2 | II-1 | II-8 | II-21 |
| Q-604 | I-2 | II-1 | II-8 | II-22 |
| Q-605 | I-2 | II-1 | II-8 | II-23 |
| Q-606 | I-2 | II-1 | II-8 | II-24 |
| Q-607 | I-2 | II-1 | II-8 | II-25 |
| Q-608 | I-2 | II-1 | II-8 | II-26 |
| Q-609 | I-2 | II-1 | II-8 | II-27 |
| Q-610 | I-2 | II-1 | II-8 | II-28 |
| Q-611 | I-2 | II-1 | II-8 | II-29 |
| Q-612 | I-2 | II-1 | II-9 | II-15 |
| Q-613 | I-2 | II-1 | II-9 | II-21 |
| Q-614 | I-2 | II-1 | II-9 | II-22 |
| Q-615 | I-2 | II-1 | II-9 | II-23 |
| Q-616 | I-2 | II-1 | II-9 | II-24 |
| Q-617 | I-2 | II-1 | II-9 | II-25 |
| Q-618 | I-2 | II-1 | II-9 | II-26 |
| Q-619 | I-2 | II-1 | II-9 | II-27 |
| Q-620 | I-2 | II-1 | II-9 | II-28 |
| Q-621 | I-2 | II-1 | II-9 | II-29 |
| Q-622 | I-2 | II-1 | II-15 | II-21 |
| Q-623 | I-2 | II-1 | II-15 | II-22 |
| Q-624 | I-2 | II-1 | II-15 | II-23 |
| Q-625 | I-2 | II-1 | II-15 | II-24 |
| Q-626 | I-2 | II-1 | II-15 | II-25 |
| Q-627 | I-2 | II-1 | II-15 | II-26 |
| Q-628 | I-2 | II-1 | II-15 | II-27 |
| Q-629 | I-2 | II-1 | II-15 | II-28 |
| Q-630 | I-2 | II-1 | II-15 | II-29 |
| Q-631 | I-2 | II-1 | II-21 | II-22 |
| Q-632 | I-2 | II-1 | II-21 | II-23 |
| Q-633 | I-2 | II-1 | II-21 | II-24 |
| Q-634 | I-2 | II-1 | II-21 | II-25 |
| Q-635 | I-2 | II-1 | II-21 | II-26 |
| Q-636 | I-2 | II-1 | II-21 | II-27 |
| Q-637 | I-2 | II-1 | II-21 | II-28 |
| Q-638 | I-2 | II-1 | II-21 | II-29 |
| Q-639 | I-2 | II-1 | II-22 | II-23 |
| Q-640 | I-2 | II-1 | II-22 | II-24 |
| Q-641 | I-2 | II-1 | II-22 | II-25 |
| Q-642 | I-2 | II-1 | II-22 | II-26 |
| Q-643 | I-2 | II-1 | II-22 | II-27 |
| Q-644 | I-2 | II-1 | II-22 | II-28 |
| Q-645 | I-2 | II-1 | II-22 | II-29 |
| Q-646 | I-2 | II-1 | II-23 | II-24 |
| Q-647 | I-2 | II-1 | II-23 | II-25 |
| Q-648 | I-2 | II-1 | II-23 | II-26 |
| Q-649 | I-2 | II-1 | II-23 | II-27 |
| Q-650 | I-2 | II-1 | II-23 | II-28 |
| Q-651 | I-2 | II-1 | II-23 | II-29 |
| Q-652 | I-2 | II-1 | II-24 | II-25 |
| Q-653 | I-2 | II-1 | II-24 | II-26 |
| Q-654 | I-2 | II-1 | II-24 | II-27 |
| Q-655 | I-2 | II-1 | II-24 | II-28 |
| Q-656 | I-2 | II-1 | II-24 | II-29 |
| Q-657 | I-2 | II-1 | II-25 | II-26 |
| Q-658 | I-2 | II-1 | II-25 | II-27 |
| Q-659 | I-2 | II-1 | II-25 | II-28 |
| Q-660 | I-2 | II-1 | II-25 | II-29 |
| Q-661 | I-2 | II-1 | II-26 | II-27 |
| Q-662 | I-2 | II-1 | II-26 | II-28 |
| Q-663 | I-2 | II-1 | II-26 | II-29 |
| Q-664 | I-2 | II-1 | II-27 | II-28 |
| Q-665 | I-2 | II-1 | II-27 | II-29 |
| Q-666 | I-2 | II-1 | II-28 | II-29 |
| Q-667 | I-2 | II-2 | II-3 | II-7 |
| Q-668 | I-2 | II-2 | II-3 | II-8 |
| Q-669 | I-2 | II-2 | II-3 | II-9 |
| Q-670 | I-2 | II-2 | II-3 | II-15 |
| Q-671 | I-2 | II-2 | II-3 | II-21 |
| Q-672 | I-2 | II-2 | II-3 | II-22 |
| Q-673 | I-2 | II-2 | II-3 | II-23 |
| Q-674 | I-2 | II-2 | II-3 | II-24 |
| Q-675 | I-2 | II-2 | II-3 | II-25 |
| Q-676 | I-2 | II-2 | II-3 | II-26 |
| Q-677 | I-2 | II-2 | II-3 | II-27 |
| Q-678 | I-2 | II-2 | II-3 | II-28 |
| Q-679 | I-2 | II-2 | II-3 | II-29 |
| Q-680 | I-2 | II-2 | II-7 | II-8 |
| Q-681 | I-2 | II-2 | II-7 | II-9 |
| Q-682 | I-2 | II-2 | II-7 | II-15 |
| Q-683 | I-2 | II-2 | II-7 | II-21 |
| Q-684 | I-2 | II-2 | II-7 | II-22 |
| Q-685 | I-2 | II-2 | II-7 | II-23 |
| Q-686 | I-2 | II-2 | II-7 | II-24 |
| Q-687 | I-2 | II-2 | II-7 | II-25 |
| Q-688 | I-2 | II-2 | II-7 | II-26 |
| Q-689 | I-2 | II-2 | II-7 | II-27 |
| Q-690 | I-2 | II-2 | II-7 | II-28 |
| Q-691 | I-2 | II-2 | II-7 | II-29 |
| Q-692 | I-2 | II-2 | II-8 | II-9 |
| Q-693 | I-2 | II-2 | II-8 | II-15 |
| Q-694 | I-2 | II-2 | II-8 | II-21 |
| Q-695 | I-2 | II-2 | II-8 | II-22 |
| Q-696 | I-2 | II-2 | II-8 | II-23 |
| Q-697 | I-2 | II-2 | II-8 | II-24 |
| Q-698 | I-2 | II-2 | II-8 | II-25 |
| Q-699 | I-2 | II-2 | II-8 | II-26 |
| Q-700 | I-2 | II-2 | II-8 | II-27 |
| Q-701 | I-2 | II-2 | II-8 | II-28 |
| Q-702 | I-2 | II-2 | II-8 | II-29 |
| Q-703 | I-2 | II-2 | II-9 | II-15 |
| Q-704 | I-2 | II-2 | II-9 | II-21 |
| Q-705 | I-2 | II-2 | II-9 | II-22 |
| Q-706 | I-2 | II-2 | II-9 | II-23 |
| Q-707 | I-2 | II-2 | II-9 | II-24 |
| Q-708 | I-2 | II-2 | II-9 | II-25 |
| Q-709 | I-2 | II-2 | II-9 | II-26 |
| Q-710 | I-2 | II-2 | II-9 | II-27 |
| Q-711 | I-2 | II-2 | II-9 | II-28 |
| Q-712 | I-2 | II-2 | II-9 | II-29 |
| Q-713 | I-2 | II-2 | II-15 | II-21 |
| Q-714 | I-2 | II-2 | II-15 | II-22 |
| Q-715 | I-2 | II-2 | II-15 | II-23 |
| Q-716 | I-2 | II-2 | II-15 | II-24 |
| Q-717 | I-2 | II-2 | II-15 | II-25 |
| Q-718 | I-2 | II-2 | II-15 | II-26 |
| Q-719 | I-2 | II-2 | II-15 | II-27 |
| Q-720 | I-2 | II-2 | II-15 | II-28 |
| Q-721 | I-2 | II-2 | II-15 | II-29 |
| Q-722 | I-2 | II-2 | II-21 | II-22 |
| Q-723 | I-2 | II-2 | II-21 | II-23 |
| Q-724 | I-2 | II-2 | II-21 | II-24 |
| Q-725 | I-2 | II-2 | II-21 | II-25 |
| Q-726 | I-2 | II-2 | II-21 | II-26 |
| Q-727 | I-2 | II-2 | II-21 | II-27 |
| Q-728 | I-2 | II-2 | II-21 | II-28 |
| Q-729 | I-2 | II-2 | II-21 | II-29 |
| Q-730 | I-2 | II-2 | II-22 | II-23 |
| Q-731 | I-2 | II-2 | II-22 | II-24 |
| Q-732 | I-2 | II-2 | II-22 | II-25 |
| Q-733 | I-2 | II-2 | II-22 | II-26 |
| Q-734 | I-2 | II-2 | II-22 | II-27 |
| Q-735 | I-2 | II-2 | II-22 | II-28 |
| Q-736 | I-2 | II-2 | II-22 | II-29 |
| Q-737 | I-2 | II-2 | II-23 | II-24 |
| Q-738 | I-2 | II-2 | II-23 | II-25 |
| Q-739 | I-2 | II-2 | II-23 | II-26 |
| Q-740 | I-2 | II-2 | II-23 | II-27 |
| Q-741 | I-2 | II-2 | II-23 | II-28 |
| Q-742 | I-2 | II-2 | II-23 | II-29 |
| Q-743 | I-2 | II-2 | II-24 | II-25 |
| Q-744 | I-2 | II-2 | II-24 | II-26 |
| Q-745 | I-2 | II-2 | II-24 | II-27 |
| Q-746 | I-2 | II-2 | II-24 | II-28 |
| Q-747 | I-2 | II-2 | II-24 | II-29 |
| Q-748 | I-2 | II-2 | II-25 | II-26 |
| Q-749 | I-2 | II-2 | II-25 | II-27 |
| Q-750 | I-2 | II-2 | II-25 | II-28 |
| Q-751 | I-2 | II-2 | II-25 | II-29 |
| Q-752 | I-2 | II-2 | II-26 | II-27 |
| Q-753 | I-2 | II-2 | II-26 | II-28 |
| Q-754 | I-2 | II-2 | II-26 | II-29 |
| Q-755 | I-2 | II-2 | II-27 | II-28 |
| Q-756 | I-2 | II-2 | II-27 | II-29 |
| Q-757 | I-2 | II-2 | II-28 | II-29 |
| Q-758 | I-2 | II-3 | II-7 | II-8 |
| Q-759 | I-2 | II-3 | II-7 | II-9 |
| Q-760 | I-2 | II-3 | II-7 | II-15 |
| Q-761 | I-2 | II-3 | II-7 | II-21 |
| Q-762 | I-2 | II-3 | II-7 | II-22 |
| Q-763 | I-2 | II-3 | II-7 | II-23 |
| Q-764 | I-2 | II-3 | II-7 | II-24 |
| Q-765 | I-2 | II-3 | II-7 | II-25 |
| Q-766 | I-2 | II-3 | II-7 | II-26 |
| Q-767 | I-2 | II-3 | II-7 | II-27 |
| Q-768 | I-2 | II-3 | II-7 | II-28 |
| Q-769 | I-2 | II-3 | II-7 | II-29 |
| Q-770 | I-2 | II-3 | II-8 | II-9 |
| Q-771 | I-2 | II-3 | II-8 | II-15 |
| Q-772 | I-2 | II-3 | II-8 | II-21 |
| Q-773 | I-2 | II-3 | II-8 | II-22 |
| Q-774 | I-2 | II-3 | II-8 | II-23 |
| Q-775 | I-2 | II-3 | II-8 | II-24 |
| Q-776 | I-2 | II-3 | II-8 | II-25 |
| Q-777 | I-2 | II-3 | II-8 | II-26 |
| Q-778 | I-2 | II-3 | II-8 | II-27 |
| Q-779 | I-2 | II-3 | II-8 | II-28 |
| Q-780 | I-2 | II-3 | II-8 | II-29 |
| Q-781 | I-2 | II-3 | II-9 | II-15 |
| Q-782 | I-2 | II-3 | II-9 | II-21 |
| Q-783 | I-2 | II-3 | II-9 | II-22 |
| Q-784 | I-2 | II-3 | II-9 | II-23 |
| Q-785 | I-2 | II-3 | II-9 | II-24 |
| Q-786 | I-2 | II-3 | II-9 | II-25 |
| Q-787 | I-2 | II-3 | II-9 | II-26 |
| Q-788 | I-2 | II-3 | II-9 | II-27 |
| Q-789 | I-2 | II-3 | II-9 | II-28 |
| Q-790 | I-2 | II-3 | II-9 | II-29 |
| Q-791 | I-2 | II-3 | II-15 | II-21 |
| Q-792 | I-2 | II-3 | II-15 | II-22 |
| Q-793 | I-2 | II-3 | II-15 | II-23 |
| Q-794 | I-2 | II-3 | II-15 | II-24 |
| Q-795 | I-2 | II-3 | II-15 | II-25 |
| Q-796 | I-2 | II-3 | II-15 | II-26 |
| Q-797 | I-2 | II-3 | II-15 | II-27 |
| Q-798 | I-2 | II-3 | II-15 | II-28 |
| Q-799 | I-2 | II-3 | II-15 | II-29 |
| Q-800 | I-2 | II-3 | II-21 | II-22 |
| Q-801 | I-2 | II-3 | II-21 | II-23 |
| Q-802 | I-2 | II-3 | II-21 | II-24 |
| Q-803 | I-2 | II-3 | II-21 | II-25 |
| Q-804 | I-2 | II-3 | II-21 | II-26 |
| Q-805 | I-2 | II-3 | II-21 | II-27 |
| Q-806 | I-2 | II-3 | II-21 | II-28 |
| Q-807 | I-2 | II-3 | II-21 | II-29 |
| Q-808 | I-2 | II-3 | II-22 | II-23 |
| Q-809 | I-2 | II-3 | II-22 | II-24 |
| Q-810 | I-2 | II-3 | II-22 | II-25 |
| Q-811 | I-2 | II-3 | II-22 | II-26 |
| Q-812 | I-2 | II-3 | II-22 | II-27 |
| Q-813 | I-2 | II-3 | II-22 | II-28 |
| Q-814 | I-2 | II-3 | II-22 | II-29 |
| Q-815 | I-2 | II-3 | II-23 | II-24 |
| Q-816 | I-2 | II-3 | II-23 | II-25 |
| Q-817 | I-2 | II-3 | II-23 | II-26 |
| Q-818 | I-2 | II-3 | II-23 | II-27 |
| Q-819 | I-2 | II-3 | II-23 | II-28 |
| Q-820 | I-2 | II-3 | II-23 | II-29 |
| Q-821 | I-2 | II-3 | II-24 | II-25 |
| Q-822 | I-2 | II-3 | II-24 | II-26 |
| Q-823 | I-2 | II-3 | II-24 | II-27 |
| Q-824 | I-2 | II-3 | II-24 | II-28 |
| Q-825 | I-2 | II-3 | II-24 | II-29 |
| Q-826 | I-2 | II-3 | II-25 | II-26 |
| Q-827 | I-2 | II-3 | II-25 | II-27 |
| Q-828 | I-2 | II-3 | II-25 | II-28 |
| Q-829 | I-2 | II-3 | II-25 | II-29 |
| Q-830 | I-2 | II-3 | II-26 | II-27 |
| Q-831 | I-2 | II-3 | II-26 | II-28 |
| Q-832 | I-2 | II-3 | II-26 | II-29 |
| Q-833 | I-2 | II-3 | II-27 | II-28 |
| Q-834 | I-2 | II-3 | II-27 | II-29 |
| Q-835 | I-2 | II-3 | II-28 | II-29 |
| Q-836 | I-2 | II-7 | II-8 | II-9 |
| Q-837 | I-2 | II-7 | II-8 | II-15 |
| Q-838 | I-2 | II-7 | II-8 | II-21 |
| Q-839 | I-2 | II-7 | II-8 | II-22 |
| Q-840 | I-2 | II-7 | II-8 | II-23 |
| Q-841 | I-2 | II-7 | II-8 | II-24 |
| Q-842 | I-2 | II-7 | II-8 | II-25 |
| Q-843 | I-2 | II-7 | II-8 | II-26 |
| Q-844 | I-2 | II-7 | II-8 | II-27 |
| Q-845 | I-2 | II-7 | II-8 | II-28 |
| Q-846 | I-2 | II-7 | II-8 | II-29 |
| Q-847 | I-2 | II-7 | II-9 | II-15 |
| Q-848 | I-2 | II-7 | II-9 | II-21 |
| Q-849 | I-2 | II-7 | II-9 | II-22 |
| Q-850 | I-2 | II-7 | II-9 | II-23 |
| Q-851 | I-2 | II-7 | II-9 | II-24 |
| Q-852 | I-2 | II-7 | II-9 | II-25 |
| Q-853 | I-2 | II-7 | II-9 | II-26 |
| Q-854 | I-2 | II-7 | II-9 | II-27 |
| Q-855 | I-2 | II-7 | II-9 | II-28 |
| Q-856 | I-2 | II-7 | II-9 | II-29 |
| Q-857 | I-2 | II-7 | II-15 | II-21 |
| Q-858 | I-2 | II-7 | II-15 | II-22 |
| Q-859 | I-2 | II-7 | II-15 | II-23 |
| Q-860 | I-2 | II-7 | II-15 | II-24 |
| Q-861 | I-2 | II-7 | II-15 | II-25 |
| Q-862 | I-2 | II-7 | II-15 | II-26 |
| Q-863 | I-2 | II-7 | II-15 | II-27 |
| Q-864 | I-2 | II-7 | II-15 | II-28 |
| Q-865 | I-2 | II-7 | II-15 | II-29 |
| Q-866 | I-2 | II-7 | II-21 | II-22 |
| Q-867 | I-2 | II-7 | II-21 | II-23 |
| Q-868 | I-2 | II-7 | II-21 | II-24 |
| Q-869 | I-2 | II-7 | II-21 | II-25 |
| Q-870 | I-2 | II-7 | II-21 | II-26 |
| Q-871 | I-2 | II-7 | II-21 | II-27 |
| Q-872 | I-2 | II-7 | II-21 | II-28 |
| Q-873 | I-2 | II-7 | II-21 | II-29 |
| Q-874 | I-2 | II-7 | II-22 | II-23 |
| Q-875 | I-2 | II-7 | II-22 | II-24 |
| Q-876 | I-2 | II-7 | II-22 | II-25 |
| Q-877 | I-2 | II-7 | II-22 | II-26 |
| Q-878 | I-2 | II-7 | II-22 | II-27 |
| Q-879 | I-2 | II-7 | II-22 | II-28 |
| Q-880 | I-2 | II-7 | II-22 | II-29 |
| Q-881 | I-2 | II-7 | II-23 | II-24 |
| Q-882 | I-2 | II-7 | II-23 | II-25 |
| Q-883 | I-2 | II-7 | II-23 | II-26 |
| Q-884 | I-2 | II-7 | II-23 | II-27 |
| Q-885 | I-2 | II-7 | II-23 | II-28 |
| Q-886 | I-2 | II-7 | II-23 | II-29 |
| Q-887 | I-2 | II-7 | II-24 | II-24 |
| Q-888 | I-2 | II-7 | II-24 | II-25 |
| Q-889 | I-2 | II-7 | II-24 | II-26 |
| Q-890 | I-2 | II-7 | II-24 | II-27 |
| Q-891 | I-2 | II-7 | II-24 | II-28 |
| Q-892 | I-2 | II-7 | II-24 | II-29 |
| Q-893 | I-2 | II-7 | II-25 | II-26 |
| Q-894 | I-2 | II-7 | II-25 | II-27 |
| Q-895 | I-2 | II-7 | II-25 | II-28 |
| Q-896 | I-2 | II-7 | II-25 | II-29 |
| Q-897 | I-2 | II-7 | II-26 | II-27 |
| Q-898 | I-2 | II-7 | II-26 | II-28 |
| Q-899 | I-2 | II-7 | II-26 | II-29 |
| Q-900 | I-2 | II-7 | II-27 | II-28 |
| Q-901 | I-2 | II-7 | II-27 | II-29 |
| Q-902 | I-2 | II-7 | II-28 | II-29 |
| Q-903 | I-2 | II-8 | II-9 | II-15 |
| Q-904 | I-2 | II-8 | II-9 | II-21 |
| Q-905 | I-2 | II-8 | II-9 | II-22 |
| Q-906 | I-2 | II-8 | II-9 | II-23 |
| Q-907 | I-2 | II-8 | II-9 | II-24 |
| Q-908 | I-2 | II-8 | II-9 | II-25 |
| Q-909 | I-2 | II-8 | II-9 | II-26 |
| Q-910 | I-2 | II-8 | II-9 | II-27 |
| Q-911 | I-2 | II-8 | II-9 | II-28 |
| Q-912 | I-2 | II-8 | II-9 | II-29 |
| Q-913 | I-2 | II-8 | II-15 | II-21 |
| Q-914 | I-2 | II-8 | II-15 | II-22 |
| Q-915 | I-2 | II-8 | II-15 | II-23 |
| Q-916 | I-2 | II-8 | II-15 | II-24 |
| Q-917 | I-2 | II-8 | II-15 | II-25 |
| Q-918 | I-2 | II-8 | II-15 | II-26 |
| Q-919 | I-2 | II-8 | II-15 | II-27 |
| Q-920 | I-2 | II-8 | II-15 | II-28 |
| Q-921 | I-2 | II-8 | II-15 | II-29 |
| Q-922 | I-2 | II-8 | II-21 | II-22 |
| Q-923 | I-2 | II-8 | II-21 | II-23 |
| Q-924 | I-2 | II-8 | II-21 | II-24 |
| Q-925 | I-2 | II-8 | II-21 | II-25 |
| Q-926 | I-2 | II-8 | II-21 | II-26 |
| Q-927 | I-2 | II-8 | II-21 | II-27 |
| Q-928 | I-2 | II-8 | II-21 | II-28 |
| Q-929 | I-2 | II-8 | II-21 | II-29 |
| Q-930 | I-2 | II-8 | II-22 | II-23 |
| Q-931 | I-2 | II-8 | II-22 | II-24 |
| Q-932 | I-2 | II-8 | II-22 | II-25 |
| Q-933 | I-2 | II-8 | II-22 | II-26 |
| Q-934 | I-2 | II-8 | II-22 | II-27 |
| Q-935 | I-2 | II-8 | II-22 | II-28 |
| Q-936 | I-2 | II-8 | II-22 | II-29 |
| Q-937 | I-2 | II-8 | II-23 | II-24 |
| Q-938 | I-2 | II-8 | II-23 | II-25 |
| Q-939 | I-2 | II-8 | II-23 | II-26 |
| Q-940 | I-2 | II-8 | II-23 | II-27 |
| Q-941 | I-2 | II-8 | II-23 | II-28 |
| Q-942 | I-2 | II-8 | II-23 | II-29 |
| Q-943 | I-2 | II-8 | II-24 | II-25 |
| Q-944 | I-2 | II-8 | II-24 | II-26 |
| Q-945 | I-2 | II-8 | II-24 | II-27 |
| Q-946 | I-2 | II-8 | II-24 | II-28 |
| Q-947 | I-2 | II-8 | II-24 | II-29 |
| Q-948 | I-2 | II-8 | II-25 | II-26 |
| Q-949 | I-2 | II-8 | II-25 | II-27 |
| Q-950 | I-2 | II-8 | II-25 | II-28 |
| Q-951 | I-2 | II-8 | II-25 | II-29 |
| Q-952 | I-2 | II-8 | II-26 | II-27 |
| Q-953 | I-2 | II-8 | II-26 | II-28 |
| Q-954 | I-2 | II-8 | II-26 | II-29 |
| Q-955 | I-2 | II-8 | II-27 | II-28 |
| Q-956 | I-2 | II-8 | II-27 | II-29 |
| Q-957 | I-2 | II-8 | II-28 | II-29 |
| Q-958 | I-2 | II-9 | II-15 | II-21 |
| Q-959 | I-2 | II-9 | II-15 | II-22 |
| Q-960 | I-2 | II-9 | II-15 | II-23 |
| Q-961 | I-2 | II-9 | II-15 | II-24 |
| Q-962 | I-2 | II-9 | II-15 | II-25 |
| Q-963 | I-2 | II-9 | II-15 | II-26 |
| Q-964 | I-2 | II-9 | II-15 | II-27 |
| Q-965 | I-2 | II-9 | II-15 | II-28 |
| Q-966 | I-2 | II-9 | II-15 | II-29 |
| Q-967 | I-2 | II-9 | II-21 | II-22 |
| Q-968 | I-2 | II-9 | II-21 | II-23 |
| Q-969 | I-2 | II-9 | II-21 | II-24 |
| Q-970 | I-2 | II-9 | II-21 | II-25 |
| Q-971 | I-2 | II-9 | II-21 | II-26 |
| Q-972 | I-2 | II-9 | II-21 | II-27 |
| Q-973 | I-2 | II-9 | II-21 | II-28 |
| Q-974 | I-2 | II-9 | II-21 | II-29 |
| Q-975 | I-2 | II-9 | II-22 | II-23 |
| Q-976 | I-2 | II-9 | II-22 | II-24 |
| Q-977 | I-2 | II-9 | II-22 | II-25 |
| Q-978 | I-2 | II-9 | II-22 | II-26 |
| Q-979 | I-2 | II-9 | II-22 | II-27 |
| Q-980 | I-2 | II-9 | II-22 | II-28 |
| Q-981 | I-2 | II-9 | II-22 | II-29 |
| Q-982 | I-2 | II-9 | II-23 | II-24 |
| Q-983 | I-2 | II-9 | II-23 | II-25 |
| Q-984 | I-2 | II-9 | II-23 | II-26 |
| Q-985 | I-2 | II-9 | II-23 | II-27 |
| Q-986 | I-2 | II-9 | II-23 | II-28 |
| Q-987 | I-2 | II-9 | II-23 | II-29 |
| Q-988 | I-2 | II-9 | II-24 | II-25 |
| Q-989 | I-2 | II-9 | II-24 | II-26 |
| Q-990 | I-2 | II-9 | II-24 | II-27 |
| Q-991 | I-2 | II-9 | II-24 | II-28 |
| Q-992 | I-2 | II-9 | II-24 | II-29 |
| Q-993 | I-2 | II-9 | II-25 | II-26 |
| Q-994 | I-2 | II-9 | II-25 | II-27 |
| Q-995 | I-2 | II-9 | II-25 | II-28 |
| Q-996 | I-2 | II-9 | II-25 | II-29 |
| Q-997 | I-2 | II-9 | II-26 | II-27 |
| Q-998 | I-2 | II-9 | II-26 | II-28 |
| Q-999 | I-2 | II-9 | II-26 | II-29 |
| Q-1000 | I-2 | II-9 | II-27 | II-28 |
| Q-1001 | I-2 | II-9 | II-27 | II-29 |
| Q-1002 | I-2 | II-9 | II-28 | II-29 |
| Q-1003 | I-2 | II-15 | II-21 | II-22 |
| Q-1004 | I-2 | II-15 | II-21 | II-23 |
| Q-1005 | I-2 | II-15 | II-21 | II-24 |
| Q-1006 | I-2 | II-15 | II-21 | II-25 |
| Q-1007 | I-2 | II-15 | II-21 | II-26 |
| Q-1008 | I-2 | II-15 | II-21 | II-27 |
| Q-1009 | I-2 | II-15 | II-21 | II-28 |
| Q-1010 | I-2 | II-15 | II-21 | II-29 |
| Q-1011 | I-2 | II-15 | II-22 | II-23 |
| Q-1012 | I-2 | II-15 | II-22 | II-24 |
| Q-1013 | I-2 | II-15 | II-22 | II-25 |
| Q-1014 | I-2 | II-15 | II-22 | II-26 |
| Q-1015 | I-2 | II-15 | II-22 | II-27 |
| Q-1016 | I-2 | II-15 | II-22 | II-28 |
| Q-1017 | I-2 | II-15 | II-22 | II-29 |
| Q-1018 | I-2 | II-15 | II-23 | II-24 |
| Q-1019 | I-2 | II-15 | II-23 | II-25 |
| Q-1020 | I-2 | II-15 | II-23 | II-26 |
| Q-1021 | I-2 | II-15 | II-23 | II-27 |
| Q-1022 | I-2 | II-15 | II-23 | II-28 |
| Q-1023 | I-2 | II-15 | II-23 | II-29 |
| Q-1024 | I-2 | II-15 | II-24 | II-25 |
| Q-1025 | I-2 | II-15 | II-24 | II-26 |
| Q-1026 | I-2 | II-15 | II-24 | II-27 |
| Q-1027 | I-2 | II-15 | II-24 | II-28 |
| Q-1028 | I-2 | II-15 | II-24 | II-29 |
| Q-1029 | I-2 | II-15 | II-25 | II-26 |
| Q-1030 | I-2 | II-15 | II-25 | II-27 |
| Q-1031 | I-2 | II-15 | II-25 | II-28 |
| Q-1032 | I-2 | II-15 | II-25 | II-29 |
| Q-1033 | I-2 | II-15 | II-26 | II-27 |
| Q-1034 | I-2 | II-15 | II-26 | II-28 |
| Q-1035 | I-2 | II-15 | II-26 | II-29 |
| Q-1036 | I-2 | II-15 | II-27 | II-28 |
| Q-1037 | I-2 | II-15 | II-27 | II-29 |
| Q-1038 | I-2 | II-15 | II-28 | II-29 |
| Q-1039 | I-2 | II-21 | II-22 | II-23 |
| Q-1040 | I-2 | II-21 | II-22 | II-24 |
| Q-1041 | I-2 | II-21 | II-22 | II-25 |
| Q-1042 | I-2 | II-21 | II-22 | II-26 |
| Q-1043 | I-2 | II-21 | II-22 | II-27 |
| Q-1044 | I-2 | II-21 | II-22 | II-28 |
| Q-1045 | I-2 | II-21 | II-22 | II-29 |
| Q-1046 | I-2 | II-21 | II-23 | II-24 |
| Q-1047 | I-2 | II-21 | II-23 | II-25 |
| Q-1048 | I-2 | II-21 | II-23 | II-26 |
| Q-1049 | I-2 | II-21 | II-23 | II-27 |
| Q-1050 | I-2 | II-21 | II-23 | II-28 |
| Q-1051 | I-2 | II-21 | II-23 | II-29 |
| Q-1052 | I-2 | II-21 | II-24 | II-25 |
| Q-1053 | I-2 | II-21 | II-24 | II-26 |
| Q-1054 | I-2 | II-21 | II-24 | II-27 |
| Q-1055 | I-2 | II-21 | II-24 | II-28 |
| Q-1056 | I-2 | II-21 | II-24 | II-29 |
| Q-1057 | I-2 | II-21 | II-25 | II-26 |
| Q-1058 | I-2 | II-21 | II-25 | II-27 |
| Q-1059 | I-2 | II-21 | II-25 | II-28 |
| Q-1060 | I-2 | II-21 | II-25 | II-29 |
| Q-1061 | I-2 | II-21 | II-26 | II-27 |
| Q-1062 | I-2 | II-21 | II-26 | II-28 |
| Q-1063 | I-2 | II-21 | II-26 | II-29 |
| Q-1064 | I-2 | II-21 | II-27 | II-28 |
| Q-1065 | I-2 | II-21 | II-27 | II-29 |
| Q-1066 | I-2 | II-21 | II-28 | II-29 |
| Q-1067 | I-2 | II-22 | II-23 | II-24 |
| Q-1068 | I-2 | II-22 | II-23 | II-25 |
| Q-1069 | I-2 | II-22 | II-23 | II-26 |
| Q-1070 | I-2 | II-22 | II-23 | II-27 |
| Q-1071 | I-2 | II-22 | II-23 | II-28 |
| Q-1072 | I-2 | II-22 | II-23 | II-29 |
| Q-1073 | I-2 | II-22 | II-24 | II-25 |
| Q-1074 | I-2 | II-22 | II-24 | II-26 |
| Q-1075 | I-2 | II-22 | II-24 | II-27 |
| Q-1076 | I-2 | II-22 | II-24 | II-28 |
| Q-1077 | I-2 | II-22 | II-24 | II-29 |
| Q-1078 | I-2 | II-22 | II-25 | II-26 |
| Q-1079 | I-2 | II-22 | II-25 | II-27 |
| Q-1080 | I-2 | II-22 | II-25 | II-28 |
| Q-1081 | I-2 | II-22 | II-25 | II-29 |
| Q-1082 | I-2 | II-22 | II-26 | II-27 |
| Q-1083 | I-2 | II-22 | II-26 | II-28 |
| Q-1084 | I-2 | II-22 | II-26 | II-29 |
| Q-1085 | I-2 | II-22 | II-27 | II-28 |
| Q-1086 | I-2 | II-22 | II-27 | II-29 |
| Q-1087 | I-2 | II-22 | II-28 | II-29 |
| Q-1088 | I-2 | II-23 | II-24 | II-25 |
| Q-1089 | I-2 | II-23 | II-24 | II-26 |
| Q-1090 | I-2 | II-23 | II-24 | II-27 |
| Q-1091 | I-2 | II-23 | II-24 | II-28 |
| Q-1092 | I-2 | II-23 | II-24 | II-29 |
| Q-1093 | I-2 | II-23 | II-25 | II-26 |
| Q-1094 | I-2 | II-23 | II-25 | II-27 |
| Q-1095 | I-2 | II-23 | II-25 | II-28 |
| Q-1096 | I-2 | II-23 | II-25 | II-29 |
| Q-1097 | I-2 | II-23 | II-26 | II-27 |
| Q-1098 | I-2 | II-23 | II-26 | II-28 |
| Q-1099 | I-2 | II-23 | II-26 | II-29 |
| Q-1100 | I-2 | II-23 | II-27 | II-28 |
| Q-1101 | I-2 | II-23 | II-27 | II-29 |
| Q-1102 | I-2 | II-23 | II-28 | II-29 |
| Q-1103 | I-2 | II-24 | II-25 | II-26 |
| Q-1104 | I-2 | II-24 | II-25 | II-27 |
| Q-1105 | I-2 | II-24 | II-25 | II-28 |
| Q-1106 | I-2 | II-24 | II-25 | II-29 |
| Q-1107 | I-2 | II-24 | II-26 | II-27 |
| Q-1108 | I-2 | II-24 | II-26 | II-28 |
| Q-1109 | I-2 | II-24 | II-26 | II-29 |
| Q-1110 | I-2 | II-24 | II-27 | II-28 |
| Q-1111 | I-2 | II-24 | II-27 | II-29 |
| Q-1112 | I-2 | II-24 | II-28 | II-29 |
| Q-1113 | I-2 | II-25 | II-26 | II-27 |
| Q-1114 | I-2 | II-25 | II-26 | II-28 |
| Q-1115 | I-2 | II-25 | II-26 | II-29 |
| Q-1116 | I-2 | II-25 | II-27 | II-28 |
| Q-1117 | I-2 | II-25 | II-27 | II-29 |
| Q-1118 | I-2 | II-25 | II-28 | II-29 |
| Q-1119 | I-2 | II-26 | II-27 | II-28 |
| Q-1120 | I-2 | II-26 | II-27 | II-29 |
| Q-1121 | I-2 | II-26 | II-28 | II-29 |
| Q-1122 | I-2 | II-27 | II-28 | II-29 |
| Q-1123 | I-6 | II-1 | II-2 | II-3 |
| Q-1124 | I-6 | II-1 | II-2 | II-7 |
| Q-1125 | I-6 | II-1 | II-2 | II-8 |
| Q-1126 | I-6 | II-1 | II-2 | II-9 |
| Q-1127 | I-6 | II-1 | II-2 | II-15 |
| Q-1128 | I-6 | II-1 | II-2 | II-21 |
| Q-1129 | I-6 | II-1 | II-2 | II-22 |
| Q-1130 | I-6 | II-1 | II-2 | II-23 |
| Q-1131 | I-6 | II-1 | II-2 | II-24 |
| Q-1132 | I-6 | II-1 | II-2 | II-25 |
| Q-1133 | I-6 | II-1 | II-2 | II-26 |
| Q-1134 | I-6 | II-1 | II-2 | II-27 |
| Q-1135 | I-6 | II-1 | II-2 | II-28 |
| Q-1136 | I-6 | II-1 | II-2 | II-29 |
| Q-1137 | I-6 | II-1 | II-3 | II-7 |
| Q-1138 | I-6 | II-1 | II-3 | II-8 |
| Q-1139 | I-6 | II-1 | II-3 | II-9 |
| Q-1140 | I-6 | II-1 | II-3 | II-15 |
| Q-1141 | I-6 | II-1 | II-3 | II-21 |
| Q-1142 | I-6 | II-1 | II-3 | II-22 |
| Q-1143 | I-6 | II-1 | II-3 | II-23 |
| Q-1144 | I-6 | II-1 | II-3 | II-24 |
| Q-1145 | I-6 | II-1 | II-3 | II-25 |
| Q-1146 | I-6 | II-1 | II-3 | II-26 |
| Q-1147 | I-6 | II-1 | II-3 | II-27 |
| Q-1148 | I-6 | II-1 | II-3 | II-28 |
| Q-1149 | I-6 | II-1 | II-3 | II-29 |
| Q-1150 | I-6 | II-1 | II-7 | II-8 |
| Q-1151 | I-6 | II-1 | II-7 | II-9 |
| Q-1152 | I-6 | II-1 | II-7 | II-15 |
| Q-1153 | I-6 | II-1 | II-7 | II-21 |
| Q-1154 | I-6 | II-1 | II-7 | II-22 |
| Q-1155 | I-6 | II-1 | II-7 | II-23 |
| Q-1156 | I-6 | II-1 | II-7 | II-24 |
| Q-1157 | I-6 | II-1 | II-7 | II-25 |
| Q-1158 | I-6 | II-1 | II-7 | II-26 |
| Q-1159 | I-6 | II-1 | II-7 | II-27 |
| Q-1160 | I-6 | II-1 | II-7 | II-28 |
| Q-1161 | I-6 | II-1 | II-7 | II-29 |
| Q-1162 | I-6 | II-1 | II-8 | II-9 |
| Q-1163 | I-6 | II-1 | II-8 | II-15 |
| Q-1164 | I-6 | II-1 | II-8 | II-21 |
| Q-1165 | I-6 | II-1 | II-8 | II-22 |
| Q-1166 | I-6 | II-1 | II-8 | II-23 |
| Q-1167 | I-6 | II-1 | II-8 | II-24 |
| Q-1168 | I-6 | II-1 | II-8 | II-25 |
| Q-1169 | I-6 | II-1 | II-8 | II-26 |
| Q-1170 | I-6 | II-1 | II-8 | II-27 |
| Q-1171 | I-6 | II-1 | II-8 | II-28 |
| Q-1172 | I-6 | II-1 | II-8 | II-29 |
| Q-1173 | I-6 | II-1 | II-9 | II-15 |
| Q-1174 | I-6 | II-1 | II-9 | II-21 |
| Q-1175 | I-6 | II-1 | II-9 | II-22 |
| Q-1176 | I-6 | II-1 | II-9 | II-23 |
| Q-1177 | I-6 | II-1 | II-9 | II-24 |
| Q-1178 | I-6 | II-1 | II-9 | II-25 |
| Q-1179 | I-6 | II-1 | II-9 | II-26 |
| Q-1180 | I-6 | II-1 | II-9 | II-27 |
| Q-1181 | I-6 | II-1 | II-9 | II-28 |
| Q-1182 | I-6 | II-1 | II-9 | II-29 |
| Q-1183 | I-6 | II-1 | II-15 | II-21 |
| Q-1184 | I-6 | II-1 | II-15 | II-22 |
| Q-1185 | I-6 | II-1 | II-15 | II-23 |
| Q-1186 | I-6 | II-1 | II-15 | II-24 |
| Q-1187 | I-6 | II-1 | II-15 | II-25 |
| Q-1188 | I-6 | II-1 | II-15 | II-26 |
| Q-1189 | I-6 | II-1 | II-15 | II-27 |
| Q-1190 | I-6 | II-1 | II-15 | II-28 |
| Q-1191 | I-6 | II-1 | II-15 | II-29 |
| Q-1192 | I-6 | II-1 | II-21 | II-22 |
| Q-1193 | I-6 | II-1 | II-21 | II-23 |
| Q-1194 | I-6 | II-1 | II-21 | II-24 |
| Q-1195 | I-6 | II-1 | II-21 | II-25 |
| Q-1196 | I-6 | II-1 | II-21 | II-26 |
| Q-1197 | I-6 | II-1 | II-21 | II-27 |
| Q-1198 | I-6 | II-1 | II-21 | II-28 |
| Q-1199 | I-6 | II-1 | II-21 | II-29 |
| Q-1200 | I-6 | II-1 | II-22 | II-23 |
| Q-1201 | I-6 | II-1 | II-22 | II-24 |
| Q-1202 | I-6 | II-1 | II-22 | II-25 |
| Q-1203 | I-6 | II-1 | II-22 | II-26 |
| Q-1204 | I-6 | II-1 | II-22 | II-27 |
| Q-1205 | I-6 | II-1 | II-22 | II-28 |
| Q-1206 | I-6 | II-1 | II-22 | II-29 |
| Q-1207 | I-6 | II-1 | II-23 | II-24 |
| Q-1208 | I-6 | II-1 | II-23 | II-25 |
| Q-1209 | I-6 | II-1 | II-23 | II-26 |
| Q-1210 | I-6 | II-1 | II-23 | II-27 |
| Q-1211 | I-6 | II-1 | II-23 | II-28 |
| Q-1212 | I-6 | II-1 | II-23 | II-29 |
| Q-1213 | I-6 | II-1 | II-24 | II-25 |
| Q-1214 | I-6 | II-1 | II-24 | II-26 |
| Q-1215 | I-6 | II-1 | II-24 | II-27 |
| Q-1216 | I-6 | II-1 | II-24 | II-28 |
| Q-1217 | I-6 | II-1 | II-24 | II-29 |
| Q-1218 | I-6 | II-1 | II-25 | II-26 |
| Q-1219 | I-6 | II-1 | II-25 | II-27 |
| Q-1220 | I-6 | II-1 | II-25 | II-28 |
| Q-1221 | I-6 | II-1 | II-25 | II-29 |
| Q-1222 | I-6 | II-1 | II-26 | II-27 |
| Q-1223 | I-6 | II-1 | II-26 | II-28 |
| Q-1224 | I-6 | II-1 | II-26 | II-29 |
| Q-1225 | I-6 | II-1 | II-27 | II-28 |
| Q-1226 | I-6 | II-1 | II-27 | II-29 |
| Q-1227 | I-6 | II-1 | II-28 | II-29 |
| Q-1228 | I-6 | II-2 | II-3 | II-7 |
| Q-1229 | I-6 | II-2 | II-3 | II-8 |
| Q-1230 | I-6 | II-2 | II-3 | II-9 |
| Q-1231 | I-6 | II-2 | II-3 | II-15 |
| Q-1232 | I-6 | II-2 | II-3 | II-21 |
| Q-1233 | I-6 | II-2 | II-3 | II-22 |
| Q-1234 | I-6 | II-2 | II-3 | II-23 |
| Q-1235 | I-6 | II-2 | II-3 | II-24 |
| Q-1236 | I-6 | II-2 | II-3 | II-25 |
| Q-1237 | I-6 | II-2 | II-3 | II-26 |
| Q-1238 | I-6 | II-2 | II-3 | II-27 |
| Q-1239 | I-6 | II-2 | II-3 | II-28 |
| Q-1240 | I-6 | II-2 | II-3 | II-29 |
| Q-1241 | I-6 | II-2 | II-7 | II-8 |
| Q-1242 | I-6 | II-2 | II-7 | II-9 |
| Q-1243 | I-6 | II-2 | II-7 | II-15 |
| Q-1244 | I-6 | II-2 | II-7 | II-21 |
| Q-1245 | I-6 | II-2 | II-7 | II-22 |
| Q-1246 | I-6 | II-2 | II-7 | II-23 |
| Q-1247 | I-6 | II-2 | II-7 | II-24 |
| Q-1248 | I-6 | II-2 | II-7 | II-25 |
| Q-1249 | I-6 | II-2 | II-7 | II-26 |
| Q-1250 | I-6 | II-2 | II-7 | II-27 |
| Q-1251 | I-6 | II-2 | II-7 | II-28 |
| Q-1252 | I-6 | II-2 | II-7 | II-29 |
| Q-1253 | I-6 | II-2 | II-8 | II-9 |
| Q-1254 | I-6 | II-2 | II-8 | II-15 |
| Q-1255 | I-6 | II-2 | II-8 | II-21 |
| Q-1256 | I-6 | II-2 | II-8 | II-22 |
| Q-1257 | I-6 | II-2 | II-8 | II-23 |
| Q-1258 | I-6 | II-2 | II-8 | II-24 |
| Q-1259 | I-6 | II-2 | II-8 | II-25 |
| Q-1260 | I-6 | II-2 | II-8 | II-26 |
| Q-1261 | I-6 | II-2 | II-8 | II-27 |
| Q-1262 | I-6 | II-2 | II-8 | II-28 |
| Q-1263 | I-6 | II-2 | II-8 | II-29 |
| Q-1264 | I-6 | II-2 | II-9 | II-15 |
| Q-1265 | I-6 | II-2 | II-9 | II-21 |
| Q-1266 | I-6 | II-2 | II-9 | II-22 |
| Q-1267 | I-6 | II-2 | II-9 | II-23 |
| Q-1268 | I-6 | II-2 | II-9 | II-24 |
| Q-1269 | I-6 | II-2 | II-9 | II-25 |
| Q-1270 | I-6 | II-2 | II-9 | II-26 |
| Q-1271 | I-6 | II-2 | II-9 | II-27 |
| Q-1272 | I-6 | II-2 | II-9 | II-28 |
| Q-1273 | I-6 | II-2 | II-9 | II-29 |
| Q-1274 | I-6 | II-2 | II-15 | II-21 |
| Q-1275 | I-6 | II-2 | II-15 | II-22 |
| Q-1276 | I-6 | II-2 | II-15 | II-23 |
| Q-1277 | I-6 | II-2 | II-15 | II-24 |
| Q-1278 | I-6 | II-2 | II-15 | II-25 |
| Q-1279 | I-6 | II-2 | II-15 | II-26 |
| Q-1280 | I-6 | II-2 | II-15 | II-27 |
| Q-1281 | I-6 | II-2 | II-15 | II-28 |
| Q-1282 | I-6 | II-2 | II-15 | II-29 |
| Q-1283 | I-6 | II-2 | II-21 | II-22 |
| Q-1284 | I-6 | II-2 | II-21 | II-23 |
| Q-1285 | I-6 | II-2 | II-21 | II-24 |
| Q-1286 | I-6 | II-2 | II-21 | II-25 |
| Q-1287 | I-6 | II-2 | II-21 | II-26 |
| Q-1288 | I-6 | II-2 | II-21 | II-27 |
| Q-1289 | I-6 | II-2 | II-21 | II-28 |
| Q-1290 | I-6 | II-2 | II-21 | II-29 |
| Q-1291 | I-6 | II-2 | II-22 | II-23 |
| Q-1292 | I-6 | II-2 | II-22 | II-24 |
| Q-1293 | I-6 | II-2 | II-22 | II-25 |
| Q-1294 | I-6 | II-2 | II-22 | II-26 |
| Q-1295 | I-6 | II-2 | II-22 | II-27 |
| Q-1296 | I-6 | II-2 | II-22 | II-28 |
| Q-1297 | I-6 | II-2 | II-22 | II-29 |
| Q-1298 | I-6 | II-2 | II-23 | II-24 |
| Q-1299 | I-6 | II-2 | II-23 | II-25 |
| Q-1300 | I-6 | II-2 | II-23 | II-26 |
| Q-1301 | I-6 | II-2 | II-23 | II-27 |
| Q-1302 | I-6 | II-2 | II-23 | II-28 |
| Q-1303 | I-6 | II-2 | II-23 | II-29 |
| Q-1304 | I-6 | II-2 | II-24 | II-25 |
| Q-1305 | I-6 | II-2 | II-24 | II-26 |
| Q-1306 | I-6 | II-2 | II-24 | II-27 |
| Q-1307 | I-6 | II-2 | II-24 | II-28 |
| Q-1308 | I-6 | II-2 | II-24 | II-29 |
| Q-1309 | I-6 | II-2 | II-25 | II-26 |
| Q-1310 | I-6 | II-2 | II-25 | II-27 |
| Q-1311 | I-6 | II-2 | II-25 | II-28 |
| Q-1312 | I-6 | II-2 | II-25 | II-29 |
| Q-1313 | I-6 | II-2 | II-26 | II-27 |
| Q-1314 | I-6 | II-2 | II-26 | II-28 |
| Q-1315 | I-6 | II-2 | II-26 | II-29 |
| Q-1316 | I-6 | II-2 | II-27 | II-28 |
| Q-1317 | I-6 | II-2 | II-27 | II-29 |
| Q-1318 | I-6 | II-2 | II-28 | II-29 |
| Q-1319 | I-6 | II-3 | II-7 | II-8 |
| Q-1320 | I-6 | II-3 | II-7 | II-9 |
| Q-1321 | I-6 | II-3 | II-7 | II-15 |
| Q-1322 | I-6 | II-3 | II-7 | II-21 |
| Q-1323 | I-6 | II-3 | II-7 | II-22 |
| Q-1324 | I-6 | II-3 | II-7 | II-23 |
| Q-1325 | I-6 | II-3 | II-7 | II-24 |
| Q-1326 | I-6 | II-3 | II-7 | II-25 |
| Q-1327 | I-6 | II-3 | II-7 | II-26 |
| Q-1328 | I-6 | II-3 | II-7 | II-27 |
| Q-1329 | I-6 | II-3 | II-7 | II-28 |
| Q-1330 | I-6 | II-3 | II-7 | II-29 |
| Q-1331 | I-6 | II-3 | II-8 | II-9 |
| Q-1332 | I-6 | II-3 | II-8 | II-15 |
| Q-1333 | I-6 | II-3 | II-8 | II-21 |
| Q-1334 | I-6 | II-3 | II-8 | II-22 |
| Q-1335 | I-6 | II-3 | II-8 | II-23 |
| Q-1336 | I-6 | II-3 | II-8 | II-24 |
| Q-1337 | I-6 | II-3 | II-8 | II-25 |
| Q-1338 | I-6 | II-3 | II-8 | II-26 |
| Q-1339 | I-6 | II-3 | II-8 | II-27 |
| Q-1340 | I-6 | II-3 | II-8 | II-28 |
| Q-1341 | I-6 | II-3 | II-8 | II-29 |
| Q-1342 | I-6 | II-3 | II-9 | II-15 |
| Q-1343 | I-6 | II-3 | II-9 | II-21 |
| Q-1344 | I-6 | II-3 | II-9 | II-22 |
| Q-1345 | I-6 | II-3 | II-9 | II-23 |
| Q-1346 | I-6 | II-3 | II-9 | II-24 |
| Q-1347 | I-6 | II-3 | II-9 | II-25 |
| Q-1348 | I-6 | II-3 | II-9 | II-26 |
| Q-1349 | I-6 | II-3 | II-9 | II-27 |
| Q-1350 | I-6 | II-3 | II-9 | II-28 |
| Q-1351 | I-6 | II-3 | II-9 | II-29 |
| Q-1352 | I-6 | II-3 | II-15 | II-21 |
| Q-1353 | I-6 | II-3 | II-15 | II-22 |
| Q-1354 | I-6 | II-3 | II-15 | II-23 |
| Q-1355 | I-6 | II-3 | II-15 | II-24 |
| Q-1356 | I-6 | II-3 | II-15 | II-25 |
| Q-1357 | I-6 | II-3 | II-15 | II-26 |
| Q-1358 | I-6 | II-3 | II-15 | II-27 |
| Q-1359 | I-6 | II-3 | II-15 | II-28 |
| Q-1360 | I-6 | II-3 | II-15 | II-29 |
| Q-1361 | I-6 | II-3 | II-21 | II-22 |
| Q-1362 | I-6 | II-3 | II-21 | II-23 |
| Q-1363 | I-6 | II-3 | II-21 | II-24 |
| Q-1364 | I-6 | II-3 | II-21 | II-25 |
| Q-1365 | I-6 | II-3 | II-21 | II-26 |
| Q-1366 | I-6 | II-3 | II-21 | II-27 |
| Q-1367 | I-6 | II-3 | II-21 | II-28 |
| Q-1368 | I-6 | II-3 | II-21 | II-29 |
| Q-1369 | I-6 | II-3 | II-22 | II-23 |
| Q-1370 | I-6 | II-3 | II-22 | II-24 |
| Q-1371 | I-6 | II-3 | II-22 | II-25 |
| Q-1372 | I-6 | II-3 | II-22 | II-26 |
| Q-1373 | I-6 | II-3 | II-22 | II-27 |
| Q-1374 | I-6 | II-3 | II-22 | II-28 |
| Q-1375 | I-6 | II-3 | II-22 | II-29 |
| Q-1376 | I-6 | II-3 | II-23 | II-24 |
| Q-1377 | I-6 | II-3 | II-23 | II-25 |
| Q-1378 | I-6 | II-3 | II-23 | II-26 |
| Q-1379 | I-6 | II-3 | II-23 | II-27 |
| Q-1380 | I-6 | II-3 | II-23 | II-28 |
| Q-1381 | I-6 | II-3 | II-23 | II-29 |
| Q-1382 | I-6 | II-3 | II-24 | II-25 |
| Q-1383 | I-6 | II-3 | II-24 | II-26 |
| Q-1384 | I-6 | II-3 | II-24 | II-27 |
| Q-1385 | I-6 | II-3 | II-24 | II-28 |
| Q-1386 | I-6 | II-3 | II-24 | II-29 |
| Q-1387 | I-6 | II-3 | II-25 | II-26 |
| Q-1388 | I-6 | II-3 | II-25 | II-27 |
| Q-1389 | I-6 | II-3 | II-25 | II-28 |
| Q-1390 | I-6 | II-3 | II-25 | II-29 |
| Q-1391 | I-6 | II-3 | II-26 | II-27 |
| Q-1392 | I-6 | II-3 | II-26 | II-28 |
| Q-1393 | I-6 | II-3 | II-26 | II-29 |
| Q-1394 | I-6 | II-3 | II-27 | II-28 |
| Q-1395 | I-6 | II-3 | II-27 | II-29 |
| Q-1396 | I-6 | II-3 | II-28 | II-29 |
| Q-1397 | I-6 | II-7 | II-8 | II-9 |
| Q-1398 | I-6 | II-7 | II-8 | II-15 |
| Q-1399 | I-6 | II-7 | II-8 | II-21 |
| Q-1400 | I-6 | II-7 | II-8 | II-22 |
| Q-1401 | I-6 | II-7 | II-8 | II-23 |
| Q-1402 | I-6 | II-7 | II-8 | II-24 |
| Q-1403 | I-6 | II-7 | II-8 | II-25 |
| Q-1404 | I-6 | II-7 | II-8 | II-26 |
| Q-1405 | I-6 | II-7 | II-8 | II-27 |
| Q-1406 | I-6 | II-7 | II-8 | II-28 |
| Q-1407 | I-6 | II-7 | II-8 | II-29 |
| Q-1408 | I-6 | II-7 | II-9 | II-15 |
| Q-1409 | I-6 | II-7 | II-9 | II-21 |
| Q-1410 | I-6 | II-7 | II-9 | II-22 |
| Q-1411 | I-6 | II-7 | II-9 | II-23 |
| Q-1412 | I-6 | II-7 | II-9 | II-24 |
| Q-1413 | I-6 | II-7 | II-9 | II-25 |
| Q-1414 | I-6 | II-7 | II-9 | II-26 |
| Q-1415 | I-6 | II-7 | II-9 | II-27 |
| Q-1416 | I-6 | II-7 | II-9 | II-28 |
| Q-1417 | I-6 | II-7 | II-9 | II-29 |
| Q-1418 | I-6 | II-7 | II-15 | II-21 |
| Q-1419 | I-6 | II-7 | II-15 | II-22 |
| Q-1420 | I-6 | II-7 | II-15 | II-23 |
| Q-1421 | I-6 | II-7 | II-15 | II-24 |
| Q-1422 | I-6 | II-7 | II-15 | II-25 |
| Q-1423 | I-6 | II-7 | II-15 | II-26 |
| Q-1424 | I-6 | II-7 | II-15 | II-27 |
| Q-1425 | I-6 | II-7 | II-15 | II-28 |
| Q-1426 | I-6 | II-7 | II-15 | II-29 |
| Q-1427 | I-6 | II-7 | II-21 | II-22 |
| Q-1428 | I-6 | II-7 | II-21 | II-23 |
| Q-1429 | I-6 | II-7 | II-21 | II-24 |
| Q-1430 | I-6 | II-7 | II-21 | II-25 |
| Q-1431 | I-6 | II-7 | II-21 | II-26 |
| Q-1432 | I-6 | II-7 | II-21 | II-27 |
| Q-1433 | I-6 | II-7 | II-21 | II-28 |
| Q-1434 | I-6 | II-7 | II-21 | II-29 |
| Q-1435 | I-6 | II-7 | II-22 | II-23 |
| Q-1436 | I-6 | II-7 | II-22 | II-24 |
| Q-1437 | I-6 | II-7 | II-22 | II-25 |
| Q-1438 | I-6 | II-7 | II-22 | II-26 |
| Q-1439 | I-6 | II-7 | II-22 | II-27 |
| Q-1440 | I-6 | II-7 | II-22 | II-28 |
| Q-1441 | I-6 | II-7 | II-22 | II-29 |
| Q-1442 | I-6 | II-7 | II-23 | II-24 |
| Q-1443 | I-6 | II-7 | II-23 | II-25 |
| Q-1444 | I-6 | II-7 | II-23 | II-26 |
| Q-1445 | I-6 | II-7 | II-23 | II-27 |
| Q-1446 | I-6 | II-7 | II-23 | II-28 |
| Q-1447 | I-6 | II-7 | II-23 | II-29 |
| Q-1448 | I-6 | II-7 | II-24 | II-24 |
| Q-1449 | I-6 | II-7 | II-24 | II-25 |
| Q-1450 | I-6 | II-7 | II-24 | II-26 |
| Q-1451 | I-6 | II-7 | II-24 | II-27 |
| Q-1452 | I-6 | II-7 | II-24 | II-28 |
| Q-1453 | I-6 | II-7 | II-24 | II-29 |
| Q-1454 | I-6 | II-7 | II-25 | II-26 |
| Q-1455 | I-6 | II-7 | II-25 | II-27 |
| Q-1456 | I-6 | II-7 | II-25 | II-28 |
| Q-1457 | I-6 | II-7 | II-25 | II-29 |
| Q-1458 | I-6 | II-7 | II-26 | II-27 |
| Q-1459 | I-6 | II-7 | II-26 | II-28 |
| Q-1460 | I-6 | II-7 | II-26 | II-29 |
| Q-1461 | I-6 | II-7 | II-27 | II-28 |
| Q-1462 | I-6 | II-7 | II-27 | II-29 |
| Q-1463 | I-6 | II-7 | II-28 | II-29 |
| Q-1464 | I-6 | II-8 | II-9 | II-15 |
| Q-1465 | I-6 | II-8 | II-9 | II-21 |
| Q-1466 | I-6 | II-8 | II-9 | II-22 |
| Q-1467 | I-6 | II-8 | II-9 | II-23 |
| Q-1468 | I-6 | II-8 | II-9 | II-24 |
| Q-1469 | I-6 | II-8 | II-9 | II-25 |
| Q-1470 | I-6 | II-8 | II-9 | II-26 |
| Q-1471 | I-6 | II-8 | II-9 | II-27 |
| Q-1472 | I-6 | II-8 | II-9 | II-28 |
| Q-1473 | I-6 | II-8 | II-9 | II-29 |
| Q-1474 | I-6 | II-8 | II-15 | II-21 |
| Q-1475 | I-6 | II-8 | II-15 | II-22 |
| Q-1476 | I-6 | II-8 | II-15 | II-23 |
| Q-1477 | I-6 | II-8 | II-15 | II-24 |
| Q-1478 | I-6 | II-8 | II-15 | II-25 |
| Q-1479 | I-6 | II-8 | II-15 | II-26 |
| Q-1480 | I-6 | II-8 | II-15 | II-27 |
| Q-1481 | I-6 | II-8 | II-15 | II-28 |
| Q-1482 | I-6 | II-8 | II-15 | II-29 |
| Q-1483 | I-6 | II-8 | II-21 | II-22 |
| Q-1484 | I-6 | II-8 | II-21 | II-23 |
| Q-1485 | I-6 | II-8 | II-21 | II-24 |
| Q-1486 | I-6 | II-8 | II-21 | II-25 |
| Q-1487 | I-6 | II-8 | II-21 | II-26 |
| Q-1488 | I-6 | II-8 | II-21 | II-27 |
| Q-1489 | I-6 | II-8 | II-21 | II-28 |
| Q-1490 | I-6 | II-8 | II-21 | II-29 |
| Q-1491 | I-6 | II-8 | II-22 | II-23 |
| Q-1492 | I-6 | II-8 | II-22 | II-24 |
| Q-1493 | I-6 | II-8 | II-22 | II-25 |
| Q-1494 | I-6 | II-8 | II-22 | II-26 |
| Q-1495 | I-6 | II-8 | II-22 | II-27 |
| Q-1496 | I-6 | II-8 | II-22 | II-28 |
| Q-1497 | I-6 | II-8 | II-22 | II-29 |
| Q-1498 | I-6 | II-8 | II-23 | II-24 |
| Q-1499 | I-6 | II-8 | II-23 | II-25 |
| Q-1500 | I-6 | II-8 | II-23 | II-26 |
| Q-1501 | I-6 | II-8 | II-23 | II-27 |
| Q-1502 | I-6 | II-8 | II-23 | II-28 |
| Q-1503 | I-6 | II-8 | II-23 | II-29 |
| Q-1504 | I-6 | II-8 | II-24 | II-25 |
| Q-1505 | I-6 | II-8 | II-24 | II-26 |
| Q-1506 | I-6 | II-8 | II-24 | II-27 |
| Q-1507 | I-6 | II-8 | II-24 | II-28 |
| Q-1508 | I-6 | II-8 | II-24 | II-29 |
| Q-1509 | I-6 | II-8 | II-25 | II-26 |
| Q-1510 | I-6 | II-8 | II-25 | II-27 |
| Q-1511 | I-6 | II-8 | II-25 | II-28 |
| Q-1512 | I-6 | II-8 | II-25 | II-29 |
| Q-1513 | I-6 | II-8 | II-26 | II-27 |
| Q-1514 | I-6 | II-8 | II-26 | II-28 |
| Q-1515 | I-6 | II-8 | II-26 | II-29 |
| Q-1516 | I-6 | II-8 | II-27 | II-28 |
| Q-1517 | I-6 | II-8 | II-27 | II-29 |
| Q-1518 | I-6 | II-8 | II-28 | II-29 |
| Q-1519 | I-6 | II-9 | II-15 | II-21 |
| Q-1520 | I-6 | II-9 | II-15 | II-22 |
| Q-1521 | I-6 | II-9 | II-15 | II-23 |
| Q-1522 | I-6 | II-9 | II-15 | II-24 |
| Q-1523 | I-6 | II-9 | II-15 | II-25 |
| Q-1524 | I-6 | II-9 | II-15 | II-26 |
| Q-1525 | I-6 | II-9 | II-15 | II-27 |
| Q-1526 | I-6 | II-9 | II-15 | II-28 |
| Q-1527 | I-6 | II-9 | II-15 | II-29 |
| Q-1528 | I-6 | II-9 | II-21 | II-22 |
| Q-1529 | I-6 | II-9 | II-21 | II-23 |
| Q-1530 | I-6 | II-9 | II-21 | II-24 |
| Q-1531 | I-6 | II-9 | II-21 | II-25 |
| Q-1532 | I-6 | II-9 | II-21 | II-26 |
| Q-1533 | I-6 | II-9 | II-21 | II-27 |
| Q-1534 | I-6 | II-9 | II-21 | II-28 |
| Q-1535 | I-6 | II-9 | II-21 | II-29 |
| Q-1536 | I-6 | II-9 | II-22 | II-23 |
| Q-1537 | I-6 | II-9 | II-22 | II-24 |
| Q-1538 | I-6 | II-9 | II-22 | II-25 |
| Q-1539 | I-6 | II-9 | II-22 | II-26 |
| Q-1540 | I-6 | II-9 | II-22 | II-27 |
| Q-1541 | I-6 | II-9 | II-22 | II-28 |
| Q-1542 | I-6 | II-9 | II-22 | II-29 |
| Q-1543 | I-6 | II-9 | II-23 | II-24 |
| Q-1544 | I-6 | II-9 | II-23 | II-25 |
| Q-1545 | I-6 | II-9 | II-23 | II-26 |
| Q-1546 | I-6 | II-9 | II-23 | II-27 |
| Q-1547 | I-6 | II-9 | II-23 | II-28 |
| Q-1548 | I-6 | II-9 | II-23 | II-29 |
| Q-1549 | I-6 | II-9 | II-24 | II-25 |
| Q-1550 | I-6 | II-9 | II-24 | II-26 |
| Q-1551 | I-6 | II-9 | II-24 | II-27 |
| Q-1552 | I-6 | II-9 | II-24 | II-28 |
| Q-1553 | I-6 | II-9 | II-24 | II-29 |
| Q-1554 | I-6 | II-9 | II-25 | II-26 |
| Q-1555 | I-6 | II-9 | II-25 | II-27 |
| Q-1556 | I-6 | II-9 | II-25 | II-28 |
| Q-1557 | I-6 | II-9 | II-25 | II-29 |
| Q-1558 | I-6 | II-9 | II-26 | II-27 |
| Q-1559 | I-6 | II-9 | II-26 | II-28 |
| Q-1560 | I-6 | II-9 | II-26 | II-29 |
| Q-1561 | I-6 | II-9 | II-27 | II-28 |
| Q-1562 | I-6 | II-9 | II-27 | II-29 |
| Q-1563 | I-6 | II-9 | II-28 | II-29 |
| Q-1564 | I-6 | II-15 | II-21 | II-22 |
| Q-1565 | I-6 | II-15 | II-21 | II-23 |
| Q-1566 | I-6 | II-15 | II-21 | II-24 |
| Q-1567 | I-6 | II-15 | II-21 | II-25 |
| Q-1568 | I-6 | II-15 | II-21 | II-26 |
| Q-1569 | I-6 | II-15 | II-21 | II-27 |
| Q-1570 | I-6 | II-15 | II-21 | II-28 |
| Q-1571 | I-6 | II-15 | II-21 | II-29 |
| Q-1572 | I-6 | II-15 | II-22 | II-23 |
| Q-1573 | I-6 | II-15 | II-22 | II-24 |
| Q-1574 | I-6 | II-15 | II-22 | II-25 |
| Q-1575 | I-6 | II-15 | II-22 | II-26 |
| Q-1576 | I-6 | II-15 | II-22 | II-27 |
| Q-1577 | I-6 | II-15 | II-22 | II-28 |
| Q-1578 | I-6 | II-15 | II-22 | II-29 |
| Q-1579 | I-6 | II-15 | II-23 | II-24 |
| Q-1580 | I-6 | II-15 | II-23 | II-25 |
| Q-1581 | I-6 | II-15 | II-23 | II-26 |
| Q-1582 | I-6 | II-15 | II-23 | II-27 |
| Q-1583 | I-6 | II-15 | II-23 | II-28 |
| Q-1584 | I-6 | II-15 | II-23 | II-29 |
| Q-1585 | I-6 | II-15 | II-24 | II-25 |
| Q-1586 | I-6 | II-15 | II-24 | II-26 |
| Q-1587 | I-6 | II-15 | II-24 | II-27 |
| Q-1588 | I-6 | II-15 | II-24 | II-28 |
| Q-1589 | I-6 | II-15 | II-24 | II-29 |
| Q-1590 | I-6 | II-15 | II-25 | II-26 |
| Q-1591 | I-6 | II-15 | II-25 | II-27 |
| Q-1592 | I-6 | II-15 | II-25 | II-28 |
| Q-1593 | I-6 | II-15 | II-25 | II-29 |
| Q-1594 | I-6 | II-15 | II-26 | II-27 |
| Q-1595 | I-6 | II-15 | II-26 | II-28 |
| Q-1596 | I-6 | II-15 | II-26 | II-29 |
| Q-1597 | I-6 | II-15 | II-27 | II-28 |
| Q-1598 | I-6 | II-15 | II-27 | II-29 |
| Q-1599 | I-6 | II-15 | II-28 | II-29 |
| Q-1600 | I-6 | II-21 | II-22 | II-23 |
| Q-1601 | I-6 | II-21 | II-22 | II-24 |
| Q-1602 | I-6 | II-21 | II-22 | II-25 |
| Q-1603 | I-6 | II-21 | II-22 | II-26 |
| Q-1604 | I-6 | II-21 | II-22 | II-27 |
| Q-1605 | I-6 | II-21 | II-22 | II-28 |
| Q-1606 | I-6 | II-21 | II-22 | II-29 |
| Q-1607 | I-6 | II-21 | II-23 | II-24 |
| Q-1608 | I-6 | II-21 | II-23 | II-25 |
| Q-1609 | I-6 | II-21 | II-23 | II-26 |
| Q-1610 | I-6 | II-21 | II-23 | II-27 |
| Q-1611 | I-6 | II-21 | II-23 | II-28 |
| Q-1612 | I-6 | II-21 | II-23 | II-29 |
| Q-1613 | I-6 | II-21 | II-24 | II-25 |
| Q-1614 | I-6 | II-21 | II-24 | II-26 |
| Q-1615 | I-6 | II-21 | II-24 | II-27 |
| Q-1616 | I-6 | II-21 | II-24 | II-28 |
| Q-1617 | I-6 | II-21 | II-24 | II-29 |
| Q-1618 | I-6 | II-21 | II-25 | II-26 |
| Q-1619 | I-6 | II-21 | II-25 | II-27 |
| Q-1620 | I-6 | II-21 | II-25 | II-28 |
| Q-1621 | I-6 | II-21 | II-25 | II-29 |
| Q-1622 | I-6 | II-21 | II-26 | II-27 |
| Q-1623 | I-6 | II-21 | II-26 | II-28 |
| Q-1624 | I-6 | II-21 | II-26 | II-29 |
| Q-1625 | I-6 | II-21 | II-27 | II-28 |
| Q-1626 | I-6 | II-21 | II-27 | II-29 |
| Q-1627 | I-6 | II-21 | II-28 | II-29 |
| Q-1628 | I-6 | II-22 | II-23 | II-24 |
| Q-1629 | I-6 | II-22 | II-23 | II-25 |
| Q-1630 | I-6 | II-22 | II-23 | II-26 |
| Q-1631 | I-6 | II-22 | II-23 | II-27 |
| Q-1632 | I-6 | II-22 | II-23 | II-28 |
| Q-1633 | I-6 | II-22 | II-23 | II-29 |
| Q-1634 | I-6 | II-22 | II-24 | II-25 |
| Q-1635 | I-6 | II-22 | II-24 | II-26 |
| Q-1636 | I-6 | II-22 | II-24 | II-27 |
| Q-1637 | I-6 | II-22 | II-24 | II-28 |
| Q-1638 | I-6 | II-22 | II-24 | II-29 |
| Q-1639 | I-6 | II-22 | II-25 | II-26 |
| Q-1640 | I-6 | II-22 | II-25 | II-27 |
| Q-1641 | I-6 | II-22 | II-25 | II-28 |
| Q-1642 | I-6 | II-22 | II-25 | II-29 |
| Q-1643 | I-6 | II-22 | II-26 | II-27 |
| Q-1644 | I-6 | II-22 | II-26 | II-28 |
| Q-1645 | I-6 | II-22 | II-26 | II-29 |
| Q-1646 | I-6 | II-22 | II-27 | II-28 |
| Q-1647 | I-6 | II-22 | II-27 | II-29 |
| Q-1648 | I-6 | II-22 | II-28 | II-29 |
| Q-1649 | I-6 | II-23 | II-24 | II-25 |
| Q-1650 | I-6 | II-23 | II-24 | II-26 |
| Q-1651 | I-6 | II-23 | II-24 | II-27 |
| Q-1652 | I-6 | II-23 | II-24 | II-28 |
| Q-1653 | I-6 | II-23 | II-24 | II-29 |
| Q-1654 | I-6 | II-23 | II-25 | II-26 |
| Q-1655 | I-6 | II-23 | II-25 | II-27 |
| Q-1656 | I-6 | II-23 | II-25 | II-28 |
| Q-1657 | I-6 | II-23 | II-25 | II-29 |
| Q-1658 | I-6 | II-23 | II-26 | II-27 |
| Q-1659 | I-6 | II-23 | II-26 | II-28 |
| Q-1660 | I-6 | II-23 | II-26 | II-29 |
| Q-1661 | I-6 | II-23 | II-27 | II-28 |
| Q-1662 | I-6 | II-23 | II-27 | II-29 |
| Q-1663 | I-6 | II-23 | II-28 | II-29 |
| Q-1664 | I-6 | II-24 | II-25 | II-26 |
| Q-1665 | I-6 | II-24 | II-25 | II-27 |
| Q-1666 | I-6 | II-24 | II-25 | II-28 |
| Q-1667 | I-6 | II-24 | II-25 | II-29 |
| Q-1668 | I-6 | II-24 | II-26 | II-27 |
| Q-1669 | I-6 | II-24 | II-26 | II-28 |
| Q-1670 | I-6 | II-24 | II-26 | II-29 |
| Q-1671 | I-6 | II-24 | II-27 | II-28 |
| Q-1672 | I-6 | II-24 | II-27 | II-29 |
| Q-1673 | I-6 | II-24 | II-28 | II-29 |
| Q-1674 | I-6 | II-25 | II-26 | II-27 |
| Q-1675 | I-6 | II-25 | II-26 | II-28 |
| Q-1676 | I-6 | II-25 | II-26 | II-29 |
| Q-1677 | I-6 | II-25 | II-27 | II-28 |
| Q-1678 | I-6 | II-25 | II-27 | II-29 |
| Q-1679 | I-6 | II-25 | II-28 | II-29 |
| Q-1680 | I-6 | II-26 | II-27 | II-28 |
| Q-1681 | I-6 | II-26 | II-27 | II-29 |
| Q-1682 | I-6 | II-26 | II-28 | II-29 |
| Q-1683 | I-6 | II-27 | II-28 | II-29 |
| Q-1684 | I-18 | II-1 | II-2 | II-3 |
| Q-1685 | I-18 | II-1 | II-2 | II-7 |
| Q-1686 | I-18 | II-1 | II-2 | II-8 |
| Q-1687 | I-18 | II-1 | II-2 | II-9 |
| Q-1688 | I-18 | II-1 | II-2 | II-15 |
| Q-1689 | I-18 | II-1 | II-2 | II-21 |
| Q-1690 | I-18 | II-1 | II-2 | II-22 |
| Q-1691 | I-18 | II-1 | II-2 | II-23 |
| Q-1692 | I-18 | II-1 | II-2 | II-24 |
| Q-1693 | I-18 | II-1 | II-2 | II-25 |
| Q-1694 | I-18 | II-1 | II-2 | II-26 |
| Q-1695 | I-18 | II-1 | II-2 | II-27 |
| Q-1696 | I-18 | II-1 | II-2 | II-28 |
| Q-1697 | I-18 | II-1 | II-2 | II-29 |
| Q-1698 | I-18 | II-1 | II-3 | II-7 |
| Q-1699 | I-18 | II-1 | II-3 | II-8 |
| Q-1700 | I-18 | II-1 | II-3 | II-9 |
| Q-1701 | I-18 | II-1 | II-3 | II-15 |
| Q-1702 | I-18 | II-1 | II-3 | II-21 |
| Q-1703 | I-18 | II-1 | II-3 | II-22 |
| Q-1704 | I-18 | II-1 | II-3 | II-23 |
| Q-1705 | I-18 | II-1 | II-3 | II-24 |
| Q-1706 | I-18 | II-1 | II-3 | II-25 |
| Q-1707 | I-18 | II-1 | II-3 | II-26 |
| Q-1708 | I-18 | II-1 | II-3 | II-27 |
| Q-1709 | I-18 | II-1 | II-3 | II-28 |
| Q-1710 | I-18 | II-1 | II-3 | II-29 |
| Q-1711 | I-18 | II-1 | II-7 | II-8 |
| Q-1712 | I-18 | II-1 | II-7 | II-9 |
| Q-1713 | I-18 | II-1 | II-7 | II-15 |
| Q-1714 | I-18 | II-1 | II-7 | II-21 |
| Q-1715 | I-18 | II-1 | II-7 | II-22 |
| Q-1716 | I-18 | II-1 | II-7 | II-23 |
| Q-1717 | I-18 | II-1 | II-7 | II-24 |
| Q-1718 | I-18 | II-1 | II-7 | II-25 |
| Q-1719 | I-18 | II-1 | II-7 | II-26 |
| Q-1720 | I-18 | II-1 | II-7 | II-27 |
| Q-1721 | I-18 | II-1 | II-7 | II-28 |
| Q-1722 | I-18 | II-1 | II-7 | II-29 |
| Q-1723 | I-18 | II-1 | II-8 | II-9 |
| Q-1724 | I-18 | II-1 | II-8 | II-15 |
| Q-1725 | I-18 | II-1 | II-8 | II-21 |
| Q-1726 | I-18 | II-1 | II-8 | II-22 |
| Q-1727 | I-18 | II-1 | II-8 | II-23 |
| Q-1728 | I-18 | II-1 | II-8 | II-24 |
| Q-1729 | I-18 | II-1 | II-8 | II-25 |
| Q-1730 | I-18 | II-1 | II-8 | II-26 |
| Q-1731 | I-18 | II-1 | II-8 | II-27 |
| Q-1732 | I-18 | II-1 | II-8 | II-28 |
| Q-1733 | I-18 | II-1 | II-8 | II-29 |
| Q-1734 | I-18 | II-1 | II-9 | II-15 |
| Q-1735 | I-18 | II-1 | II-9 | II-21 |
| Q-1736 | I-18 | II-1 | II-9 | II-22 |
| Q-1737 | I-18 | II-1 | II-9 | II-23 |
| Q-1738 | I-18 | II-1 | II-9 | II-24 |
| Q-1739 | I-18 | II-1 | II-9 | II-25 |
| Q-1740 | I-18 | II-1 | II-9 | II-26 |
| Q-1741 | I-18 | II-1 | II-9 | II-27 |
| Q-1742 | I-18 | II-1 | II-9 | II-28 |
| Q-1743 | I-18 | II-1 | II-9 | II-29 |
| Q-1744 | I-18 | II-1 | II-15 | II-21 |
| Q-1745 | I-18 | II-1 | II-15 | II-22 |
| Q-1746 | I-18 | II-1 | II-15 | II-23 |
| Q-1747 | I-18 | II-1 | II-15 | II-24 |
| Q-1748 | I-18 | II-1 | II-15 | II-25 |
| Q-1749 | I-18 | II-1 | II-15 | II-26 |
| Q-1750 | I-18 | II-1 | II-15 | II-27 |
| Q-1751 | I-18 | II-1 | II-15 | II-28 |
| Q-1752 | I-18 | II-1 | II-15 | II-29 |
| Q-1753 | I-18 | II-1 | II-21 | II-22 |
| Q-1754 | I-18 | II-1 | II-21 | II-23 |
| Q-1755 | I-18 | II-1 | II-21 | II-24 |
| Q-1756 | I-18 | II-1 | II-21 | II-25 |
| Q-1757 | I-18 | II-1 | II-21 | II-26 |
| Q-1758 | I-18 | II-1 | II-21 | II-27 |
| Q-1759 | I-18 | II-1 | II-21 | II-28 |
| Q-1760 | I-18 | II-1 | II-21 | II-29 |
| Q-1761 | I-18 | II-1 | II-22 | II-23 |
| Q-1762 | I-18 | II-1 | II-22 | II-24 |
| Q-1763 | I-18 | II-1 | II-22 | II-25 |
| Q-1764 | I-18 | II-1 | II-22 | II-26 |
| Q-1765 | I-18 | II-1 | II-22 | II-27 |
| Q-1766 | I-18 | II-1 | II-22 | II-28 |
| Q-1767 | I-18 | II-1 | II-22 | II-29 |
| Q-1768 | I-18 | II-1 | II-23 | II-24 |
| Q-1769 | I-18 | II-1 | II-23 | II-25 |
| Q-1770 | I-18 | II-1 | II-23 | II-26 |
| Q-1771 | I-18 | II-1 | II-23 | II-27 |
| Q-1772 | I-18 | II-1 | II-23 | II-28 |
| Q-1773 | I-18 | II-1 | II-23 | II-29 |
| Q-1774 | I-18 | II-1 | II-24 | II-25 |
| Q-1775 | I-18 | II-1 | II-24 | II-26 |
| Q-1776 | I-18 | II-1 | II-24 | II-27 |
| Q-1777 | I-18 | II-1 | II-24 | II-28 |
| Q-1778 | I-18 | II-1 | II-24 | II-29 |
| Q-1779 | I-18 | II-1 | II-25 | II-26 |
| Q-1780 | I-18 | II-1 | II-25 | II-27 |
| Q-1781 | I-18 | II-1 | II-25 | II-28 |
| Q-1782 | I-18 | II-1 | II-25 | II-29 |
| Q-1783 | I-18 | II-1 | II-26 | II-27 |
| Q-1784 | I-18 | II-1 | II-26 | II-28 |
| Q-1785 | I-18 | II-1 | II-26 | II-29 |
| Q-1786 | I-18 | II-1 | II-27 | II-28 |
| Q-1787 | I-18 | II-1 | II-27 | II-29 |
| Q-1788 | I-18 | II-1 | II-28 | II-29 |
| Q-1789 | I-18 | II-2 | II-3 | II-7 |
| Q-1790 | I-18 | II-2 | II-3 | II-8 |
| Q-1791 | I-18 | II-2 | II-3 | II-9 |
| Q-1792 | I-18 | II-2 | II-3 | II-15 |
| Q-1793 | I-18 | II-2 | II-3 | II-21 |
| Q-1794 | I-18 | II-2 | II-3 | II-22 |
| Q-1795 | I-18 | II-2 | II-3 | II-23 |
| Q-1796 | I-18 | II-2 | II-3 | II-24 |
| Q-1797 | I-18 | II-2 | II-3 | II-25 |
| Q-1798 | I-18 | II-2 | II-3 | II-26 |
| Q-1799 | I-18 | II-2 | II-3 | II-27 |
| Q-1800 | I-18 | II-2 | II-3 | II-28 |
| Q-1801 | I-18 | II-2 | II-3 | II-29 |
| Q-1802 | I-18 | II-2 | II-7 | II-8 |
| Q-1803 | I-18 | II-2 | II-7 | II-9 |
| Q-1804 | I-18 | II-2 | II-7 | II-15 |
| Q-1805 | I-18 | II-2 | II-7 | II-21 |
| Q-1806 | I-18 | II-2 | II-7 | II-22 |
| Q-1807 | I-18 | II-2 | II-7 | II-23 |
| Q-1808 | I-18 | II-2 | II-7 | II-24 |
| Q-1809 | I-18 | II-2 | II-7 | II-25 |
| Q-1810 | I-18 | II-2 | II-7 | II-26 |
| Q-1811 | I-18 | II-2 | II-7 | II-27 |
| Q-1812 | I-18 | II-2 | II-7 | II-28 |
| Q-1813 | I-18 | II-2 | II-7 | II-29 |
| Q-1814 | I-18 | II-2 | II-8 | II-9 |
| Q-1815 | I-18 | II-2 | II-8 | II-15 |
| Q-1816 | I-18 | II-2 | II-8 | II-21 |
| Q-1817 | I-18 | II-2 | II-8 | II-22 |
| Q-1818 | I-18 | II-2 | II-8 | II-23 |
| Q-1819 | I-18 | II-2 | II-8 | II-24 |
| Q-1820 | I-18 | II-2 | II-8 | II-25 |
| Q-1821 | I-18 | II-2 | II-8 | II-26 |
| Q-1822 | I-18 | II-2 | II-8 | II-27 |
| Q-1823 | I-18 | II-2 | II-8 | II-28 |
| Q-1824 | I-18 | II-2 | II-8 | II-29 |
| Q-1825 | I-18 | II-2 | II-9 | II-15 |
| Q-1826 | I-18 | II-2 | II-9 | II-21 |
| Q-1827 | I-18 | II-2 | II-9 | II-22 |
| Q-1828 | I-18 | II-2 | II-9 | II-23 |
| Q-1829 | I-18 | II-2 | II-9 | II-24 |
| Q-1830 | I-18 | II-2 | II-9 | II-25 |
| Q-1831 | I-18 | II-2 | II-9 | II-26 |
| Q-1832 | I-18 | II-2 | II-9 | II-27 |
| Q-1833 | I-18 | II-2 | II-9 | II-28 |
| Q-1834 | I-18 | II-2 | II-9 | II-29 |
| Q-1835 | I-18 | II-2 | II-15 | II-21 |
| Q-1836 | I-18 | II-2 | II-15 | II-22 |
| Q-1837 | I-18 | II-2 | II-15 | II-23 |
| Q-1838 | I-18 | II-2 | II-15 | II-24 |
| Q-1839 | I-18 | II-2 | II-15 | II-25 |
| Q-1840 | I-18 | II-2 | II-15 | II-26 |
| Q-1841 | I-18 | II-2 | II-15 | II-27 |
| Q-1842 | I-18 | II-2 | II-15 | II-28 |
| Q-1843 | I-18 | II-2 | II-15 | II-29 |
| Q-1844 | I-18 | II-2 | II-21 | II-22 |
| Q-1845 | I-18 | II-2 | II-21 | II-23 |
| Q-1846 | I-18 | II-2 | II-21 | II-24 |
| Q-1847 | I-18 | II-2 | II-21 | II-25 |
| Q-1848 | I-18 | II-2 | II-21 | II-26 |
| Q-1849 | I-18 | II-2 | II-21 | II-27 |
| Q-1850 | I-18 | II-2 | II-21 | II-28 |
| Q-1851 | I-18 | II-2 | II-21 | II-29 |
| Q-1852 | I-18 | II-2 | II-22 | II-23 |
| Q-1853 | I-18 | II-2 | II-22 | II-24 |
| Q-1854 | I-18 | II-2 | II-22 | II-25 |
| Q-1855 | I-18 | II-2 | II-22 | II-26 |
| Q-1856 | I-18 | II-2 | II-22 | II-27 |
| Q-1857 | I-18 | II-2 | II-22 | II-28 |
| Q-1858 | I-18 | II-2 | II-22 | II-29 |
| Q-1859 | I-18 | II-2 | II-23 | II-24 |
| Q-1860 | I-18 | II-2 | II-23 | II-25 |
| Q-1861 | I-18 | II-2 | II-23 | II-26 |
| Q-1862 | I-18 | II-2 | II-23 | II-27 |
| Q-1863 | I-18 | II-2 | II-23 | II-28 |
| Q-1864 | I-18 | II-2 | II-23 | II-29 |
| Q-1865 | I-18 | II-2 | II-24 | II-25 |
| Q-1866 | I-18 | II-2 | II-24 | II-26 |
| Q-1867 | I-18 | II-2 | II-24 | II-27 |
| Q-1868 | I-18 | II-2 | II-24 | II-28 |
| Q-1869 | I-18 | II-2 | II-24 | II-29 |
| Q-1870 | I-18 | II-2 | II-25 | II-26 |
| Q-1871 | I-18 | II-2 | II-25 | II-27 |
| Q-1872 | I-18 | II-2 | II-25 | II-28 |
| Q-1873 | I-18 | II-2 | II-25 | II-29 |
| Q-1874 | I-18 | II-2 | II-26 | II-27 |
| Q-1875 | I-18 | II-2 | II-26 | II-28 |
| Q-1876 | I-18 | II-2 | II-26 | II-29 |
| Q-1877 | I-18 | II-2 | II-27 | II-28 |
| Q-1878 | I-18 | II-2 | II-27 | II-29 |
| Q-1879 | I-18 | II-2 | II-28 | II-29 |
| Q-1880 | I-18 | II-3 | II-7 | II-8 |
| Q-1881 | I-18 | II-3 | II-7 | II-9 |
| Q-1882 | I-18 | II-3 | II-7 | II-15 |
| Q-1883 | I-18 | II-3 | II-7 | II-21 |
| Q-1884 | I-18 | II-3 | II-7 | II-22 |
| Q-1885 | I-18 | II-3 | II-7 | II-23 |
| Q-1886 | I-18 | II-3 | II-7 | II-24 |
| Q-1887 | I-18 | II-3 | II-7 | II-25 |
| Q-1888 | I-18 | II-3 | II-7 | II-26 |
| Q-1889 | I-18 | II-3 | II-7 | II-27 |
| Q-1890 | I-18 | II-3 | II-7 | II-28 |
| Q-1891 | I-18 | II-3 | II-7 | II-29 |
| Q-1892 | I-18 | II-3 | II-8 | II-9 |
| Q-1893 | I-18 | II-3 | II-8 | II-15 |
| Q-1894 | I-18 | II-3 | II-8 | II-21 |
| Q-1895 | I-18 | II-3 | II-8 | II-22 |
| Q-1896 | I-18 | II-3 | II-8 | II-23 |
| Q-1897 | I-18 | II-3 | II-8 | II-24 |
| Q-1898 | I-18 | II-3 | II-8 | II-25 |
| Q-1899 | I-18 | II-3 | II-8 | II-26 |
| Q-1900 | I-18 | II-3 | II-8 | II-27 |
| Q-1901 | I-18 | II-3 | II-8 | II-28 |
| Q-1902 | I-18 | II-3 | II-8 | II-29 |
| Q-1903 | I-18 | II-3 | II-9 | II-15 |
| Q-1904 | I-18 | II-3 | II-9 | II-21 |
| Q-1905 | I-18 | II-3 | II-9 | II-22 |
| Q-1906 | I-18 | II-3 | II-9 | II-23 |
| Q-1907 | I-18 | II-3 | II-9 | II-24 |
| Q-1908 | I-18 | II-3 | II-9 | II-25 |
| Q-1909 | I-18 | II-3 | II-9 | II-26 |
| Q-1910 | I-18 | II-3 | II-9 | II-27 |
| Q-1911 | I-18 | II-3 | II-9 | II-28 |
| Q-1912 | I-18 | II-3 | II-9 | II-29 |
| Q-1913 | I-18 | II-3 | II-15 | II-21 |
| Q-1914 | I-18 | II-3 | II-15 | II-22 |
| Q-1915 | I-18 | II-3 | II-15 | II-23 |
| Q-1916 | I-18 | II-3 | II-15 | II-24 |
| Q-1917 | I-18 | II-3 | II-15 | II-25 |
| Q-1918 | I-18 | II-3 | II-15 | II-26 |
| Q-1919 | I-18 | II-3 | II-15 | II-27 |
| Q-1920 | I-18 | II-3 | II-15 | II-28 |
| Q-1921 | I-18 | II-3 | II-15 | II-29 |
| Q-1922 | I-18 | II-3 | II-21 | II-22 |
| Q-1923 | I-18 | II-3 | II-21 | II-23 |
| Q-1924 | I-18 | II-3 | II-21 | II-24 |
| Q-1925 | I-18 | II-3 | II-21 | II-25 |
| Q-1926 | I-18 | II-3 | II-21 | II-26 |
| Q-1927 | I-18 | II-3 | II-21 | II-27 |
| Q-1928 | I-18 | II-3 | II-21 | II-28 |
| Q-1929 | I-18 | II-3 | II-21 | II-29 |
| Q-1930 | I-18 | II-3 | II-22 | II-23 |
| Q-1931 | I-18 | II-3 | II-22 | II-24 |
| Q-1932 | I-18 | II-3 | II-22 | II-25 |
| Q-1933 | I-18 | II-3 | II-22 | II-26 |
| Q-1934 | I-18 | II-3 | II-22 | II-27 |
| Q-1935 | I-18 | II-3 | II-22 | II-28 |
| Q-1936 | I-18 | II-3 | II-22 | II-29 |
| Q-1937 | I-18 | II-3 | II-23 | II-24 |
| Q-1938 | I-18 | II-3 | II-23 | II-25 |
| Q-1939 | I-18 | II-3 | II-23 | II-26 |
| Q-1940 | I-18 | II-3 | II-23 | II-27 |
| Q-1941 | I-18 | II-3 | II-23 | II-28 |
| Q-1942 | I-18 | II-3 | II-23 | II-29 |
| Q-1943 | I-18 | II-3 | II-24 | II-25 |
| Q-1944 | I-18 | II-3 | II-24 | II-26 |
| Q-1945 | I-18 | II-3 | II-24 | II-27 |
| Q-1946 | I-18 | II-3 | II-24 | II-28 |
| Q-1947 | I-18 | II-3 | II-24 | II-29 |
| Q-1948 | I-18 | II-3 | II-25 | II-26 |
| Q-1949 | I-18 | II-3 | II-25 | II-27 |
| Q-1950 | I-18 | II-3 | II-25 | II-28 |
| Q-1951 | I-18 | II-3 | II-25 | II-29 |
| Q-1952 | I-18 | II-3 | II-26 | II-27 |
| Q-1953 | I-18 | II-3 | II-26 | II-28 |
| Q-1954 | I-18 | II-3 | II-26 | II-29 |
| Q-1955 | I-18 | II-3 | II-27 | II-28 |
| Q-1956 | I-18 | II-3 | II-27 | II-29 |
| Q-1957 | I-18 | II-3 | II-28 | II-29 |
| Q-1958 | I-18 | II-7 | II-8 | II-9 |
| Q-1959 | I-18 | II-7 | II-8 | II-15 |
| Q-1960 | I-18 | II-7 | II-8 | II-21 |
| Q-1961 | I-18 | II-7 | II-8 | II-22 |
| Q-1962 | I-18 | II-7 | II-8 | II-23 |
| Q-1963 | I-18 | II-7 | II-8 | II-24 |
| Q-1964 | I-18 | II-7 | II-8 | II-25 |
| Q-1965 | I-18 | II-7 | II-8 | II-26 |
| Q-1966 | I-18 | II-7 | II-8 | II-27 |
| Q-1967 | I-18 | II-7 | II-8 | II-28 |
| Q-1968 | I-18 | II-7 | II-8 | II-29 |
| Q-1969 | I-18 | II-7 | II-9 | II-15 |
| Q-1970 | I-18 | II-7 | II-9 | II-21 |
| Q-1971 | I-18 | II-7 | II-9 | II-22 |
| Q-1972 | I-18 | II-7 | II-9 | II-23 |
| Q-1973 | I-18 | II-7 | II-9 | II-24 |
| Q-1974 | I-18 | II-7 | II-9 | II-25 |
| Q-1975 | I-18 | II-7 | II-9 | II-26 |
| Q-1976 | I-18 | II-7 | II-9 | II-27 |
| Q-1977 | I-18 | II-7 | II-9 | II-28 |
| Q-1978 | I-18 | II-7 | II-9 | II-29 |
| Q-1979 | I-18 | II-7 | II-15 | II-21 |
| Q-1980 | I-18 | II-7 | II-15 | II-22 |
| Q-1981 | I-18 | II-7 | II-15 | II-23 |
| Q-1982 | I-18 | II-7 | II-15 | II-24 |
| Q-1983 | I-18 | II-7 | II-15 | II-25 |
| Q-1984 | I-18 | II-7 | II-15 | II-26 |
| Q-1985 | I-18 | II-7 | II-15 | II-27 |
| Q-1986 | I-18 | II-7 | II-15 | II-28 |
| Q-1987 | I-18 | II-7 | II-15 | II-29 |
| Q-1988 | I-18 | II-7 | II-21 | II-22 |
| Q-1989 | I-18 | II-7 | II-21 | II-23 |
| Q-1990 | I-18 | II-7 | II-21 | II-24 |
| Q-1991 | I-18 | II-7 | II-21 | II-25 |
| Q-1992 | I-18 | II-7 | II-21 | II-26 |
| Q-1993 | I-18 | II-7 | II-21 | II-27 |
| Q-1994 | I-18 | II-7 | II-21 | II-28 |
| Q-1995 | I-18 | II-7 | II-21 | II-29 |
| Q-1996 | I-18 | II-7 | II-22 | II-23 |
| Q-1997 | I-18 | II-7 | II-22 | II-24 |
| Q-1998 | I-18 | II-7 | II-22 | II-25 |
| Q-1999 | I-18 | II-7 | II-22 | II-26 |
| Q-2000 | I-18 | II-7 | II-22 | II-27 |
| Q-2001 | I-18 | II-7 | II-22 | II-28 |
| Q-2002 | I-18 | II-7 | II-22 | II-29 |
| Q-2003 | I-18 | II-7 | II-23 | II-24 |
| Q-2004 | I-18 | II-7 | II-23 | II-25 |
| Q-2005 | I-18 | II-7 | II-23 | II-26 |
| Q-2006 | I-18 | II-7 | II-23 | II-27 |
| Q-2007 | I-18 | II-7 | II-23 | II-28 |
| Q-2008 | I-18 | II-7 | II-23 | II-29 |
| Q-2009 | I-18 | II-7 | II-24 | II-24 |
| Q-2010 | I-18 | II-7 | II-24 | II-25 |
| Q-2011 | I-18 | II-7 | II-24 | II-26 |
| Q-2012 | I-18 | II-7 | II-24 | II-27 |
| Q-2013 | I-18 | II-7 | II-24 | II-28 |
| Q-2014 | I-18 | II-7 | II-24 | II-29 |
| Q-2015 | I-18 | II-7 | II-25 | II-26 |
| Q-2016 | I-18 | II-7 | II-25 | II-27 |
| Q-2017 | I-18 | II-7 | II-25 | II-28 |
| Q-2018 | I-18 | II-7 | II-25 | II-29 |
| Q-2019 | I-18 | II-7 | II-26 | II-27 |
| Q-2020 | I-18 | II-7 | II-26 | II-28 |
| Q-2021 | I-18 | II-7 | II-26 | II-29 |
| Q-2022 | I-18 | II-7 | II-27 | II-28 |
| Q-2023 | I-18 | II-7 | II-27 | II-29 |
| Q-2024 | I-18 | II-7 | II-28 | II-29 |
| Q-2025 | I-18 | II-8 | II-9 | II-15 |
| Q-2026 | I-18 | II-8 | II-9 | II-21 |
| Q-2027 | I-18 | II-8 | II-9 | II-22 |
| Q-2028 | I-18 | II-8 | II-9 | II-23 |
| Q-2029 | I-18 | II-8 | II-9 | II-24 |
| Q-2030 | I-18 | II-8 | II-9 | II-25 |
| Q-2031 | I-18 | II-8 | II-9 | II-26 |
| Q-2032 | I-18 | II-8 | II-9 | II-27 |
| Q-2033 | I-18 | II-8 | II-9 | II-28 |
| Q-2034 | I-18 | II-8 | II-9 | II-29 |
| Q-2035 | I-18 | II-8 | II-15 | II-21 |
| Q-2036 | I-18 | II-8 | II-15 | II-22 |
| Q-2037 | I-18 | II-8 | II-15 | II-23 |
| Q-2038 | I-18 | II-8 | II-15 | II-24 |
| Q-2039 | I-18 | II-8 | II-15 | II-25 |
| Q-2040 | I-18 | II-8 | II-15 | II-26 |
| Q-2041 | I-18 | II-8 | II-15 | II-27 |
| Q-2042 | I-18 | II-8 | II-15 | II-28 |
| Q-2043 | I-18 | II-8 | II-15 | II-29 |
| Q-2044 | I-18 | II-8 | II-21 | II-22 |
| Q-2045 | I-18 | II-8 | II-21 | II-23 |
| Q-2046 | I-18 | II-8 | II-21 | II-24 |
| Q-2047 | I-18 | II-8 | II-21 | II-25 |
| Q-2048 | I-18 | II-8 | II-21 | II-26 |
| Q-2049 | I-18 | II-8 | II-21 | II-27 |
| Q-2050 | I-18 | II-8 | II-21 | II-28 |
| Q-2051 | I-18 | II-8 | II-21 | II-29 |
| Q-2052 | I-18 | II-8 | II-22 | II-23 |
| Q-2053 | I-18 | II-8 | II-22 | II-24 |
| Q-2054 | I-18 | II-8 | II-22 | II-25 |
| Q-2055 | I-18 | II-8 | II-22 | II-26 |
| Q-2056 | I-18 | II-8 | II-22 | II-27 |
| Q-2057 | I-18 | II-8 | II-22 | II-28 |
| Q-2058 | I-18 | II-8 | II-22 | II-29 |
| Q-2059 | I-18 | II-8 | II-23 | II-24 |
| Q-2060 | I-18 | II-8 | II-23 | II-25 |
| Q-2061 | I-18 | II-8 | II-23 | II-26 |
| Q-2062 | I-18 | II-8 | II-23 | II-27 |
| Q-2063 | I-18 | II-8 | II-23 | II-28 |
| Q-2064 | I-18 | II-8 | II-23 | II-29 |
| Q-2065 | I-18 | II-8 | II-24 | II-25 |
| Q-2066 | I-18 | II-8 | II-24 | II-26 |
| Q-2067 | I-18 | II-8 | II-24 | II-27 |
| Q-2068 | I-18 | II-8 | II-24 | II-28 |
| Q-2069 | I-18 | II-8 | II-24 | II-29 |
| Q-2070 | I-18 | II-8 | II-25 | II-26 |
| Q-2071 | I-18 | II-8 | II-25 | II-27 |
| Q-2072 | I-18 | II-8 | II-25 | II-28 |
| Q-2073 | I-18 | II-8 | II-25 | II-29 |
| Q-2074 | I-18 | II-8 | II-26 | II-27 |
| Q-2075 | I-18 | II-8 | II-26 | II-28 |
| Q-2076 | I-18 | II-8 | II-26 | II-29 |
| Q-2077 | I-18 | II-8 | II-27 | II-28 |
| Q-2078 | I-18 | II-8 | II-27 | II-29 |
| Q-2079 | I-18 | II-8 | II-28 | II-29 |
| Q-2080 | I-18 | II-9 | II-15 | II-21 |
| Q-2081 | I-18 | II-9 | II-15 | II-22 |
| Q-2082 | I-18 | II-9 | II-15 | II-23 |
| Q-2083 | I-18 | II-9 | II-15 | II-24 |
| Q-2084 | I-18 | II-9 | II-15 | II-25 |
| Q-2085 | I-18 | II-9 | II-15 | II-26 |
| Q-2086 | I-18 | II-9 | II-15 | II-27 |
| Q-2087 | I-18 | II-9 | II-15 | II-28 |
| Q-2088 | I-18 | II-9 | II-15 | II-29 |
| Q-2089 | I-18 | II-9 | II-21 | II-22 |
| Q-2090 | I-18 | II-9 | II-21 | II-23 |
| Q-2091 | I-18 | II-9 | II-21 | II-24 |
| Q-2092 | I-18 | II-9 | II-21 | II-25 |
| Q-2093 | I-18 | II-9 | II-21 | II-26 |
| Q-2094 | I-18 | II-9 | II-21 | II-27 |
| Q-2095 | I-18 | II-9 | II-21 | II-28 |
| Q-2096 | I-18 | II-9 | II-21 | II-29 |
| Q-2097 | I-18 | II-9 | II-22 | II-23 |
| Q-2098 | I-18 | II-9 | II-22 | II-24 |
| Q-2099 | I-18 | II-9 | II-22 | II-25 |
| Q-2100 | I-18 | II-9 | II-22 | II-26 |
| Q-2101 | I-18 | II-9 | II-22 | II-27 |
| Q-2102 | I-18 | II-9 | II-22 | II-28 |
| Q-2103 | I-18 | II-9 | II-22 | II-29 |
| Q-2104 | I-18 | II-9 | II-23 | II-24 |
| Q-2105 | I-18 | II-9 | II-23 | II-25 |
| Q-2106 | I-18 | II-9 | II-23 | II-26 |
| Q-2107 | I-18 | II-9 | II-23 | II-27 |
| Q-2108 | I-18 | II-9 | II-23 | II-28 |
| Q-2109 | I-18 | II-9 | II-23 | II-29 |
| Q-2110 | I-18 | II-9 | II-24 | II-25 |
| Q-2111 | I-18 | II-9 | II-24 | II-26 |
| Q-2112 | I-18 | II-9 | II-24 | II-27 |
| Q-2113 | I-18 | II-9 | II-24 | II-28 |
| Q-2114 | I-18 | II-9 | II-24 | II-29 |
| Q-2115 | I-18 | II-9 | II-25 | II-26 |
| Q-2116 | I-18 | II-9 | II-25 | II-27 |
| Q-2117 | I-18 | II-9 | II-25 | II-28 |
| Q-2118 | I-18 | II-9 | II-25 | II-29 |
| Q-2119 | I-18 | II-9 | II-26 | II-27 |
| Q-2120 | I-18 | II-9 | II-26 | II-28 |
| Q-2121 | I-18 | II-9 | II-26 | II-29 |
| Q-2122 | I-18 | II-9 | II-27 | II-28 |
| Q-2123 | I-18 | II-9 | II-27 | II-29 |
| Q-2124 | I-18 | II-9 | II-28 | II-29 |
| Q-2125 | I-18 | II-15 | II-21 | II-22 |
| Q-2126 | I-18 | II-15 | II-21 | II-23 |
| Q-2127 | I-18 | II-15 | II-21 | II-24 |
| Q-2128 | I-18 | II-15 | II-21 | II-25 |
| Q-2129 | I-18 | II-15 | II-21 | II-26 |
| Q-2130 | I-18 | II-15 | II-21 | II-27 |
| Q-2131 | I-18 | II-15 | II-21 | II-28 |
| Q-2132 | I-18 | II-15 | II-21 | II-29 |
| Q-2133 | I-18 | II-15 | II-22 | II-23 |
| Q-2134 | I-18 | II-15 | II-22 | II-24 |
| Q-2135 | I-18 | II-15 | II-22 | II-25 |
| Q-2136 | I-18 | II-15 | II-22 | II-26 |
| Q-2137 | I-18 | II-15 | II-22 | II-27 |
| Q-2138 | I-18 | II-15 | II-22 | II-28 |
| Q-2139 | I-18 | II-15 | II-22 | II-29 |
| Q-2140 | I-18 | II-15 | II-23 | II-24 |
| Q-2141 | I-18 | II-15 | II-23 | II-25 |
| Q-2142 | I-18 | II-15 | II-23 | II-26 |
| Q-2143 | I-18 | II-15 | II-23 | II-27 |
| Q-2144 | I-18 | II-15 | II-23 | II-28 |
| Q-2145 | I-18 | II-15 | II-23 | II-29 |
| Q-2146 | I-18 | II-15 | II-24 | II-25 |
| Q-2147 | I-18 | II-15 | II-24 | II-26 |
| Q-2148 | I-18 | II-15 | II-24 | II-27 |
| Q-2149 | I-18 | II-15 | II-24 | II-28 |
| Q-2150 | I-18 | II-15 | II-24 | II-29 |
| Q-2151 | I-18 | II-15 | II-25 | II-26 |
| Q-2152 | I-18 | II-15 | II-25 | II-27 |
| Q-2153 | I-18 | II-15 | II-25 | II-28 |
| Q-2154 | I-18 | II-15 | II-25 | II-29 |
| Q-2155 | I-18 | II-15 | II-26 | II-27 |
| Q-2156 | I-18 | II-15 | II-26 | II-28 |
| Q-2157 | I-18 | II-15 | II-26 | II-29 |
| Q-2158 | I-18 | II-15 | II-27 | II-28 |
| Q-2159 | I-18 | II-15 | II-27 | II-29 |
| Q-2160 | I-18 | II-15 | II-28 | II-29 |
| Q-2161 | I-18 | II-21 | II-22 | II-23 |
| Q-2162 | I-18 | II-21 | II-22 | II-24 |
| Q-2163 | I-18 | II-21 | II-22 | II-25 |
| Q-2164 | I-18 | II-21 | II-22 | II-26 |
| Q-2165 | I-18 | II-21 | II-22 | II-27 |
| Q-2166 | I-18 | II-21 | II-22 | II-28 |
| Q-2167 | I-18 | II-21 | II-22 | II-29 |
| Q-2168 | I-18 | II-21 | II-23 | II-24 |
| Q-2169 | I-18 | II-21 | II-23 | II-25 |
| Q-2170 | I-18 | II-21 | II-23 | II-26 |
| Q-2171 | I-18 | II-21 | II-23 | II-27 |
| Q-2172 | I-18 | II-21 | II-23 | II-28 |
| Q-2173 | I-18 | II-21 | II-23 | II-29 |
| Q-2174 | I-18 | II-21 | II-24 | II-25 |
| Q-2175 | I-18 | II-21 | II-24 | II-26 |
| Q-2176 | I-18 | II-21 | II-24 | II-27 |
| Q-2177 | I-18 | II-21 | II-24 | II-28 |
| Q-2178 | I-18 | II-21 | II-24 | II-29 |
| Q-2179 | I-18 | II-21 | II-25 | II-26 |
| Q-2180 | I-18 | II-21 | II-25 | II-27 |
| Q-2181 | I-18 | II-21 | II-25 | II-28 |
| Q-2182 | I-18 | II-21 | II-25 | II-29 |
| Q-2183 | I-18 | II-21 | II-26 | II-27 |
| Q-2184 | I-18 | II-21 | II-26 | II-28 |
| Q-2185 | I-18 | II-21 | II-26 | II-29 |
| Q-2186 | I-18 | II-21 | II-27 | II-28 |
| Q-2187 | I-18 | II-21 | II-27 | II-29 |
| Q-2188 | I-18 | II-21 | II-28 | II-29 |
| Q-2189 | I-18 | II-22 | II-23 | II-24 |
| Q-2190 | I-18 | II-22 | II-23 | II-25 |
| Q-2191 | I-18 | II-22 | II-23 | II-26 |
| Q-2192 | I-18 | II-22 | II-23 | II-27 |
| Q-2193 | I-18 | II-22 | II-23 | II-28 |
| Q-2194 | I-18 | II-22 | II-23 | II-29 |
| Q-2195 | I-18 | II-22 | II-24 | II-25 |
| Q-2196 | I-18 | II-22 | II-24 | II-26 |
| Q-2197 | I-18 | II-22 | II-24 | II-27 |
| Q-2198 | I-18 | II-22 | II-24 | II-28 |
| Q-2199 | I-18 | II-22 | II-24 | II-29 |
| Q-2200 | I-18 | II-22 | II-25 | II-26 |
| Q-2201 | I-18 | II-22 | II-25 | II-27 |
| Q-2202 | I-18 | II-22 | II-25 | II-28 |
| Q-2203 | I-18 | II-22 | II-25 | II-29 |
| Q-2204 | I-18 | II-22 | II-26 | II-27 |
| Q-2205 | I-18 | II-22 | II-26 | II-28 |
| Q-2206 | I-18 | II-22 | II-26 | II-29 |
| Q-2207 | I-18 | II-22 | II-27 | II-28 |
| Q-2208 | I-18 | II-22 | II-27 | II-29 |
| Q-2209 | I-18 | II-22 | II-28 | II-29 |
| Q-2210 | I-18 | II-23 | II-24 | II-25 |
| Q-2211 | I-18 | II-23 | II-24 | II-26 |
| Q-2212 | I-18 | II-23 | II-24 | II-27 |
| Q-2213 | I-18 | II-23 | II-24 | II-28 |
| Q-2214 | I-18 | II-23 | II-24 | II-29 |
| Q-2215 | I-18 | II-23 | II-25 | II-26 |
| Q-2216 | I-18 | II-23 | II-25 | II-27 |
| Q-2217 | I-18 | II-23 | II-25 | II-28 |
| Q-2218 | I-18 | II-23 | II-25 | II-29 |
| Q-2219 | I-18 | II-23 | II-26 | II-27 |
| Q-2220 | I-18 | II-23 | II-26 | II-28 |
| Q-2221 | I-18 | II-23 | II-26 | II-29 |
| Q-2222 | I-18 | II-23 | II-27 | II-28 |
| Q-2223 | I-18 | II-23 | II-27 | II-29 |
| Q-2224 | I-18 | II-23 | II-28 | II-29 |
| Q-2225 | I-18 | II-24 | II-25 | II-26 |
| Q-2226 | I-18 | II-24 | II-25 | II-27 |
| Q-2227 | I-18 | II-24 | II-25 | II-28 |
| Q-2228 | I-18 | II-24 | II-25 | II-29 |
| Q-2229 | I-18 | II-24 | II-26 | II-27 |
| Q-2230 | I-18 | II-24 | II-26 | II-28 |
| Q-2231 | I-18 | II-24 | II-26 | II-29 |
| Q-2232 | I-18 | II-24 | II-27 | II-28 |
| Q-2233 | I-18 | II-24 | II-27 | II-29 |
| Q-2234 | I-18 | II-24 | II-28 | II-29 |
| Q-2235 | I-18 | II-25 | II-26 | II-27 |
| Q-2236 | I-18 | II-25 | II-26 | II-28 |
| Q-2237 | I-18 | II-25 | II-26 | II-29 |
| Q-2238 | I-18 | II-25 | II-27 | II-28 |
| Q-2239 | I-18 | II-25 | II-27 | II-29 |
| Q-2240 | I-18 | II-25 | II-28 | II-29 |
| Q-2241 | I-18 | II-26 | II-27 | II-28 |
| Q-2242 | I-18 | II-26 | II-27 | II-29 |
| Q-2243 | I-18 | II-26 | II-28 | II-29 |
| Q-2244 | I-18 | II-27 | II-28 | II-29 |

“C” denotes “component”,
“M” denotes “mixture”.

According to a further embodiment of the invention, preference is given to quaternary mixtures in which component 2 is selected from the group of the following compounds:

| | |
|---|---|
| II-8 | kresoxim-methyl |
| II-9 | pyraclostrobin |
| II-25 | N-(3′,4′,5′-trifluorobiphenyl-2-yl)-3-difluoromethyl-1-methyl-1H- |
| | pyrazole-4-carboxamide |
| II-26 | sedaxane |
| II-28 | fluopyram |
| II-29 | penflufen |
| II-49 | azoxystrobin |
| II-50 | trifloxystrobin |
| II-51 | penthiopyrad |

and in which component 3 is a compound II selected from the group of the following compounds:

| | |
|---|---|
| II-32a | clothianidin |
| II-34a | imidacloprid |
| II-35a | thiamethoxam |

and where component 4 is fipronil (II-30a).

TABLE Q1
The quaternary mixtures Q1-1 to Q1-297 below comprise, as component
1, a preferred compound I, a component 2 selected from preferred
active compounds of groups A to F, a component 3 selected from
preferred active compounds of group I) and, as component 4,
fipronil. In the table below, each row corresponds to a quaternary
mixture according to the invention having the mixture components
1 to 4 listed in the row in question.

| M | C 1 | C 2 | C 3 | C 4 |
|---|---|---|---|---|
| Q1-1 | I-1 | II-8 | II-32a | II-30a |
| Q1-2 | I-1 | II-8 | II-34a | II-30a |
| Q1-3 | I-1 | II-8 | II-35a | II-30a |
| Q1-4 | I-1 | II-9 | II-32a | II-30a |
| Q1-5 | I-1 | II-9 | II-34a | II-30a |
| Q1-6 | I-1 | II-9 | II-35a | II-30a |
| Q1-7 | I-1 | II-25 | II-32a | II-30a |
| Q1-8 | I-1 | II-25 | II-34a | II-30a |
| Q1-9 | I-1 | II-25 | II-35a | II-30a |
| Q1-10 | I-1 | II-26 | II-32a | II-30a |
| Q1-11 | I-1 | II-26 | II-34a | II-30a |
| Q1-12 | I-1 | II-26 | II-35a | II-30a |
| Q1-13 | I-1 | II-28 | II-32a | II-30a |
| Q1-14 | I-1 | II-28 | II-34a | II-30a |
| Q1-15 | I-1 | II-28 | II-35a | II-30a |
| Q1-16 | I-1 | II-29 | II-32a | II-30a |
| Q1-17 | I-1 | II-29 | II-34a | II-30a |
| Q1-18 | I-1 | II-29 | II-35a | II-30a |
| Q1-19 | I-1 | II-49 | II-32a | II-30a |
| Q1-20 | I-1 | II-49 | II-34a | II-30a |
| Q1-21 | I-1 | II-49 | II-35a | II-30a |
| Q1-22 | I-1 | II-50 | II-32a | II-30a |
| Q1-23 | I-1 | II-50 | II-34a | II-30a |
| Q1-24 | I-1 | II-50 | II-35a | II-30a |
| Q1-25 | I-1 | II-51 | II-32a | II-30a |
| Q1-26 | I-1 | II-51 | II-34a | II-30a |
| Q1-27 | I-1 | II-51 | II-35a | II-30a |
| Q1-28 | I-2 | II-8 | II-32a | II-30a |
| Q1-29 | I-2 | II-8 | II-34a | II-30a |
| Q1-30 | I-2 | II-8 | II-35a | II-30a |
| Q1-31 | I-2 | II-9 | II-32a | II-30a |
| Q1-32 | I-2 | II-9 | II-34a | II-30a |
| Q1-33 | I-2 | II-9 | II-35a | II-30a |
| Q1-34 | I-2 | II-25 | II-32a | II-30a |
| Q1-35 | I-2 | II-25 | II-34a | II-30a |
| Q1-36 | I-2 | II-25 | II-35a | II-30a |
| Q1-37 | I-2 | II-26 | II-32a | II-30a |
| Q1-38 | I-2 | II-26 | II-34a | II-30a |
| Q1-39 | I-2 | II-26 | II-35a | II-30a |
| Q1-40 | I-2 | II-28 | II-32a | II-30a |
| Q1-41 | I-2 | II-28 | II-34a | II-30a |
| Q1-42 | I-2 | II-28 | II-35a | II-30a |
| Q1-43 | I-2 | II-29 | II-32a | II-30a |
| Q1-44 | I-2 | II-29 | II-34a | II-30a |
| Q1-45 | I-2 | II-29 | II-35a | II-30a |
| Q1-46 | I-2 | II-49 | II-32a | II-30a |
| Q1-47 | I-2 | II-49 | II-34a | II-30a |
| Q1-48 | I-2 | II-49 | II-35a | II-30a |
| Q1-49 | I-2 | II-50 | II-32a | II-30a |
| Q1-50 | I-2 | II-50 | II-34a | II-30a |
| Q1-51 | I-2 | II-50 | II-35a | II-30a |
| Q1-52 | I-2 | II-51 | II-32a | II-30a |
| Q1-53 | I-2 | II-51 | II-34a | II-30a |
| Q1-54 | I-2 | II-51 | II-35a | II-30a |
| Q1-55 | I-6 | II-8 | II-32a | II-30a |
| Q1-56 | I-6 | II-8 | II-34a | II-30a |
| Q1-57 | I-6 | II-8 | II-35a | II-30a |
| Q1-58 | I-6 | II-9 | II-32a | II-30a |
| Q1-59 | I-6 | II-9 | II-34a | II-30a |
| Q1-60 | I-6 | II-9 | II-35a | II-30a |
| Q1-61 | I-6 | II-25 | II-32a | II-30a |
| Q1-62 | I-6 | II-25 | II-34a | II-30a |
| Q1-63 | I-6 | II-25 | II-35a | II-30a |
| Q1-64 | I-6 | II-26 | II-32a | II-30a |
| Q1-65 | I-6 | II-26 | II-34a | II-30a |
| Q1-66 | I-6 | II-26 | II-35a | II-30a |
| Q1-67 | I-6 | II-28 | II-32a | II-30a |
| Q1-68 | I-6 | II-28 | II-34a | II-30a |
| Q1-69 | I-6 | II-28 | II-35a | II-30a |
| Q1-70 | I-6 | II-29 | II-32a | II-30a |
| Q1-71 | I-6 | II-29 | II-34a | II-30a |
| Q1-72 | I-6 | II-29 | II-35a | II-30a |
| Q1-73 | I-6 | II-49 | II-32a | II-30a |
| Q1-74 | I-6 | II-49 | II-34a | II-30a |
| Q1-75 | I-6 | II-49 | II-35a | II-30a |
| Q1-76 | I-6 | II-50 | II-32a | II-30a |
| Q1-77 | I-6 | II-50 | II-34a | II-30a |
| Q1-78 | I-6 | II-50 | II-35a | II-30a |
| Q1-79 | I-6 | II-51 | II-32a | II-30a |
| Q1-80 | I-6 | II-51 | II-34a | II-30a |
| Q1-81 | I-6 | II-51 | II-35a | II-30a |
| Q1-82 | I-7 | II-8 | II-32a | II-30a |
| Q1-83 | I-7 | II-8 | II-34a | II-30a |
| Q1-84 | I-7 | II-8 | II-35a | II-30a |
| Q1-85 | I-7 | II-9 | II-32a | II-30a |
| Q1-86 | I-7 | II-9 | II-34a | II-30a |
| Q1-87 | I-7 | II-9 | II-35a | II-30a |
| Q1-88 | I-7 | II-25 | II-32a | II-30a |
| Q1-89 | I-7 | II-25 | II-34a | II-30a |
| Q1-90 | I-7 | II-25 | II-35a | II-30a |
| Q1-91 | I-7 | II-26 | II-32a | II-30a |
| Q1-92 | I-7 | II-26 | II-34a | II-30a |
| Q1-93 | I-7 | II-26 | II-35a | II-30a |
| Q1-94 | I-7 | II-28 | II-32a | II-30a |
| Q1-95 | I-7 | II-28 | II-34a | II-30a |
| Q1-96 | I-7 | II-28 | II-35a | II-30a |
| Q1-97 | I-7 | II-29 | II-32a | II-30a |
| Q1-98 | I-7 | II-29 | II-34a | II-30a |
| Q1-99 | I-7 | II-29 | II-35a | II-30a |
| Q1-100 | I-7 | II-49 | II-32a | II-30a |
| Q1-101 | I-7 | II-49 | II-34a | II-30a |
| Q1-102 | I-7 | II-49 | II-35a | II-30a |
| Q1-103 | I-7 | II-50 | II-32a | II-30a |
| Q1-104 | I-7 | II-50 | II-34a | II-30a |
| Q1-105 | I-7 | II-50 | II-35a | II-30a |
| Q1-106 | I-7 | II-51 | II-32a | II-30a |
| Q1-107 | I-7 | II-51 | II-34a | II-30a |
| Q1-108 | I-7 | II-51 | II-35a | II-30a |
| Q1-109 | I-8 | II-8 | II-32a | II-30a |
| Q1-110 | I-8 | II-8 | II-34a | II-30a |
| Q1-111 | I-8 | II-8 | II-35a | II-30a |
| Q1-112 | I-8 | II-9 | II-32a | II-30a |
| Q1-113 | I-8 | II-9 | II-34a | II-30a |
| Q1-114 | I-8 | II-9 | II-35a | II-30a |
| Q1-115 | I-8 | II-25 | II-32a | II-30a |
| Q1-116 | I-8 | II-25 | II-34a | II-30a |
| Q1-117 | I-8 | II-25 | II-35a | II-30a |
| Q1-118 | I-8 | II-26 | II-32a | II-30a |
| Q1-119 | I-8 | II-26 | II-34a | II-30a |
| Q1-120 | I-8 | II-26 | II-35a | II-30a |
| Q1-121 | I-8 | II-28 | II-32a | II-30a |
| Q1-122 | I-8 | II-28 | II-34a | II-30a |
| Q1-123 | I-8 | II-28 | II-35a | II-30a |
| Q1-124 | I-8 | II-29 | II-32a | II-30a |
| Q1-125 | I-8 | II-29 | II-34a | II-30a |
| Q1-126 | I-8 | II-29 | II-35a | II-30a |
| Q1-127 | I-8 | II-49 | II-32a | II-30a |
| Q1-128 | I-8 | II-49 | II-34a | II-30a |
| Q1-129 | I-8 | II-49 | II-35a | II-30a |
| Q1-130 | I-8 | II-50 | II-32a | II-30a |
| Q1-131 | I-8 | II-50 | II-34a | II-30a |
| Q1-132 | I-8 | II-50 | II-35a | II-30a |
| Q1-133 | I-8 | II-51 | II-32a | II-30a |
| Q1-134 | I-8 | II-51 | II-34a | II-30a |
| Q1-135 | I-8 | II-51 | II-35a | II-30a |
| Q1-136 | I-9 | II-8 | II-32a | II-30a |
| Q1-137 | I-9 | II-8 | II-34a | II-30a |
| Q1-138 | I-9 | II-8 | II-35a | II-30a |
| Q1-139 | I-9 | II-9 | II-32a | II-30a |
| Q1-140 | I-9 | II-9 | II-34a | II-30a |
| Q1-141 | I-9 | II-9 | II-35a | II-30a |
| Q1-142 | I-9 | II-25 | II-32a | II-30a |
| Q1-143 | I-9 | II-25 | II-34a | II-30a |
| Q1-144 | I-9 | II-25 | II-35a | II-30a |
| Q1-145 | I-9 | II-26 | II-32a | II-30a |
| Q1-146 | I-9 | II-26 | II-34a | II-30a |
| Q1-147 | I-9 | II-26 | II-35a | II-30a |
| Q1-148 | I-9 | II-28 | II-32a | II-30a |
| Q1-149 | I-9 | II-28 | II-34a | II-30a |
| Q1-150 | I-9 | II-28 | II-35a | II-30a |
| Q1-151 | I-9 | II-29 | II-32a | II-30a |
| Q1-152 | I-9 | II-29 | II-34a | II-30a |
| Q1-153 | I-9 | II-29 | II-35a | II-30a |
| Q1-154 | I-9 | II-49 | II-32a | II-30a |
| Q1-155 | I-9 | II-49 | II-34a | II-30a |
| Q1-156 | I-9 | II-49 | II-35a | II-30a |
| Q1-157 | I-9 | II-50 | II-32a | II-30a |
| Q1-158 | I-9 | II-50 | II-34a | II-30a |
| Q1-159 | I-9 | II-50 | II-35a | II-30a |
| Q1-160 | I-9 | II-51 | II-32a | II-30a |
| Q1-161 | I-9 | II-51 | II-34a | II-30a |
| Q1-162 | I-9 | II-51 | II-35a | II-30a |
| Q1-163 | I-11 | II-8 | II-32a | II-30a |
| Q1-164 | I-11 | II-8 | II-34a | II-30a |
| Q1-165 | I-11 | II-8 | II-35a | II-30a |
| Q1-166 | I-11 | II-9 | II-32a | II-30a |
| Q1-167 | I-11 | II-9 | II-34a | II-30a |
| Q1-168 | I-11 | II-9 | II-35a | II-30a |
| Q1-169 | I-11 | II-25 | II-32a | II-30a |
| Q1-170 | I-11 | II-25 | II-34a | II-30a |
| Q1-171 | I-11 | II-25 | II-35a | II-30a |
| Q1-172 | I-11 | II-26 | II-32a | II-30a |
| Q1-173 | I-11 | II-26 | II-34a | II-30a |
| Q1-174 | I-11 | II-26 | II-35a | II-30a |
| Q1-175 | I-11 | II-28 | II-32a | II-30a |
| Q1-176 | I-11 | II-28 | II-34a | II-30a |
| Q1-177 | I-11 | II-28 | II-35a | II-30a |
| Q1-178 | I-11 | II-29 | II-32a | II-30a |
| Q1-179 | I-11 | II-29 | II-34a | II-30a |
| Q1-180 | I-11 | II-29 | II-35a | II-30a |
| Q1-181 | I-11 | II-49 | II-32a | II-30a |
| Q1-182 | I-11 | II-49 | II-34a | II-30a |
| Q1-183 | I-11 | II-49 | II-35a | II-30a |
| Q1-184 | I-11 | II-50 | II-32a | II-30a |
| Q1-185 | I-11 | II-50 | II-34a | II-30a |
| Q1-186 | I-11 | II-50 | II-35a | II-30a |
| Q1-187 | I-11 | II-51 | II-32a | II-30a |
| Q1-188 | I-11 | II-51 | II-34a | II-30a |
| Q1-189 | I-11 | II-51 | II-35a | II-30a |
| Q1-190 | I-12 | II-8 | II-32a | II-30a |
| Q1-191 | I-12 | II-8 | II-34a | II-30a |
| Q1-192 | I-12 | II-8 | II-35a | II-30a |
| Q1-193 | I-12 | II-9 | II-32a | II-30a |
| Q1-194 | I-12 | II-9 | II-34a | II-30a |
| Q1-195 | I-12 | II-9 | II-35a | II-30a |
| Q1-196 | I-12 | II-25 | II-32a | II-30a |
| Q1-197 | I-12 | II-25 | II-34a | II-30a |
| Q1-198 | I-12 | II-25 | II-35a | II-30a |
| Q1-199 | I-12 | II-26 | II-32a | II-30a |
| Q1-200 | I-12 | II-26 | II-34a | II-30a |
| Q1-201 | I-12 | II-26 | II-35a | II-30a |
| Q1-202 | I-12 | II-28 | II-32a | II-30a |
| Q1-203 | I-12 | II-28 | II-34a | II-30a |
| Q1-204 | I-12 | II-28 | II-35a | II-30a |
| Q1-205 | I-12 | II-29 | II-32a | II-30a |
| Q1-206 | I-12 | II-29 | II-34a | II-30a |
| Q1-207 | I-12 | II-29 | II-35a | II-30a |
| Q1-208 | I-12 | II-49 | II-32a | II-30a |
| Q1-209 | I-12 | II-49 | II-34a | II-30a |
| Q1-210 | I-12 | II-49 | II-35a | II-30a |
| Q1-211 | I-12 | II-50 | II-32a | II-30a |
| Q1-212 | I-12 | II-50 | II-34a | II-30a |
| Q1-213 | I-12 | II-50 | II-35a | II-30a |
| Q1-214 | I-12 | II-51 | II-32a | II-30a |
| Q1-215 | I-12 | II-51 | II-34a | II-30a |
| Q1-216 | I-12 | II-51 | II-35a | II-30a |
| Q1-217 | I-13 | II-8 | II-32a | II-30a |
| Q1-218 | I-13 | II-8 | II-34a | II-30a |
| Q1-219 | I-13 | II-8 | II-35a | II-30a |
| Q1-220 | I-13 | II-9 | II-32a | II-30a |
| Q1-221 | I-13 | II-9 | II-34a | II-30a |
| Q1-222 | I-13 | II-9 | II-35a | II-30a |
| Q1-223 | I-13 | II-25 | II-32a | II-30a |
| Q1-224 | I-13 | II-25 | II-34a | II-30a |
| Q1-225 | I-13 | II-25 | II-35a | II-30a |
| Q1-226 | I-13 | II-26 | II-32a | II-30a |
| Q1-227 | I-13 | II-26 | II-34a | II-30a |
| Q1-228 | I-13 | II-26 | II-35a | II-30a |
| Q1-229 | I-13 | II-28 | II-32a | II-30a |
| Q1-230 | I-13 | II-28 | II-34a | II-30a |
| Q1-231 | I-13 | II-28 | II-35a | II-30a |
| Q1-232 | I-13 | II-29 | II-32a | II-30a |
| Q1-233 | I-13 | II-29 | II-34a | II-30a |
| Q1-234 | I-13 | II-29 | II-35a | II-30a |
| Q1-235 | I-13 | II-49 | II-32a | II-30a |
| Q1-236 | I-13 | II-49 | II-34a | II-30a |
| Q1-237 | I-13 | II-49 | II-35a | II-30a |
| Q1-238 | I-13 | II-50 | II-32a | II-30a |
| Q1-239 | I-13 | II-50 | II-34a | II-30a |
| Q1-240 | I-13 | II-50 | II-35a | II-30a |
| Q1-241 | I-13 | II-51 | II-32a | II-30a |
| Q1-242 | I-13 | II-51 | II-34a | II-30a |
| Q1-243 | I-13 | II-51 | II-35a | II-30a |
| Q1-244 | I-14 | II-8 | II-32a | II-30a |
| Q1-245 | I-14 | II-8 | II-34a | II-30a |
| Q1-246 | I-14 | II-8 | II-35a | II-30a |
| Q1-247 | I-14 | II-9 | II-32a | II-30a |
| Q1-248 | I-14 | II-9 | II-34a | II-30a |
| Q1-249 | I-14 | II-9 | II-35a | II-30a |
| Q1-250 | I-14 | II-25 | II-32a | II-30a |
| Q1-251 | I-14 | II-25 | II-34a | II-30a |
| Q1-252 | I-14 | II-25 | II-35a | II-30a |
| Q1-253 | I-14 | II-26 | II-32a | II-30a |
| Q1-254 | I-14 | II-26 | II-34a | II-30a |
| Q1-255 | I-14 | II-26 | II-35a | II-30a |
| Q1-256 | I-14 | II-28 | II-32a | II-30a |
| Q1-257 | I-14 | II-28 | II-34a | II-30a |
| Q1-258 | I-14 | II-28 | II-35a | II-30a |
| Q1-259 | I-14 | II-29 | II-32a | II-30a |
| Q1-260 | I-14 | II-29 | II-34a | II-30a |
| Q1-261 | I-14 | II-29 | II-35a | II-30a |
| Q1-262 | I-14 | II-49 | II-32a | II-30a |
| Q1-263 | I-14 | II-49 | II-34a | II-30a |
| Q1-264 | I-14 | II-49 | II-35a | II-30a |
| Q1-265 | I-14 | II-50 | II-32a | II-30a |
| Q1-266 | I-14 | II-50 | II-34a | II-30a |
| Q1-267 | I-14 | II-50 | II-35a | II-30a |
| Q1-268 | I-14 | II-51 | II-32a | II-30a |
| Q1-269 | I-14 | II-51 | II-34a | II-30a |
| Q1-270 | I-14 | II-51 | II-35a | II-30a |
| Q1-271 | I-18 | II-8 | II-32a | II-30a |
| Q1-272 | I-18 | II-8 | II-34a | II-30a |
| Q1-273 | I-18 | II-8 | II-35a | II-30a |
| Q1-274 | I-18 | II-9 | II-32a | II-30a |
| Q1-275 | I-18 | II-9 | II-34a | II-30a |
| Q1-276 | I-18 | II-9 | II-35a | II-30a |
| Q1-277 | I-18 | II-25 | II-32a | II-30a |
| Q1-278 | I-18 | II-25 | II-34a | II-30a |
| Q1-279 | I-18 | II-25 | II-35a | II-30a |
| Q1-280 | I-18 | II-26 | II-32a | II-30a |
| Q1-281 | I-18 | II-26 | II-34a | II-30a |
| Q1-282 | I-18 | II-26 | II-35a | II-30a |
| Q1-283 | I-18 | II-28 | II-32a | II-30a |
| Q1-284 | I-18 | II-28 | II-34a | II-30a |
| Q1-285 | I-18 | II-28 | II-35a | II-30a |
| Q1-286 | I-18 | II-29 | II-32a | II-30a |
| Q1-287 | I-18 | II-29 | II-34a | II-30a |
| Q1-288 | I-18 | II-29 | II-35a | II-30a |
| Q1-289 | I-18 | II-49 | II-32a | II-30a |
| Q1-290 | I-18 | II-49 | II-34a | II-30a |
| Q1-291 | I-18 | II-49 | II-35a | II-30a |
| Q1-292 | I-18 | II-50 | II-32a | II-30a |
| Q1-293 | I-18 | II-50 | II-34a | II-30a |
| Q1-294 | I-18 | II-50 | II-35a | II-30a |
| Q1-295 | I-18 | II-51 | II-32a | II-30a |
| Q1-296 | I-18 | II-51 | II-34a | II-30a |
| Q1-297 | I-18 | II-51 | II-35a | II-30a |

“C” denotes “component”, “M” denotes “mixture”.

The mixtures of the compounds I and II (=mixtures according to the invention) I and the compositions thereof (=compositions according to the invention) are suitable as fungicides for controlling harmful fungi. They are distinguished by excellent activity against a broad spectrum of phytopathogenic fungi including soilborne pathogens which originate in particular from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some of them are systemically active and can be used in crop protection as foliar fungicides, fungicides for seed dressing and soil fungicides. In addition, they are suitable for controlling fungi which, inter alia, attack the wood or the roots of plants.

The mixtures according to the invention and the compositions according to the invention are of particular importance for the control of a large number of pathogenic fungi on various crop plants such as cereals, for example wheat, rye, barley, triticale, oats or rice; beets, for example sugar beets or fodder beets; pomaceous fruits, stone fruits and soft fruits, for example apples, pears, plums, peaches, almonds, cherries, strawberries, raspberries, currants or gooseberries; leguminous plants, for example beans, lentils, peas, lucerne or soybeans; oil plants, for example oilseed rape, mustard, olives, sunflowers, coconut, cocoa, castor beans, oil palms, peanuts or soybeans; cucurbits, for example pumpkins, cucumbers or melons; fiber plants, for example cotton, flax, hemp or jute; citrus fruits, for example oranges, lemons, grapefruits or mandarins; vegetable plants, for example spinach, lettuce, asparagus, cabbage plants, carrots, onions, tomatoes, potatoes, pumpkins or bell peppers; laurel plants, for example avocados, cinnamon or camphor; energy and raw material plants, for example corn, soybeans, wheat, oilseed rape, sugar cane or oil palms; corn; tobacco; nuts; coffee; tea; bananas; grapevines (grapes for eating and grapes for wine making); hops; grass, for example lawns; rubber plants; ornamental and forest plants, for example flowers, shrubs, deciduous trees and coniferous trees, and also on the propagation material, for example seeds, and on the harvested products of these plants.

Preferably, the mixtures and compositions according to the invention are used for controlling a large number of fungal pathogens in agricultural crops, for example potatoes, sugar beets, tobacco, wheat, rye, barley, oats, rice, corn, cotton, soybeans, oilseed rape, leguminous plants, sunflowers, coffee or sugar cane; fruit plants, grapevines and ornamental plants and vegetable plants, for example cucumbers, tomatoes, beans and pumpkins and also on the propagation material, for example seeds, and the harvested products of these plants.

The term plant propagation materials comprises all generative parts of the plant, for example seeds, and vegetative plant parts, such as seedlings and tubers (for example potatoes) which can be utilized for propagating a plant. These include seeds, roots, fruits, tubers, bulbs, rhizomes, shoots and other plant parts including seedlings and young plants which are transplanted after germination or after emergence. The young plants can be protected by partial or complete treatment, for example by immersion or watering, against harmful fungi.

The treatment of plant propagation materials with mixtures or compositions according to the invention is used for controlling a large number of fungal pathogens in cereal crops, for example wheat, rye, barley or oats; rice, corn, cotton and soybeans.

The term crop plants also includes those plants which have been modified by breeding, mutagenesis or genetic engineering methods including the biotechnological agricultural products which are on the market or under development (see, for example, http://www.bio.org/speeches/pubs/er/agri_products.asp). Genetically modified plants are plants whose genetic material has been modified in a manner which does not occur under natural conditions by crossing, mutations or by natural recombination (that is to say, a recombination of the genetic information). In general, one or more genes are integrated into the genetic material of the plant in order to improve the properties of the plant. Such modifications by genetic engineering include post-translational modifications of proteins, oligopeptides or polypeptides, for example by glycosylation or attachment of polymers such as, for example, prenylated, acetylated or farnesylated radicals or PEG radicals.

By way of example, mention may be made of plants which, by breeding and genetic engineering, have acquired tolerance to certain classes of herbicides, such as hydroxyphenylpyruvate dioxygenase (HPPD) inhibitors, acetolactate synthase (ALS) inhibitors, such as, for example, sulfonylureas (EP-A 257 993, U.S. Pat. No. 5,013,659) or imidazolinones (for example U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), enolpyruvylshikimate 3 phosphate synthase (EPSPS) inhibitors, such as, for example, glyphosate (see, for example, WO 92/00377), glutamine synthetase (GS) inhibitors, such as, for example, glufosinate (see, for example, EP-A 242 236, EP-A 242 246) or oxynil herbicides (see, for example, U.S. Pat. No. 5,559,024). Clearfield oilseed rape (BASF SE, Germany), for example, which is tolerant to imidazolinones, for example imazamox, was generated by breeding and mutagenesis. With the aid of genetic engineering methods, crop plants such as soybeans, cotton, corn, beets and oilseed rape were generated which are resistant to glyphosate or glufosinate, and which are obtainable under the trade names RoundupReady® (glyphosate-resistant, Monsanto, U.S.A.) and Liberty Link® (glufosinate-resistant, Bayer CropScience, Germany).

Also included are plants which, owing to interventions by genetic engineering, produce one or more toxins, for example those of the bacterial strain Bacillus. Toxins which are produced by such genetically modified plants include, for example, insecticidal proteins of Bacillus spp., in particular B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), for example VIP1, VIP2, VIP3, or VIP3A; insecticidal proteins of nematode-colonizing bacteria, for example Photorhabdus spp. or Xenorhabdus spp.; toxins of animal organisms, for example wasp, spider or scorpion toxins; fungal toxins, for example from Streptomycetes; plant lectins, for example from peas or barley; agglutinins; proteinase inhibitors, for example trypsin inhibitors, serine protease inhibitors, patatin, cystatin or papain inhibitors; ribosome-inactivating proteins (RIPs), for example ricin, corn-RIP, abrin, luffin, saporin or bryodin; steroid-metabolizing enzymes, for example 3-hydroxysteroid oxidase, ecdysteroid-IDP glycosyl transferase, cholesterol oxidase, ecdyson inhibitors, or HMG-CoA reductase; ion channel blockers, for example inhibitors of sodium channels or calcium channels; juvenile hormone esterase; receptors of the diuretic hormone (helicokinin receptors); stilbene synthase, bibenzyl synthase, chitinases and glucanases. In the plants, these toxins may also be produced as pretoxins, hybrid proteins or truncated or otherwise modified proteins. Hybrid proteins are characterized by a novel combination of different protein domains (see, for example, WO 2002/015701). Further examples of such toxins or genetically modified plants which produce these toxins are disclosed in EP-A 374 753, WO 93/07278, WO 95/34656, EP-A 427 529, EP-A 451 878, WO 03/18810 and WO 03/52073. The methods for producing these genetically modified plants are known to the person skilled in the art and disclosed, for example, in the publications mentioned above. Many of the toxins mentioned above bestow, upon the plants by which they are produced, tolerance to pests from all taxonomic classes of arthropods, in particular to beetles (Coeleropta), dipterans (Diptera) and butterflies (Lepidoptera) and to nematodes (Nematoda). Genetically modified plants which produce one or more genes coding for insecticidal toxins are described, for example, in the publications mentioned above, and some of them are commercially available, such as, for example, YieldGard® (corn varieties which produce the toxin Cry1Ab), YieldGard® Plus (corn varieties which produce the toxins Cry1Ab and Cry3Bb1), Starlink® (corn varieties which produce the toxin Cry9c), Herculex® RW (corn varieties which produce the toxins Cry34Ab1, Cry35Ab1 and the enzyme phosphinothricin-N-acetyltransferase [PAT]); NuCOTN® 33B (cotton varieties which produce the toxin Cry1Ac), Bollgard® I (cotton varieties which produce the toxin Cry1Ac), Bollgard® II (cotton varieties which produce the toxins Cry1Ac and Cry2Ab2); VIPCOT® (cotton varieties which produce a VIP toxin); NewLeaf® (potato varieties which produce the toxin Cry3A); Bt-Xtra®, NatureGard®, KnockOut®, BiteGard®, Protecta®, Bt11 (for example Agrisure® CB) and Bt176 from Syngenta Seeds SAS, France (corn varieties which produce the toxin Cry1Ab and the PAT enzyme), MIR604 from Syngenta Seeds SAS, France (corn varieties which produce a modified version of the toxin Cry3A, see WO 03/018810), MON 863 from Monsanto Europe S.A., Belgium (corn varieties which produce the toxin Cry3Bb1), IPC 531 from Monsanto Europe S.A., Belgium (cotton varieties which produce a modified version of the toxin Cry1Ac) and 1507 from Pioneer Overseas Corporation, Belgium (corn varieties which produce the toxin Cry1F and the PAT enzyme).

Also included are plants which, with the aid of genetic engineering, produce one or more proteins which have increased resistance to bacterial, viral or fungal pathogens, such as, for example, pathogenesis-related proteins (PR proteins, see EP-A 0 392 225), resistance proteins (for example potato varieties producing two resistance genes against Phytophthora infestans from the wild Mexican potato Solanum buibocastanum) or T4 lysozyme (for example potato varieties which, by producing this protein, are resistant to bacteria such as Erwinia amyivora).

Also included are plants whose productivity has been improved with the aid of genetic engineering methods, for example by enhancing the potential yield (for example biomass, grain yield, starch, oil or protein content), tolerance to drought, salt or other limiting environmental factors or resistance to pests and fungal, bacterial and viral pathogens.

Also included are plants whose ingredients have been modified with the aid of genetic engineering methods in particular for improving human or animal diet, for example by oil plants producing health-promoting long-chain omega 3 fatty acids or monounsaturated omega 9 fatty acids (for example Nexera® oilseed rape, DOW Agro Sciences, Canada).

Also included are plants which have been modified with the aid of genetic engineering methods for improving the production of raw materials, for example by increasing the amylopectin content of potatoes (Amflora® potato, BASF SE, Germany).

Specifically, the mixtures and compositions according to the invention are suitable for controlling the following plant diseases:

Albugo spp. (white rust) on ornamental plants, vegetable crops (for example A. candida) and sunflowers (for example A. tragopogonis); Alternaria spp. (black spot disease, black blotch) on vegetables, oilseed rape (for example A. brassicola or A. brassicae), sugar beet (for example A. tenuis), fruit, rice, soybeans and also on potatoes (for example A. solani or A. alternata) and tomatoes (for example A. solani or A. alternata) and Alternaria spp. (black head) on wheat; Aphanomyces spp. on sugar beets and vegetables; Ascochyta spp. on cereals and vegetables, for example A. tritici (Ascochyta leaf blight) on wheat and A. hordei on barley; Bipolaris and Drechslera spp. (teleomorph: Cochliobolus spp.), for example leaf spot diseases (for example D. maydis and B. zeicola) on corn, for example glume blotch (B. sorokiniana) on cereals and for example B. oryzae on rice and on lawn; Blumeria (old name: Erysiphe) graminis (powdery mildew) on cereals (for example wheat or barley); Botryosphaeria spp. (‘Black Dead Arm Disease’) on grapevines (for example B. obtusa); Botrytis cinerea (teleomorph: Botryotinia fuckeliana: gray mold, gray rot) on soft fruit and pomaceous fruit (inter alia strawberries), vegetables (inter alia lettuce, carrots, celeriac and cabbage), oilseed rape, flowers, grapevines, forest crops and wheat (ear mold); Bremia lactucae (downy mildew) on lettuce; Ceratocystis (syn. Ophiostoma) spp. (blue stain fungus) on deciduous trees and coniferous trees, for example C. ulmi (Dutch elm disease) on elms; Cercospora spp. (Cercospora leaf spot) on corn (for example C. zeae-maydis), rice, sugar beets (for example C. beticola), sugar cane, vegetables, coffee, soybeans (for example C. sojina or C. kikuchii) and rice; Cladosporium spp. on tomato (for example C. fulvum tomato leaf mold) and cereals, for example C. herbarum (ear rot) on wheat; Claviceps purpurea (ergot) on cereals; Cochilobolus (anamorph: Helminthosporium or Bipolaris) spp. (leaf spot) on corn (for example C. carbonum), cereals (for example C. sativus, anamorph: B. sorokiniana: glume blotch) and rice (for example C. miyabeanus, anamorph: H. oryzae); Colletotrichum (teleomorph: Glomerella) spp. (anthracnosis) on cotton (for example C. gossypii), corn (for example C. graminicola: stem rot and anthracnosis), soft fruit, potatoes (for example C. coccodes: wilt disease), beans (for example C. lindemuthianum) and soybeans (for example C. truncatum); Corticium spp., for example C. sasakii (sheath blight) on rice; Corynespora cassiicola (leaf spot) on soybeans and ornamental plants; Cycloconium spp., for example C. oleaginum on olive; Cylindrocarpon spp. (for example fruit tree cancer or black foot disease of grapevine, teleomorph: Nectria or Neonectria spp.) on fruit trees, grapevines (for example C. liriodendri; teleomorph: Neonectria liriodendri, black foot disease) and many ornamental trees; Dematophora (teleomorph: Rosellinia) necatrix (root/stem rot) on soybeans; Diaporthe spp.) for example D. phaseolorum (stem disease) on soybeans; Drechslera (syn. Helminthosporium, teleomorph: Pyrenophora) spp. on corn, cereals, such as barley (for example D. teres, net blotch) and on wheat (for example D. tritici-repentis: DTR leaf spot), rice and lawn; Esca disease (dieback of grapevine, apoplexia) on grapevines, caused by Formitiporia (syn. Phellinus) punctata, F. mediterranea, Phaeomoniella chlamydospora (old name Phaeoacremonium chlamydosporum), Phaeoacremonium aleophllum and/or Botryosphaeria obtusa; Elsinoe spp. on pome fruit (E. pyri) and soft fruit (E. veneta: anthracnosis) and also grapevines (E. ampelina: anthracnosis); Entyloma oryzae (leaf smut) on rice; Epkoccum spp. (black head) on wheat; Erysiphe spp. (powdery mildew) on sugar beet (E. betae), vegetables (for example E. pisi), such as cucumber species (for example E. cichoracearum) and cabbage species, such as oilseed rape (for example E. cruciferarum); Eutypa lata (Eutypa cancer or dieback, anamorph: Cytosporina lata, syn. Libertella blepharis) on fruit trees, grapevines and many ornamental trees; Exserohilum (syn. Helminthosporium) spp. on corn (for example E. turcicum); Fusarium (teleomorph: Gibberella) spp. (wilt disease, root and stem rot) on various plants, such as for example F. graminearum or F. culmorum (root rot and silver-top) on cereals (for example wheat or barley), F. oxysporum on tomatoes, F. solani on soybeans and F. verficillioides on corn; Gaeumannomyces graminis (take-all) on cereals (for example wheat or barley) and corn; Gibberella spp. on cereals (for example G. zeae) and rice (for example G. fujikuroi: bakanae disease); Glomerella cingulata on grapevines, pomaceous fruit and other plants and G. gossypii on cotton; grainstaining complex on rice; Guignardia bidwellii (black rot) on grapevines; Gymnosporangium spp. on Rosaceae and juniper, for example G. sabinae (pear rust) on pears; Helminthosporium spp. (syn. Drechslera, teleomorph: Cochliobolus) on corn, cereals and rice; Hemileia spp., for example H. vastatrix (coffee leaf rust) on coffee; Isariopsis clavispora (syn. Cladosporium vitis) on grapevines; Macrophomina phaseolina (syn. phaseoli) (root/stem rot) on soybeans and cotton; Microdochium (syn. Fusarium) nivale (pink snow mold) on cereals (for example wheat or barley); Microsphaera diffusa (powdery mildew) on soybeans; Monilinia spp., for example M. laxer, M. fructicola and M. fructigena (blossom and twig blight) on stone fruit and other Rosaceae; Mycosphaerella spp. on cereals, bananas, soft fruit and peanuts, such as for example M. graminicola (anamorph: Septoria tritici, Septoria leaf blotch) on wheat or M. fijiensis (sigatoka disease) on bananas; Peronospora spp. (downy mildew) on cabbage (for example P. brassicae), oilseed rape (for example P. parasitica), bulbous plants (for example P. destructor), tobacco (P. tabacina) and soybeans (for example P. manshurica); Phakopsora pachyrhizi and P. meibomiae (soybean rust) on soybeans; Phialophora spp., for example on grapevines (for example P. tracheiphila and P. tetraspora) and soybeans (for example P. gregata: stem disease); Phoma lingam (root and stem rot) on oilseed rape and cabbage and P. betae (leaf spot) on sugar beets; Phomopsis spp. on sunflowers, grapevines (for example P. viticola: dead-arm disease) and soybeans (for example stem canker/stem blight: P. phaseoli, teleomorph: Diaporthe phaseolorum); Physoderma maydis (brown spot) on corn; Phytophthora spp. (wilt disease, root, leaf, stem and fruit rot) on various plants, such as on bell peppers and cucumber species (for example P. capsici), soybeans (for example P. megasperma, syn. P. sojae), potatoes and tomatoes (for example P. infestans: late blight and brown rot) and deciduous trees (for example P. ramorum: sudden oak death); Plasmodiophora brassicae (club-root) on cabbage, oilseed rape, radish and other plants; Plasmopara spp., for example P. viticola (peronospora of grapevines, downy mildew) on grapevines and P. halstedii on sunflowers; Podosphaera spp. (powdery mildew) on Rosaceae, hops, pomaceous fruit and soft fruit, for example P. leucotricha on apple; Polymyxa spp., for example on cereals, such as barley and wheat (P. graminis) and sugar beets (P. betae) and the viral diseases transmitted thereby; Pseudocercosporella herpotrichoides (eyespot/stern break, teleomorph: Tapesia yallundae) on cereals, for example wheat or barley; Pseudoperonospora (downy mildew) on various plants, for example P. cubensis on cucumber species or P. humili on hops; Pseudopezicula tracheiphila (angular leaf scorch, anamorph: Phialophora) on grapevines; Puccinia spp. (rust disease) on various plants, for example P. triticina (brown rust of wheat), P. striiformis (yellow rust), P. hordei (dwarf leaf rust), P. graminis (black rust) or P. recondita (brown rust of rye) on cereals, such as for example wheat, barley or rye, and on asparagus (for example P. asparagi); Pyrenophora (anamorph: Drechslera) tritici-repentis (speckled leaf blotch) on wheat or P. teres (net blotch) on barley; Pyriculana spp., for example P. oryzae (teleomorph: Magnaporthe grisea, rice blast) on rice and P. grisea on lawn and cereals; Pythium spp. (damping-off disease) on lawn, rice, corn, wheat, cotton, oilseed rape, sunflowers, sugar beets, vegetables and other plants (for example P. ultimum or P. aphanidermatum); Ramularia spp., for example R. colla-cygni (Ramularia leaf and lawn spot/physiological leaf spots) on barley and R. beticola on sugar beets; Rhizoctonia spp. on cotton, rice, potatoes, lawn, corn, oilseed rape, potatoes, sugar beet, vegetables and on various other plants, for example R. solani (root and stem rot) on soybeans, R. solani (sheath blight) on rice or R. cerealis (sharp eyespot) on wheat or barley; Rhizopus stolonifer (soft rot) on strawberries, carrots, cabbage, grapevines and tomato; Rhynchosporium secalis (leaf spot) on barley, rye and triticale; Sarocladium oryzae and S. attenuatum (sheath rot) on rice; Sclerotinia spp. (stem or white rot) on vegetable and field crops, such as oilseed rape, sunflowers (for example Sclerotinia sclerotiorum) and soybeans (for example S. rolfsii), Septona spp. on various plants, for example S. glycines (leaf spot) on soybeans, S. tritici (Septoria leaf blotch) on wheat and S. (syn. Stagonospora) nodorum (leaf blotch and glume blotch) on cereals; Uncinula (syn. Erysiphe) necator (powdery mildew, anamorph: Oidium tuckeri) on grapevines; Setospaeria spp. (leaf spot) on corn (for example S. turcicum, syn. Helminthosporium turcicum) and lawn; Sphacelotheca spp. (head smut) on corn, (for example S. reiliana: kernel smut), millet and sugar cane; Sphaerotheca fuliginea (powdery mildew) on cucumber species; Spongospora subterranea (powdery scab) on potatoes and the viral diseases transmitted thereby; Stagonospora spp. on cereals, for example S. nodorum (leaf blotch and glume blotch, teleomorph: Leptosphaeria [syn. Phaeosphaeria] nodorum) on wheat; Synchytrium endobioticum on potatoes (potato wart disease); Taphrina spp., for example T. deformans (curly-leaf disease) on peach and T. pruni (plum-pocket disease) on plums; Thielaviopsis spp. (black root rot) on tobacco, pome fruit, vegetable crops, soybeans and cotton, for example T. basicola (syn. Chalara elegans); Tilletia spp. (bunt or stinking smut) on cereals, such as for example T. tritici (syn. T. caries, wheat bunt) and T. controversy (dwarf bunt) on wheat; Typhula incarnata (gray snow mold) on barley or wheat; Urocystis spp., for example U. occulta (flag smut) on rye; Uromyces spp. (rust) on vegetable plants, such as beans (for example U. appendiculatus, syn. U. phaseoli) and sugar beets (for example U. betae); Ustilago spp. (loose smut) on cereals (for example U. nuda and U. avaenae), corn (for example U. maydis: corn smut) and sugar cane; Venturia spp. (scab) on apples (for example V. inaequalis) and pears; and Verticillium spp. (leaf and shoot wilt) on various plants, such as fruit trees and ornamental trees, grapevines, soft fruit, vegetable and field crops, such as for example V. dahliae on strawberries, oilseed rape, potatoes and tomatoes.

Moreover, the mixtures and compositions according to the invention are suitable for controlling harmful fungi in the protection of stored products (and of harvested products) and in the protection of materials and buildings. The term “protection of materials and buildings” encompasses the protection of industrial and non-living materials such as, for example, adhesives, glues, wood, paper and cardboard, textiles, leather, paint dispersions, plastic, cooling lubricants, fibers and tissues, against attack and destruction by unwanted microorganisms such as fungi and bacteria. In the protection of wood and materials, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of materials to the following yeast fungi: Candida spp. and Saccharomyces cerevisae.

The compounds of the formula I, and also those of the formula II, may be present in various crystal modifications which may differ in their biological activity. Their mixtures are included in the scope of the present invention.

The mixtures and compositions according to the invention are suitable for improving plant health. Moreover, the invention relates to a method for improving plant health by treating the plants, the plant propagation material and/or the site at which the plants grow or are intended to grow with an effective amount of the compounds I or the compositions according to the invention.

The term “plant health” comprises those states of a plant and/or its harvested material which are determined by various indicators individually or in combination, such as, for example, yield (for example increased biomass and/or increased content of utilizable ingredients), plant vitality (for example increased plant growth and/or greener leaves (“greening effect”)), quality (for example increased content or composition of certain ingredients) and tolerance to biotic and/or abiotic stress. The indicators mentioned here for a state of plant health may occur independently of one another or may influence each other.

The mixtures according to the invention are employed as such or in the form of a composition by treating the harmful fungi, their habitat or the plants or plant propagation materials, for example seed materials, to be protected from fungal attack, for example seed, the soil, areas, materials or spaces with a fungicidally effective amount of the mixture according to the invention. The application can be carried out both before and after the infection of the plants, plant propagation materials, for example seed materials, the soil, the areas, materials or spaces by the fungi.

Plant propagation materials can be treated prophylactically during or even before sowing or during or even before transplanting with the mixtures according to the invention or a composition thereof (a composition comprising at least one compound I and at least one compound II, preferably one or two compounds II).

The invention furthermore relates to agrochemical compositions comprising a solvent or solid carrier and the mixture according to the invention, and also to their use for controlling harmful fungi.

In this context, the term “preparation” has the same meaning as “composition”, in particular “agrochemical composition”, and “formulation”.

An agrochemical composition comprises a fungicidally effective amount of the mixture according to the invention. The term “effective amount” refers to an amount of the agrochemical composition or of the mixture according to the invention which is sufficient for controlling harmful fungi on crop plants or in the protection of materials and buildings and does not cause any significant damage to the treated crop plants. Such an amount may vary within a wide range and is influenced by numerous factors, such as, for example, the harmful fungus to be controlled, the respective crop plant or materials treated, the climatic conditions and compounds.

The compounds I and the one or more compounds II can be applied simultaneously, that is jointly or separately, or in succession, the order, in the case of separate application, generally not having any effect on the control results. The method for controlling harmful fungi is carried out by separate or joint application of the compound I and the compound(s) II or of mixtures of the compound I and the compounds) II by spraying or dusting the seed, the plants or the soil before or after sowing of the plants or before or after emergence of the plants.

The compounds I and II may be present in a joint composition or in separate compositions. Here, type and preparation of the composition in question corresponds to type and preparation as described here in a general manner for compositions.

The compounds I and the compounds II, and also their N-oxides and salts and their mixtures, can be converted into the types customary for agrochemical compositions, for example solutions, emulsions, suspensions, dusts, powders, pastes and granules. The type of composition depends on the respective intended purpose; in each case, it should ensure a fine and even distribution of the compounds of the mixtures according to the invention.

Here, examples of types of compositions are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG) which may either be water-soluble or -dispersible (wettable), and also gels for treating plant propagation materials such as seed (GF).

In general, the composition types (for example EC, SC, OD, FS, WG, SG, WP, SP, SS, WS, GE) are used in diluted form. Composition types such as DP, DS, GR, FG, GG and MG are generally employed in undiluted form.

The agrochemical compositions are prepared in a known manner (see, for example, U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th edition, McGraw-Hill, New York, 1963, 8-57 and ff., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (John Wiley & Sons, New York, 1961), Hance et al.: Weed Control Handbook (8th Ed., Blackwell Scientific Publications, Oxford, 1989) and Mollet, H. and Grubemann, A.: Formulation technology (Wiley VCH Verlag, Weinheim, 2001).

The agrochemical compositions may furthermore also comprise auxiliaries customary for crop protection compositions, the selection of the auxiliaries depending on the specific use form or the active compound.

Examples of suitable auxiliaries are solvents, solid carriers, surfactants (such as further solubilizers, protective colloids, wetting agents and tackifiers), organic and inorganic thickeners, bactericides, antifreeze agents, antifoams, if appropriate colorants and adhesives (for example for the treatment of seed).

Suitable solvents are water, organic solvents, such as mineral oil fractions having a medium to high boiling point, such as kerosene and diesel oil, furthermore coal tar oils, and also oils of vegetable or animal origin, aliphatic, cyclic and aromatic hydrocarbons, for example paraffins, tetrahydronaphthalene, alkylated naphthalenes and derivatives thereof, alkylated benzenes and derivatives thereof, alcohols, such as methanol, ethanol, propanol, butanol and cyclohexanol, glycols, ketones, such as cyclohexanone, gamma-butyrolactone, dimethyl fatty amides, fatty acids and fatty acid esters and strongly polar solvents, for example amines, such as N-methylpyrrolidone. In principle, it is also possible to use solvent mixtures, and also mixtures of the solvents mentioned above and water.

Solid carriers are mineral earths such as silicas, silica gels, silicates, talc, kaolin, limestone, lime, chalk, bolus, loess, clay, dolomite, diatomaceous earth, calcium sulfate and magnesium sulfate, magnesium oxide, ground plastics, fertilizers, such as ammonium sulfate, ammonium phosphate, ammonium nitrate, ureas and vegetable products such as cornmeal, bark dust, sawdust, nutshell meal, cellulose powder or other solid carriers.

Suitable surfactants (adjuvants, wetting agents, tackifiers, dispersants or emulsifiers) are the alkali metal, alkaline earth metal and ammonium salts of aromatic sulfonic acids, for example of lignosulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany), and also of fatty acids, alkyl- and alkylarylsulfonates, alkyl, lauryl ether and fatty alcohol sulfates, and also salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octyl phenol ether, ethoxylated isooctylphenol, octylphenol or nonylphenol, alkylphenyl polyglycol ether, tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors, and also proteins, denatured proteins, polysaccharides (for example methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and copolymers thereof.

Examples of thickeners (i.e. compounds which impart modified flow properties to the composition, i.e. high viscosity in the state of rest and low viscosity in motion) are polysaccharides and also organic and inorganic sheet minerals, such as xanthan gum (Kelzan®, CP Kelco, USA), Rhodopol® 23 (Rhodia, France) or Veegum® (R.T. Vanderbilt, USA) or Attaclay® (Engelhard Corp., NJ, USA).

Bactericides can be added for stabilizing the composition. Examples of bactericides are bactericides based on dichlorophen and benzyl alcohol hemiformal (Proxel^{∃} from ICI or Acticide^{∃} RS from Thor Chemie and Kathon^{∃} MK from Rohm & Haas), and also isothiazolinone derivatives, such as alkylisothiazolinones and benzisothiazolinones (Acticid^{∃} MBS from Thor Chemie).

Examples of suitable antifreeze agents are ethylene glycol, propylene glycol, urea and glycerol.

Examples of antifoams are silicone emulsions (such as, for example, Silikon® SRE, Wacker, Germany or Rhodorsil®, Rhodia, France), long-chain alcohols, fatty acids, salts of fatty acids, organofluorine compounds and mixtures thereof.

Examples of colors are both pigments, which are sparingly soluble in water, and dyes, which are soluble in water. Examples which may be mentioned are the dyes and pigments known under the names Rhodamin B, C. I. Pigment Red 112 and C. I. Solvent Red 1, Pigment blue 15:4, Pigment blue 15:3, Pigment blue 15:2, Pigment blue 15:1, Pigment blue 80, Pigment yellow 1, Pigment yellow 13, Pigment red 48:2, Pigment red 48:1, Pigment red 57:1, Pigment red 53:1, Pigment orange 43, Pigment orange 34, Pigment orange 5, Pigment green 36, Pigment green 7, Pigment white 6, Pigment brown 25, Basic violet 10, Basic violet 49, Acid red 51, Acid red 52, Acid red 14, Acid blue 9, Acid yellow 23, Basic red 10, Basic red 108.

Examples of adhesives are polyvinylpyrrolidone, polyvinyl acetate, polyvinyl alcohol and cellulose ether (Tylose®, Shin-Etsu, Japan).

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the compounds I and the further active compounds II with at least one solid carrier.

Granules, for example coated granules, impregnated granules and homogeneous granules, can be prepared by binding the active compounds to at least one solid carrier. Solid carriers are, for example, mineral earths, such as silica gels, silicates, talc, kaolin, attaclay, limestone, lime, chalk, bole, loess, clay, dolomite, diatomaceous earth, calcium sulfate and magnesium sulfate, magnesium oxide, ground synthetic substances, fertilizers, such as ammonium sulfate, ammonium phosphate, ammonium nitrate, ureas and vegetable products, such as cereal meal, tree bark meal, sawdust and nutshell meal, cellulose powder or other solid carriers.

The following are examples of types of composition:

1. Types of Composition for Dilution with Water

i) Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a composition having an active compound content of 10% by weight.

ii) Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight.

iii) Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The composition has an active compound content of 15% by weight.

iv) Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The composition has an active compound content of 25% by weight.

v) Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the composition is 20% by weight.

vI) Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The composition has an active compound content of 50% by weight.

vii) Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the composition is 75% by weight.

viii) Gels (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

### 2. Types of Composition to be Applied Undiluted

ix) Dusts (DP, DS)

5 parts by weight of the active compounds are ground finely and mixed intimately with 95 parts by weight of finely divided kaolin. This gives a dustable product with an active compound content of 5% by weight.

x) Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

xi) ULV Solutions (UL)

10 parts by weight of the active compounds are dissolved in 90 parts by weight of an organic solvent, for example xylene. This gives a composition with an active compound content of 10% by weight to be applied undiluted.

In general, the compositions of the mixtures according to the invention comprise from 0.01 to 95% by weight, preferably from 0.1 to 90% by weight, of the compounds I and II or their mixtures. The compounds I and II are preferably employed in a purity of from 90% to 100%, preferably from 95% to 100% (NMR spectrum).

Water-soluble concentrates (LS), suspensions (FS), dusts (DS), water-dispersible and water-soluble powders (WS, SS), emulsions (ES), emulsifiable concentrates (EC) and gels (GF) are usually used for the treatment of plant propagation materials, in particular seed. These compositions can be applied to the propagation materials, in particular seed, in undiluted or, preferably, diluted form. In this case, the corresponding composition can be diluted 2 to 10 times so that in the compositions used for the seed dressing from 0.01 to 60% by weight, preferably from 0.1 to 40% by weight of active compound are present. The application can be carried out before or during sowing. The treatment of plant propagation material, in particular the treatment of seed, is known to the person skilled in the art and is carried out by dusting, coating, pelleting, dipping or drenching the plant propagation material, the treatment preferably being carried out by pelleting, coating and dusting or by furrow treatment, such that, for example, premature germination of the seed is prevented.

For seed treatment, preference is given to using suspensions. Such compositions usually comprise from 1 to 800 g of active compound/I, from 1 to 200 g of surfactants/I, from 0 to 200 g of antifreeze agents/I, from 0 to 400 g of binders/I, from 0 to 200 g of colorants/I and solvents, preferably water.

The compounds I and II or their mixtures can be used as such or in the form of their compositions, for example in the form of directly sprayable solutions, powders, suspensions, dispersions, emulsions, oil dispersions, pastes, dustable products, materials for spreading or granules, by means of spraying, atomizing, dusting, spreading, raking in, immersing or pouring. The types of composition depend entirely on the intended purposes; the intention is to ensure in each case the finest possible distribution of the active compounds or active compound mixtures according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply compositions comprising over 95% by weight of active compound, or even to apply the active compound without additives.

When used in crop protection, the application rates are from 0.001 to 2.0 kg of active compound per ha, preferably from 0.005 to 2 kg per ha, particularly preferably from 0.05 to 0.9 kg per ha, especially from 0.1 to 0.75 kg per ha, depending on the nature of the desired effect.

In the treatment of plant propagation materials, for example seed, the amounts of active compound (or amounts of active compound mixtures) used are generally from 0.1 to 1000 g/100 kg of propagation material or seed, preferably from 1 to 1000 g/100 kg, particularly preferably from 1 to 100 g/100 kg, especially from 5 to 100 g/100 kg.

When used in the protection of materials or stored products, the amount of active compound or active compound mixture applied depends on the kind of application area and on the desired effect. Amounts typically applied in the protection of materials are, for example, from 0.001 g to 2 kg, preferably from 0.005 g to 1 kg, of active compound per cubic meter of treated material.

Various types of oils, wetters, adjuvants, herbicides, bactericides, other fungicides and/or pesticides may be added to the active compounds or active compound mixtures or the compositions comprising them, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus® 245, Atplus® MBA 1303, Plurafac® LF 300 and Lutensol® ON 30; EO-PO block polymers, for example Pluronic® RPE 2035 and Genapol® B; alcohol ethoxylates, for example Lutensol® XP 80; and sodium dioctylsulfosuccinate, for example Leophen® RA.

### SYNTHESIS EXAMPLES

The synthesis of the compounds of the formula I is carried out as described in DE19520097, WO97/41107, WO97/42178, WO97/43269, WO97/44331, WO97/44332 or WO99/05149, or by various routes analogously to prior art processes known per se (see, for example, the prior art cited at the outset and Pflanzenschutz-Nachrichten Bayer 57/2004, 2, pages 145-162), or as described below.

With appropriate modification of the starting materials, the procedures given in the synthesis examples below can be used to obtain further compounds of the formula I or the precursors thereof.

Melting points were obtained on a MeI-Temp II instrument and are uncorrected. 1H-NMR spectra were recorded on a Bruker AC 300 spectrometer at 300 MHz and are referenced to tetramethylsilane as internal standard (from Aldrich or Cambridge Isotope Laboratories).

ESI mass spectra were recorded on a Shimadzu LCMS-2010 EV mass spectrometer. APCI mass spectra were recorded on a Shimadzu LCMS-2010 EV mass spectrometer.

### Example I-6

### Synthesis of 1-[(2S,3R)-3-(2-chlorophenyl)-2-(4-fluorophenyl)oxiran-2-ylmethyl]-5-thiocyanato-1H-1,2,4-triazole

At −78° C., n-BuLi (0.46 ml, 0.91 mmol, 2.0 M in THF) was added dropwise to a solution of 1-[(2S,3R)-3-(2-chlorophenyl)-2-(4-fluorophenyl)oxiran-2-ylmethyl]-1H-[1,2,4]triazole (250 mg, 0.76 mmol) in anhydrous THF (5 ml). After 30 minutes, elemental sulfur (49 mg, 1.53 mmol) was added, and the mixture was stirred at −78° C. for another 4 hours. BrCN (96 mg, 0.91 mmol) was then added at −78° C., the reaction mixture was allowed to thaw to room temperature and stirring was continued overnight. NH₄Cl (saturated, aqueous, 30 ml) was added, and the mixture was extracted with EtOAc (30 ml). The organic phase was washed with saturated sodium chloride solution (3×20 ml), separated off, dried over sodium sulfate and concentrated under reduced pressure. The residue was purified by column chromatography (silica gel, 10:1 to 4:1 CH₂Cl₂/EtOAc). This gave the thiocyanate 1-6 as a white solid; yield 42 mg (15%). ¹H NMR (300 MHz, CDCl₃) δ 7.83 (s, 1H), 7.51-7.50 (m, 1H), 7.50-7.40 (m, 1H), 7.40-7.32 (m, 4H), 7.10-7.00 (m, 2H), 4.85 (ABq, J=14.7 Hz, 1H), 4.21 (s, 1H), 4.11 (ABq, J=15.0 Hz, 1H); ESI MS, m/z 387 [M+H]⁺. Melting point: 146° C.

The starting material 1-[(2S,3R)-3-(2-chlorophenyl)-2-(4-fluorophenyl)oxiran-2-ylmethyl]-1H-[1,2,4]triazole can be prepared as described, for example, in the prior art cited at the outset or in WO 2007/147841 (PCT/EP2007/056124), WO 2007/147769 (PCT/EP2007/055870) and WO 2007/147778 (PCT/EP2007/055932), or analogously thereto.

### Example I-18

### Preparation of 1-rel-[(2S,3R)-3-(2-chlorophenyl)-2-(4-fluorophenyl)-oxiranylmethyl]-5-ethylsulfanyl-1H-[1,2,4]triazole

At −78° C., LDA (150 mmol, 75 ml, 2.0 M in THF) was added dropwise with stirring to a solution of 1-rel-[(2S,3R)-3-(2-chlorophenyl)-2-(4-fluorophenyl)oxiran-2-ylmethyl]-1H-[1,2,4]triazole (40 g, 121.3 mmol) in anhydrous THF (700 ml). After 30 min, diethyl disulfide (18 ml, 146.2 mmol) was added, and the reaction mixture was stirred at −78° C. for 4 h. The reaction mixture was quenched with NH₄Cl (saturated, 150 ml) and extracted with EtOAc (500 ml). The organic phase was washed with saturated sodium chloride solution (3×200 ml), dried with sodium sulfate and concentrated. The residue was purified by column chromatography (silica gel, mobile phase: hexane/EtOAc using a gradient from 3:1 to 1:1) to give the title compound as a white solid (34.8 g, 81%). ¹H NMR (300 MHz, CDCl₃) δ 7.74 (s, 1H), 7.66-7.62 (m, 1H), 7.48-7.34 (m, 5H), 7.03-6.97 (m, 2H), 4.59 (d, 1H, J=15.0 Hz), 4.19 (s, 1H), 3.87 (d, 1H, J=15.0 Hz), 3.04 (q, 2H J=7.5 Hz), 1.22 (t, 3H, J=7.5 Hz); APCI MS, m/z 391 [M+H]⁺; mp=80-82° C. The ¹H NMR spectrum was obtained on a Bruker AC 300 spectrometer at 300 MHz. The internal standard was tetramethylsilane. The APCI mass spectrum was obtained on a Shimadzu LCMS-2010 EV mass spectrometer. The melting point was measured on a MeI-Temp II apparatus and is uncorrected.

### USE EXAMPLES

The fungicidal action of the mixtures according to the invention can be demonstrated by the following tests:

### Microtest

### Preparation of Active Compound

The active compounds were formulated separately or jointly as a stock solution having a concentration of 10 000 ppm in DMSO.

The active compound orysastrobin was used as a commercial formulation and, with respect to the active compound, diluted with water.

The determined values (measured parameters) for the percentage of infection on the leaves were compared to the growth of the active compound-free control variant and the fungus- and active compound-free blank value to determine the relative growth in % of the pathogens in the individual active compounds and were thus converted into efficacy % of the untreated control. An efficacy of 0 means the same degree of infection as in the untreated control; an efficacy of 100 means 0% infection. The expected efficacies for active compound combinations were determined using the Colby formula (Colby, S. R. “Calculating synergistic and antagonistic responses of herbicide combinations”, Weeds, 15, pp. 20-22, 1967) and compared to the observed efficacies.

The efficacy (E) is calculated as follows using Abbot's formula:

E=(1−α/β)·100

α corresponds to the fungal infection of the treated plants in % and
β corresponds to the fungal infection of the untreated (control) plants in %

At an efficacy of 0 the degree of infection of the treated plants corresponds to that of the untreated control plants; at an efficacy of 100 the treated plants are not infected.

The expected efficacies for active compound combinations were determined using the Colby formula (Colby, S. R. “Calculating synergistic and antagonistic responses of herbicide combinations”, Weeds, 15, pp. 20-22, 1967) and compared to the observed efficacies.

Colby's formula:

E=x+y−x·y/100
E expected efficacy, expressed in % of the untreated control, when using the mixture of the active compounds A and B at the concentrations a and b
x efficacy, expressed in % of the untreated control, when using the active compound A at the concentration a
y efficacy, expressed in % of the untreated control, when using the active compound B at the concentration b

### Use Example No. M1

### Activity Against the Late Blight Pathogen Phytophthora infestans in the Microtiter Test (Phytin)

The stock solution was pipetted into a microtiter plate (MTP) and diluted to the stated active compound concentration with water. An aqueous pea juice-based zoospore suspension of Phytophthora infestans was then added. The plates were placed in a water vapor-saturated chamber at a temperature of 18° C. Using an absorption photometer, the MTPs were measured at 405 nm on day 7 after the inoculation.

### Binary Mixtures

| | | | | Effect | |
|---|---|---|---|---|---|
| Active compound/ | | | | calculated | |
| active compound | Concentration | | Observed | according | Synergism |
| combination | (ppm) | Mixture | effect | to Colby | (%) |
| I-3 | 63 | — | 43 | | |
| I-10 | 16 | — | 30 | | |
| I-14 | 16 | — | 26 | | |
| boscalid | 16 | — | 20 | | |
| pyraclostrobin | 16 | — | 16 | | |
| I-3 | 63 | 4:1 | 100 | 54 | 46 |
| boscalid | 16 | | | | |
| I-10 | 16 | 250:1 | 68 | 41 | 27 |
| pyraclostrobin | 0.063 | | | | |
| I-14 | 16 | 250:1 | 87 | 38 | 49 |
| pyraclostrobin | 0.063 | | | | |

### Ternary Mixtures

| | | | | Effect | |
|---|---|---|---|---|---|
| Active compound/ | | | | calculated | |
| active compound | Concentration | | Observed | according | Synergism |
| combination | (ppm) | Mixture | effect | to Colby | (%) |
| I-1 + epoxiconazole | 63 + 1 | 63:1 | 41 | | |
| I-3 + pyraclostrobin | 16 + 0.063 | 250:1 | 34 | | |
| I-3 + epoxiconazole | 16 + 0.25 | 63:1 | 20 | | |
| I-8 + pyraclostrobin | 16 + 0.063 | 250:1 | 27 | | |
| I-10 + pyraclostrobin | 16 + 0.063 | 250:1 | 68 | | |
| I-12 + pyraclostrobin | 16 + 0.063 | 250:1 | 24 | | |
| boscalid | 16 | — | 20 | | |
| | 4 | — | 4 | | |
| orysastrobin | 0.25 | — | 5 | | |
| I-1 + epoxiconazole | 63 + 1 | 63:1:16 | 97 | 53 | 44 |
| boscalid | 16 | | | | |
| I-3 + pyraclostrobin | 16 + 0.063 | 250:1:63 | 60 | 37 | 23 |
| boscalid | 4 | | | | |
| I-3 + epoxiconazole | 16 + 0.25 | 63:1:1 | 60 | 24 | 36 |
| orysastrobin | 0.25 | | | | |
| I-8 + pyraclostrobin | 16 + 0.063 | 250:1:63 | 57 | 30 | 27 |
| boscalid | 4 | | | | |
| I-10 + pyraclostrobin | 16 + 0.063 | 250:1:63 | 94 | 69 | 25 |
| boscalid | 4 | | | | |
| I-12 + pyraclostrobin | 16 + 0.063 | 250:1:63 | 58 | 27 | 31 |
| boscalid | 4 | | | | |

### Use Example No. M2

### Activity Against the Gray Mold Pathogen Botrytis cinerea in the Microtiter Test (Botrci)

The stock solution was pipetted into a microtiter plate (MTP) and diluted to the stated active compound concentration with water. An aqueous malt-based spore suspension of Botrytis cinerea was then added. The plates were placed in a water vapor-saturated chamber at a temperature of 18° C. Using an absorption photometer, the MTPs were measured at 405 nm on day 7 after the inoculation.

### Binary Mixtures

| | | | | Effect | |
|---|---|---|---|---|---|
| Active compound/ | | | | calculated | |
| active compound | Concentration | | Observed | according | Synergism |
| combination | (ppm) | Mixture | effect | to Colby | (%) |
| I-3 | 16 | — | 15 | | |
| I-8 | 63 | — | 24 | | |
| epoxiconazole | 0.25 | — | 57 | | |
| orysastrobin | 1 | — | 2 | | |
| I-3 | 16 | 63:1 | 84 | 64 | 20 |
| epoxiconazole | 0.25 | | | | |
| I-8 | 63 | 63:1 | 83 | 26 | 57 |
| orysastrobin | 1 | | | | |

### Ternary Mixtures

| | | | | Effect | |
|---|---|---|---|---|---|
| Active compound/ | | | | calculated | |
| active compound | Concentration | | Observed | according | Synergism |
| combination | (ppm) | Mixture | effect | to Colby | (%) |
| I-1 + pyraclostrobin | 16 + 0.063 | 250:1 | 26 | | |
| I-3 + pyraclostrobin | 16 + 0.063 | 250:1 | 4 | | |
| I-3 + epoxiconazole | 1 + 0.016 | 63:1 | 12 | | |
| I-8 + pyraclostrobin | 63 + 0.25 | 250:1 | 32 | | |
| I-8 + epoxiconazole | 16 + 0.25 | 63:1 | 50 | | |
| I-10 + pyraclostrobin | 16 + 0.063 | 250:1 | 27 | | |
| I-12 + pyraclostrobin | 16 + 0.63 | 250:1 | 11 | | |
| epoxiconazole | 0.25 | — | 57 | | |
| orysastrobin | 1 | — | 2 | | |
| | 0.25 | — | 4 | | |
| | 0.016 | — | 1 | | |
| I-1 + pyraclostrobin | 16 + 0.063 | 250:1:4 | 94 | 68 | 26 |
| epoxiconazole | 0.25 | | | | |
| I-3 + pyraclostrobin | 16 + 0.063 | 250:1:4 | 86 | 59 | 27 |
| epoxiconazole | 0.25 | | | | |
| I-3 + epoxiconazole | 1 + 0.016 | 63:1:1 | 62 | 13 | 49 |
| orysastrobin | 0.016 | | | | |
| I-8 + pyraclostrobin | 63 + 0.25 | 250:1:4 | 87 | 33 | 54 |
| orysastrobin | 1 | | | | |
| I-8 + epoxiconazole | 16 + 0.25 | 63:1:1 | 78 | 51 | 27 |
| orysastrobin | 0.25 | | | | |
| I-10 + pyraclostrobin | 16 + 0.063 | 250:1:4 | 90 | 69 | 21 |
| epoxiconazole | 0.25 | | | | |
| I-12 + pyraclostrobin | 16 + 0.63 | 250:1:4 | 81 | 62 | 19 |
| epoxiconazole | 0.25 | | | | |

### Use Example No. M3

### Activity Against the Rice Blast Pathogen Pyricularia oryzae in the Microtiter Test (Pyrior)

The stock solution was pipetted into a microtiter plate (MTP) and diluted to the stated active compound concentration with water. An aqueous malt-based spore suspension of Pyricularia oryzae was then added. The plates were placed in a water vapor-saturated chamber at a temperature of 18° C. Using an absorption photometer, the MTPs were measured at 405 nm on day 7 after the inoculation.

### Binary Mixtures

| | | | | Effect | |
|---|---|---|---|---|---|
| Active compound/ | | | | calculated | |
| active compound | Concentration | | Observed | according | Synergism |
| combination | (ppm) | Mixture | effect | to Colby | (%) |
| I-1 | 16 | — | 3 | | |
| I-3 | 63 | — | 62 | | |
| | 16 | — | 8 | | |
| I-10 | 16 | — | 35 | | |
| I-12 | 63 | — | 16 | | |
| | 16 | — | 7 | | |
| I-14 | 16 | — | 18 | | |
| pyrimethanil | 4 | — | 47 | | |
| boscalid | 16 | — | 8 | | |
| epoxiconazole | 0.25 | — | 5 | | |
| I-1 | 16 | 4:1 | 83 | 48 | 35 |
| pyrimethanil | 4 | | | | |
| I-3 | 63 | 4:1 | 100 | 65 | 35 |
| boscalid | 16 | | | | |
| I-3 | 16 | 4:1 | 83 | 51 | 32 |
| pyrimethanil | 4 | | | | |
| I-10 | 16 | 63:1 | 67 | 39 | 28 |
| epoxiconazole | 0.25 | | | | |
| I-10 | 16 | 4:1 | 89 | 65 | 24 |
| pyrimethanil | 4 | | | | |
| I-12 | 16 | 63:1 | 30 | 11 | 19 |
| epoxiconazole | 0.25 | | | | |
| I-12 | 63 | 4:1 | 63 | 23 | 40 |
| boscalid | 16 | | | | |
| I-12 | 16 | 4:1 | 74 | 50 | 24 |
| pyrimethanil | 4 | | | | |
| I-14 | 16 | 4:1 | 90 | 56 | 34 |
| pyrimethanil | 4 | | | | |

### Ternary Mixtures

| | | | | Effect | |
|---|---|---|---|---|---|
| Active compound/ | | | | calculated | |
| active compound | Concentration | | Observed | according | Synergism |
| combination | (ppm) | Mixture | effect | to Colby | (%) |
| I-1 + pyraclostrobin | 1 + 0.004 | 250:1 | 15 | | |
| I-1 + epoxiconazole | 16 + 0.25 | 63:1 | 15 | | |
| I-3 + pyraclostrobin | 1 + 0.004 | 250:1 | 21 | | |
| I-3 + epoxiconazole | 16 + 0.25 | 63:1 | 24 | | |
| I-8 + pyraclostrobin | 1 + 0.004 | 250:1 | 19 | | |
| I-8 + epoxiconazole | 16 + 0.25 | 63:1 | 29 | | |
| I-10 + pyraclostrobin | 1 + 0.004 | 250:1 | 40 | | |
| I-12 + pyraclostrobin | 1 + 0.004 | 250:1 | 16 | | |
| chlorothalonil | 0.063 | — | 4 | | |
| boscalid | 4 | — | 0 | | |
| | 0.25 | — | 0 | | |
| orysastrobin | 0.25 | — | 56 | | |
| | 0.016 | — | 2 | | |
| epoxiconazole | 0.016 | — | 0 | | |
| I-1 + pyraclostrobin | 1 + 0.004 | 250:1:16 | 40 | 19 | 21 |
| chlorothalonil | 0.063 | | | | |
| I-1 + epoxiconazole | 16 + 0.25 | 63:1:16 | 83 | 15 | 68 |
| boscalid | 4 | | | | |
| I-3 + pyraclostrobin | 1 + 0.004 | 250:1:16 | 44 | 24 | 20 |
| chlorothalonil | 0.063 | | | | |
| I-3 + epoxiconazole | 16 + 0.25 | 63:1:16 | 43 | 24 | 19 |
| boscalid | 4 | | | | |
| I-3 + epoxiconazole | 16 + 0.25 | 63:1:1 | 97 | 66 | 31 |
| orysastrobin | 0.25 | | | | |
| I-8 + pyraclostrobin | 1 + 0.004 | 250:1:16 | 56 | 22 | 34 |
| chlorothalonil | 0.063 | | | | |
| I-8 + epoxiconazole | 16 + 0.25 | 63:1:16 | 57 | 29 | 28 |
| boscalid | 4 | | | | |
| I-10 + pyraclostrobin | 1 + 0.004 | 250:1:4 | 75 | 40 | 35 |
| epoxiconazole | 0.016 | | | | |
| I-10 + pyraclostrobin | 1 + 0.004 | 250:1:63 | 67 | 40 | 27 |
| boscalid | 0.25 | | | | |
| I-10 + pyraclostrobin | 1 + 0.004 | 250:1:4 | 66 | 41 | 25 |
| orysastrobin | 0.016 | | | | |
| I-12 + pyraclostrobin | 1 + 0.004 | 250:1:4 | 44 | 18 | 26 |
| orysastrobin | 0.016 | | | | |
| I-12 + pyraclostrobin | 1 + 0.004 | 250:1:16 | 38 | 19 | 19 |
| chlorothalonil | 0.063 | | | | |

### Use Example No. M4

### Activity Against the Septoria Leaf Spot Pathogen Septoria tritici in the Microtiter Test (Septtr)

The stock solution was pipetted into a microtiter plate (MTP) and diluted to the stated active compound concentration with water. An aqueous malt-based spore suspension of Septoria tritici was then added. The plates were placed in a water vapor-saturated chamber at a temperature of 18° C. Using an absorption photometer, the MTPs were measured at 405 nm on day 7 after the inoculation.

### Binary Mixtures

| | | | | Effect | |
|---|---|---|---|---|---|
| Active compound/ | | | | calculated | |
| active compound | Concentration | | Observed | according | Synergism |
| combination | (ppm) | Mixture | effect | to Colby | (%) |
| I-3 | 1 | — | 3 | | |
| I-8 | 0.25 | — | 0 | | |
| I-14 | 1 | — | 0 | | |
| epoxiconazole | 0.016 | — | 22 | | |
| | 0.004 | — | 6 | | |
| chlorothalonil | 0.063 | — | 3 | | |
| I-3 | 1 | 16:1 | 26 | 6 | 20 |
| chlorothalonil | 0.063 | | | | |
| I-8 | 0.25 | 63:1 | 28 | 6 | 22 |
| epoxiconazole | 0.004 | | | | |
| I-14 | 1 | 63:1 | 51 | 22 | 29 |
| epoxiconazole | 0.016 | | | | |
| I-14 | 1 | 16:1 | 26 | 3 | 23 |
| chlorothalonil | 0.063 | | | | |

### Ternary Mixtures

| | | | | Effect | |
|---|---|---|---|---|---|
| Active compound/ | | | | calculated | |
| active compound | Concentration | | Observed | according | Synergism |
| combination | (ppm) | Mixture | effect | to Colby | (%) |
| I-1 + epoxiconazole | 4 + 0.063 | 63:1 | 74 | | |
| I-1 + boscalid | 1 + 0.25 | 4:1 | 52 | | |
| I-3 + epoxiconazole | 4 + 0.063 | 63:1 | 66 | | |
| | 0.25 + 0.004 | 63:1 | 7 | | |
| I-10 + pyraclostrobin | 0.25 + 0.001 | 250:1 | 21 | | |
| chlorothalonil | 0.25 | — | 12 | | |
| | 0.063 | — | 3 | | |
| boscalid | 0.063 | — | 10 | | |
| orysastrobin | 0.004 | — | 2 | | |
| I-1 + epoxiconazole | 4 + 0.063 | 63:1:4 | 100 | 78 | 22 |
| chlorothalonil | 0.25 | | | | |
| I-1 + boscalid | 1 + 0.25 | 16:4:1 | 76 | 54 | 22 |
| chlorothalonil | 0.063 | | | | |
| I-3 + epoxiconazole | 0.25 + 0.004 | 63:1:16 | 37 | 16 | 21 |
| boscalid | 0.063 | | | | |
| I-3 + epoxiconazole | 0.25 + 0.004 | 63:1:1 | 30 | 9 | 21 |
| orysastrobin | 0.004 | | | | |
| I-3 + epoxiconazole | 4 + 0.063 | 63:1:4 | 95 | 71 | 24 |
| chlorothalonil | 0.25 | | | | |
| I-10 + pyraclostrobin | 0.25 + 0.001 | 250:1:63 | 47 | 28 | 19 |
| boscalid | 0.063 | | | | |

### Use Example No. M5

### Activity Against Pyrenophora teres in the Microtiter Test (Pymte)

The stock solution was pipetted into a microtiter plate (MTP) and diluted to the stated active compound concentration with water. An aqueous malt-based spore suspension of Pyrenophora teres was then added. The plates were placed in a water vapor-saturated chamber at a temperature of 18° C. Using an absorption photometer, the MTPs were measured at 405 nm on day 7 after the inoculation. The measured parameters were compared to the growth of the active compound-free control variant (=100%) and the fungus- and active compound-free blank value to determine the relative growth in % of the pathogens in the individual active compounds.

| Active | | | | Effect | |
|---|---|---|---|---|---|
| compound/active | | | | calculated | |
| compound | Concentration | Mixing | Observed | according | Degree of |
| combination | (ppm) | ratio | effect (%) | to Colby (%) | synergism |
| I-1 | 0.016 | — | 5 | | |
| | 0.004 | | 2 | | |
| epoxiconazole | 0.016 | — | 0 | | |
| | 0.004 | | 0 | | |
| pyraclostrobin | 0.00025 | — | 5 | | |
| carbendazim | 0.004 | — | 0 | | |
| dimoxystrobin | 0.004 | — | 4 | | |
| I-1 | 0.004 | 16:1 | 20 | 7 | 13 |
| pyraclostrobin | 0.00025 | | | | |
| epoxiconazole | 0.004 | 16:1 | 10 | 5 | 5 |
| pyraclostrobin | 0.00025 | | | | |
| I-1 | 0.004 | 1:1 | 15 | 2 | 13 |
| carbendazim | 0.004 | | | | |
| epoxiconazole | 0.004 | 1:1 | 1 | 0 | 1 |
| carbendazim | 0.004 | | | | |
| I-1 | 0.016 | 4:1 | 21 | 8 | 13 |
| dimoxystrobin | 0.004 | | | | |
| epoxiconazole | 0.016 | 4:1 | 6 | 4 | 1 |
| dimoxystrobin | 0.004 | | | | |

### Use Example No. M6

### Activity Against the Rice Blast Pathogen Pyricularia oryzae in the Microtiter Test (Pyrior)

The stock solution was pipetted into a microtiter plate (MTP) and diluted to the stated active compound concentration with water. An aqueous malt-based spore suspension of Pyricularia oryzae was then added. The plates were placed in a water vapor-saturated chamber at a temperature of 18° C. Using an absorption photometer, the MTPs were measured at 405 nm on day 7 after the inoculation. The measured parameters were compared to the growth of the active compound-free control variant (=100%) and the fungus- and active compound-free blank value to determine the relative growth in % of the pathogens in the individual active compounds. Compounds I-18 had a growth of 8% at 31 ppm.

### Use Example No. M7

### Activity Against the Septoria Leaf Spot Pathogen Septoria tritici in the Microtiter Test (Septtr)

The stock solution was pipetted into a microtiter plate (MTP) and diluted to the stated active compound concentration with water. An aqueous malt-based spore suspension of Septoria tritici was then added. The plates were placed in a water vapor-saturated chamber at a temperature of 18° C. Using an absorption photometer, the MTPs were measured at 405 nm on day 7 after the innoculation. The measured parameters were compared to the growth of the active compound-free control variant (100%) and the fungus- and active compound-free blank value to determine the relative growth in % of the pathogens in the individual active compounds. Compounds I-18 had a growth of 11% at 31 ppm.

The determined values for the relative growth in % were determined initially and then converted into efficacies in % of the active compound-free control variant. An efficacy of 0 is the same growth as in the active compound-free control variant, an efficacy of 100 is 0% growth. The expected efficacies for active compound combinations were determined using Colby's formula (Colby, S. R. “Calculating synergistic and antagonistic responses of herbicide combinations”, Weeds, 15, pp. 20-22, 1967) and compared to the observed efficacies.

### Greenhouse

### Active Compound Preparation

The active compounds were prepared separately or jointly as a stock solution with 25 mg of active compound which was made up to 10 ml using a mixture of acetone and/or DMSO and the emulsifier Wettol EM 31 (wetting agent having emulsifying and dispersing action based on ethoxylated alkylphenols) in a volume ratio of solvent:emulsifier of 99:1. The mixture was then made up with water to 100 ml. This stock solution was diluted with the solvent/emulsifier/water mixture described to give the active compound concentration stated below. Alternatively, the active compounds were used as a commercial finished formulation and diluted with water to the stated active compound concentration.

The visually determined values for the percentage of infected leaf area were initially converted into a mean value and then converted into efficancies in % of the untreated control. An efficacy of 0 means the same degree of infection as in the untreated control, an efficacy of 100 means 0% infection. The expected efficacies for active compound combinations were determined using Colby's formula (Colby, S. R. (Calculating synergistic and antagonistic responses of herbicide combinations”, Weeds, 15, pp. 20-22, 1967) and compared to the observed efficacies.

### Use Example G1

### Activity Against Gray Mold on Bell Pepper Leaves Caused by Botrytis cinerea, Protective Application (Botrci P1)

Bell pepper seedlings were after 2-3 leaves were well developed, sprayed to runoff point with an aqueous suspension having the active compound concentration stated below. The next day, the treated plants were inoculated with a spore suspension of Botrytis cinerea in a 2% biomalt solution. The test plants were then placed in a dark climatized chamber at 22 to 24° C. and high atmospheric humidity. After 5 days, the extent of the fungal infection on the leaves could be determined visually in %.

### Botrci P1

| Active | | | | Effect | |
|---|---|---|---|---|---|
| compound/active | | | | calculated | |
| compound | Concentration | Mixing | Observed | according | Degree of |
| combination | (ppm) | ratio | effect (%) | to Colby (%) | synergism |
| Untreated | — | | 86 (% | | |
| control | | | Infection) | | |
| I-1 | 0.25 | — | 1 | | |
| epoxiconazole | 0.25 | — | 0 | | |
| pyraclostrobin | 1 | — | 1 | | |
| I-1 | 0.25 | 1:4 | 30 | 2 | 28 |
| pyraclostrobin | 1 | | | | |
| epoxiconazole | 0.25 | 1:4 | 13 | 1 | 12 |
| pyraclostrobin | 1 | | | | |

### Use Example G2

### Activity Against Late Blight on Tomatoes Caused by Phytophthora infestans, Protective Application (Phytin P1)

Leaves of potted tomato plants were sprayed to runoff point with an aqueous suspension having the active compound concentration stated below. The next day, the leaves were inoculated with an aqueous sporangia suspension of Phytophthora infestans. The plants were then placed in a water vapor-saturated chamber at temperatures between 18 and 20° C. After 6 days, the late blight on the untreated, but infected control plants had developed to such an extent that the infection could be determined visually in %.

### Phytin P1

| Active | | | | Effect | |
|---|---|---|---|---|---|
| compound/active | | | | calculated | |
| compound | Concentration | Mixing | Observed | according | Degree of |
| combination | (ppm) | ratio | effect (%) | to Colby (%) | synergism |
| Untreated | — | | 86 (% | | |
| control | | | infection) | | |
| I-1 | 1 | — | 0 | | |
| epoxiconazole | 1 | — | 1 | | |
| thiophanate-methyl | 63 | — | 0 | | |
| I-1 | 1 | 1:63 | 13 | 0 | 13 |
| thiophanate-methyl | 63 | | | | |
| epoxiconazole | 1 | 1:63 | 7 | 1 | 6 |
| thiophanate-methyl | 63 | | | | |

### Use Example G3

### Curative Activity Against Soybean Rust Caused by Phakopsora pachyrhizi (Phakpa K4)

Leaves of potted soybean seedlings were inoculated with a spore suspension of soybean rust (Phakpsora pachyrhizi). The pots were then placed in a chamber of high atmospheric humidity (90 to 95%) and 20 to 24° C. for 24 hours. During this time, the spores germinated and the germ tubes penetrated into the leaf tissue. The infected plants were then cultivated further in a greenhouse at temperatures between 23 and 27° C. and 60 to 80% relative atmospheric humidity. After three days, the plants were sprayed to runoff point with the active compound solution described above at the active compound concentration stated below. After the spray coating had dried on, the test plants were cultivated in a greenhouse at temperatures between 23 and 27° C. and 60 to 80% relative atmospheric humidity for a further 10 days. The extent of the rust fungus development on the leaves was then determined visually in % infection.

### Phakpa K4

| Active | | | | Effect | |
|---|---|---|---|---|---|
| compound/active | | | | calculated | |
| compound | Concentration | Mixing | Observed | according | Degree of |
| combination | (ppm) | ratio | effect (%) | to Colby (%) | synergism |
| Untreated | — | | 76 (% | | |
| control | | | infection) | | |
| I-1 | 0.063 | — | 17 | | |
| epoxiconazole | 0.063 | — | 63 | | |
| kresoxim-methyl | 1 | — | 39 | | |
| thiophanate- | 4 | — | 8 | | |
| methyl | | | | | |
| I-1 | 0.063 | 1:16 | 88 | 49 | 39 |
| kresoxim-methyl | 1 | | | | |
| epoxiconazole | 0.063 | 1:16 | 94 | 77 | 17 |
| kresoxim-methyl | 1 | | | | |
| I-1 | 0.063 | 1:64 | 61 | 23 | 38 |
| thiophanate- | 4 | | | | |
| methyl | | | | | |
| epoxiconazol | 0.063 | 1:64 | 76 | 66 | 10 |
| thiophanate- | 4 | | | | |
| methyl | | | | | |

### Use Example G4

### Protective Activity Against Puccinia recondita on Wheat (Brown Rust of Wheat) (Puccrt P1)

Leaves of potted wheat seedlings were sprayed to runoff point with an aqueous suspension having the active compound concentration stated below. The next day, the treated plants were inoculated with a spore suspension of brown rust of wheat (Puccinia recondita). The plants were then placed in a chamber of high atmospheric humidity (90 to 95%) at 20 to 24° C. for 24 hours. During this time, the spores germinated and the germ tubes penetrated into the leaf tissue. The next day, the test plants were returned to the greenhouse and cultivated at temperatures between 20 and 24° C. and 65 to 70% relative atmospheric humidity for a further 7 days. The extent of the rust fungus development on the leaves was then determined visually.

### Puccrt P1

| Active | | | | Effect | |
|---|---|---|---|---|---|
| compound/active | | | | calculated | |
| compound | Concentration | Mixing | Observed | according | Degree of |
| combination | (ppm) | ratio | effect (%) | to Colby (%) | synergism |
| Untreated | — | | 79 (% | | |
| control | | | infection) | | |
| I-1 | 0.016 | — | 0 | | |
| epoxiconazole | 0.016 | — | 0 | | |
| boscalid | 0.063 | — | 0 | | |
| I-1 | 0.016 | 1:4 | 18 | 0 | 18 |
| boscalid | 0.063 | | | | |
| epoxiconazole | 0.016 | 1:4 | 0 | 0 | 0 |
| boscalid | 0.063 | | | | |

### Use Example G5

### Protective Activity Against Septoria Leaf Blotch of Wheat Caused by Septoria tritici (Septtr P1)

Leaves of potted wheat seedlings were sprayed to runoff point with an aqueous suspension having the active compound concentration stated below. 24 hours after the spray coating had dried on, they were inoculated with an aqueous spore suspension of Septoria tritici. The test plants were then placed in a greenhouse at temperatures between 18 and 22° C. and a relative atmospheric humidity close to 100% for 4 days and then at temperatures between 18 and 22° C. and an atmospheric humidity of about 70%. After 21 days, the extent of the development of the disease was determined visually in % infection of the total leaf area.

### Septtr P1

| Active | | | | Effect | |
|---|---|---|---|---|---|
| compound/active | | | | calculated | |
| compound | Concentration | Mixing | Observed | according | Degree of |
| combination | (ppm) | ratio | effect (%) | to Colby (%) | synergism |
| Untreated | — | | 83 (% | | |
| control | | | infection) | | |
| I-1 | 1 | — | 8 | | |
| epoxiconazole | 1 | — | 24 | | |
| boscalid | 4 | — | 10 | | |
| I-1 | 1 | 1:4 | 82 | 17 | 65 |
| boscalid | 4 | | | | |
| epoxiconazole | 1 | 1:4 | 83 | 31 | 52 |
| boscalid | 4 | | | | |

The test results show that, by virtue of the synergism, the mixtures according to the invention are considerably more effective than had been calculated in advance using Colby's formula.

### Use Example G6

### Protective Activity Against Septoria Leaf Blotch of Wheat Caused by Septoria tritici (Septtr P1)

Leaves of potted wheat seedlings were sprayed to runoff point with an aqueous suspension having the active compound concentration stated below. 24 hours after the spray coating had dried on, they were inoculated with an aqueous spore suspension of Septoria tritici. The test plants were then placed in a greenhouse at temperatures between 18 and 22° C. and a relative atmospheric humidity close to 100% for 4 days and then at temperatures between 18 and 22° C. and an atmospheric humidity of about 70%. After 21 days, the extent of the development of the disease was determined visually in % infection of the total leaf area. At 150 ppm, the compound I-6 (diastereomer mixture “trans”) showed an infection of 0% whereas the untreated control was 60% infected.

### Use Example G7

### Curative Activity Against Soybean Rust Caused by Phakopsora pachyrhizi (Phakpa K3)

Leaves of potted soybean seedlings were inoculated with a spore suspension of soybean rust (Phakpsora pachyrhizi). The pots were then placed in a chamber of high atmospheric humidity (90 to 95%) and 20 to 24° C. for 24 hours. During this time, the spores germinated and the germ tubes penetrated into the leaf tissue. The infected plants were then cultivated further in a greenhouse at temperatures between 23 and 27° C. and 60 to 80% relative atmospheric humidity. After two days, the plants were sprayed to runoff point with the active compound solution described above at the active compound concentration stated below. After the spray coating had dried on, the test plants were cultivated in a greenhouse at temperatures between 23 and 27° C. and 60 to 80% relative atmospheric humidity for a further 10 days. The extent of the rust fungus development on the leaves was then determined visually in % infection. At 150 ppm, the compound I-6 (diastereomer mixture “trans”) showed an infection of 0% whereas the untreated control was 80% infected.

### Comparative Test:

### Microtest

The active compounds were formulated separately as a stock solution having a concentration of 10 000 ppm in DMSO.

Comparison No. C1—Activity Against the Septoria Leaf Blotch Pathogen Septoria tritici in the Microtiter Test (Septtr)

The stock solution was pipetted into a microtiter plate (MTP) and diluted to the stated active compound concentration with water. An aqueous malt-based spore suspension of Septoria tritici was then added. The plates were placed in a water vapor-saturated chamber at a temperature of 18° C. Using an absorption photometer, the MTPs were measured at 405 nm on day 7 after the innoculation. The measured parameters were compared to the growth of the active compound-free control variant (100%) and the fungus- and active compound-free blank value to determine the relative growth in % of the pathogens in the individual active compounds.

| | | Growth in % |
|---|---|---|
| | | at 8 ppm for |
| | Structure | Septtr |
| Compound according to the invention I-18 | 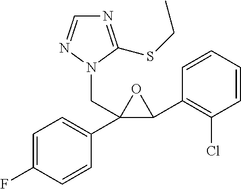 | 39 |
| Prior art (WO 96/38440) | 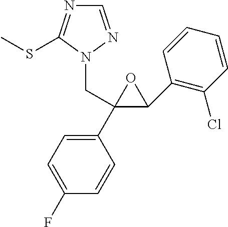 | 65 |

1-15. (canceled)

16. A fungicidal mixture comprising, as active components,
1) a compound of formula I 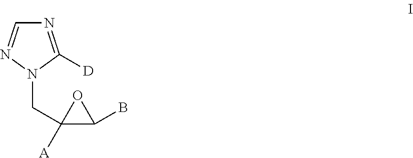
in which:
A is phenyl, 2-fluorophenyl, 2-chlorophenyl, 4-fluorophenyl, 4-chlorophenyl, 4-bromophenyl, 3-chlorophenyl, 2,4-dichlorophenyl, 3,4-dichlorophenyl, 3,5-dichlorophenyl, 4-methylphenyl, or 4-tert-butylphenyl;
B is 2-fluorophenyl, 2-chlorophenyl, or 2-bromophenyl;
D is —S—R, where
R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
R³ is C₁-C₈-alkyl, C₁-C₈-haloalkyl, C₁-C₈-alkoxy, C₁-C₈-haloalkoxy or NA³A⁴; and
R⁴ is C₁-C₈-alkyl, phenyl-C₁-C₈-alkyl or phenyl, where the phenyl groups are in each case unsubstituted or substituted by one, two or three groups independently of one another selected from the group consisting of halogen and C₁-C₄-alkyl;
a group DI 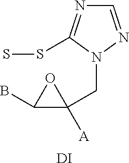
where A and B are as defined above;
a group DII 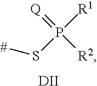
where # denotes the point of attachment to the triazolyl ring and Q, R¹ and K are R² as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl; or
a group SM, where:
M is an alkali metal cation, an equivalent of an alkaline earth metal cation, an equivalent of a copper, zinc, iron or nickel cation or an ammonium cation of the formula (E) 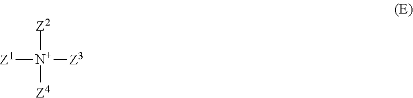
in which
Z¹ and Z² independently are hydrogen or C₁-C₈-alkyl;
Z³ and Z⁴ independently are hydrogen, C₁-C₈-alkyl, benzyl or phenyl;
where the phenyl groups are in each case unsubstituted or substituted by one, two or three groups independently selected from the group consisting of halogen and C₁-C₄-alkyl;
and agriculturally acceptable salts thereof, and
2) a compound II selected from the group consisting of:
A) a strobilurin selected from the group consisting of
azoxystrobin, dimoxystrobin, coumoxystrobin, coumethoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, methyl 2-(ortho-((2,5-dimethylphenyloxymethylene)phenyl)-3-methoxyacrylate, methyl 3-methoxy-2-(2-(N-(4-methoxyphenyl)-cyclopropanecarboximidoylsulfanylmethyl)phenyl)acrylate, and 2-(2-(3-(2,6-dichlorophenyl)-1-methylallylideneaminooxymethyl)phenyl)-2-methoxyimino-N-methylacetamide;
B) a carboxamide selected from the group consisting of
a carboxanilide selected from the group consisting of benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methylthiazole-5-carboxanilide, 2-chloro-N-(1,1,3-trimethylindan-4-yDnicotinamide, N-(3′,4′,5′-trifluorobiphenyl-2-yl)-3-difluoromethyl-1-methyl-1H-pyrazole-4-carboxamide (fluxapyroxad), N-(4′-trifluoromethylthiobiphenyl-2-yl)-3-difluoromethyl-1-methyl-1H-pyrazole-4-carboxamide, N-(2-(1,3-dimethylbutyl)phenyl)-1,3-dimethyl-5-fluoro-1H-pyrazole-4-carboxamide (penflufen), and N-(2-(1,3,3-trimethylbutyl)phenyl)-1,3-dimethyl-5-fluoro-1H-pyrazole-4-carboxamide;
a carboxylic acid morpholide selected from the group consisting of dimethomorph, flumorph, and pyrimorph;
a benzamide selected from the group consisting of flumetover, fluopicolide, fluopyram, zoxamide, and N-(3-ethyl-3,5,5-trimethylcyclohexyl)-3-formylamino-2-hydroxybenzamide; and
another carboxamide selected from the group consisting of carpropamid, diclocymet, mandipropamid, oxytetracyclin, silthiofam, and N-(6-methoxypyridin-3-yl)cyclopropanecarboxamide;
C) an azole selected from the group consisting of
a triazole selected from the group consisting of azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, and 1-(4-chlorophenyl)-2-([1,2,4]triazol-1-yl)cycloheptanol;
an imidazole selected from the group consisting of cyazofamid, imazalil, imazalil sulfate, pefurazoate, prochloraz, and triflumizole;
a benzimidazole selected from the group consisting of benomyl, carbendazim, fuberidazole, and thiabendazole; and
another azole selected from the group consisting of ethaboxam, etridiazole, hymexazole, and 2-(4-chlorophenyl)-N-[4-(3,4-dimethoxyphenyl)isoxazol-5-yl]-2-prop-2-ynyloxyacetamide;
D) a nitrogenous heterocyclyl compound selected from the group consisting of
a pyridine selected from the group consisting of fluazinam, pyrifenox, 3-[5-(4-chlorophenyl)-2,3-dimethylisoxazolidin-3-yl]pyridine, 3-[5-(4-methylphenyl)-2,3-dimethylisoxazolidin-3-yl]pyridine, 2,3,5,6-tetrachloro-4-methanesulfonylpyridine, 3,4,5-trichloropyridine-2,6-dicarbonitrile, N-(1-(5-bromo-3-chloropyridin-2-yl)ethyl)-2,4-dichloronicotinamide, and N-((5-bromo-3-chloro-pyridin-2-yl)methyl)-2,4-dichloronicotinamide;
a pyrimidine selected from the group consisting of bupirimate, cyprodinil, diflumetorim, fenarimol, ferimzone, mepanipyrim, nitrapyrin, nuarimol, and pyrimethanil;
triforine;
a pyrrole selected from the group consisting of fludioxoniland fenpiclonil;
a morpholine selected from the group consisting of aldimorph, dodemorph, dodemorph acetate, fenpropimorph, and tridemorph;
fenpropidin;
a dicarboximide selected from the group consisting of fluoroimide, iprodione, procymidone, and vinclozolin;
a nonaromatic 5-membered heterocycle selected from the group consisting of famoxadone, fenamidone, flutianil, octhilinone, probenazole, and S-allyl 5-amino-2-isopropyl-3-oxo-4-ortho-tolyl-2,3-dihydropyrazole-1-thiocarboxylate; and
another nitrogenous heterocyclyl compound selected from the group consisting of acibenzolar-S-methyl, amisulbrom, anilazine, blasticidin-S, captafol, captan, chinomethionat, dazomet, debacarb, diclomezine, difenzoquat, difenzoquat-methylsulfate, fenoxanil, folpet, oxolinic acid, piperalin, proquinazid, pyroquilon, quinoxyfen, triazoxide, tricyclazol, 2-butoxy-6-iodo-3-propylchromen-4-one, 5-chloro-1-(4,6-dimethoxypyrimidin-2-yl)-2-methyl-1H-benzoimidazole, 5-chloro-7-(4-methylpiperidin-1-yl)-6-(2,4,6-trifluorophenyl)-[1,2,4]triazolo[1,5-a]pyrimidine, and 5-ethyl-6-octyl-[1,2,4]triazolo-[1,5-a]pyrimidin-7-ylamine;
E) a carbamateor dithiocarbamate selected from the group consisting of
a thio- or dithiocarbamate selected from the group consisting of ferbam, mancozeb, maneb, metam, methasulfocarb, metiram, propineb, thiram, zineb, and ziram; and
a carbamate selected from the group consisting of diethofencarb, benthiavalicarb, iprovalicarb, propamocarb, propamocarb hydrochloride, valiphenal, and 4-fluorophenyl N-(1-(1-(4-cyanophenyl)ethanesulfonyl)but-2-yl)carbamate;
F) another fungicide selected from the group consisting of
a guanidine selected from the group consisting of dodine, dodine free base, guazatine, guazatine acetate, iminoctadine, iminoctadine triacetate, and iminoctadine tris(albesilate);
an antibiotic selected from the group consisting of kasugamycin, kasugamycin hydrochloride hydrate, polyoxins, streptomycin, and validamycin A;
a nitrophenyl derivative selected from the group consisting of binapacryl, dicloran, dinobuton, dinocap, nitrothal isopropyl, and tecnazene;
an organometallic compound selected from the group consisting of fentin salts;
a sulfur-containing heterocyclyl compound selected from the group consisting of dithianonand isoprothiolane;
an organophosphorus compound selected from the group consisting of edifenphos, fosetyl, fosetyl aluminum, iprobenfos, phosphorous acid and its salts, pyrazophos, and tolclofosmethyl;
an organochlorine compound selected from the group consisting of chlorothalonil, dichlofluanid, dichlorophen, flusulfamide, hexachlorobenzene, pencycuron, pentachlorophenol and its salts, phthalide, quintozene, thiophanate methyl, tolylfluanid, and N-(4-chloro-2-nitrophenyl)-N-ethyl-4-methylbenzenesulfonamide;
an inorganic active compound selected from the group consisting of phosphorous acid and its salts, Bordeaux mixture, copper salts, and sulfur; and
another fungicide selected from the group consisting of biphenyl, bronopol, cyflufenamid, cymoxanil, diphenylamine, metrafenone, pyriofenone, mildiomycin, oxine-copper, prohexadione-calcium, spiroxamine, tolylfluanid, N-(cyclopropylmethoxyimino-(6-difluoromethoxy-2,3-difluorophenyl)methyl)-2-phenylacetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine, N′-(5-difluoromethyl-2-methyl-4-(3-trimethylsilanylpropoxy)phenyl)-N-ethyl-N-methylformamidine, methyl N-(1,2,3,4-tetrahydronaphthalen-1-yl)-2-{1-[2-(5-methyl-3-trifluoromethylpyrazol-1-yl)acetyl]piperidin-4-yl}thiazole-4-carboxamide, methyl(R)-N-(1,2,3,4-tetrahydronaphthalen-1-yl)-2-{1-[2-(5-methyl-3-trifluoromethylpyrazol-1-yl)acetyl]piperidin-4-yl}thiazole-4-carboxamide, 6-tert-butyl-8-fluoro-2,3-dimethylquinolin-4-yl acetate, 6-tert-butyl-8-fluoro-2,3-dimethylquinolin-4-yl methoxyacetate, and N-methyl-2-{1-[2-(5-methyl-3-trifluoromethyl-1H-pyrazol-1-yl)acetyl]piperidin-4-yl}-N-[(1R)-1,2,3,4-tetrahydronaphthalen-1-yl]-4-thiazolecarboxamide;
G) a growth regulator selected from the group consisting of
abscisic acid, amidochlor, ancym idol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfid, indole-3-acetic acid, maleic hydrazide, mefluidide, mepiquat (mepiquat chloride), metconazole, naphthalene acetic acid, N-6-benzyladenine, paclobutrazole, prohexadione (prohexadione-calcium), prohydrojasmon, thidiazuron, triapenthenol, tributyl phosphorotrithioate, 2,3,5-triiodobenzoic acid, trinexapacethyl and uniconazole;
H) an herbicide selected from the group consisting of
an acetamide selected from the group consisting of acetochlor, alachlor, butachlor, dimethachlor, dimethenamid, flufenacet, mefenacet, metolachlor, metazachlor, napropamide, naproanilide, pethoxamid, pretilachlor, propachlor, and thenylchlor;
an amino acid analog selected from the group consisting of bilanafos, glyphosate, glufosinate, and sulfosate;
an aryloxyphenoxypropionate selected from the group consisting of clodinafop, cyhalofopbutyl, fenoxaprop, fluazifop, haloxyfop, metamifop, propaquizafop, quizalofop, and quizalofop-p-tefuryl;
a bipyridyl selected from the group consisting of diquatand paraquat;
a carbamates or a thiocarbamate selected from the group consisting of asulam, butylate, carbetamide, desmedipham, dimepiperate, eptam (EPTC), esprocarb, molinate, orbencarb, phenmedipham, prosulfocarb, pyributicarb, thiobencarb, and triallate;
a cyclohexanedione selected from the group consisting of butroxydim, clethodim, cycloxydim, profoxydim, sethoxydim, tepraloxydim, and tralkoxydim;
a dinitroaniline selected from the group consisting of benfluralin, ethalfluralin, oryzalin, pendimethalin, prodiamine, and trifluralin;
a diphenyl ether selected from the group consisting of acifluorfen, aclonifen, bifenox, diclofop, ethoxyfen, fomesafen, lactofen, and oxyfluorfen;
a hydroxybenzonitrile selected from the group consisting of bromoxynil, dichlobenil, and ioxynil;
an imidazolinone selected from the group consisting of imazamethabenz, imazamox, imazapic, imazapyr, imazaquin, and imazethapyr;
a phenoxyacetic acid selected from the group consisting of clomeprop, 2,4-dichlorophenoxyacetic acid (2,4-D), 2,4-DB, dichlorprop, MCPA, MCPA-thioethyl, MCPB, and mecoprop;
a pyrazine selected from the group consisting of chloridazon, flufenpyr-ethyl, fluthiacet, norflurazone, and pyridate;
a pyridine selected from the group consisting of aminopyralid, clopyralid, diflufenican, dithiopyr, fluridone, fluoroxypyr, picloram, picolinafen, and thiazopyr;
a sulfonylurea selected from the group consisting of amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, and 1-((2-chloro-6-propylimidazo[1,2-b]pyridazin-3-yl)sulfonyl)-3-(4,6-dimethoxypyrimidin-2-yl)urea;
a triazine selected from the group consisting of ametryn, atrazine, cyanazine, dimethametryn, ethiozine, hexazinone, metamitron, metribuzin, prometryn, simazine, terbuthylazine, terbutryn, and triaziflam;
a urea selected from the group consisting of chlorotoluron, daimuron, diuron, fluometuron, isoproturon, linuron, methabenzthiazuron, and tebuthiuron;
another inhibitor of acetolactate synthase selected from the group consisting of bispyribacsodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, and pyroxsulam; and
an herbicide selected from the group consisting of amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methylarsenic acid, naptalam, oxadiargyl, oxadiazon, oxaziclomefone, pentoxazone, pinoxaden, pyraclonil, pyraflufen-ethyl, pyrasulfotol, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxyethoxymethyl)-6-trifluoromethylpyridine-3-carbonyl]bicyclo[3.2.1]oct-3-en-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclopropylpyrimidine-4-carboxylate, 6-chloro-3-(2-cyclopropyl-6-methylphenoxy)pyridazin-4-ol, 4-amino-3-chloro-6-(4-chlorophenyl)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate; and
I) an insecticide selected from the group consisting of
an organo(thio)phosphate selected from the group consisting of acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon;
a carbamate selected from the group consisting of alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate;
a pyrethroid selected from the group consisting of allethrin, betacyfluthrin, bifenthrin, cyfluthrin, cyhalothrin, cyphenothrin, cypermethrin, alpha-cypermethrin, betacypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, and dimefluthrin,
an inhibitor of insect growth selected from the group consisting of a) benzoylurea chitin synthesis inhibitors selected from the group consisting of chlorfluazuron, cyromazine, diflubenzuron, flucycloxuron, flufenoxuron, hexaflumuron, lufenuron, novaluron, teflubenzuron, triflumuron; buprofezin, diofenolan, hexythiazox, etoxazole, and clofentazin; b) an ecdysone antagonist selected from the group consisting of halofenozide, methoxyfenozide, tebufenozide, and azadirachtin; c) a juvenoid selected from the group consisting of pyriproxyfen, methoprene, and fenoxycarb; and d) a lipid biosynthesis inhibitor selected from the group consisting of spirodiclofen, spiromesifen, and spirotetramate;
a nicotine receptor agonist/antagonist selected from the group consisting of clothianidin, dinotefuran, imidacloprid, thiamethoxam, nitenpyram, acetamiprid, thiacloprid, and 1-(2-chlorothiazol-5-ylmethyl)-2-nitrimino-3,5-dimethyl-[1,3,5]triazinane;
a GABA antagonist selected from the group consisting of endosulfan, ethiprol, fipronil, vaniliprol, pyrafluprol, pyriprol, and 5-amino-1-(2,6-dichloro-4-methylphenyl)-4-sulfinamoyl-1H-pyrazole-3-thiocarboxamide;
a macrocyclic lactone selected from the group consisting of abamectin, emamectin, milbemectin, lepimectin, spinosad, and spinetoram;
a mitochondrial electron transport chain inhibitor (METI) I acaricide selected from the group consisting of fenazaquin, pyridaben, tebufenpyrad, tolfenpyrad, and flufenerim;
a METI II and III substance selected from the group consisting of acequinocyl, fluacyprim, and hydramethylnon;
chlorfenapyr;
an inhibitor of oxidative phosphorylation selected from the group consisting of cyhexatin, diafenthiuron, fenbutatin oxide, and propargite;
cryomazine;
piperonyl butoxide;
a sodium channel blocker selected from the group consisting of indoxacarb and metaflumizone; and
another insecticide selected from the group consisting of benclothiaz, bifenazate, cartap, flonicamid, pyridalyl, pymetrozine, sulfur, thiocyclam, flubendiamid, chiorantraniliprol, cyazypyr (HGW86); cyenopyrafen, flupyrazofos, cyflumetofen, amidoflumet, imicyafos, bistrifluoron, pyrifluquinazon, pirimicarb, and [(3S,4R,4aR,6S,6aS,12R,12aS,12bS)-3-cyclopropanecarbonyloxy)-6,12-dihydroxy-4,6a,12b-trimethyl-11-oxo-9-(pyridin-3-yl)-1,2,3,4,4a,5,6,6a,12a,12b-decahydro-11H,12H-benzo[f]pyrano[4,3-b]chromen-4-yl]methyl cyclopropanecarboxylate;
wherein the compound of formula I and compound II are present in a weight ratio of from 100:1 to 1:100,
in a synergistically effective amount.

17. The fungicidal mixture of claim 16 wherein the compound of formula I is selected from the group consisting of:
I-1 A is 4-fluorophenyl, B is 2-chlorophenyl, and D is SH;
I-2 A is 4-fluorophenyl, B is 2-chlorophenyl, and D is S-Me;
I-3 A is 4-fluorophenyl, B is 2-chlorophenyl, and D is S—CO-Me;
I-4 A is 4-fluorophenyl, B is 2-chlorophenyl, and D is S—Na;
I-5 A is 4-fluorophenyl, B is 2-chlorophenyl, and D is S—CO-OMe;
I-6 A is 4-fluorophenyl, B is 2-chlorophenyl, and D is S—CN;
I-7 A is 2-chlorophenyl, B is 2-chlorophenyl, and D is SMe;
I-8 A is 4-fluorophenyl, B is 2-fluorophenyl, and D is SH;
I-9 A is 4-fluorophenyl, B is 2-fluorophenyl, and D is S-Me;
I-10 A is 2-chlorophenyl, B is 2-chlorophenyl, and D is S—Na;
I-11 A is phenyl, B is 2-chlorophenyl, and D is S-Me;
I-12 A is phenyl, B is 2-chlorophenyl, and D is SH;
I-13 A is 2-fluorophenyl, B is 2-chlorophenyl, and D is S-Me;
I-14 A is 2-fluorophenyl, B is 2-chlorophenyl, and D is SH;
I-15 A is phenyl, B is 2-chlorophenyl, and D is DI;
I-16 A is 2-chlorophenyl, B is 2-chlorophenyl, and D is S—CO-Me;
I-17 A is 4-fluorophenyl, B is 2-fluorophenyl, and D is S—CO-Me; and
I-18 A is 4-fluorophenyl, B is 2-chlorophenyl, and D is SC₂H₅.

18. The fungicidal mixture of claim 16 wherein the compound of formula I is selected from the group consisting of:
I-1 trans-2-[3-(2-chlorophenyl)-2-(4-fluorophenyl)oxiranylmethyl]-2,4-dihydro-[1,2,4]triazole-3-thione;
I-2 trans-1-[3-(2-chlorophenyl)-2-(4-fluorophenyl)oxiranylmethyl]-5-methylsulfanyl-1H-[1,2,4]triazole;
I-3 trans-S-{2-[3-(2-chlorophenyl)-2-(4-fluorophenyl)oxiranylmethyl]-2H-[1,2,4]triazol-3-yl}thioacetate;
1-4 sodium trans-2-[3-(2-chlorophenyl)-2-(4-fluorophenyl)oxiranylmethyl]-2H-[1,2,4]triazole-3-thiolate;
I-5 {2-[3-(2-chlorophenyl)-2-(4-fluorophenyl)oxiranylmethyl]-2H-[1,2,4]triazol-3-yl}methyl thiocarbonate;
I-6 trans-1-[3-(2-chlorophenyl)-2-(4-fluorophenyl)oxiranylmethyl]-5-thiocyanato-1H-[1,2,4]triazole;
I-7 trans-1-[2,3-bis-(2-chlorophenyl)oxiranylmethyl]-5-methylsulfanyl-1H-[1,2,4]triazole;
I-8 trans-2-[2-(4-fluorophenyl)-3-(2-fluorophenyl)oxiranylmethyl]-2H-[1,2,4]triazole-3-thiole;
I-9 trans-1-[2-(4-fluorophenyl)-3-(2-fluorophenyl)oxiranylmethyl]-5-methylsulfanyl-1H-[1,2,4]triazole;
I-10 sodium 2-[trans-2,3-bis-(2-chlorophenyl)oxiranylmethyl]-2H-[1,2,4]triazole-3-thiolate;
I-11 trans-1-[-3-(2-chlorophenyl)-2-phenyloxiranylmethyl]-5-methylsulfanyl-1H-[1,2,4]triazole;
I-12 2-[trans-3-(2-chlorophenyl)-2-phenyloxiranylmethyl]-2,4-dihydro-[1,2,4]triazole-3-thione;
I-13 1-[trans-3-(2-chlorophenyl)-2-(2-fluorophenyl)oxiranylmethyl]-5-methylsulfanyl-1H-[1,2,4]triazole;
I-14 2-[trans-3-(2-chlorophenyl)-2-(2-fluorophenyl)oxiranylmethyl]-2,4-dihydro-[1,2,4]triazole-3-thione;
I-15 bis-{2-[trans-3-(2-chlorophenyl)-2-phenyloxiranylmethyl]-2H-[1,2,4]triazol-3-yl}disulfane;
I-16 S-{2-[trans-2,3-bis-(2-chlorophenyl)oxiranylmethyl]-2H-[1,2,4]triazol-3-yl}thioacetate; and
I-17 S-{2-[trans-3-(2-fluorophenyl)-2-(4-fluorophenyl)oxiranylmethyl]-2H-[1,2,4]triazol-3-yl}thioacetate.

19. The fungicidal mixture of claim 16 wherein compound II is selected from the group consisting of:
II-1 epoxiconazole,
II-2 metconazole,
II-3 tebuconazole,
II-4 fluquinconazole,
II-5 flutriafol,
II-6 triticonazole,
II-7 prothioconazole,
II-8 kresoxim-methyl,
II-9 pyraclostrobin,
II-10 orysastrobin,
II-11 dimethomorph,
II-12 5-ethyl-6-octyl-[1,2,4]triazolo[1,5-a]pyrimidin-7-ylamine,
II-13 pyrimethanil,
II-14 metalaxyl,
II-15 fenpropimorph,
II-16 dodemorph,
II-17 iprodione,
II-1 8 mancozeb,
II-19 metiram,
II-20 thiophanate-methyl,
II-21 chlorothalonil,
II-22 metrafenone,
II-23 bixafen,
II-24 boscalid,
II-25 N-(3′,4′,5′-trifluorobiphenyl-2-yl)-3-difluoromethyl-1-methyl-1H-pyrazole-4-carboxamide,
II-26 sedaxane,
II-27 isopyrazam,
II-28 fluopyram, and
II-29 penflufen.

20. The fungicidal mixture of claim 16 wherein the mixture is selected from the group consisting of:
I-3 trans-S-{2-[3-(2-chlorophenyl)-2-(4-fluorophenyl)oxiranylmethyl]-2H-[1,2,4]triazol-3-yl}thioacetate and boscalid II-24;
I-10 sodium 2-[trans-2,3-bis-(2-chlorophenyl)oxiranylmethyl]-2H-[1,2,4]triazole-3-thiolate and pyraclostrobin 11-9;
I-14 2-[trans-3-(2-chlorophenyl)-2-(2-fluorophenyl)oxiranylmethyl]-2,4-dihydro-[1,2,4]triazole-3-thione and pyraclostrobin 11-9;
I-3 trans-S-{2-[3-(2-chlorophenyl)-2-(4-fluorophenyl)oxiranylmethyl]-2H-[1,2,4]triazol-3-yl}thioacetate and epoxiconazole II-1;
I-8 trans-2-[2-(4-fluorophenyl)-3-(2-fluorophenyl)oxiranylmethyl]-2H-[1,2,4]triazole-3-thiole and orysastrobin II-10;
I-1 trans-2-[3-(2-chlorophenyl)-2-(4-fluorophenyl)oxiranylmethyl]-2,4-dihydro-[1,2,4]triazole-3-thione and pyrimethanil II-13;
I-3 trans-S-{2-[3-(2-chlorophenyl)-2-(4-fluorophenyl)oxiranylmethyl]-2H-[1,2,4]triazol-3-yl}thioacetate and pyrimethanil II-13;
I-10 sodium 2-[trans-2,3-bis-(2-chlorophenyl)oxiranylmethyl]-2H-[1,2,4]triazole-3-thiolate and epoxiconazole II-1;
I-10 sodium 2-[trans-2,3-bis-(2-chlorophenyl)oxiranylmethyl]-2H-[1,2,4]triazole-3-thiolate and pyrimethanil II-13;
I-12 2-[trans-3-(2-chlorophenyl)-2-phenyloxiranylmethyl]-2,4-dihydro-[1,2,4]triazole-3-thione and epoxiconazole II-1;
I-12 2-[trans-3-(2-chlorophenyl)-2-phenyloxiranylmethyl]-2,4-dihydro-[1,2,4]triazole-3-thione and boscalid II-24;
I-12 2-[trans-3-(2-chlorophenyl)-2-phenyloxiranylmethyl]-2,4-dihydro-[1,2,4]triazole-3-thione and pyrimethanil II-13;
I-14 2-[trans-3-(2-chlorophenyl)-2-(2-fluorophenyl)oxiranylmethyl]-2,4-dihydro-[1,2,4]triazole-3-thione and pyrimethanil II-13;
I-3 trans-S-{2-[3-(2-chlorophenyl)-2-(4-fluorophenyl)oxiranylmethyl]-2H-[1,2,4]triazol-3-yl}thioacetate and chlorothalonil II-21;
I-8 trans-2-[2-(4-fluorophenyl)-3-(2-fluorophenyl)oxiranylmethyl]-2H-[1,2,4]triazole-3-thiole and epoxiconazole II-1;
I-14 2-[trans-3-(2-chlorophenyl)-2-(2-fluorophenyl)oxiranylmethyl]-2,4-dihydro-[1,2,4]triazole-3-thione and epoxiconazole II-1; and
I-14 2-[trans-3-(2-chlorophenyl)-2-(2-fluorophenyl)oxiranylmethyl]-2,4-dihydro-[1,2,4]triazole-3-thione and chlorothalonil II-21.

21. The fungicidal mixture of claim 16, further comprising a second compound II selected from the group consisting of compounds of groups A to I, with the proviso that the two compounds II are not identical.

22. The fungicidal mixture of claim 21 where the second compound II is selected from the group consisting of:
II-1 epoxiconazole,
II-2 metconazole,
II-3 tebuconazole,
II-6 triticonazole,
II-7 prothioconazole,
II-8 kresoxim-methyl,
II-9 pyraclostrobin,
II-11 dimethomorph,
II-12 5-ethyl-6-octyl-[1,2,4]triazolo[1,5-a]pyrimidin-7-ylamine,
II-13 pyrimethanil,
II-14 metalaxyl,
II-15 fenpropimorph,
II-18 mancozeb,
II-19 metiram,
II-20 thiophanate-methyl,
II-21 chlorothalonil,
II-22 metrafenone,
II-23 bixafen,
II-24 boscalid,
II-25 N-(3′,4′,5′-trifluorobiphenyl-2-yl)-3-difluoromethyl-1-methyl-1H-pyrazole-4-carboxamide,
II-26 sedaxane,
II-27 isopyrazam,
II-28 fluopyram, and
II-29 penflufen.

23. The fungicidal mixture of claim 21, wherein the two compounds II are present in a weight ratio of from 100:1 to 1:100.

24. A compound of formula I: 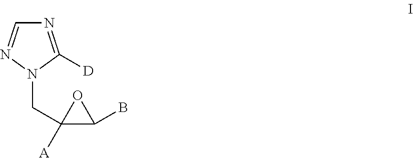
in which:
A is 4-fluorophenyl;
B is 2-chlorophenyl; and
D is S—CN.

25. The compound of claim 24, which is 1-[(2S,3R)-3-(2-chlorophenyl)-2-(4-fluorophenyl)oxiran-2-ylmethyl]-5-thiocyanato-1H-1,2,4-triazole, or an agriculturally acceptable salt thereof.

26. A compound of the formula I-A 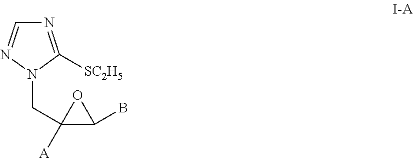
where:
A is phenyl, 2-fluorophenyl, 2-chlorophenyl, 4-fluorophenyl, 4-chlorophenyl, 4-bromophenyl, 3-chlorophenyl, 2,4-dichlorophenyl, 3,4-dichlorophenyl, 3,5-dichlorophenyl, 4-methylphenyl, or 4-tert-butylphenyl;
B is 2-fluorophenyl, 2-chlorophenyl, or 2-bromophenyl;
or an agriculturally acceptable salt thereof.

27. An agrochemical composition comprising a solvent or a solid carrier and a fungicidal mixture of claim 16.

28. An agrochemical composition comprising a solvent or a solid carrier and the compound of claim 24.

29. An agrochemical composition comprising a solvent or a solid carrier and a compound of claim 26.

30. A seed treated with a fungicidal mixture of claim 16.

31. A method for controlling phytopathogenic fungi, comprising treating the fungi or the materials, plants, the soil or seeds to be protected from fungal attack with an effective amount of a fungicidal mixture of claim 16.

32. The method of claim 31, wherein the compound of formula I is selected from the group consisting of:
I-1 A is 4-fluorophenyl, B is 2-chlorophenyl, and D is SH;
I-2 A is 4-fluorophenyl, B is 2-chlorophenyl, and D is S-Me;
I-3 A is 4-fluorophenyl, B is 2-chlorophenyl, and D is S—CO-Me;
I-4 A is 4-fluorophenyl, B is 2-chlorophenyl, and D is S—Na;
I-5 A is 4-fluorophenyl, B is 2-chlorophenyl, and D is S—CO-OMe;
I-6 A is 4-fluorophenyl, B is 2-chlorophenyl, and D is S—CN;
I-7 A is 2-chlorophenyl, B is 2-chlorophenyl, and D is SMe;
I-8 A is 4-fluorophenyl, B is 2-fluorophenyl, and D is SH;
I-9 A is 4-fluorophenyl, B is 2-fluorophenyl, and D is S-Me;
I-10 A is 2-chlorophenyl, B is 2-chlorophenyl, and D is S—Na;
I-11 A is phenyl, B is 2-chlorophenyl, and D is S-Me;
I-12 A is phenyl, B is 2-chlorophenyl, and D is SH;
I-13 A is 2-fluorophenyl, B is 2-chlorophenyl, and D is S-Me;
I-14 A is 2-fluorophenyl, B is 2-chlorophenyl, and D is SH;
I-15 A is phenyl, B is 2-chlorophenyl, and D is DI;
I-16 A is 2-chlorophenyl, B is 2-chlorophenyl, and D is S—CO-Me;
I-17 A is 4-fluorophenyl, B is 2-fluorophenyl, and D is S—CO-Me; and
I-18 A is 4-fluorophenyl, B is 2-chlorophenyl, and D is SC₂H₅.

33. The method of claim 31, wherein the compound of formula I is selected from the group consisting of:
I-1 trans-2-[3-(2-chlorophenyl)-2-(4-fluorophenyl)oxiranylmethyl]-2,4-dihydro-[1,2,4]triazole-3-thione;
I-2 trans-1-[3-(2-chlorophenyl)-2-(4-fluorophenyl)oxiranylmethyl]-5-methylsulfanyl-1H-[1,2,4]triazole;
I-3 trans-S-{2-[3-(2-chlorophenyl)-2-(4-fluorophenyl)oxiranylmethyl]-2H-[1,2,4]triazol-3-yl}thioacetate;
I-4 sodium trans-2-[3-(2-chlorophenyl)-2-(4-fluorophenyl)oxiranylmethyl]-2H-[1,2,4]triazole-3-thiolate;
I-5 {2-[3-(2-chlorophenyl)-2-(4-fluorophenyl)oxiranylmethyl]-2H-[1,2,4]triazol-3-yl}methyl thiocarbonate;
I-6 trans-1-[3-(2-chlorophenyl)-2-(4-fluorophenyl)oxiranylmethyl]-5-thiocyanato-1H-[1,2,4]triazole;
I-7 trans-1-[2,3-bis-(2-chlorophenyl)oxiranylmethyl]-5-methylsulfanyl-1H-[1,2,4]triazole;
I-8 trans-2-[2-(4-fluorophenyl)-3-(2-fluorophenyl)oxiranylmethyl]-2H-[1,2,4]triazole-3-thiole;
I-9 trans-1-[2-(4-fluorophenyl)-3-(2-fluorophenyl)oxiranylmethyl]-5-methylsulfanyl-1H-[1,2,4]triazole;
I-10 sodium 2-[trans-2,3-bis-(2-chlorophenyl)oxiranylmethyl]-2H-[1,2,4]triazole-3-thiolate;
I-11 trans-1-[-3-(2-chlorophenyl)-2-phenyloxiranylmethyl]-5-methylsulfanyl-1H-[1,2,4]triazole;
I-12 2-[trans-3-(2-chlorophenyl)-2-phenyloxiranylmethyl]-2,4-dihydro-[1,2,4]triazole-3-thione;
I-13 1-[trans-3-(2-chlorophenyl)-2-(2-fluorophenyl)oxiranylmethyl]-5-methylsulfanyl-1H-[1,2,4]triazole;
I-14 2-[trans-3-(2-chlorophenyl)-2-(2-fluorophenyl)oxiranylmethyl]-2,4-dihydro-[1,2,4]triazole-3-thione;
I-15 bis-{2-[trans-3-(2-chlorophenyl)-2-phenyloxiranylmethyl]-2H-[1,2,4]triazol-3-yl}disulfane;
I-16 S-{2-[trans-2,3-bis-(2-chlorophenyl)oxiranylmethyl]-2H-[1,2,4]triazol-3-yl}thioacetate; and
I-17 S-{2-[trans-3-(2-fluorophenyl)-2-(4-fluorophenyl)oxiranylmethyl]-2H-[1,2,4]triazol-3-yl}thioacetate.

34. The method of claim 31, wherein the compound II is selected from the group consisting of:
II-1 epoxiconazole,
II-2 metconazole,
II-3 tebuconazole,
II-4 fluquinconazole,
II-5 flutriafol,
II-6 triticonazole,
II-7 prothioconazole,
II-8 kresoxim-methyl,
II-9 pyraclostrobin,
II-10 orysastrobin,
II-11 dimethomorph,
II-12 5-ethyl-6-octyl-[1,2,4]triazolo[1,5-a]pyrimidin-7-ylamine,
II-13 pyrimethanil,
II-14 metalaxyl,
II-15 fenpropimorph,
II-16 dodemorph,
II-17 iprodione,
II-18 mancozeb,
II-19 metiram,
II-20 thiophanate-methyl,
II-21 chlorothalonil,
II-22 metrafenone,
II-23 bixafen,
II-24 boscalid,
II-25 N-(3′,4′,5′-trifluorobiphenyl-2-yl)-3-difluoromethyl-1-methyl-1H-pyrazole-4-carboxamide,
II-26 sedaxane,
II-27 isopyrazam,
II-28 fluopyram, and
II-29 penflufen.

35. The method of claim 31, wherein the fungicidal mixture is selected from the group consisting of:
I-3 trans-S-{2-[3-(2-chlorophenyl)-2-(4-fluorophenyl)oxiranylmethyl]-2H-[1,2,4]triazol-3-yl}thioacetate and boscalid II-24;
I-10 sodium 2-[trans-2,3-bis-(2-chlorophenyl)oxiranylmethyl]-2H-[1,2,4]triazole-3-thiolate and pyraclostrobin II-9;
I-14 2-[trans-3-(2-chlorophenyl)-2-(2-fluorophenyl)oxiranylmethyl]-2,4-dihydro-[1,2,4]triazole-3-thione and pyraclostrobin II-9;
I-3 trans-S-{2-[3-(2-chlorophenyl)-2-(4-fluorophenyl)oxiranylmethyl]-2H-[1,2,4]triazol-3-yl}thioacetate and epoxiconazole II-1;
I-8 trans-2-[2-(4-fluorophenyl)-3-(2-fluorophenyl)oxiranylmethyl]-2H-[1,2,4]triazole-3-thiole and orysastrobin II-10;
I-1 trans-2-[3-(2-chlorophenyl)-2-(4-fluorophenyl)oxiranylmethyl]-2,4-dihydro-[1,2,4]triazole-3-thione and pyrimethanil II-13;
I-3 trans-S-{2-[3-(2-chlorophenyl)-2-(4-fluorophenyl)oxiranylmethyl]-2H-[1,2,4]triazol-3-yl}thioacetate and pyrimethanil II-13;
I-10 sodium 2-[trans-2,3-bis-(2-chlorophenyl)oxiranylmethyl]-2H-[1,2,4]triazole-3-thiolate and epoxiconazole II-1;
I-10 sodium 2-[trans-2,3-bis-(2-chlorophenyl)oxiranylmethyl]-2H-[1,2,4]triazole-3-thiolate and pyrimethanil II-13;
I-12 2-[trans-3-(2-chlorophenyl)-2-phenyloxiranylmethyl]-2,4-dihydro-[1,2,4]triazole-3-thione and epoxiconazole II-1;
I-12 2-[trans-3-(2-chlorophenyl)-2-phenyloxiranylmethyl]-2,4-dihydro-[1,2,4]triazole-3-thione and boscalid II-24;
I-12 2-[trans-3-(2-chlorophenyl)-2-phenyloxiranylmethyl]-2,4-dihydro-[1,2,4]triazole-3-thione and pyrimethanil II-13;
I-14 2-[trans-3-(2-chlorophenyl)-2-(2-fluorophenyl)oxiranylmethyl]-2,4-dihydro-[1,2,4]triazole-3-thione and pyrimethanil II-13;
I-3 trans-S-{2-[3-(2-chlorophenyl)-2-(4-fluorophenyl)oxiranylmethyl]-2H-[1,2,4]triazol-3-yl}thioacetate and chlorothalonil II-21;
I-8 trans-2-[2-(4-fluorophenyl)-3-(2-fluorophenyl)oxiranylmethyl]-2H-[1,2,4]triazole-3-thiole and epoxiconazole II-1;
I-14 2-[trans-3-(2-chlorophenyl)-2-(2-fluorophenyl)oxiranylmethyl]-2,4-dihydro-[1,2,4]triazole-3-thione and epoxiconazole II-1; and
I-14 2-[trans-3-(2-chlorophenyl)-2-(2-fluorophenyl)oxiranylmethyl]-2,4-dihydro-[1,2,4]triazole-3-thione and chlorothalonil II-21.
